SEQUENCE LISTING (63 sequences)

SEQ ID NO: 1 - protein, 133 aa, Homo sapiens [sequence omitted]

SEQ ID NO: 2 - protein, 133 aa, Homo sapiens
APTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 3 - protein, 133 aa, Homo sapiens
APTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFCQSIISTLT

SEQ ID NO: 4 - protein, 133 aa, Homo sapiens
APTSSSTKKTQLQLEHLLLDLQMILNGINNYKNPKLTRMLTFKFYMPKKATELKHLQCLEEELKPLEEVLNLAQSKNFHLRPRDLISNINKIVLELKGSETTFMCEYADETATIVEFLNRWITFCQSIISTLT

SEQ ID NO: 5 - protein, 133 aa, Homo sapiens
APTSSSTKKTQLQLEHLLLDLQMILNGINNYKNPKLTRMLTFKFYMPKKATELKHLQCLEEELKPLEEALNLAPSKNFHLRPRDLISNINKIVLELKGSETTFMCEYADETATIVEFLNRWITFCQSIISTLT

SEQ ID NO: 6 - protein, 133 aa, Homo sapiens
APTSSSTKKTQLQLEHLLLTLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 7 - RNA, 822 nt, Homo sapiens
  source 1..822: mol_type = genomic RNA
agttccctatcactctctttaatcactactcacagtaacctcaactcctgccacaatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgataattaagtgcttcccacttaaaacatatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaaaaaaaaaaaaaaaaaaaaaaaaa

SEQ ID NO: 8 - protein, 584 aa, Homo sapiens
AHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 9 - protein, 746 aa, synthetic construct
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 10 - protein, 746 aa, synthetic construct
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 11 - protein, 746 aa, synthetic construct
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFCQSIISTLT

SEQ ID NO: 12 - protein, 746 aa, synthetic construct
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEVLNLAQSKNFHLRPRDLISNINKIVLELKGSETTFMCEYADETATIVEFLNRWITFCQSIISTLT

SEQ ID NO: 13 - protein, 746 aa, synthetic construct
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEALNLAPSKNFHLRPRDLISNINKIVLELKGSETTFMCEYADETATIVEFLNRWITFCQSIISTLT

SEQ ID NO: 14 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 15 - DNA, 489 bp, synthetic construct
  source 1..489: mol_type = other DNA
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 16 - protein, 267 aa, synthetic construct
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 17 - protein, 118 aa, synthetic construct
EVQLVQTGGGLSQFGESLRLSCAVSGFNVSNNYMSWVRQAPGKGLEWVSIIYSGGGTHYADSVKGRFTISRDNSKNTLFLQMNSLRAEDTAVYYCARAVPVPHGTDIWGQGTMVTVSS

SEQ ID NO: 18 - protein, 884 aa, synthetic construct
MPLLLLLPLLWAGALAEVQLVQTGGGLSQFGESLRLSCAVSGFNVSNNYMSWVRQAPGKGLEWVSIIYSGGGTHYADSVKGRFTISRDNSKNTLFLQMNSLRAEDTAVYYCARAVPVPHGTDIWGQGTMVTVSSGGGGSGGGGSGGGGSAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGKPIPNPLLGLDST

SEQ ID NO: 19 - protein, 884 aa, synthetic construct
MPLLLLLPLLWAGALAEVQLVQTGGGLSQFGESLRLSCAVSGFNVSNNYMSWVRQAPGKGLEWVSIIYSGGGTHYADSVKGRFTISRDNSKNTLFLQMNSLRAEDTAVYYCARAVPVPHGTDIWGQGTMVTVSSGGGGSGGGGSGGGGSAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLTLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGKPIPNPLLGLDST

SEQ ID NO: 20 - protein, 883 aa, synthetic construct
MPLLLLLPLLWAGALAEVQLVQTGGGLSQFGESLRLSCAVSGFNVSNNYMSWVRQAPGKGLEWVSIIYSGGGTHYADSVKGRFTISRDNSKNTLFLQMNSLRAEDTAVYYCARAVPVPHGTDIWGQGTMVTVSSGGGGSGGGGSGGGGSAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLTGKPIPNPLLGLDS

SEQ ID NO: 21 - RNA, 2652 nt, synthetic construct
  source 1..2652: mol_type = other RNA
atgcctctgttactgctgctcccacttctgtgggccggcgctctggctgaggtgcagcttgtgcagaccggcggaggtctgtcacagttcggcgaatccctaaggctctcttgcgccgtgtctggtttcaacgtgagcaacaactatatgagttgggttcgccaggctccaggcaagggtctggagtgggtgtccattatctattccggcggtggcacacattacgccgactcagtcaagggtagattcactatcagtagagacaactcaaagaacactcttttcctacaaatgaacagcttgcgggcagaggatacggccgtgtattattgtgcccgagctgtcccagtgcctcacggaacagatatttggggacaaggtacaatggtgaccgtctcttccggcggaggaggctctggaggcggaggatctggcggaggcggcagcgcccataagagcgaggtagcccatagattcaaggaccttggcgaagagaacttcaaggccttagttctcatcgccttcgcccaatacttgcagcagtgtccattcgaagatcacgttaagcttgtgaacgaggtgaccgaattcgctaagacgtgcgtggccgacgagtccgctgagaattgtgataagtcactgcacactttgttcggcgacaagctgtgcaccgtcgccaccctgcgcgaaacatacggcgaaatggcggactgttgtgctaagcaggagccagaacggaacgagtgtttcctgcagcataaggacgacaatccaaacctccctaggttggtgcgcccagaagtggacgtcatgtgtaccgctttccacgataacgaggagacattccttaagaagtatctgtacgagattgcacggcggcacccttacttctacgcccctgagctcctgttcttcgcaaagcggtataaggctgcattcaccgagtgttgccaggcggcagacaaggccgcctgcttgctccctaagcttgacgaacttagggacgagggaaaggctagtagcgccaagcagagactcaagtgtgcatcgctacagaagttcggagagagagctttcaaggcgtgggctgtcgcgcgcctgtcccagaggttcccaaaggccgaattcgccgaggtgagcaagttagtgacagatctcaccaaggtgcacactgagtgttgccacggcgacttattggagtgtgcagacgatcgggctgacttggcaaagtacatctgtgagaaccaggactcaatttccagtaagctgaaggagtgttgcgagaagccactcttggagaagtcccattgtatagccgaagtggagaacgacgagatgccggctgacctgccgtcactggcagctgatttcgtagagtcgaaggacgtgtgtaagaactacgctgaagctaaggacgtattcctgggaatgttcctttacgagtacgcccgacggcatccagactactcagtagtgctgcttctgagactggccaagacttacgagactaccctggagaagtgttgtgcagcggccgatccacacgagtgttacgccaaggtgttcgacgaattcaagcctctggtggaggagccgcagaacctgatcaagcagaattgcgaactgttcgagcagttgggagaatataagttccagaacgcactgctagtgcggtataccaagaaggtcccacaggtatccactccgacactggtggaagtgtccaggaacctcggcaaggtgggaagcaagtgctgtaagcacccggaggctaagcggatgccgtgcgccgaagactacctcagcgtggtccttaatcagctgtgcgtgctgcacgagaagaccccagtaagtgaccgggttacgaagtgctgtactgaatcacttgtgaacagaagaccttgtttctccgccctcgaggtggacgaaacgtacgttccgaaggaattcaacgccgaaacattcaccttccacgctgacatttgtacgctttccgagaaggagagacaaatcaagaagcagactgccctggtggaattagtgaagcacaagccgaaggccacaaaggagcagctgaaggcagttatggacgacttcgctgcattcgtagagaagtgctgcaaggcagacgacaaggaaacttgcttcgcagaggagggaaagaagctcgttgctgcgtctcaggcggctctgggactgggaggaggatccgctcctacaagcagttccacaaagaagacccagctccagcttgaacatctgcttctcgacctgcaaatgatcctcaacggtatcaataattacaagaatcctaagttgactcgcatgctgactttcaagttctatatgccaaagaaggccactgagttgaagcacctgcagtgcctcgaggaagaactgaagcctctggaggaagtcttaaacctggcacaaagcaagaacttccatctgaggcctagggacctcatttccaatatcaacgttattgtgctggagctcaagggtagcgagacgacattcatgtgcgagtacgcagacgagacagcgactatcgtcgagttcctaaataggtggataacgttctgccagtcaatcatctccactctgaccggcaagccgatccctaacccgttgcttggattggattctaca

SEQ ID NO: 22 - RNA, 2652 nt, synthetic construct
  source 1..2652: mol_type = other RNA
atgcctctgctgctcttactgccgctgctttgggcaggagctctggccgaggtccagctagtgcagacaggcggtggactcagccagttcggtgagtcactgcgtctatcctgcgcggttagtggcttcaacgtgtccaacaattatatgagttgggtgagacaagcacctggcaagggccttgagtgggtgtccattatatatagcggtggcggcacccactacgctgactcggtgaagggtcgattcacaattagcagagacaattccaagaatactctgttcctgcagatgaactctctcagggcggaggacaccgccgtgtactactgtgcccgtgcggtgccggtccctcacggtacagacatctggggccagggtaccatggtcaccgtgtcatccggaggaggcggttctggtggaggaggaagtggaggcggcggcagtgcacataagagcgaggtggcccatagattcaaggatttaggagaggagaacttcaaggcactggtcttgatcgccttcgcacaatatcttcagcagtgtccgttcgaagatcacgtgaagctcgtcaacgaggtgactgagttcgccaagacctgcgtggcagacgagtcggctgagaattgtgataagagtctgcacaccctcttcggagataagctgtgtaccgtggccactctgagggagacatacggcgagatggctgactgttgtgcaaagcaggagccagagaggaacgagtgtttcttgcagcataaggacgacaacccaaatctgcctcgtctggttagacctgaagtggacgtaatgtgcaccgctttccacgacaacgaggagacgttcctgaagaagtatctgtacgagattgctagaaggcacccatacttctacgcaccagagctgttgttcttcgctaagagatacaaggccgctttcaccgagtgctgccaagcagcagacaaggctgcttgcctgctccctaagctggacgagctgagagacgagggtaaggcttcatcagccaagcaaagattgaagtgtgcatcactgcagaagttcggcgaaagagcattcaaggcttgggccgtcgccaggctgtctcagaggttcccgaaggccgagttcgctgaagtgtctaagttagtgaccgatctaacaaaggtgcacacagagtgttgccacggagatctgcttgagtgcgccgacgatcgagccgatcttgcaaagtacatctgtgagaaccaggattccatctcctctaagttgaaggagtgctgtgagaagccacttctcgagaagtctcactgcatcgcagaagtcgagaacgacgaaatgccagctgacctgccgagcttggccgcagatttcgtcgagagtaaggacgtttgcaagaattacgctgaggctaaggacgtcttcctgggcatgttcttgtacgaatacgccaggaggcacccggattactcagtggtacttttactgcgactggctaagacctacgaaacgacactggagaagtgctgcgcagccgccgatcctcacgagtgctacgctaaggttttcgacgagttcaagccgctcgtggaagaacctcagaacttaatcaagcagaattgtgagctattcgaacagctcggagagtataagttccagaacgccctcctggtgcggtacacaaagaaggttccacaggtcagcaccccgaccctcgttgaggtgagccgcaacctcggcaaggttggctctaagtgttgtaagcatccggaagcgaagcgcatgccttgtgcagaagattatctttcagttgtgttgaaccagctctgtgtcctgcacgagaagaccccagtttccgaccgcgttaccaagtgttgcacagaaagcttggtcaaccggaggccttgcttcagcgcattggaggtagacgaaacctacgtccctaaggaattcaacgccgaaacattcacgttccacgcggacatttgcacactctccgagaaggaacgccagattaagaagcagactgccttggttgagctggtaaagcacaagcctaaggcgactaaggagcagctaaaggcagttatggacgacttcgctgcattcgttgagaagtgctgtaaggccgacgataaggaaacctgcttcgccgaggaaggaaagaagcttgttgccgctagccaggccgcactgggattgggtggaggcagcgcaccaacttcctctagcaccaagaagactcaattacaactggaacatctcttactaacactccaaatgatactgaacggcataaacaactataagaacccgaagttgaccagaatgttaaccttcaagttctacatgcctaagaaggctacggaactgaagcaccttcagtgcctagaggaggagctcaagcctctcgaggaggttctgaacctcgcacagagtaagaatttccacctgcgcccaagggacctgatttccaatattaacgtgatcgtccttgaacttaagggcagcgaaaccactttcatgtgcgaatacgcggacgaaactgctaccatagttgagttccttaaccgctggatcacattctgccagtcaatcatctctacattgaccggaaagccgattccgaacccactcctgggacttgatagcaca

SEQ ID NO: 23 - RNA, 2652 nt, synthetic construct
  source 1..2652: mol_type = other RNA
atgcctctgctcctgcttcttcctctgctctgggctggcgccctagccgaggtgcagctggtgcagaccggcggcggcctgagccagttcggcgagagcctgagactgagctgcgccgtgagcggcttcaacgtgagcaacaactacatgagctgggtgagacaggcccctggcaagggcctggagtgggtgagcatcatctacagtggcggaggtacccactacgccgacagcgtgaagggcagattcaccatcagcagagacaacagcaagaacaccctgttcctgcagatgaacagcctcagggccgaggacaccgccgtgtactactgcgccagagccgtgcctgtgcctcacggcaccgacatctggggccagggcaccatggtgaccgtgagtagcggtggaggcggaagcggaggcggtggctctggaggtggcggcagcgcccacaagagcgaggtggcccacagattcaaggacctgggcgaggagaacttcaaggccctggtgctgatcgccttcgcccagtacctgcagcagtgccctttcgaggaccacgtgaagctggtgaacgaggtgaccgagttcgccaagacctgcgtggccgacgagagcgccgagaactgcgacaagagcctgcacacactcttcggcgacaagctgtgcaccgtggccaccctgagagagacttacggcgagatggccgactgctgcgccaagcaggaaccagagaggaacgagtgcttccttcagcacaaggacgacaaccctaacctgcctagactggttcggcctgaggtggacgtgatgtgcactgccttccacgacaacgaggagacgttcctgaagaagtacctgtacgagatcgccagaagacacccttacttctacgcccctgagcttctgttcttcgcaaagagatacaaggccgccttcaccgagtgctgccaggccgccgacaaggctgcctgcctcctgccaaagctggacgagctgagagacgagggcaaggccagctctgcgaagcagaggctgaagtgcgccagcctgcagaagttcggagaaagagctttcaaggcctgggccgtggcccgcttgtctcaaagattccctaaggcggagttcgcagaagtgagcaagctggtcaccgacctgaccaaggtgcatactgagtgttgccacggcgacctgctggagtgcgccgacgacagagccgacctggccaagtacatctgcgagaaccaggactctattagtagcaagttgaaggagtgttgcgagaagccgctactcgagaagagtcactgcattgcggaggttgagaacgacgagatgcctgccgacctcccaagcctggcagccgatttcgtggagagcaaggacgtgtgcaagaattacgctgaggctaaggacgttttcctgggcatgttcttatacgaatacgctagacggcacccagactacagcgtggtcctcctcctgcggcttgcaaagacgtacgaaactaccctggagaagtgctgcgcggctgctgaccctcacgagtgctacgccaaggtgttcgacgagttcaagcctctcgtggaggagcctcagaacctgatcaagcagaattgcgagctgttcgagcagttaggtgagtacaagttccagaacgccctgcttgtgcgctacaccaagaaggtgcctcaggtttccacccctaccctggtagaggtgtccagaaacctgggcaaggtgggcagcaagtgttgcaagcatccagaagctaagagaatgccttgcgccgaagattacctgtctgtcgtgctgaaccagctgtgcgtgctgcacgagaagacccctgtgagcgacagagtgaccaagtgctgtacagaatccctagtaaatagaagaccttgcttctccgccctagaggtggacgaaacctacgtgcctaaggagttcaacgccgagactttcacgttccacgcggatatttgtacactcagcgagaaggagagacagatcaagaagcagaccgcacttgtggagctcgtcaagcacaagccaaaggccaccaaggagcaactcaaggccgtgatggacgacttcgcagccttcgtcgagaagtgttgtaaggctgacgataaggaaacctgcttcgctgaggaaggcaagaagcttgtggccgccagtcaggctgctcttggtcttggcggcggatcagcacctaccagcagctctacaaagaagacgcagctgcaactggagcacttgctgcttgatctgcaaatgatcctgaacggcatcaacaattacaagaaccctaagctgaccagaatgctgaccttcaagttctacatgcctaagaaggccacagagctgaagcatttacagtgcctggaggaggagcttaagccacttgaagaggtgctcaatctggcccagtctaagaacttccacctcaggcctcgcgatctgattagcgacatcaacgtaatcgtgctggaacttaagggatctgaaactacgttcatgtgcgagtacgccgacgaaacagccaccatcgtggagttcctgaaccgatggatcaccttctgccagagtattatttcaaccctcaccggcaagcctatccctaacccactgctcggactcgacagcacc

SEQ ID NO: 24 - DNA, 804 bp, synthetic construct
  source 1..804: mol_type = other DNA
atggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatga

SEQ ID NO: 25 - RNA, 2238 nt, synthetic construct
  source 1..2238: mol_type = other RNA
  modified_base 2: mod_base = OTHER; note = N1-methylpseudouracil
  modified_base 7: mod_base = OTHER; note = N1-methylpseudouracil
  modified_base 11: mod_base = OTHER; note = N1-methylpseudouracil
  modified_base 16: mod_base = OTHER; note = N1-methylpseudouracil
  modified_base 17: mod_base = OTHER; note = N1-methylpseudouracil
  modified_base 20: mod_base = OTHER; note = N1-methylpseudouracil
  modified_base 26: mod_base = OTHER; note = N1-methylpseudouracil
  modified_base 29: mod_base = OTHER; note = N1-methylpseudouracil
  modified_base 31: mod_base = OTHER; note = N1-methylpseudouracil
  modified_base 32: mod_base = OTHER; note = N1-methylpseudouracil
  modified_base 35: mod_base = OTHER; note = N1-methylpseudouracil
  modified_base 37: mod_base = OTHER; note = N1-methylpseudouracil
  modified_base 38: mod_base = OTHER; note = N1-methylpseudouracil
  modified_base 49: mod_base = OTHER; note = N1-methylpseudouracil
  modified_base 62: mod_base = OTHER; note = N1-methylpseudouracil
  modified_base 64: mod_base = OTHER; note = N1-methylpseudouracil
  modified_base 65: mod_base = OTHER; note = N1-methylpseudouracil
  modified_base 92: mod_base = OTHER; note = N1-methylpseudouracil
  modified_base 103: mod_base = OTHER; note = N1-methylpseudouracil
  modified_base 104: mod_base = OTHER; note = N1-methylpseudouracil
  modified_base 113: mod_base = OTHER; note = N1-methylpseudouracil
  modified_base 127: mod_base = OTHER; note = N1-methylpseudouracil
  modified_base 128: mod_base = OTHER; note = N1-methylpseudouracil
  modified_base 137: mod_base = OTHER; note = N1-methylpseudouracil
  modified_base 140: mod_base = OTHER; note = N1-methylpseudouracil
  modified_base 143: mod_base = OTHER; note = N1-methylpseudouracil
  modified_base 146: mod_base = OTHER; note = N1-methylpseudouracil
  modified_base 151: mod_base = OTHER; note = N1-methylpseudouracil
  modified_base 152: mod_base = OTHER; note = N1-methylpseudouracil
  modified_base 160: mod_base = OTHER; note = N1-methylpseudouracil
  modified_base 164: mod_base = OTHER; note = N1-methylpseudouracil
  modified_base 172: mod_base = OTHER; note = N1-methylpseudouracil
  modified_base 177: mod_base = OTHER; note = N1-methylpseudouracil
  modified_base 178: mod_base = OTHER; note = N1-methylpseudouracil
  modified_base 179: mod_base = OTHER; note = N1-methylpseudouracil
  modified_base 191: mod_base = OTHER; note = N1-methylpseudouracil
  modified_base 197: mod_base = OTHER; note = N1-methylpseudouracil
  modified_base 200: mod_base = OTHER; note = N1-methylpseudouracil
  modified_base 209: mod_base = OTHER; note = N1-methylpseudouracil
  modified_base 217: mod_base = OTHER; note = N1-methylpseudouracil
  modified_base 218: mod_base = OTHER; note = N1-methylpseudouracil
  modified_base 229: mod_base = OTHER; note = N1-methylpseudouracil
  modified_base 233: mod_base = OTHER; note = N1-methylpseudouracil
  modified_base 256: mod_base = OTHER; note = N1-methylpseudouracil
  modified_base 269: mod_base = OTHER; note = N1-methylpseudouracil
  modified_base 278: mod_base = OTHER; note = N1-methylpseudouracil
  modified_base 280: mod_base = OTHER; note = N1-methylpseudouracil
  modified_base 281: mod_base = OTHER; note = N1-methylpseudouracil
  modified_base 293: mod_base = OTHER; note = N1-methylpseudouracil
  modified_base 295: mod_base = OTHER; note = N1-methylpseudouracil
  modified_base 302: mod_base = OTHER; note = N1-methylpseudouracil
  modified_base 311: mod_base = OTHER; note = N1-methylpseudouracil
  modified_base 321: mod_base = OTHER; note = N1-methylpseudouracil
  modified_base 322: mod_base = OTHER; note = N1-methylpseudouracil
  modified_base 332: mod_base = OTHER; note = N1-methylpseudouracil
  modified_base 340: mod_base = OTHER; note = N1-methylpseudouracil
  modified_base 343: mod_base = OTHER; note = N1-methylpseudouracil
  modified_base 373: mod_base = OTHER; note = N1-methylpseudouracil
  modified_base 376: mod_base = OTHER; note = N1-methylpseudouracil
  modified_base 377: mod_base = OTHER; note = N1-methylpseudouracil
  modified_base 380: mod_base = OTHER; note = N1-methylpseudouracil
  modified_base 402: mod_base = OTHER; note = N1-methylpseudouracil
  modified_base 407: mod_base = OTHER; note = N1-methylpseudouracil
  modified_base 411: mod_base = OTHER; note = N1-methylpseudouracil
  modified_base 416: mod_base = OTHER; note = N1-methylpseudouracil
  modified_base 419: mod_base = OTHER; note = N1-methylpseudouracil
  modified_base 426: mod_base = OTHER; note = N1-methylpseudouracil
  modified_base 431: mod_base = OTHER; note = N1-methylpseudouracil
  modified_base 437: mod_base = OTHER; note = N1-methylpseudouracil
  modified_base 440: mod_base = OTHER; note = N1-methylpseudouracil
  modified_base 442: mod_base = OTHER; note = N1-methylpseudouracil
  modified_base 444: mod_base = OTHER; note = N1-methylpseudouracil
  modified_base 451: mod_base = OTHER; note = N1-methylpseudouracil
  modified_base 452: mod_base = OTHER; note = N1-methylpseudouracil
  modified_base 472: mod_base = OTHER; note = N1-methylpseudouracil
  modified_base 473: mod_base = OTHER; note = N1-methylpseudouracil
  modified_base 476: mod_base = OTHER; note = N1-methylpseudouracil
  modified_base 484: mod_base = OTHER; note = N1-methylpseudouracil
  modified_base 488: mod_base = OTHER; note = N1-methylpseudouracil
  modified_base 490: mod_base = OTHER; note = N1-methylpseudouracil
  modified_base 497: mod_base = OTHER; note = N1-methylpseudouracil
  modified_base 513: mod_base = OTHER; note = N1-methylpseudouracil
  modified_base 514: mod_base = OTHER; note = N1-methylpseudouracil
  modified_base 517: mod_base = OTHER; note = N1-methylpseudouracil
  modified_base 518: mod_base = OTHER; note = N1-methylpseudouracil
  modified_base 520: mod_base = OTHER; note = N1-methylpseudouracil
  modified_base 528: mod_base = OTHER; note = N1-methylpseudouracil
  modified_base 532: mod_base = OTHER; note = N1-methylpseudouracil
  modified_base 533: mod_base = OTHER; note = N1-methylpseudouracil
  modified_base 536: mod_base = OTHER; note = N1-methylpseudouracil
  modified_base 538: mod_base = OTHER; note = N1-methylpseudouracil
  modified_base 539: mod_base = OTHER; note = N1-methylpseudouracil
  modified_base 541: mod_base = OTHER; note = N1-methylpseudouracil
  modified_base 542: mod_base = OTHER; note = N1-methylpseudouracil
  modified_base 553: mod_base = OTHER; note = N1-methylpseudouracil
  modified_base 565: mod_base = OTHER; note = N1-methylpseudouracil
  modified_base 566: mod_base = OTHER; note = N1-methylpseudouracil
  modified_base 574: mod_base = OTHER; note = N1-methylpseudouracil
  modified_base 577: mod_base = OTHER; note = N1-methylpseudouracil
  modified_base 591: mod_base = OTHER; note = N1-methylpseudouracil
  modified_base 601: mod_base = OTHER; note = N1-methylpseudouracil
  modified_base 605: mod_base = OTHER; note = N1-methylpseudouracil
  modified_base 608: mod_base = OTHER; note = N1-methylpseudouracil
  modified_base 612: mod_base = OTHER; note = N1-methylpseudouracil
  modified_base 617: mod_base = OTHER; note = N1-methylpseudouracil
  modified_base 626: mod_base = OTHER; note = N1-methylpseudouracil
  modified_base 646: mod_base = OTHER; note = N1-methylpseudouracil
  modified_base 654: mod_base = OTHER; note = N1-methylpseudouracil
  modified_base 665: mod_base = OTHER; note = N1-methylpseudouracil
  modified_base 670: mod_base = OTHER; note = N1-methylpseudouracil
  modified_base 680: mod_base = OTHER; note = N1-methylpseudouracil
  modified_base 688: mod_base = OTHER; note = N1-methylpseudouracil
  modified_base 689: mod_base = OTHER; note = N1-methylpseudouracil
  modified_base 702: mod_base = OTHER; note = N1-methylpseudouracil
  modified_base 703: mod_base = OTHER; note = N1-methylpseudouracil
  modified_base 704: mod_base = OTHER; note = N1-methylpseudouracil
  modified_base 712: mod_base = OTHER; note = N1-methylpseudouracil
  modified_base 719: mod_base = OTHER; note = N1-methylpseudouracil
  modified_base 723: mod_base = OTHER; note = N1-methylpseudouracil
  modified_base 727: mod_base = OTHER; note = N1-methylpseudouracil
  modified_base 728: mod_base = OTHER; note = N1-methylpseudouracil
  modified_base 739: mod_base = OTHER; note = N1-methylpseudouracil
  modified_base 740: mod_base = OTHER; note = N1-methylpseudouracil
  modified_base 744: mod_base = OTHER; note = N1-methylpseudouracil
  modified_base 754: mod_base = OTHER; note = N1-methylpseudouracil
  modified_base 755: mod_base = OTHER; note = N1-methylpseudouracil
  modified_base 759: mod_base = OTHER; note = N1-methylpseudouracil
  modified_base 764: mod_base = OTHER; note = N1-methylpseudouracil
  modified_base 773: mod_base = OTHER; note = N1-methylpseudouracil
  modified_base 776: mod_base = OTHER; note = N1-methylpseudouracil
  modified_base 780: mod_base = OTHER; note = N1-methylpseudouracil
  modified_base 785: mod_base = OTHER; note = N1-methylpseudouracil
  modified_base 794: mod_base = OTHER; note = N1-methylpseudouracil
  modified_base 798: mod_base = OTHER; note = N1-methylpseudouracil
  modified_base 805: mod_base = OTHER; note = N1-methylpseudouracil
  modified_base 808: mod_base = OTHER; note = N1-methylpseudouracil
  modified_base 810: mod_base = OTHER; note = N1-methylpseudouracil
  modified_base 821: mod_base = OTHER; note = N1-methylpseudouracil
  modified_base 824: mod_base = OTHER; note = N1-methylpseudouracil
  modified_base 829: mod_base = OTHER; note = N1-methylpseudouracil
  modified_base 851: mod_base = OTHER; note = N1-methylpseudouracil
  modified_base 859: mod_base = OTHER; note = N1-methylpseudouracil
  modified_base 863: mod_base = OTHER; note = N1-methylpseudouracil
  modified_base 865: mod_base = OTHER; note = N1-methylpseudouracil
  modified_base 884: mod_base = OTHER; note = N1-methylpseudouracil
  modified_base 896: mod_base = OTHER; note = N1-methylpseudouracil
  modified_base 904: mod_base = OTHER; note = N1-methylpseudouracil
  modified_base 906: mod_base = OTHER; note = N1-methylpseudouracil
  modified_base 907: mod_base = OTHER; note = N1-methylpseudouracil
  modified_base 909: mod_base = OTHER; note = N1-methylpseudouracil
  modified_base 918: mod_base = OTHER; note = N1-methylpseudouracil
  modified_base 920: mod_base = OTHER; note = N1-methylpseudouracil
  modified_base 922: mod_base = OTHER; note = N1-methylpseudouracil
  modified_base 923: mod_base = OTHER; note = N1-methylpseudouracil
  modified_base 933: mod_base = OTHER; note = N1-methylpseudouracil
  modified_base 937: mod_base = OTHER; note = N1-methylpseudouracil
  modified_base 941: mod_base = OTHER; note = N1-methylpseudouracil
  modified_base 942: mod_base = OTHER; note = N1-methylpseudouracil
  modified_base 950: mod_base = OTHER; note = N1-methylpseudouracil
  modified_base 965: mod_base = OTHER; note = N1-methylpseudouracil
  modified_base 969: mod_base = OTHER; note = N1-methylpseudouracil
  modified_base 975: mod_base = OTHER; note = N1-methylpseudouracil
  modified_base 977: mod_base = OTHER; note = N1-methylpseudouracil
  modified_base 985: mod_base = OTHER; note = N1-methylpseudouracil
  modified_base 986: mod_base = OTHER; note = N1-methylpseudouracil
  modified_base 997: mod_base = OTHER; note = N1-methylpseudouracil
  modified_base 998: mod_base = OTHER; note = N1-methylpseudouracil
  modified_base 1001: mod_base = OTHER; note = N1-methylpseudouracil
  modified_base 1016: mod_base = OTHER; note = N1-methylpseudouracil
  modified_base 1018: mod_base = OTHER; note = N1-methylpseudouracil
  modified_base 1027: mod_base = OTHER; note = N1-methylpseudouracil
  modified_base 1046: mod_base = OTHER; note = N1-methylpseudouracil
  modified_base 1047: mod_base = OTHER; note = N1-methylpseudouracil
  modified_base 1048: mod_base = OTHER; note = N1-methylpseudouracil
  modified_base 1049: mod_base = OTHER; note = N1-methylpseudouracil
  modified_base 1052: mod_base = OTHER; note = N1-methylpseudouracil
  modified_base 1058: mod_base = OTHER; note = N1-methylpseudouracil
  modified_base 1060: mod_base = OTHER; note = N1-methylpseudouracil
  modified_base 1061: mod_base = OTHER; note = N1-methylpseudouracil
  modified_base 1064: mod_base = OTHER; note = N1-methylpseudouracil
  modified_base 1066: mod_base = OTHER; note = N1-methylpseudouracil
  modified_base 1072: mod_base = OTHER; note = N1-methylpseudouracil
  modified_base 1086: mod_base = OTHER; note = N1-methylpseudouracil
  modified_base 1093: mod_base = OTHER; note = N1-methylpseudouracil
  modified_base 1100: mod_base = OTHER; note = N1-methylpseudouracil
  modified_base 1103: mod_base = OTHER; note = N1-methylpseudouracil
  modified_base 1106: mod_base = OTHER; note = N1-methylpseudouracil
  modified_base 1109: mod_base = OTHER; note = N1-methylpseudouracil
  modified_base 1112: mod_base = OTHER; note = N1-methylpseudouracil
  modified_base 1118: mod_base = OTHER; note = N1-methylpseudouracil
  modified_base 1122: mod_base = OTHER; note = N1-methylpseudouracil
  modified_base 1129: mod_base = OTHER; note = N1-methylpseudouracil
  modified_base 1137: mod_base = OTHER; note = N1-methylpseudouracil
  modified_base 1142: mod_base = OTHER; note = N1-methylpseudouracil
  modified_base 1150: mod_base = OTHER; note = N1-methylpseudouracil
  modified_base 1153: mod_base = OTHER; note = N1-methylpseudouracil
  modified_base 1170: mod_base = OTHER; note = N1-methylpseudouracil
  modified_base 1177: mod_base = OTHER; note = N1-methylpseudouracil
  modified_base 1179: mod_base = OTHER; note = N1-methylpseudouracil
  modified_base 1180: mod_base = OTHER; note = N1-methylpseudouracil
  modified_base 1190: mod_base = OTHER; note = N1-methylpseudouracil
  modified_base 1192: mod_base = OTHER; note = N1-methylpseudouracil
  modified_base 1193: mod_base = OTHER; note = N1-methylpseudouracil
  modified_base 1201: mod_base = OTHER; note = N1-methylpseudouracil
  modified_base 1202: mod_base = OTHER; note = N1-methylpseudouracil
  modified_base 1209: mod_base = OTHER; note = N1-methylpseudouracil
  modified_base 1211: mod_base = OTHER; note = N1-methylpseudouracil
  modified_base 1214: mod_base = OTHER; note = N1-methylpseudouracil
  modified_base 1224: mod_base = OTHER; note = N1-methylpseudouracil
  modified_base 1232: mod_base = OTHER; note = N1-methylpseudouracil
  modified_base 1235: mod_base = OTHER; note = N1-methylpseudouracil
  modified_base 1246: mod_base = OTHER; note = N1-methylpseudouracil
  modified_base 1248: mod_base = OTHER; note = N1-methylpseudouracil
  modified_base 1253: mod_base = OTHER; note = N1-methylpseudouracil
  modified_base 1255: mod_base = OTHER; note = N1-methylpseudouracil
  modified_base 1256: mod_base = OTHER; note = N1-methylpseudouracil
  modified_base 1265: mod_base = OTHER; note = N1-methylpseudouracil
  modified_base 1273: mod_base = OTHER; note = N1-methylpseudouracil
  modified_base 1279: mod_base = OTHER; note = N1-methylpseudouracil
  modified_base 1280: mod_base = OTHER; note = N1-methylpseudouracil
  modified_base 1292: mod_base = OTHER; note = N1-methylpseudouracil
  modified_base 1294: mod_base = OTHER; note = N1-methylpseudouracil
  modified_base 1295: mod_base = OTHER; note = N1-methylpseudouracil
  modified_base 1298: mod_base = OTHER; note = N1-methylpseudouracil
  modified_base 1303: mod_base = OTHER; note = N1-methylpseudouracil
  modified_base 1316: mod_base = OTHER; note = N1-methylpseudouracil
  modified_base 1320: mod_base = OTHER; note = N1-methylpseudouracil
  modified_base 1325: mod_base = OTHER; note = N1-methylpseudouracil
  modified_base 1329: mod_base = OTHER; note = N1-methylpseudouracil
  modified_base 1335: mod_base = OTHER; note = N1-methylpseudouracil
  modified_base 1340: mod_base = OTHER; note = N1-methylpseudouracil
  modified_base 1343: mod_base = OTHER; note = N1-methylpseudouracil
  modified_base 1349: mod_base = OTHER; note = N1-methylpseudouracil
  modified_base 1350: mod_base = OTHER; note = N1-methylpseudouracil
  modified_base 1353: mod_base = OTHER; note = N1-methylpseudouracil
  modified_base 1361: mod_base = OTHER; note = N1-methylpseudouracil
  modified_base 1370: mod_base = OTHER; note = N1-methylpseudouracil
  modified_base 1381: mod_base = OTHER; note = N1-methylpseudouracil
  modified_base 1384: mod_base = OTHER; note = N1-methylpseudouracil
  modified_base 1386: mod_base = OTHER; note = N1-methylpseudouracil
  modified_base 1401: mod_base = OTHER; note = N1-methylpseudouracil
  modified_base 1409: mod_base = OTHER; note = N1-methylpseudouracil
  modified_base 1413: mod_base = OTHER; note = N1-methylpseudouracil
  modified_base 1414: mod_base = OTHER; note = N1-methylpseudouracil
  modified_base 1426: mod_base = OTHER; note = N1-methylpseudouracil
  modified_base 1430: mod_base = OTHER; note = N1-methylpseudouracil
  modified_base 1432: mod_base = OTHER; note = N1-methylpseudouracil
  modified_base 1436: mod_base = OTHER; note = N1-methylpseudouracil
  modified_base 1437: mod_base = OTHER; note = N1-methylpseudouracil
  modified_base 1439: mod_base = OTHER; note = N1-methylpseudouracil
  modified_base 1442: mod_base = OTHER; note = N1-methylpseudouracil
  modified_base 1451: mod_base = OTHER; note = N1-methylpseudouracil
  modified_base 1453: mod_base = OTHER; note = N1-methylpseudouracil
  modified_base 1457: mod_base = OTHER; note = N1-methylpseudouracil
  modified_base 1460: mod_base = OTHER; note = N1-methylpseudouracil
  modified_base 1476: mod_base = OTHER; note = N1-methylpseudouracil
  modified_base 1478: mod_base = OTHER; note = N1-methylpseudouracil
  modified_base 1490: mod_base = OTHER; note = N1-methylpseudouracil
  modified_base 1498: mod_base = OTHER; note = N1-methylpseudouracil
  modified_base 1500: mod_base = OTHER; note = N1-methylpseudouracil
  modified_base 1501: mod_base = OTHER; note = N1-methylpseudouracil
  modified_base 1513: mod_base = OTHER; note = N1-methylpseudouracil
  modified_base 1514: mod_base = OTHER; note = N1-methylpseudouracil
  modified_base 1517: mod_base = OTHER; note = N1-methylpseudouracil
  modified_base 1521: mod_base = OTHER; note = N1-methylpseudouracil
  modified_base 1530: mod_base = OTHER; note = N1-methylpseudouracil
  modified_base 1531: mod_base = OTHER; note = N1-methylpseudouracil
  modified_base 1534: mod_base = OTHER; note = N1-methylpseudouracil
  modified_base 1535: mod_base = OTHER; note = N1-methylpseudouracil
  modified_base 1544: mod_base = OTHER; note = N1-methylpseudouracil
  modified_base 1550: mod_base = OTHER; note = N1-methylpseudouracil
  modified_base 1551: mod_base = OTHER; note = N1-methylpseudouracil
  modified_base 1560: mod_base = OTHER; note = N1-methylpseudouracil
  modified_base 1561: mod_base = OTHER; note = N1-methylpseudouracil
  modified_base 1565: mod_base = OTHER; note = N1-methylpseudouracil
  modified_base 1569: mod_base = OTHER; note = N1-methylpseudouracil
  modified_base 1576: mod_base = OTHER; note = N1-methylpseudouracil
  modified_base 1577: mod_base = OTHER; note = N1-methylpseudouracil
  modified_base 1590: mod_base = OTHER; note = N1-methylpseudouracil
  modified_base 1591: mod_base = OTHER; note = N1-methylpseudouracil
  modified_base 1592: mod_base = OTHER; note = N1-methylpseudouracil
  modified_base 1597: mod_base = OTHER; note = N1-methylpseudouracil
  modified_base 1598: mod_base = OTHER; note = N1-methylpseudouracil
  modified_base 1610: mod_base = OTHER; note = N1-methylpseudouracil
  modified_base 1612: mod_base = OTHER; note = N1-methylpseudouracil
  modified_base 1614: mod_base = OTHER; note = N1-methylpseudouracil
  modified_base 1617: mod_base = OTHER; note = N1-methylpseudouracil
  modified_base 1619: mod_base = OTHER; note = N1-methylpseudouracil
  modified_base 1640: mod_base = OTHER; note = N1-methylpseudouracil
  modified_base 1658: mod_base = OTHER; note = N1-methylpseudouracil
  modified_base 1661: mod_base = OTHER; note = N1-methylpseudouracil
  modified_base 1666: mod_base = OTHER; note = N1-methylpseudouracil
  modified_base 1667: mod_base = OTHER; note = N1-methylpseudouracil
  modified_base 1670: mod_base = OTHER; note = N1-methylpseudouracil
  modified_base 1703: mod_base = OTHER; note = N1-methylpseudouracil
  modified_base 1704: mod_base = OTHER; note = N1-methylpseudouracil
  modified_base 1712: mod_base = OTHER; note = N1-methylpseudouracil
  modified_base 1715: mod_base = OTHER; note = N1-methylpseudouracil
  modified_base 1723: mod_base = OTHER; note = N1-methylpseudouracil
  modified_base 1724: mod_base = OTHER; note = N1-methylpseudouracil
  modified_base 1732: mod_base = OTHER; note = N1-methylpseudouracil
  modified_base 1733: mod_base = OTHER; note = N1-methylpseudouracil
  modified_base 1736: mod_base = OTHER; note = N1-methylpseudouracil
  modified_base 1744: mod_base = OTHER; note = N1-methylpseudouracil
  modified_base 1746: mod_base = OTHER; note = N1-methylpseudouracil
  modified_base 1747: mod_base = OTHER; note = N1-methylpseudouracil
  modified_base 1749: mod_base = OTHER; note = N1-methylpseudouracil
  modified_base 1770: mod_base = OTHER; note = N1-methylpseudouracil
  modified_base 1771: mod_base = OTHER; note = N1-methylpseudouracil
  modified_base 1774: mod_base = OTHER; note = N1-methylpseudouracil
  modified_base 1775: mod_base = OTHER; note = N1-methylpseudouracil
  modified_base 1795: mod_base = OTHER; note = N1-methylpseudouracil
  modified_base 1796: mod_base = OTHER; note = N1-methylpseudouracil
  modified_base 1799: mod_base = OTHER; note = N1-methylpseudouracil
  modified_base 1807: mod_base = OTHER; note = N1-methylpseudouracil
  modified_base 1809: mod_base = OTHER; note = N1-methylpseudouracil
  modified_base 1815: mod_base = OTHER; note = N1-methylpseudouracil
  modified_base 1819: mod_base = OTHER; note = N1-methylpseudouracil
  modified_base 1820: mod_base = OTHER; note = N1-methylpseudouracil
  modified_base 1826: mod_base = OTHER; note = N1-methylpseudouracil
  modified_base 1830: mod_base = OTHER; note = N1-methylpseudouracil
  modified_base 1845: mod_base = OTHER; note = N1-methylpseudouracil
  modified_base 1855: mod_base = OTHER; note = N1-methylpseudouracil
  modified_base 1874: mod_base = OTHER; note = N1-methylpseudouracil
  modified_base 1880: mod_base = OTHER; note = N1-methylpseudouracil
  modified_base 1889: mod_base = OTHER; note = N1-methylpseudouracil
  modified_base 1892: mod_base = OTHER; note = N1-methylpseudouracil
  modified_base 1895: mod_base = OTHER; note = N1-methylpseudouracil
  modified_base 1900: mod_base = OTHER; note = N1-methylpseudouracil
  modified_base 1901: mod_base = OTHER; note = N1-methylpseudouracil
  modified_base 1907: mod_base = OTHER; note = N1-methylpseudouracil
  modified_base 1910: mod_base = OTHER; note = N1-methylpseudouracil
  modified_base 1913: mod_base = OTHER; note = N1-methylpseudouracil
  modified_base 1922: mod_base = OTHER; note = N1-methylpseudouracil
  modified_base 1930: mod_base = OTHER; note = N1-methylpseudouracil
  modified_base 1946: mod_base = OTHER; note = N1-methylpseudouracil
  modified_base 1955: mod_base = OTHER; note = N1-methylpseudouracil
  modified_base 1958: mod_base = OTHER; note = N1-methylpseudouracil
  modified_base 1963: mod_base = OTHER; note = N1-methylpseudouracil
  modified_base 1964: mod_base = OTHER; note = N1-methylpseudouracil
  modified_base 1969: mod_base = OTHER; note = N1-methylpseudouracil
  modified_base 1970: mod_base = OTHER; note = N1-methylpseudouracil
  modified_base 1972: mod_base = OTHER; note = N1-methylpseudouracil
  modified_base 1976: mod_base = OTHER; note = N1-methylpseudouracil
  modified_base 1980: mod_base = OTHER; note = N1-methylpseudouracil
  modified_base 1992: mod_base = OTHER; note = N1-methylpseudouracil
  modified_base 1997: mod_base = OTHER; note = N1-methylpseudouracil
  modified_base 2006: mod_base = OTHER; note = N1-methylpseudouracil
  modified_base 2007: mod_base = OTHER; note = N1-methylpseudouracil
  modified_base 2011: mod_base = OTHER; note = N1-methylpseudouracil
  modified_base 2015: mod_base = OTHER; note = N1-methylpseudouracil
  modified_base 2027: mod_base = OTHER; note = N1-methylpseudouracil
  modified_base 2028: mod_base = OTHER; note = N1-methylpseudouracil
  modified_base 2036: mod_base = OTHER; note = N1-methylpseudouracil
  modified_base 2048: mod_base = OTHER; note = N1-methylpseudouracil
  modified_base 2054: mod_base = OTHER; note = N1-methylpseudouracil
  modified_base 2061: mod_base = OTHER; note = N1-methylpseudouracil
  modified_base 2064: mod_base = OTHER; note = N1-methylpseudouracil
  modified_base 2070: mod_base = OTHER; note = N1-methylpseudouracil
  modified_base 2071: mod_base = OTHER; note = N1-methylpseudouracil
  modified_base 2072: mod_base = OTHER; note = N1-methylpseudouracil
  modified_base 2078: mod_base = OTHER; note = N1-methylpseudouracil
  modified_base 2085: mod_base = OTHER; note = N1-methylpseudouracil
  modified_base 2093: mod_base = OTHER; note = N1-methylpseudouracil
  modified_base 2094: mod_base = OTHER; note = N1-methylpseudouracil
  modified_base 2096: mod_base = OTHER; note = N1-methylpseudouracil
  modified_base 2097: mod_base = OTHER; note = N1-methylpseudouracil
  modified_base 2105: mod_base = OTHER; note = N1-methylpseudouracil
  modified_base 2111: mod_base = OTHER; note = N1-methylpseudouracil
  modified_base 2114: mod_base = OTHER; note = N1-methylpseudouracil
  modified_base 2117: mod_base = OTHER; note = N1-methylpseudouracil
  modified_base 2120: mod_base = OTHER; note = N1-methylpseudouracil
  modified_base 2125: mod_base = OTHER; note = N1-methylpseudouracil
  modified_base 2126: mod_base = OTHER; note = N1-methylpseudouracil
  modified_base 2134: mod_base = OTHER; note = N1-methylpseudouracil
  modified_base 2146: mod_base = OTHER; note = N1-methylpseudouracil
  modified_base 2147: mod_base = OTHER; note = N1-methylpseudouracil
  modified_base 2150: mod_base = OTHER; note = N1-methylpseudouracil
  modified_base 2152: mod_base = OTHER; note = N1-methylpseudouracil
  modified_base 2158: mod_base = OTHER; note = N1-methylpseudouracil
  modified_base 2180: mod_base = OTHER; note = N1-methylpseudouracil
  modified_base 2183: mod_base = OTHER; note = N1-methylpseudouracil
  modified_base 2188: mod_base = OTHER; note = N1-methylpseudouracil
  modified_base 2189: mod_base = OTHER; note = N1-methylpseudouracil
  modified_base 2192: mod_base = OTHER; note = N1-methylpseudouracil
  modified_base 2200: mod_base = OTHER; note = N1-methylpseudouracil
  modified_base 2204: mod_base = OTHER; note = N1-methylpseudouracil
  modified_base 2209: mod_base = OTHER; note = N1-methylpseudouracil
  modified_base 2210: mod_base = OTHER; note = N1-methylpseudouracil
  modified_base 2212: mod_base = OTHER; note = N1-methylpseudouracil
  modified_base 2222: mod_base = OTHER; note = N1-methylpseudouracil
  modified_base 2225: mod_base = OTHER; note = N1-methylpseudouracil
  modified_base 2227: mod_base = OTHER; note = N1-methylpseudouracil
  modified_base 2229: mod_base = OTHER; note = N1-methylpseudouracil
  modified_base 2232: mod_base = OTHER; note = N1-methylpseudouracil
  modified_base 2234: mod_base = OTHER; note = N1-methylpseudouracil
angaagngggngaccnncancagccngcngnnccngnncagcagcgccnacagcagaggcgngnncagaagagacgcccacaagagcgaggnggcccacaganncaaggaccngggcgaggagaacnncaaggcccnggngcngancgccnncgcccagnaccngcagcagngcccnnncgaggaccacgngaagcnggngaacgaggngaccgagnncgccaagaccngcgnggccgacgagagcgccgagaacngcgacaagagccngcacacccngnncggcgacaagcngngcaccgnggccacccngagagaaacnnacggcgaganggccgacngcngcgccaagcaggagccagagcggaacgagngcnnccnccagcacaaggacgacaacccnaaccngccnagacnggnaaggccngaggnggacgngangngnaccgccnnccacgacaacgaggagacannccngaagaagnaccngnacgagancgccagaagacacccnnacnncnacgccccngagnngcngnncnncgcgaagaganacaaggccgccnncaccgagngcngccaggccgccganaaggccgcgngccngcngccnaagcnggacgagcngagagacgagggcaaggcanccagcgcnaagcagagacngaagngcgccagccngcagaagnncggagagagagcnnncaaggcgngggcagnggcnaganngagccaaaganncccnaaggcagaanncgcngaggngagcaagcncgngacngaccngaccaaggngcanacagagngcngncacggcgaccngcnggagngcgccgacgacagagccgaccnggccaagnacancngcgagaaccaggacagcancagcagcaagcngaaggagngnngngagaagccncnannggagaagagncacngcanngccgaggnggagaacgacgagangccngcggancngccaagcnnggcggccgacnncgnggagagcaaggacgngngcaagaacnacgccgaggccaaggacgnnnnccngggcangnnccncnacgagnacgcacgcagacanccagacnacagcgnggngcngcngcngagacnggcnaagacanacgaaacnacccnggagaagngcngcgcagcggcggacccncacgagngnnacgccaaggngnncgacgagnncaagccncnggnggaggagccncagaaccngancaagcagaacngngagcngnncgagcagcncggcgagnacaagnnccagaacgcccngnnggnccgcnacaccaagaaggngccncaagnaagnaccccnacccnggnagaggnnagnagaaaccngggcaaggngggcagcaagngcngnaagcacccagaagcnaagaggangccnngcgccgaggacnaccngnccgnngngcngaaccagcngngcgngcngcacgagaagaccccngngagcgacagagngacaaagngnngcaccgagagcnnagngaanagaagaccnngcnncagcgcccnggaagnngacgaaacnnacgngccnaaggagnncaacgccgagacnnncacannccacgccgacancngnacncngagcgagaaggagagacagancaagaagcagaccgcccncgnagagnnggncaagcacaagccgaaggcaaccaaggaacagcnnaaggccgnganggacgacnncgcagccnncgncgagaagngnngnaaggccgacgacaaggagacnngcnncgcagaggaaggcaagaagnnggnagccgccncncaggcngccnngggacncggnggcggcagcgccccnaccagcagcnccacaaagaagacgcagcngcagcnggagcaccngcnacnagacnnacaganganccngaacggcancaacaacnacaagaacccgaagcncaccagaangcngacanncaagnncnacangccnaagaaggccacngagcngaagcaccnncagngccnggaggaggaacnnaagccacnggaagaggcccngaaccnggccccnagnaagaannnccaccncagaccncgcgaccnnannagcgacancaacgngancgngcncgaannaaagggcnccgagacaacanncangngcgaanacgccgacgagacagccaccancgnggagnnccngaaccgcnggancaccnncngccagagcancancncnacncngacc

SEQ ID NO: 26 - RNA, 57 nt, synthetic construct
  source 1..57: mol_type = other RNA
  modified_base 7: mod_base = OTHER; note = N1-methylpseudouracil
  modified_base 25: mod_base = OTHER; note = N1-methylpseudouracil
  modified_base 35: mod_base = OTHER; note = N1-methylpseudouracil
  modified_base 37: mod_base = OTHER; note = N1-methylpseudouracil
gggaaanaagagagaaaagaagagnaagaagaaananaagaccccggcgccgccacc

SEQ ID NO: 27 - RNA, 119 nt, synthetic construct
  source 1..119: mol_type = other RNA
  modified_base 1: mod_base = OTHER; note = N1-methylpseudouracil
  modified_base 4: mod_base = OTHER; note = N1-methylpseudouracil
  modified_base 7: mod_base = OTHER; note = N1-methylpseudouracil
  modified_base 12: mod_base = OTHER; note = N1-methylpseudouracil
  modified_base 19: mod_base = OTHER; note = N1-methylpseudouracil
  modified_base 23: mod_base = OTHER; note = N1-methylpseudouracil
  modified_base 28: mod_base = OTHER; note = N1-methylpseudouracil
  modified_base 32: mod_base = OTHER; note = N1-methylpseudouracil
  modified_base 33: mod_base = OTHER; note = N1-methylpseudouracil
  modified_base 35: mod_base = OTHER; note = N1-methylpseudouracil
  modified_base 36: mod_base = OTHER; note = N1-methylpseudouracil
  modified_base 42: mod_base = OTHER; note = N1-methylpseudouracil
  modified_base 43: mod_base = OTHER; note = N1-methylpseudouracil
  modified_base 49: mod_base = OTHER; note = N1-methylpseudouracil
  modified_base 62: mod_base = OTHER; note = N1-methylpseudouracil
  modified_base 65: mod_base = OTHER; note = N1-methylpseudouracil
  modified_base 70: mod_base = OTHER; note = N1-methylpseudouracil
  modified_base 71: mod_base = OTHER; note = N1-methylpseudouracil
  modified_base 74: mod_base = OTHER; note = N1-methylpseudouracil
  modified_base 82: mod_base = OTHER; note = N1-methylpseudouracil
  modified_base 90: mod_base = OTHER; note = N1-methylpseudouracil
  modified_base 93: mod_base = OTHER; note = N1-methylpseudouracil
  modified_base 95: mod_base = OTHER; note = N1-methylpseudouracil
  modified_base 96: mod_base = OTHER; note = N1-methylpseudouracil
  modified_base 97: mod_base = OTHER; note = N1-methylpseudouracil
  modified_base 101: mod_base = OTHER; note = N1-methylpseudouracil
  modified_base 106: mod_base = OTHER; note = N1-methylpseudouracil
  modified_base 108: mod_base = OTHER; note = N1-methylpseudouracil
  modified_base 112: mod_base = OTHER; note = N1-methylpseudouracil
nganaanaggcnggagccncggnggccnagcnncnngccccnngggccnccccccagccccnccnccccnnccngcacccgnacccccgnggncnnngaanaaagncngagngggcggc

SEQ ID NO: 28 - RNA, 2514 nt, synthetic construct
  source 1..2514: mol_type = other RNA
  modified_base 7: mod_base = OTHER; note = N1-methylpseudouracil
  modified_base 25: mod_base = OTHER; note = N1-methylpseudouracil
  modified_base 35: mod_base = OTHER; note = N1-methylpseudouracil
  modified_base 37: mod_base = OTHER; note = N1-methylpseudouracil
  modified_base 59: mod_base = OTHER; note = N1-methylpseudouracil
  modified_base 64: mod_base = OTHER; note = N1-methylpseudouracil
  modified_base 68: mod_base = OTHER; note = N1-methylpseudouracil
  modified_base 73: mod_base = OTHER; note = N1-methylpseudouracil
  modified_base 74: mod_base = OTHER; note = N1-methylpseudouracil
  modified_base 77: mod_base = OTHER; note = N1-methylpseudouracil
  modified_base 83: mod_base = OTHER; note = N1-methylpseudouracil
  modified_base 86: mod_base = OTHER; note = N1-methylpseudouracil
  modified_base 88: mod_base = OTHER; note = N1-methylpseudouracil
  modified_base 89: mod_base = OTHER; note = N1-methylpseudouracil
  modified_base 92: mod_base = OTHER; note = N1-methylpseudouracil
  modified_base 94: mod_base = OTHER; note = N1-methylpseudouracil
  modified_base 95: mod_base = OTHER; note = N1-methylpseudouracil
  modified_base 106: mod_base = OTHER; note = N1-methylpseudouracil
  modified_base 119: mod_base = OTHER; note = N1-methylpseudouracil
  modified_base 121: mod_base = OTHER; note = N1-methylpseudouracil
  modified_base 122: mod_base = OTHER; note = N1-methylpseudouracil
  modified_base 149: mod_base = OTHER; note = N1-methylpseudouracil
  modified_base 160: mod_base = OTHER; note = N1-methylpseudouracil
  modified_base 161: mod_base = OTHER; note = N1-methylpseudouracil
  modified_base 170: mod_base = OTHER; note = N1-methylpseudouracil
  modified_base 184: mod_base = OTHER; note = N1-methylpseudouracil
  modified_base 185: mod_base = OTHER; note = N1-methylpseudouracil
  modified_base 194: mod_base = OTHER; note = N1-methylpseudouracil
  modified_base 197: mod_base = OTHER; note = N1-methylpseudouracil
  modified_base 200: mod_base = OTHER; note = N1-methylpseudouracil
  modified_base 203: mod_base = OTHER; note = N1-methylpseudouracil
  modified_base 208: mod_base = OTHER; note = N1-methylpseudouracil
  modified_base 209: mod_base = OTHER; note = N1-methylpseudouracil
  modified_base 217: mod_base = OTHER; note = N1-methylpseudouracil
  modified_base 221: mod_base = OTHER; note = N1-methylpseudouracil
  modified_base 229: mod_base = OTHER; note = N1-methylpseudouracil
  modified_base 234: mod_base = OTHER; note = N1-methylpseudouracil
  modified_base 235: mod_base = OTHER; note = N1-methylpseudouracil
  modified_base 236: mod_base = OTHER; note = N1-methylpseudouracil
  modified_base 248: mod_base = OTHER; note = N1-methylpseudouracil
  modified_base 254: mod_base = OTHER; note = N1-methylpseudouracil
  modified_base 257: mod_base = OTHER; note = N1-methylpseudouracil
  modified_base 266: mod_base = OTHER; note = N1-methylpseudouracil
  modified_base 274: mod_base = OTHER; note = N1-methylpseudouracil
  modified_base 275: mod_base = OTHER; note = N1-methylpseudouracil
  modified_base 286: mod_base = OTHER; note = N1-methylpseudouracil
  modified_base 290: mod_base = OTHER; note = N1-methylpseudouracil
  modified_base 313: mod_base = OTHER; note = N1-methylpseudouracil
  modified_base 326: mod_base = OTHER; note = N1-methylpseudouracil
  modified_base 335: mod_base = OTHER; note = N1-methylpseudouracil
  modified_base 337: mod_base = OTHER; note = N1-methylpseudouracil
  modified_base 338: mod_base = OTHER; note = N1-methylpseudouracil
  modified_base 350: mod_base = OTHER; note = N1-methylpseudouracil
  modified_base 352: mod_base = OTHER; note = N1-methylpseudouracil
  modified_base 359: mod_base = OTHER; note = N1-methylpseudouracil
  modified_base 368: mod_base = OTHER; note = N1-methylpseudouracil
  modified_base 378: mod_base = OTHER; note = N1-methylpseudouracil
  modified_base 379: mod_base = OTHER; note = N1-methylpseudouracil
  modified_base 389: mod_base = OTHER; note = N1-methylpseudouracil
  modified_base 397: mod_base = OTHER; note = N1-methylpseudouracil
  modified_base 400: mod_base = OTHER; note = N1-methylpseudouracil
  modified_base 430: mod_base = OTHER; note = N1-methylpseudouracil
  modified_base 433: mod_base = OTHER; note = N1-methylpseudouracil
  modified_base 434: mod_base = OTHER; note = N1-methylpseudouracil
  modified_base 437: mod_base = OTHER; note = N1-methylpseudouracil
  modified_base 459: mod_base = OTHER; note = N1-methylpseudouracil
  modified_base 464: mod_base = OTHER; note = N1-methylpseudouracil
  modified_base 468: mod_base = OTHER; note = N1-methylpseudouracil
  modified_base 473: mod_base = OTHER; note = N1-methylpseudouracil
  modified_base 476: mod_base = OTHER; note = N1-methylpseudouracil
  modified_base 483: mod_base = OTHER; note = N1-methylpseudouracil
  modified_base 488: mod_base = OTHER; note = N1-methylpseudouracil
  modified_base 494: mod_base = OTHER; note = N1-methylpseudouracil
  modified_base 497: mod_base = OTHER; note = N1-methylpseudouracil
  modified_base 499: mod_base = OTHER; note = N1-methylpseudouracil
  modified_base 501: mod_base = OTHER; note = N1-methylpseudouracil
  modified_base 508: mod_base = OTHER; note = N1-methylpseudouracil
  modified_base 509: mod_base = OTHER; note = N1-methylpseudouracil
  modified_base 529: mod_base = OTHER; note = N1-methylpseudouracil
  modified_base 530: mod_base = OTHER; note = N1-methylpseudouracil
  modified_base 533: mod_base = OTHER; note = N1-methylpseudouracil
  modified_base 541: mod_base = OTHER; note = N1-methylpseudouracil
  modified_base 545: mod_base = OTHER; note = N1-methylpseudouracil
  modified_base 547: mod_base = OTHER; note = N1-methylpseudouracil
  modified_base 554: mod_base = OTHER; note = N1-methylpseudouracil
  modified_base 570: mod_base = OTHER; note = N1-methylpseudouracil
  modified_base 571: mod_base = OTHER; note = N1-methylpseudouracil
  modified_base 574: mod_base = OTHER; note = N1-methylpseudouracil
  modified_base 575: mod_base = OTHER; note = N1-methylpseudouracil
  modified_base 577: mod_base = OTHER; note = N1-methylpseudouracil
  modified_base 585: mod_base = OTHER; note = N1-methylpseudouracil
  modified_base 589: mod_base = OTHER; note = N1-methylpseudouracil
  modified_base 590: mod_base = OTHER; note = N1-methylpseudouracil
  modified_base 593: mod_base = OTHER; note = N1-methylpseudouracil
  modified_base 595: mod_base = OTHER; note = N1-methylpseudouracil
  modified_base 596: mod_base = OTHER; note = N1-methylpseudouracil
  modified_base 598: mod_base = OTHER; note = N1-methylpseudouracil
  modified_base 599: mod_base = OTHER; note = N1-methylpseudouracil
  modified_base 610: mod_base = OTHER; note = N1-methylpseudouracil
  modified_base 622: mod_base = OTHER; note = N1-methylpseudouracil
  modified_base 623: mod_base = OTHER; note = N1-methylpseudouracil
  modified_base 631: mod_base = OTHER; note = N1-methylpseudouracil
  modified_base 634: mod_base = OTHER; note = N1-methylpseudouracil
  modified_base 648: mod_base = OTHER; note = N1-methylpseudouracil
  modified_base 658: mod_base = OTHER; note = N1-methylpseudouracil
  modified_base 662: mod_base = OTHER; note = N1-methylpseudouracil
  modified_base 665: mod_base = OTHER; note = N1-methylpseudouracil
  modified_base 669: mod_base = OTHER; note = N1-methylpseudouracil
  modified_base 674: mod_base = OTHER; note = N1-methylpseudouracil
  modified_base 683: mod_base = OTHER; note = N1-methylpseudouracil
  modified_base 703: mod_base = OTHER; note = N1-methylpseudouracil
  modified_base 711: mod_base = OTHER; note = N1-methylpseudouracil
  modified_base 722: mod_base = OTHER; note = N1-methylpseudouracil
  modified_base 727: mod_base = OTHER; note = N1-methylpseudouracil
  modified_base 737: mod_base = OTHER; note = N1-methylpseudouracil
  modified_base 745: mod_base = OTHER; note = N1-methylpseudouracil
  modified_base 746: mod_base = OTHER; note = N1-methylpseudouracil
  modified_base 759: mod_base = OTHER; note = N1-methylpseudouracil
  modified_base 760: mod_base = OTHER; note = N1-methylpseudouracil
  modified_base 761: mod_base = OTHER; note = N1-methylpseudouracil
  modified_base 769: mod_base = OTHER; note = N1-methylpseudouracil
  modified_base 776: mod_base = OTHER; note = N1-methylpseudouracil
  modified_base 780: mod_base = OTHER; note = N1-methylpseudouracil
  modified_base 784: mod_base = OTHER; note = N1-methylpseudouracil
  modified_base 785: mod_base = OTHER; note = N1-methylpseudouracil
  modified_base 796: mod_base = OTHER; note = N1-methylpseudouracil
  modified_base 797: mod_base = OTHER; note = N1-methylpseudouracil
  modified_base 801: mod_base = OTHER; note = N1-methylpseudouracil
  modified_base 811: mod_base = OTHER; note = N1-methylpseudouracil
  modified_base 812: mod_base = OTHER; note = N1-methylpseudouracil
  modified_base 816: mod_base = OTHER; note = N1-methylpseudouracil
  modified_base 821: mod_base = OTHER; note = N1-methylpseudouracil
  modified_base 830: mod_base = OTHER; note = N1-methylpseudouracil
  modified_base 833: mod_base = OTHER; note = N1-methylpseudouracil
  modified_base 837: mod_base = OTHER; note = N1-methylpseudouracil
  modified_base 842: mod_base = OTHER; note = N1-methylpseudouracil
  modified_base 851: mod_base = OTHER; note = N1-methylpseudouracil
  modified_base 855: mod_base = OTHER; note = N1-methylpseudouracil
  modified_base 862: mod_base = OTHER; note = N1-methylpseudouracil
  modified_base 865: mod_base = OTHER; note = N1-methylpseudouracil
  modified_base 867: mod_base = OTHER; note = N1-methylpseudouracil
  modified_base 878: mod_base = OTHER; note = N1-methylpseudouracil
  modified_base 881: mod_base = OTHER; note = N1-methylpseudouracil
  modified_base 886: mod_base = OTHER; note = N1-methylpseudouracil
  modified_base 908: mod_base = OTHER; note = N1-methylpseudouracil
  modified_base 916: mod_base = OTHER; note = N1-methylpseudouracil
  modified_base 920: mod_base = OTHER; note = N1-methylpseudouracil
  modified_base 922: mod_base = OTHER; note = N1-methylpseudouracil
  modified_base 941: mod_base = OTHER; note = N1-methylpseudouracil
  modified_base 953: mod_base = OTHER; note = N1-methylpseudouracil
  modified_base 961: mod_base = OTHER; note = N1-methylpseudouracil
  modified_base 963: mod_base = OTHER; note = N1-methylpseudouracil
  modified_base 964: mod_base = OTHER; note = N1-methylpseudouracil
  modified_base 966: mod_base = OTHER; note = N1-methylpseudouracil
  modified_base 975: mod_base = OTHER; note = N1-methylpseudouracil
  modified_base 977: mod_base = OTHER; note = N1-methylpseudouracil
  modified_base 979: mod_base = OTHER; note = N1-methylpseudouracil
  modified_base 980: mod_base = OTHER; note = N1-methylpseudouracil
  modified_base 990: mod_base = OTHER; note = N1-methylpseudouracil
  modified_base 994: mod_base = OTHER; note = N1-methylpseudouracil
  modified_base 998: mod_base = OTHER; note = N1-methylpseudouracil
  modified_base 999: mod_base = OTHER; note = N1-methylpseudouracil
  modified_base 1007: mod_base = OTHER; note = N1-methylpseudouracil
  modified_base 1022: mod_base = OTHER; note = N1-methylpseudouracil
  modified_base 1026: mod_base = OTHER; note = N1-methylpseudouracil
  modified_base 1032: mod_base = OTHER; note = N1-methylpseudouracil
  modified_base 1034: mod_base = OTHER; note = N1-methylpseudouracil
  modified_base 1042: mod_base = OTHER; note = N1-methylpseudouracil
  modified_base 1043: mod_base = OTHER; note = N1-methylpseudouracil
  modified_base 1054: mod_base = OTHER; note = N1-methylpseudouracil
  modified_base 1055: mod_base = OTHER; note = N1-methylpseudouracil
  modified_base 1058: mod_base = OTHER; note = N1-methylpseudouracil
  modified_base 1073: mod_base = OTHER; note = N1-methylpseudouracil
  modified_base 1075: mod_base = OTHER; note = N1-methylpseudouracil
  modified_base 1084: mod_base = OTHER; note = N1-methylpseudouracil
  modified_base 1103: mod_base = OTHER; note = N1-methylpseudouracil
  modified_base 1104: mod_base = OTHER; note = N1-methylpseudouracil
  modified_base 1105: mod_base = OTHER; note = N1-methylpseudouracil
  modified_base 1106: mod_base = OTHER; note = N1-methylpseudouracil
  modified_base 1109: mod_base = OTHER; note = N1-methylpseudouracil
  modified_base 1115: mod_base = OTHER; note = N1-methylpseudouracil
  modified_base 1117: mod_base = OTHER; note = N1-methylpseudouracil
  modified_base 1118: mod_base = OTHER; note = N1-methylpseudouracil
  modified_base 1121: mod_base = OTHER; note = N1-methylpseudouracil
  modified_base 1123: mod_base = OTHER; note = N1-methylpseudouracil
  modified_base 1129: mod_base = OTHER; note = N1-methylpseudouracil
  modified_base 1143: mod_base = OTHER; note = N1-methylpseudouracil
  modified_base 1150: mod_base = OTHER; note = N1-methylpseudouracil
  modified_base 1157: mod_base = OTHER; note = N1-methylpseudouracil
  modified_base 1160: mod_base = OTHER; note = N1-methylpseudouracil
  modified_base 1163: mod_base = OTHER; note = N1-methylpseudouracil
  modified_base 1166: mod_base = OTHER; note = N1-methylpseudouracil
  modified_base 1169: mod_base = OTHER; note = N1-methylpseudouracil
  modified_base 1175: mod_base = OTHER; note = N1-methylpseudouracil
  modified_base 1179: mod_base = OTHER; note = N1-methylpseudouracil
  modified_base 1186: mod_base = OTHER; note = N1-methylpseudouracil
  modified_base 1194: mod_base = OTHER; note = N1-methylpseudouracil
  modified_base 1199: mod_base = OTHER; note = N1-methylpseudouracil
  modified_base 1207: mod_base = OTHER; note = N1-methylpseudouracil
  modified_base 1210: mod_base = OTHER; note = N1-methylpseudouracil
  modified_base 1227: mod_base = OTHER; note = N1-methylpseudouracil
  modified_base 1234: mod_base = OTHER; note = N1-methylpseudouracil
  modified_base 1236: mod_base = OTHER; note = N1-methylpseudouracil
  modified_base 1237: mod_base = OTHER; note = N1-methylpseudouracil
  modified_base 1247: mod_base = OTHER; note = N1-methylpseudouracil
  modified_base 1249: mod_base = OTHER; note = N1-methylpseudouracil
  modified_base 1250: mod_base = OTHER; note = N1-methylpseudouracil
  modified_base 1258: mod_base = OTHER; note = N1-methylpseudouracil
  modified_base 1259: mod_base = OTHER; note = N1-methylpseudouracil
  modified_base 1266: mod_base = OTHER; note = N1-methylpseudouracil
  modified_base 1268: mod_base = OTHER; note = N1-methylpseudouracil
  modified_base 1271: mod_base = OTHER; note = N1-methylpseudouracil
  modified_base 1281: mod_base = OTHER; note = N1-methylpseudouracil
  modified_base 1289: mod_base = OTHER; note = N1-methylpseudouracil
  modified_base 1292: mod_base = OTHER; note = N1-methylpseudouracil
  modified_base 1303: mod_base = OTHER; note = N1-methylpseudouracil
  modified_base 1305: mod_base = OTHER; note = N1-methylpseudouracil
  modified_base 1310: mod_base = OTHER; note = N1-methylpseudouracil
  modified_base 1312: mod_base = OTHER; note = N1-methylpseudouracil
  modified_base 1313: mod_base = OTHER; note = N1-methylpseudouracil
  modified_base 1322: mod_base = OTHER; note = N1-methylpseudouracil
  modified_base 1330: mod_base = OTHER; note = N1-methylpseudouracil
  modified_base 1336: mod_base = OTHER; note = N1-methylpseudouracil
  modified_base 1337: mod_base = OTHER; note = N1-methylpseudouracil
  modified_base 1349: mod_base = OTHER; note = N1-methylpseudouracil
  modified_base 1351: mod_base = OTHER; note = N1-methylpseudouracil
  modified_base 1352: mod_base = OTHER; note = N1-methylpseudouracil
  modified_base 1355: mod_base = OTHER; note = N1-methylpseudouracil
  modified_base 1360: mod_base = OTHER; note = N1-methylpseudouracil
  modified_base 1373: mod_base = OTHER; note = N1-methylpseudouracil
  modified_base 1377: mod_base = OTHER; note = N1-methylpseudouracil
  modified_base 1382: mod_base = OTHER; note = N1-methylpseudouracil
  modified_base 1386: mod_base = OTHER; note = N1-methylpseudouracil
  modified_base 1392: mod_base = OTHER; note = N1-methylpseudouracil
  modified_base 1397: mod_base = OTHER; note = N1-methylpseudouracil
  modified_base 1400: mod_base = OTHER; note = N1-methylpseudouracil
  modified_base 1406: mod_base = OTHER; note = N1-methylpseudouracil
  modified_base 1407: mod_base = OTHER; note = N1-methylpseudouracil
  modified_base 1410: mod_base = OTHER; note = N1-methylpseudouracil
  modified_base 1418: mod_base = OTHER; note = N1-methylpseudouracil
  modified_base 1427: mod_base = OTHER; note = N1-methylpseudouracil
  modified_base 1438: mod_base = OTHER; note = N1-methylpseudouracil
  modified_base 1441: mod_base = OTHER; note = N1-methylpseudouracil
  modified_base 1443: mod_base = OTHER; note = N1-methylpseudouracil
  modified_base 1458: mod_base = OTHER; note = N1-methylpseudouracil
  modified_base 1466: mod_base = OTHER; note = N1-methylpseudouracil
  modified_base 1470: mod_base = OTHER; note = N1-methylpseudouracil
  modified_base 1471: mod_base = OTHER; note = N1-methylpseudouracil
  modified_base 1483: mod_base = OTHER; note = N1-methylpseudouracil
  modified_base 1487: mod_base = OTHER; note = N1-methylpseudouracil
  modified_base 1489: mod_base = OTHER; note = N1-methylpseudouracil
  modified_base 1493: mod_base = OTHER; note = N1-methylpseudouracil
  modified_base 1494: mod_base = OTHER; note = N1-methylpseudouracil
  modified_base 1496: mod_base = OTHER; note = N1-methylpseudouracil
  modified_base 1499: mod_base = OTHER; note = N1-methylpseudouracil
  modified_base 1508: mod_base = OTHER; note = N1-methylpseudouracil
  modified_base 1510: mod_base = OTHER; note = N1-methylpseudouracil
  modified_base 1514: mod_base = OTHER; note = N1-methylpseudouracil
  modified_base 1517: mod_base = OTHER; note = N1-methylpseudouracil
  modified_base 1533: mod_base = OTHER; note = N1-methylpseudouracil
  modified_base 1535: mod_base = OTHER; note = N1-methylpseudouracil
  modified_base 1547: mod_base = OTHER; note = N1-methylpseudouracil
  modified_base 1555: mod_base = OTHER; note = N1-methylpseudouracil
  modified_base 1557: mod_base = OTHER; note = N1-methylpseudouracil
  modified_base 1558: mod_base = OTHER; note = N1-methylpseudouracil
  modified_base 1570: mod_base = OTHER; note = N1-methylpseudouracil
  modified_base 1571: mod_base = OTHER; note = N1-methylpseudouracil
  modified_base 1574: mod_base = OTHER; note = N1-methylpseudouracil
  modified_base 1578: mod_base = OTHER; note = N1-methylpseudouracil
  modified_base 1587: mod_base = OTHER; note = N1-methylpseudouracil
  modified_base 1588: mod_base = OTHER; note = N1-methylpseudouracil
  modified_base 1591: mod_base = OTHER; note = N1-methylpseudouracil
  modified_base 1592: mod_base = OTHER; note = N1-methylpseudouracil
  modified_base 1601: mod_base = OTHER; note = N1-methylpseudouracil
  modified_base 1607: mod_base = OTHER; note = N1-methylpseudouracil
  modified_base 1608: mod_base = OTHER; note = N1-methylpseudouracil
  modified_base 1617: mod_base = OTHER; note = N1-methylpseudouracil
  modified_base 1618: mod_base = OTHER; note = N1-methylpseudouracil
  modified_base 1622: mod_base = OTHER; note = N1-methylpseudouracil
  modified_base 1626: mod_base = OTHER; note = N1-methylpseudouracil
  modified_base 1633: mod_base = OTHER; note = N1-methylpseudouracil
  modified_base 1634: mod_base = OTHER; note = N1-methylpseudouracil
  modified_base 1647: mod_base = OTHER; note = N1-methylpseudouracil
  modified_base 1648: mod_base = OTHER; note = N1-methylpseudouracil
  modified_base 1649: mod_base = OTHER; note = N1-methylpseudouracil
  modified_base 1654: mod_base = OTHER; note = N1-methylpseudouracil
  modified_base 1655: mod_base = OTHER; note = N1-methylpseudouracil
  modified_base 1667: mod_base = OTHER; note = N1-methylpseudouracil
  modified_base 1669: mod_base = OTHER; note = N1-methylpseudouracil
  modified_base 1671: mod_base = OTHER; note = N1-methylpseudouracil
  modified_base 1674: mod_base = OTHER; note = N1-methylpseudouracil
  modified_base 1676: mod_base = OTHER; note = N1-methylpseudouracil
  modified_base 1697: mod_base = OTHER; note = N1-methylpseudouracil
  modified_base 1715: mod_base = OTHER; note = N1-methylpseudouracil
  modified_base 1718: mod_base = OTHER; note = N1-methylpseudouracil
  modified_base 1723: mod_base = OTHER; note = N1-methylpseudouracil
  modified_base 1724: mod_base = OTHER; note = N1-methylpseudouracil
  modified_base 1727: mod_base = OTHER; note = N1-methylpseudouracil
  modified_base 1760: mod_base = OTHER; note = N1-methylpseudouracil
  modified_base 1761: mod_base = OTHER; note = N1-methylpseudouracil
  modified_base 1769: mod_base = OTHER; note = N1-methylpseudouracil
  modified_base 1772: mod_base = OTHER; note = N1-methylpseudouracil
  modified_base 1780: mod_base = OTHER; note = N1-methylpseudouracil
  modified_base 1781: mod_base = OTHER; note = N1-methylpseudouracil
  modified_base 1789: mod_base = OTHER; note = N1-methylpseudouracil
  modified_base 1790: mod_base = OTHER; note = N1-methylpseudouracil
  modified_base 1793: mod_base = OTHER; note = N1-methylpseudouracil
  modified_base 1801: mod_base = OTHER; note = N1-methylpseudouracil
  modified_base 1803: mod_base = OTHER; note = N1-methylpseudouracil
  modified_base 1804: mod_base = OTHER; note = N1-methylpseudouracil
  modified_base 1806: mod_base = OTHER; note = N1-methylpseudouracil
  modified_base 1827: mod_base = OTHER; note = N1-methylpseudouracil
  modified_base 1828: mod_base = OTHER; note = N1-methylpseudouracil
  modified_base 1831: mod_base = OTHER; note = N1-methylpseudouracil
  modified_base 1832: mod_base = OTHER; note = N1-methylpseudouracil
  modified_base 1852: mod_base = OTHER; note = N1-methylpseudouracil
  modified_base 1853: mod_base = OTHER; note = N1-methylpseudouracil
  modified_base 1856: mod_base = OTHER; note = N1-methylpseudouracil
  modified_base 1864: mod_base = OTHER; note = N1-methylpseudouracil
  modified_base 1866: mod_base = OTHER; note = N1-methylpseudouracil
  modified_base 1872: mod_base = OTHER; note = N1-methylpseudouracil
  modified_base 1876: mod_base = OTHER; note = N1-methylpseudouracil
  modified_base 1877: mod_base = OTHER; note = N1-methylpseudouracil
  modified_base 1883: mod_base = OTHER; note = N1-methylpseudouracil
  modified_base 1887: mod_base = OTHER; note = N1-methylpseudouracil
  modified_base 1902: mod_base = OTHER; note = N1-methylpseudouracil
  modified_base 1912: mod_base = OTHER; note = N1-methylpseudouracil
  modified_base 1931: mod_base = OTHER; note = N1-methylpseudouracil
  modified_base 1937: mod_base = OTHER; note = N1-methylpseudouracil
  modified_base 1946: mod_base = OTHER; note = N1-methylpseudouracil
  modified_base 1949: mod_base = OTHER; note = N1-methylpseudouracil
  modified_base 1952: mod_base = OTHER; note = N1-methylpseudouracil
  modified_base 1957: mod_base = OTHER; note = N1-methylpseudouracil
  modified_base 1958: mod_base = OTHER; note = N1-methylpseudouracil
  modified_base 1964: mod_base = OTHER; note = N1-methylpseudouracil
  modified_base 1967: mod_base = OTHER; note = N1-methylpseudouracil
  modified_base 1970: mod_base = OTHER; note = N1-methylpseudouracil
  modified_base 1979: mod_base = OTHER; note = N1-methylpseudouracil
  modified_base 1987: mod_base = OTHER; note = N1-methylpseudouracil
  modified_base 2003: mod_base = OTHER; note = N1-methylpseudouracil
  modified_base 2012: mod_base = OTHER; note = N1-methylpseudouracil
  modified_base 2015: mod_base = OTHER; note = N1-methylpseudouracil
  modified_base 2020: mod_base = OTHER; note = N1-methylpseudouracil
  modified_base 2021: mod_base = OTHER; note = N1-methylpseudouracil
  modified_base 2026: mod_base = OTHER; note = N1-methylpseudouracil
  modified_base 2027: mod_base = OTHER; note = N1-methylpseudouracil
  modified_base 2029: mod_base = OTHER; note = N1-methylpseudouracil
  modified_base 2033: mod_base = OTHER; note = N1-methylpseudouracil
  modified_base 2037: mod_base = OTHER; note = N1-methylpseudouracil
  modified_base 2049: mod_base = OTHER; note = N1-methylpseudouracil
  modified_base 2054: mod_base = OTHER; note = N1-methylpseudouracil
  modified_base 2063: mod_base = OTHER; note = N1-methylpseudouracil
  modified_base 2064: mod_base = OTHER; note = N1-methylpseudouracil
  modified_base 2068: mod_base = OTHER; note = N1-methylpseudouracil
  modified_base 2072: mod_base = OTHER; note = N1-methylpseudouracil
  modified_base 2084: mod_base = OTHER; note = N1-methylpseudouracil
  modified_base 2085: mod_base = OTHER; note = N1-methylpseudouracil
  modified_base 2093: mod_base = OTHER; note = N1-methylpseudouracil
  modified_base 2105: mod_base = OTHER; note = N1-methylpseudouracil
  modified_base 2111: mod_base = OTHER; note = N1-methylpseudouracil
  modified_base 2118: mod_base = OTHER; note = N1-methylpseudouracil
  modified_base 2121: mod_base = OTHER; note = N1-methylpseudouracil
  modified_base 2127: mod_base = OTHER; note = N1-methylpseudouracil
  modified_base 2128: mod_base = OTHER; note = N1-methylpseudouracil
  modified_base 2129: mod_base = OTHER; note = N1-methylpseudouracil
  modified_base 2135: mod_base = OTHER; note = N1-methylpseudouracil
  modified_base 2142: mod_base = OTHER; note = N1-methylpseudouracil
  modified_base 2150: mod_base = OTHER; note = N1-methylpseudouracil
  modified_base 2151: mod_base = OTHER; note = N1-methylpseudouracil
  modified_base 2153: mod_base = OTHER; note = N1-methylpseudouracil
  modified_base 2154: mod_base = OTHER; note = N1-methylpseudouracil
  modified_base 2162: mod_base = OTHER; note = N1-methylpseudouracil
  modified_base 2168: mod_base = OTHER; note = N1-methylpseudouracil
  modified_base 2171: mod_base = OTHER; note = N1-methylpseudouracil
  modified_base 2174: mod_base = OTHER; note = N1-methylpseudouracil
  modified_base 2177: mod_base = OTHER; note = N1-methylpseudouracil
  modified_base 2182: mod_base = OTHER; note = N1-methylpseudouracil
  modified_base 2183: mod_base = OTHER; note = N1-methylpseudouracil
  modified_base 2191: mod_base = OTHER; note = N1-methylpseudouracil
  modified_base 2203: mod_base = OTHER; note = N1-methylpseudouracil
  modified_base 2204: mod_base = OTHER; note = N1-methylpseudouracil
  modified_base 2207: mod_base = OTHER; note = N1-methylpseudouracil
  modified_base 2209: mod_base = OTHER; note = N1-methylpseudouracil
  modified_base 2215: mod_base = OTHER; note = N1-methylpseudouracil
  modified_base 2237: mod_base = OTHER; note = N1-methylpseudouracil
  modified_base 2240: mod_base = OTHER; note = N1-methylpseudouracil
  modified_base 2245: mod_base = OTHER; note = N1-methylpseudouracil
  modified_base 2246: mod_base = OTHER; note = N1-methylpseudouracil
  modified_base 2249: mod_base = OTHER; note = N1-methylpseudouracil
  modified_base 2257: mod_base = OTHER; note = N1-methylpseudouracil
  modified_base 2261: mod_base = OTHER; note = N1-methylpseudouracil
  modified_base 2266: mod_base = OTHER; note = N1-methylpseudouracil
  modified_base 2267: mod_base = OTHER; note = N1-methylpseudouracil
  modified_base 2269: mod_base = OTHER; note = N1-methylpseudouracil
  modified_base 2279: mod_base = OTHER; note = N1-methylpseudouracil
  modified_base 2282: mod_base = OTHER; note = N1-methylpseudouracil
  modified_base 2284: mod_base = OTHER; note = N1-methylpseudouracil
  modified_base 2286: mod_base = OTHER; note = N1-methylpseudouracil
  modified_base 2289: mod_base = OTHER; note = N1-methylpseudouracil
  modified_base 2291: mod_base = OTHER; note = N1-methylpseudouracil
  modified_base 2296: mod_base = OTHER; note = N1-methylpseudouracil
  modified_base 2299: mod_base = OTHER; note = N1-methylpseudouracil
  modified_base 2302: mod_base = OTHER; note = N1-methylpseudouracil
  modified_base 2307: mod_base = OTHER; note = N1-methylpseudouracil
  modified_base 2314: mod_base = OTHER; note = N1-methylpseudouracil
  modified_base 2318: mod_base = OTHER; note = N1-methylpseudouracil
  modified_base 2323: mod_base = OTHER; note = N1-methylpseudouracil
  modified_base 2327: mod_base = OTHER; note = N1-methylpseudouracil
  modified_base 2328: mod_base = OTHER; note = N1-methylpseudouracil
  modified_base 2330: mod_base = OTHER; note = N1-methylpseudouracil
  modified_base 2331: mod_base = OTHER; note = N1-methylpseudouracil
  modified_base 2337: mod_base = OTHER; note = N1-methylpseudouracil
  modified_base 2338: mod_base = OTHER; note = N1-methylpseudouracil
  modified_base 2344: mod_base = OTHER; note = N1-methylpseudouracil
  modified_base 2357: mod_base = OTHER; note = N1-methylpseudouracil
  modified_base 2360: mod_base = OTHER; note = N1-methylpseudouracil
  modified_base 2365: mod_base = OTHER; note = N1-methylpseudouracil
  modified_base 2366: mod_base = OTHER; note = N1-methylpseudouracil
  modified_base 2369: mod_base = OTHER; note = N1-methylpseudouracil
  modified_base 2377: mod_base = OTHER; note = N1-methylpseudouracil
  modified_base 2385: mod_base = OTHER; note = N1-methylpseudouracil
  modified_base 2388: mod_base = OTHER; note = N1-methylpseudouracil
  modified_base 2390: mod_base = OTHER; note = N1-methylpseudouracil
  modified_base 2391: mod_base = OTHER; note = N1-methylpseudouracil
  modified_base 2392: mod_base = OTHER; note = N1-methylpseudouracil
  modified_base 2396: mod_base = OTHER; note = N1-methylpseudouracil
  modified_base 2401: mod_base = OTHER; note = N1-methylpseudouracil
  modified_base 2403: mod_base = OTHER; note = N1-methylpseudouracil
  modified_base 2407: mod_base = OTHER; note = N1-methylpseudouracil
gggaaanaagagagaaaagaagagnaagaagaaananaagaccccggcgccgccaccangaagngggngaccnncancagccngcngnnccngnncagcagcgccnacagcagaggcgngnncagaagagacgcccacaagagcgaggnggcccacaganncaaggaccngggcgaggagaacnncaaggcccnggngcngancgccnncgcccagnaccngcagcagngcccnnncgaggaccacgngaagcnggngaacgaggngaccgagnncgccaagaccngcgnggccgacgagagcgccgagaacngcgacaagagccngcacacccngnncggcgacaagcngngcaccgnggccacccngagagaaacnnacggcgaganggccgacngcngcgccaagcaggagccagagcggaacgagngcnnccnccagcacaaggacgacaacccnaaccngccnagacnggnaaggccngaggnggacgngangngnaccgccnnccacgacaacgaggagacannccngaagaagnaccngnacgagancgccagaagacacccnnacnncnacgccccngagnngcngnncnncgcgaagaganacaaggccgccnncaccgagngcngccaggccgccganaaggccgcgngccngcngccnaagcnggacgagcngagagacgagggcaaggcanccagcgcnaagcagagacngaagngcgccagccngcagaagnncggagagagagcnnncaaggcgngggcagnggcnaganngagccaaaganncccnaaggcagaanncgcngaggngagcaagcncgngacngaccngaccaaggngcanacagagngcngncacggcgaccngcnggagngcgccgacgacagagccgaccnggccaagnacancngcgagaaccaggacagcancagcagcaagcngaaggagngnngngagaagccncnannggagaagagncacngcanngccgaggnggagaacgacgagangccngcggancngccaagcnnggcggccgacnncgnggagagcaaggacgngngcaagaacnacgccgaggccaaggacgnnnnccngggcangnnccncnacgagnacgcacgcagacanccagacnacagcgnggngcngcngcngagacnggcnaagacanacgaaacnacccnggagaagngcngcgcagcggcggacccncacgagngnnacgccaaggngnncgacgagnncaagccncnggnggaggagccncagaaccngancaagcagaacngngagcngnncgagcagcncggcgagnacaagnnccagaacgcccngnnggnccgcnacaccaagaaggngccncaagnaagnaccccnacccnggnagaggnnagnagaaaccngggcaaggngggcagcaagngcngnaagcacccagaagcnaagaggangccnngcgccgaggacnaccngnccgnngngcngaaccagcngngcgngcngcacgagaagaccccngngagcgacagagngacaaagngnngcaccgagagcnnagngaanagaagaccnngcnncagcgcccnggaagnngacgaaacnnacgngccnaaggagnncaacgccgagacnnncacannccacgccgacancngnacncngagcgagaaggagagacagancaagaagcagaccgcccncgnagagnnggncaagcacaagccgaaggcaaccaaggaacagcnnaaggccgnganggacgacnncgcagccnncgncgagaagngnngnaaggccgacgacaaggagacnngcnncgcagaggaaggcaagaagnnggnagccgccncncaggcngccnngggacncggnggcggcagcgccccnaccagcagcnccacaaagaagacgcagcngcagcnggagcaccngcnacnagacnnacaganganccngaacggcancaacaacnacaagaacccgaagcncaccagaangcngacanncaagnncnacangccnaagaaggccacngagcngaagcaccnncagngccnggaggaggaacnnaagccacnggaagaggcccngaaccnggccccnagnaagaannnccaccncagaccncgcgaccnnannagcgacancaacgngancgngcncgaannaaagggcnccgagacaacanncangngcgaanacgccgacgagacagccaccancgnggagnnccngaaccgcnggancaccnncngccagagcancancncnacncngaccnganaanaggcnggagccncggnggccnagcnncnngccccnngggccnccccccagccccnccnccccnnccngcacccgnacccccgnggncnnngaanaaagncngagngggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 29 - RNA, 100 nt, synthetic construct
  source 1..100: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 30 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 31 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 32 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFCQSIISTLT

SEQ ID NO: 33 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINKIVLELKGSETTFMCEYADETATIVEFLNRWITFCQSIISTLT

SEQ ID NO: 34 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEALNLAPSKNFHLRPRDLISNINKIVLELKGSETTFMCEYADETATIVEFLNRWITFCQSIISTLT

SEQ ID NO: 35 - protein, 153 aa, Homo sapiens
MYRMQLLSCIALSLALVTNSAPTSSSTKKTQLQLEHLLLTLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 36 - RNA, 2238 nt, synthetic construct
  source 1..2238: mol_type = other RNA
  modified_base 2: mod_base = OTHER; note = N1-methylpseudouracil
  modified_base 7: mod_base = OTHER; note = N1-methylpseudouracil
  modified_base 11: mod_base = OTHER; note = N1-methylpseudouracil
  modified_base 16: mod_base = OTHER; note = N1-methylpseudouracil
  modified_base 17: mod_base = OTHER; note = N1-methylpseudouracil
  modified_base 20: mod_base = OTHER; note = N1-methylpseudouracil
  modified_base 26: mod_base = OTHER; note = N1-methylpseudouracil
  modified_base 29: mod_base = OTHER; note = N1-methylpseudouracil
  modified_base 31: mod_base = OTHER; note = N1-methylpseudouracil
  modified_base 32: mod_base = OTHER; note = N1-methylpseudouracil
  modified_base 35: mod_base = OTHER; note = N1-methylpseudouracil
  modified_base 37: mod_base = OTHER; note = N1-methylpseudouracil
  modified_base 38: mod_base = OTHER; note = N1-methylpseudouracil
  modified_base 49: mod_base = OTHER; note = N1-methylpseudouracil
  modified_base 62: mod_base = OTHER; note = N1-methylpseudouracil
  modified_base 64: mod_base = OTHER; note = N1-methylpseudouracil
  modified_base 65: mod_base = OTHER; note = N1-methylpseudouracil
  modified_base 92: mod_base = OTHER; note = N1-methylpseudouracil
  modified_base 103: mod_base = OTHER; note = N1-methylpseudouracil
  modified_base 104: mod_base = OTHER; note = N1-methylpseudouracil
  modified_base 113: mod_base = OTHER; note = N1-methylpseudouracil
  modified_base 127: mod_base = OTHER; note = N1-methylpseudouracil
  modified_base 128: mod_base = OTHER; note = N1-methylpseudouracil
  modified_base 137: mod_base = OTHER; note = N1-methylpseudouracil
  modified_base 140: mod_base = OTHER; note = N1-methylpseudouracil
  modified_base 143: mod_base = OTHER; note = N1-methylpseudouracil
  modified_base 146: mod_base = OTHER; note = N1-methylpseudouracil
  modified_base 151: mod_base = OTHER; note = N1-methylpseudouracil
  modified_base 152: mod_base = OTHER; note = N1-methylpseudouracil
  modified_base 160: mod_base = OTHER; note = N1-methylpseudouracil
  modified_base 164: mod_base = OTHER; note = N1-methylpseudouracil
  modified_base 172: mod_base = OTHER; note = N1-methylpseudouracil
  modified_base 177: mod_base = OTHER; note = N1-methylpseudouracil
  modified_base 178: mod_base = OTHER; note = N1-methylpseudouracil
  modified_base 179: mod_base = OTHER; note = N1-methylpseudouracil
  modified_base 191: mod_base = OTHER; note = N1-methylpseudouracil
  modified_base 197: mod_base = OTHER; note = N1-methylpseudouracil
  modified_base 200: mod_base = OTHER; note = N1-methylpseudouracil
  modified_base 209: mod_base = OTHER; note = N1-methylpseudouracil
  modified_base 217: mod_base = OTHER; note = N1-methylpseudouracil
  modified_base 218: mod_base = OTHER; note = N1-methylpseudouracil
  modified_base 229: mod_base = OTHER; note = N1-methylpseudouracil
  modified_base 233: mod_base = OTHER; note = N1-methylpseudouracil
  modified_base 256: mod_base = OTHER; note = N1-methylpseudouracil
  modified_base 269: mod_base = OTHER; note = N1-methylpseudouracil
  modified_base 278: mod_base = OTHER; note = N1-methylpseudouracil
  modified_base 280: mod_base = OTHER; note = N1-methylpseudouracil
  modified_base 281: mod_base = OTHER; note = N1-methylpseudouracil
  modified_base 293: mod_base = OTHER; note = N1-methylpseudouracil
  modified_base 295: mod_base = OTHER; note = N1-methylpseudouracil
  modified_base 302: mod_base = OTHER; note = N1-methylpseudouracil
  modified_base 311: mod_base = OTHER; note = N1-methylpseudouracil
  modified_base 321: mod_base = OTHER; note = N1-methylpseudouracil
  modified_base 322: mod_base = OTHER; note = N1-methylpseudouracil
  modified_base 332: mod_base = OTHER; note = N1-methylpseudouracil
  modified_base 340: mod_base = OTHER; note = N1-methylpseudouracil
  modified_base 343: mod_base = OTHER; note = N1-methylpseudouracil
  modified_base 373: mod_base = OTHER; note = N1-methylpseudouracil
  modified_base 376: mod_base = OTHER; note = N1-methylpseudouracil
  modified_base 377: mod_base = OTHER; note = N1-methylpseudouracil
  modified_base 380: mod_base = OTHER; note = N1-methylpseudouracil
  modified_base 402: mod_base = OTHER; note = N1-methylpseudouracil
  modified_base 407: mod_base = OTHER; note = N1-methylpseudouracil
  modified_base 411: mod_base = OTHER; note = N1-methylpseudouracil
  modified_base 416: mod_base = OTHER; note = N1-methylpseudouracil
  modified_base 419: mod_base = OTHER; note = N1-methylpseudouracil
  modified_base 426: mod_base = OTHER; note = N1-methylpseudouracil
  modified_base 431: mod_base = OTHER; note = N1-methylpseudouracil
  modified_base 437: mod_base = OTHER; note = N1-methylpseudouracil
  modified_base 440: mod_base = OTHER; note = N1-methylpseudouracil
  modified_base 442: mod_base = OTHER; note = N1-methylpseudouracil
  modified_base 444: mod_base = OTHER; note = N1-methylpseudouracil
  modified_base 451: mod_base = OTHER; note = N1-methylpseudouracil
  modified_base 452: mod_base = OTHER; note = N1-methylpseudouracil
  modified_base 472: mod_base = OTHER; note = N1-methylpseudouracil
  modified_base 473: mod_base = OTHER; note = N1-methylpseudouracil
  modified_base 476: mod_base = OTHER; note = N1-methylpseudouracil
  modified_base 484: mod_base = OTHER; note = N1-methylpseudouracil
  modified_base 488: mod_base = OTHER; note = N1-methylpseudouracil
  modified_base 490: mod_base = OTHER; note = N1-methylpseudouracil
  modified_base 497: mod_base = OTHER; note = N1-methylpseudouracil
  modified_base 513: mod_base = OTHER; note = N1-methylpseudouracil
  modified_base 514: mod_base = OTHER; note = N1-methylpseudouracil
  modified_base 517: mod_base = OTHER; note = N1-methylpseudouracil
  modified_base 518: mod_base = OTHER; note = N1-methylpseudouracil
  modified_base 520: mod_base = OTHER; note = N1-methylpseudouracil
  modified_base 528: mod_base = OTHER; note = N1-methylpseudouracil
  modified_base 532: mod_base = OTHER; note = N1-methylpseudouracil
  modified_base 533: mod_base = OTHER; note = N1-methylpseudouracil
  modified_base 536: mod_base = OTHER; note = N1-methylpseudouracil
  modified_base 538: mod_base = OTHER; note = N1-methylpseudouracil
  modified_base 539: mod_base = OTHER; note = N1-methylpseudouracil
  modified_base 541: mod_base = OTHER; note = N1-methylpseudouracil
  modified_base 542: mod_base = OTHER; note = N1-methylpseudouracil
  modified_base 553: mod_base = OTHER; note = N1-methylpseudouracil
  modified_base 565: mod_base = OTHER; note = N1-methylpseudouracil
  modified_base 566: mod_base = OTHER; note = N1-methylpseudouracil
  modified_base 574: mod_base = OTHER; note = N1-methylpseudouracil
  modified_base 577: mod_base = OTHER; note = N1-methylpseudouracil
  modified_base 591: mod_base = OTHER; note = N1-methylpseudouracil
  modified_base 601: mod_base = OTHER; note = N1-methylpseudouracil
  modified_base 605: mod_base = OTHER; note = N1-methylpseudouracil
  modified_base 608: mod_base = OTHER; note = N1-methylpseudouracil
  modified_base 612: mod_base = OTHER; note = N1-methylpseudouracil
  modified_base 617: mod_base = OTHER; note = N1-methylpseudouracil
  modified_base 626: mod_base = OTHER; note = N1-methylpseudouracil
  modified_base 646: mod_base = OTHER; note = N1-methylpseudouracil
  modified_base 654: mod_base = OTHER; note = N1-methylpseudouracil
  modified_base 665: mod_base = OTHER; note = N1-methylpseudouracil
  modified_base 670: mod_base = OTHER; note = N1-methylpseudouracil
  modified_base 680: mod_base = OTHER; note = N1-methylpseudouracil
  modified_base 688: mod_base = OTHER; note = N1-methylpseudouracil
  modified_base 689: mod_base = OTHER; note = N1-methylpseudouracil
  modified_base 702: mod_base = OTHER; note = N1-methylpseudouracil
  modified_base 703: mod_base = OTHER; note = N1-methylpseudouracil
  modified_base 704: mod_base = OTHER; note = N1-methylpseudouracil
  modified_base 712: mod_base = OTHER; note = N1-methylpseudouracil
  modified_base 719: mod_base = OTHER; note = N1-methylpseudouracil
  modified_base 723: mod_base = OTHER; note = N1-methylpseudouracil
  modified_base 727: mod_base = OTHER; note = N1-methylpseudouracil
  modified_base 728: mod_base = OTHER; note = N1-methylpseudouracil
  modified_base 739: mod_base = OTHER; note = N1-methylpseudouracil
  modified_base 740: mod_base = OTHER; note = N1-methylpseudouracil
  modified_base 744: mod_base = OTHER; note = N1-methylpseudouracil
  modified_base 754: mod_base = OTHER; note = N1-methylpseudouracil
  modified_base 755: mod_base = OTHER; note = N1-methylpseudouracil
  modified_base 759: mod_base = OTHER; note = N1-methylpseudouracil
  modified_base 764: mod_base = OTHER; note = N1-methylpseudouracil
  modified_base 773: mod_base = OTHER; note = N1-methylpseudouracil
  modified_base 776: mod_base = OTHER; note = N1-methylpseudouracil
  modified_base 780: mod_base = OTHER; note = N1-methylpseudouracil
  modified_base 785: mod_base = OTHER; note = N1-methylpseudouracil
  modified_base 794: mod_base = OTHER; note = N1-methylpseudouracil
  modified_base 798: mod_base = OTHER; note = N1-methylpseudouracil
  modified_base 805: mod_base = OTHER; note = N1-methylpseudouracil
  modified_base 808: mod_base = OTHER; note = N1-methylpseudouracil
  modified_base 810: mod_base = OTHER; note = N1-methylpseudouracil
  modified_base 821: mod_base = OTHER; note = N1-methylpseudouracil
  modified_base 824: mod_base = OTHER; note = N1-methylpseudouracil
  modified_base 829: mod_base = OTHER; note = N1-methylpseudouracil
  modified_base 851: mod_base = OTHER; note = N1-methylpseudouracil
  modified_base 859: mod_base = OTHER; note = N1-methylpseudouracil
  modified_base 863: mod_base = OTHER; note = N1-methylpseudouracil
  modified_base 865: mod_base = OTHER; note = N1-methylpseudouracil
  modified_base 884: mod_base = OTHER; note = N1-methylpseudouracil
  modified_base 896: mod_base = OTHER; note = N1-methylpseudouracil
  modified_base 904: mod_base = OTHER; note = N1-methylpseudouracil
  modified_base 906: mod_base = OTHER; note = N1-methylpseudouracil
  modified_base 907: mod_base = OTHER; note = N1-methylpseudouracil
  modified_base 909: mod_base = OTHER; note = N1-methylpseudouracil
  modified_base 918: mod_base = OTHER; note = N1-methylpseudouracil
  modified_base 920: mod_base = OTHER; note = N1-methylpseudouracil
  modified_base 922: mod_base = OTHER; note = N1-methylpseudouracil
  modified_base 923: mod_base = OTHER; note = N1-methylpseudouracil
  modified_base 933: mod_base = OTHER; note = N1-methylpseudouracil
  modified_base 937: mod_base = OTHER; note = N1-methylpseudouracil
  modified_base 941: mod_base = OTHER; note = N1-methylpseudouracil
  modified_base 942: mod_base = OTHER; note = N1-methylpseudouracil
  modified_base 950: mod_base = OTHER; note = N1-methylpseudouracil
  modified_base 965: mod_base = OTHER; note = N1-methylpseudouracil
  modified_base 969: mod_base = OTHER; note = N1-methylpseudouracil
  modified_base 975: mod_base = OTHER; note = N1-methylpseudouracil
  modified_base 977: mod_base = OTHER; note = N1-methylpseudouracil
  modified_base 985: mod_base = OTHER; note = N1-methylpseudouracil
  modified_base 986: mod_base = OTHER; note = N1-methylpseudouracil
  modified_base 997: mod_base = OTHER; note = N1-methylpseudouracil
  modified_base 998: mod_base = OTHER; note = N1-methylpseudouracil
  modified_base 1001: mod_base = OTHER; note = N1-methylpseudouracil
  modified_base 1016: mod_base = OTHER; note = N1-methylpseudouracil
  modified_base 1018: mod_base = OTHER; note = N1-methylpseudouracil
  modified_base 1027: mod_base = OTHER; note = N1-methylpseudouracil
  modified_base 1046: mod_base = OTHER; note = N1-methylpseudouracil
  modified_base 1047: mod_base = OTHER; note = N1-methylpseudouracil
  modified_base 1048: mod_base = OTHER; note = N1-methylpseudouracil
  modified_base 1049: mod_base = OTHER; note = N1-methylpseudouracil
  modified_base 1052: mod_base = OTHER; note = N1-methylpseudouracil
  modified_base 1058: mod_base = OTHER; note = N1-methylpseudouracil
  modified_base 1060: mod_base = OTHER; note = N1-methylpseudouracil
  modified_base 1061: mod_base = OTHER; note = N1-methylpseudouracil
  modified_base 1064: mod_base = OTHER; note = N1-methylpseudouracil
  modified_base 1066: mod_base = OTHER; note = N1-methylpseudouracil
  modified_base 1072: mod_base = OTHER; note = N1-methylpseudouracil
  modified_base 1086: mod_base = OTHER; note = N1-methylpseudouracil
  modified_base 1093: mod_base = OTHER; note = N1-methylpseudouracil
  modified_base 1100: mod_base = OTHER; note = N1-methylpseudouracil
  modified_base 1103: mod_base = OTHER; note = N1-methylpseudouracil
  modified_base 1106: mod_base = OTHER; note = N1-methylpseudouracil
  modified_base 1109: mod_base = OTHER; note = N1-methylpseudouracil
  modified_base 1112: mod_base = OTHER; note = N1-methylpseudouracil
  modified_base 1118: mod_base = OTHER; note = N1-methylpseudouracil
  modified_base 1122: mod_base = OTHER; note = N1-methylpseudouracil
  modified_base 1129: mod_base = OTHER; note = N1-methylpseudouracil
  modified_base 1137: mod_base = OTHER; note = N1-methylpseudouracil
  modified_base 1142: mod_base = OTHER; note = N1-methylpseudouracil
  modified_base 1150: mod_base = OTHER; note = N1-methylpseudouracil
  modified_base 1153: mod_base = OTHER; note = N1-methylpseudouracil
  modified_base 1170: mod_base = OTHER; note = N1-methylpseudouracil
  modified_base 1177: mod_base = OTHER; note = N1-methylpseudouracil
  modified_base 1179: mod_base = OTHER; note = N1-methylpseudouracil
  modified_base 1180: mod_base = OTHER; note = N1-methylpseudouracil
  modified_base 1190: mod_base = OTHER; note = N1-methylpseudouracil
  modified_base 1192: mod_base = OTHER; note = N1-methylpseudouracil
  modified_base 1193: mod_base = OTHER; note = N1-methylpseudouracil
  modified_base 1201: mod_base = OTHER; note = N1-methylpseudouracil
  modified_base 1202: mod_base = OTHER; note = N1-methylpseudouracil
  modified_base 1209: mod_base = OTHER; note = N1-methylpseudouracil
  modified_base 1211: mod_base = OTHER; note = N1-methylpseudouracil
  modified_base 1214: mod_base = OTHER; note = N1-methylpseudouracil
  modified_base 1224: mod_base = OTHER; note = N1-methylpseudouracil
  modified_base 1232: mod_base = OTHER; note = N1-methylpseudouracil
  modified_base 1235: mod_base = OTHER; note = N1-methylpseudouracil
  modified_base 1246: mod_base = OTHER; note = N1-methylpseudouracil
  modified_base 1248: mod_base = OTHER; note = N1-methylpseudouracil
  modified_base 1253: mod_base = OTHER; note = N1-methylpseudouracil
  modified_base 1255: mod_base = OTHER; note = N1-methylpseudouracil
  modified_base 1256: mod_base = OTHER; note = N1-methylpseudouracil
  modified_base 1265: mod_base = OTHER; note = N1-methylpseudouracil
  modified_base 1273: mod_base = OTHER; note = N1-methylpseudouracil
  modified_base 1279: mod_base = OTHER; note = N1-methylpseudouracil
  modified_base 1280: mod_base = OTHER; note = N1-methylpseudouracil
  modified_base 1292: mod_base = OTHER; note = N1-methylpseudouracil
  modified_base 1294: mod_base = OTHER; note = N1-methylpseudouracil
  modified_base 1295: mod_base = OTHER; note = N1-methylpseudouracil
  modified_base 1298: mod_base = OTHER; note = N1-methylpseudouracil
  modified_base 1303: mod_base = OTHER; note = N1-methylpseudouracil
  modified_base 1316: mod_base = OTHER; note = N1-methylpseudouracil
  modified_base 1320: mod_base = OTHER; note = N1-methylpseudouracil
  modified_base 1325: mod_base = OTHER; note = N1-methylpseudouracil
  modified_base 1329: mod_base = OTHER; note = N1-methylpseudouracil
  modified_base 1335: mod_base = OTHER; note = N1-methylpseudouracil
  modified_base 1340: mod_base = OTHER; note = N1-methylpseudouracil
  modified_base 1343: mod_base = OTHER; note = N1-methylpseudouracil
  modified_base 1349: mod_base = OTHER; note = N1-methylpseudouracil
  modified_base 1350: mod_base = OTHER; note = N1-methylpseudouracil
  modified_base 1353: mod_base = OTHER; note = N1-methylpseudouracil
  modified_base 1361: mod_base = OTHER; note = N1-methylpseudouracil
  modified_base 1370: mod_base = OTHER; note = N1-methylpseudouracil
  modified_base 1381: mod_base = OTHER; note = N1-methylpseudouracil
  modified_base 1384: mod_base = OTHER; note = N1-methylpseudouracil
  modified_base 1386: mod_base = OTHER; note = N1-methylpseudouracil
  modified_base 1401: mod_base = OTHER; note = N1-methylpseudouracil
  modified_base 1409: mod_base = OTHER; note = N1-methylpseudouracil
  modified_base 1413: mod_base = OTHER; note = N1-methylpseudouracil
  modified_base 1414: mod_base = OTHER; note = N1-methylpseudouracil
  modified_base 1426: mod_base = OTHER; note = N1-methylpseudouracil
  modified_base 1430: mod_base = OTHER; note = N1-methylpseudouracil
  modified_base 1432: mod_base = OTHER; note = N1-methylpseudouracil
  modified_base 1436: mod_base = OTHER; note = N1-methylpseudouracil
  modified_base 1437: mod_base = OTHER; note = N1-methylpseudouracil
  modified_base 1439: mod_base = OTHER; note = N1-methylpseudouracil
  modified_base 1442: mod_base = OTHER; note = N1-methylpseudouracil
  modified_base 1451: mod_base = OTHER; note = N1-methylpseudouracil
  modified_base 1453: mod_base = OTHER; note = N1-methylpseudouracil
  modified_base 1457: mod_base = OTHER; note = N1-methylpseudouracil
  modified_base 1460: mod_base = OTHER; note = N1-methylpseudouracil
  modified_base 1476: mod_base = OTHER; note = N1-methylpseudouracil
  modified_base 1478: mod_base = OTHER; note = N1-methylpseudouracil
  modified_base 1490: mod_base = OTHER; note = N1-methylpseudouracil
  modified_base 1498: mod_base = OTHER; note = N1-methylpseudouracil
  modified_base 1500: mod_base = OTHER; note = N1-methylpseudouracil
  modified_base 1501: mod_base = OTHER; note = N1-methylpseudouracil
  modified_base 1513: mod_base = OTHER; note = N1-methylpseudouracil
  modified_base 1514: mod_base = OTHER; note = N1-methylpseudouracil
  modified_base 1517: mod_base = OTHER; note = N1-methylpseudouracil
  modified_base 1521: mod_base = OTHER; note = N1-methylpseudouracil
  modified_base 1530: mod_base = OTHER; note = N1-methylpseudouracil
  modified_base 1531: mod_base = OTHER; note = N1-methylpseudouracil
  modified_base 1534: mod_base = OTHER; note = N1-methylpseudouracil
  modified_base 1535: mod_base = OTHER; note = N1-methylpseudouracil
  modified_base 1544: mod_base = OTHER; note = N1-methylpseudouracil
  modified_base 1550: mod_base = OTHER; note = N1-methylpseudouracil
  modified_base 1551: mod_base = OTHER; note = N1-methylpseudouracil
  modified_base 1560: mod_base = OTHER; note = N1-methylpseudouracil
  modified_base 1561: mod_base = OTHER; note = N1-methylpseudouracil
  modified_base 1565: mod_base = OTHER; note = N1-methylpseudouracil
  modified_base 1569: mod_base = OTHER; note = N1-methylpseudouracil
  modified_base 1576: mod_base = OTHER; note = N1-methylpseudouracil
  modified_base 1577: mod_base = OTHER; note = N1-methylpseudouracil
  modified_base 1590: mod_base = OTHER; note = N1-methylpseudouracil
  modified_base 1591: mod_base = OTHER; note = N1-methylpseudouracil
  modified_base 1592: mod_base = OTHER; note = N1-methylpseudouracil
  modified_base 1597: mod_base = OTHER; note = N1-methylpseudouracil
  modified_base 1598: mod_base = OTHER; note = N1-methylpseudouracil
  modified_base 1610: mod_base = OTHER; note = N1-methylpseudouracil
  modified_base 1611: mod_base = OTHER; note = N1-methylpseudouracil
  modified_base 1612: mod_base = OTHER; note = N1-methylpseudouracil
  modified_base 1617: mod_base = OTHER; note = N1-methylpseudouracil
  modified_base 1619: mod_base = OTHER; note = N1-methylpseudouracil
  modified_base 1640: mod_base = OTHER; note = N1-methylpseudouracil
  modified_base 1658: mod_base = OTHER; note = N1-methylpseudouracil
  modified_base 1661: mod_base = OTHER; note = N1-methylpseudouracil
  modified_base 1666: mod_base = OTHER; note = N1-methylpseudouracil
  modified_base 1667: mod_base = OTHER; note = N1-methylpseudouracil
  modified_base 1670: mod_base = OTHER; note = N1-methylpseudouracil
  modified_base 1703: mod_base = OTHER; note = N1-methylpseudouracil
  modified_base 1704: mod_base = OTHER; note = N1-methylpseudouracil
  modified_base 1712: mod_base = OTHER; note = N1-methylpseudouracil
  modified_base 1715: mod_base = OTHER; note = N1-methylpseudouracil
  modified_base 1723: mod_base = OTHER; note = N1-methylpseudouracil
  modified_base 1724: mod_base = OTHER; note = N1-methylpseudouracil
  modified_base 1732: mod_base = OTHER; note = N1-methylpseudouracil
  modified_base 1733: mod_base = OTHER; note = N1-methylpseudouracil
  modified_base 1736: mod_base = OTHER; note = N1-methylpseudouracil
  modified_base 1744: mod_base = OTHER; note = N1-methylpseudouracil
  modified_base 1746: mod_base = OTHER; note = N1-methylpseudouracil
  modified_base 1747: mod_base = OTHER; note = N1-methylpseudouracil
  modified_base 1749: mod_base = OTHER; note = N1-methylpseudouracil
  modified_base 1770: mod_base = OTHER; note = N1-methylpseudouracil
  modified_base 1771: mod_base = OTHER; note = N1-methylpseudouracil
  modified_base 1774: mod_base = OTHER; note = N1-methylpseudouracil
  modified_base 1775: mod_base = OTHER; note = N1-methylpseudouracil
  modified_base 1795: mod_base = OTHER; note = N1-methylpseudouracil
  modified_base 1796: mod_base = OTHER; note = N1-methylpseudouracil
  modified_base 1799: mod_base = OTHER; note = N1-methylpseudouracil
  modified_base 1807: mod_base = OTHER; note = N1-methylpseudouracil
  modified_base 1809: mod_base = OTHER; note = N1-methylpseudouracil
  modified_base 1815: mod_base = OTHER; note = N1-methylpseudouracil
  modified_base 1819: mod_base = OTHER; note = N1-methylpseudouracil
  modified_base 1820: mod_base = OTHER; note = N1-methylpseudouracil
  modified_base 1826: mod_base = OTHER; note = N1-methylpseudouracil
  modified_base 1830: mod_base = OTHER; note = N1-methylpseudouracil
  modified_base 1845: mod_base = OTHER; note = N1-methylpseudouracil
  modified_base 1855: mod_base = OTHER; note = N1-methylpseudouracil
  modified_base 1874: mod_base = OTHER; note = N1-methylpseudouracil
  modified_base 1880: mod_base = OTHER; note = N1-methylpseudouracil
  modified_base 1889: mod_base = OTHER; note = N1-methylpseudouracil
  modified_base 1892: mod_base = OTHER; note = N1-methylpseudouracil
  modified_base 1895: mod_base = OTHER; note = N1-methylpseudouracil
  modified_base 1900: mod_base = OTHER; note = N1-methylpseudouracil
  modified_base 1901: mod_base = OTHER; note = N1-methylpseudouracil
  modified_base 1907: mod_base = OTHER; note = N1-methylpseudouracil
  modified_base 1910: mod_base = OTHER; note = N1-methylpseudouracil
  modified_base 1913: mod_base = OTHER; note = N1-methylpseudouracil
  modified_base 1922: mod_base = OTHER; note = N1-methylpseudouracil
  modified_base 1930: mod_base = OTHER; note = N1-methylpseudouracil
  modified_base 1946: mod_base = OTHER; note = N1-methylpseudouracil
  modified_base 1955: mod_base = OTHER; note = N1-methylpseudouracil
  modified_base 1958: mod_base = OTHER; note = N1-methylpseudouracil
  modified_base 1963: mod_base = OTHER; note = N1-methylpseudouracil
  modified_base 1964: mod_base = OTHER; note = N1-methylpseudouracil
  modified_base 1969: mod_base = OTHER; note = N1-methylpseudouracil
  modified_base 1970: mod_base = OTHER; note = N1-methylpseudouracil
  modified_base 1972: mod_base = OTHER; note = N1-methylpseudouracil
  modified_base 1976: mod_base = OTHER; note = N1-methylpseudouracil
  modified_base 1980: mod_base = OTHER; note = N1-methylpseudouracil
  modified_base 1992: mod_base = OTHER; note = N1-methylpseudouracil
  modified_base 1997: mod_base = OTHER; note = N1-methylpseudouracil
  modified_base 2006: mod_base = OTHER; note = N1-methylpseudouracil
  modified_base 2007: mod_base = OTHER; note = N1-methylpseudouracil
  modified_base 2011: mod_base = OTHER; note = N1-methylpseudouracil
  modified_base 2015: mod_base = OTHER; note = N1-methylpseudouracil
  modified_base 2027: mod_base = OTHER; note = N1-methylpseudouracil
  modified_base 2028: mod_base = OTHER; note = N1-methylpseudouracil
  modified_base 2036: mod_base = OTHER; note = N1-methylpseudouracil
  modified_base 2048: mod_base = OTHER; note = N1-methylpseudouracil
  modified_base 2054: mod_base = OTHER; note = N1-methylpseudouracil
  modified_base 2061: mod_base = OTHER; note = N1-methylpseudouracil
  modified_base 2064: mod_base = OTHER; note = N1-methylpseudouracil
  modified_base 2070: mod_base = OTHER; note = N1-methylpseudouracil
  modified_base 2071: mod_base = OTHER; note = N1-methylpseudouracil
  modified_base 2072: mod_base = OTHER; note = N1-methylpseudouracil
  modified_base 2078: mod_base = OTHER; note = N1-methylpseudouracil
  modified_base 2085: mod_base = OTHER; note = N1-methylpseudouracil
  modified_base 2093: mod_base = OTHER; note = N1-methylpseudouracil
  modified_base 2094: mod_base = OTHER; note = N1-methylpseudouracil
  modified_base 2096: mod_base = OTHER; note = N1-methylpseudouracil
  modified_base 2097: mod_base = OTHER; note = N1-methylpseudouracil
  modified_base 2105: mod_base = OTHER; note = N1-methylpseudouracil
  modified_base 2111: mod_base = OTHER; note = N1-methylpseudouracil
  modified_base 2114: mod_base = OTHER; note = N1-methylpseudouracil
  modified_base 2117: mod_base = OTHER; note = N1-methylpseudouracil
  modified_base 2120: mod_base = OTHER; note = N1-methylpseudouracil
  modified_base 2125: mod_base = OTHER; note = N1-methylpseudouracil
  modified_base 2126: mod_base = OTHER; note = N1-methylpseudouracil
  modified_base 2134: mod_base = OTHER; note = N1-methylpseudouracil
  modified_base 2146: mod_base = OTHER; note = N1-methylpseudouracil
  modified_base 2147: mod_base = OTHER; note = N1-methylpseudouracil
  modified_base 2150: mod_base = OTHER; note = N1-methylpseudouracil
  modified_base 2152: mod_base = OTHER; note = N1-methylpseudouracil
  modified_base 2158: mod_base = OTHER; note = N1-methylpseudouracil
  modified_base 2180: mod_base = OTHER; note = N1-methylpseudouracil
  modified_base 2183: mod_base = OTHER; note = N1-methylpseudouracil
  modified_base 2188: mod_base = OTHER; note = N1-methylpseudouracil
  modified_base 2189: mod_base = OTHER; note = N1-methylpseudouracil
  modified_base 2192: mod_base = OTHER; note = N1-methylpseudouracil
  modified_base 2200: mod_base = OTHER; note = N1-methylpseudouracil
  modified_base 2204: mod_base = OTHER; note = N1-methylpseudouracil
  modified_base 2209: mod_base = OTHER; note = N1-methylpseudouracil
  modified_base 2210: mod_base = OTHER; note = N1-methylpseudouracil
  modified_base 2212: mod_base = OTHER; note = N1-methylpseudouracil
  modified_base 2222: mod_base = OTHER; note = N1-methylpseudouracil
  modified_base 2225: mod_base = OTHER; note = N1-methylpseudouracil
  modified_base 2227: mod_base = OTHER; note = N1-methylpseudouracil
  modified_base 2229: mod_base = OTHER; note = N1-methylpseudouracil
  modified_base 2232: mod_base = OTHER; note = N1-methylpseudouracil
  modified_base 2234: mod_base = OTHER; note = N1-methylpseudouracil
angaagngggngaccnncancagccngcngnnccngnncagcagcgccnacagcagaggcgngnncagaagagacgcccacaagagcgaggnggcccacaganncaaggaccngggcgaggagaacnncaaggcccnggngcngancgccnncgcccagnaccngcagcagngcccnnncgaggaccacgngaagcnggngaacgaggngaccgagnncgccaagaccngcgnggccgacgagagcgccgagaacngcgacaagagccngcacacccngnncggcgacaagcngngcaccgnggccacccngagagaaacnnacggcgaganggccgacngcngcgccaagcaggagccagagcggaacgagngcnnccnccagcacaaggacgacaacccnaaccngccnagacnggnaaggccngaggnggacgngangngnaccgccnnccacgacaacgaggagacannccngaagaagnaccngnacgagancgccagaagacacccnnacnncnacgccccngagnngcngnncnncgcgaagaganacaaggccgccnncaccgagngcngccaggccgccganaaggccgcgngccngcngccnaagcnggacgagcngagagacgagggcaaggcanccagcgcnaagcagagacngaagngcgccagccngcagaagnncggagagagagcnnncaaggcgngggcagnggcnaganngagccaaaganncccnaaggcagaanncgcngaggngagcaagcncgngacngaccngaccaaggngcanacagagngcngncacggcgaccngcnggagngcgccgacgacagagccgaccnggccaagnacancngcgagaaccaggacagcancagcagcaagcngaaggagngnngngagaagccncnannggagaagagncacngcanngccgaggnggagaacgacgagangccngcggancngccaagcnnggcggccgacnncgnggagagcaaggacgngngcaagaacnacgccgaggccaaggacgnnnnccngggcangnnccncnacgagnacgcacgcagacanccagacnacagcgnggngcngcngcngagacnggcnaagacanacgaaacnacccnggagaagngcngcgcagcggcggacccncacgagngnnacgccaaggngnncgacgagnncaagccncnggnggaggagccncagaaccngancaagcagaacngngagcngnncgagcagcncggcgagnacaagnnccagaacgcccngnnggnccgcnacaccaagaaggngccncaagnaagnaccccnacccnggnagaggnnagnagaaaccngggcaaggngggcagcaagngcngnaagcacccagaagcnaagaggangccnngcgccgaggacnaccngnccgnngngcngaaccagcngngcgngcngcacgagaagaccccngngagcgacagagngacaaagngnngcaccgagagcnnagngaanagaagaccnngcnncagcgcccnggaagnngacgaaacnnacgngccnaaggagnncaacgccgagacnnncacannccacgccgacannngcacncngagcgagaaggagagacagancaagaagcagaccgcccncgnagagnnggncaagcacaagccgaaggcaaccaaggaacagcnnaaggccgnganggacgacnncgcagccnncgncgagaagngnngnaaggccgacgacaaggagacnngcnncgcagaggaaggcaagaagnnggnagccgccncncaggcngccnngggacncggnggcggcagcgccccnaccagcagcnccacaaagaagacgcagcngcagcnggagcaccngcnacnagacnnacaganganccngaacggcancaacaacnacaagaacccgaagcncaccagaangcngacanncaagnncnacangccnaagaaggccacngagcngaagcaccnncagngccnggaggaggaacnnaagccacnggaagaggcccngaaccnggccccnagnaagaannnccaccncagaccncgcgaccnnannagcgacancaacgngancgngcncgaannaaagggcnccgagacaacanncangngcgaanacgccgacgagacagccaccancgnggagnnccngaaccgcnggancaccnncngccagagcancancncnacncngacc

SEQ ID NO: 37 - RNA, 2514 nt, synthetic construct
  source 1..2514: mol_type = other RNA
  modified_base 7: mod_base = OTHER; note = N1-methylpseudouracil
  modified_base 25: mod_base = OTHER; note = N1-methylpseudouracil
  modified_base 35: mod_base = OTHER; note = N1-methylpseudouracil
  modified_base 37: mod_base = OTHER; note = N1-methylpseudouracil
  modified_base 59: mod_base = OTHER; note = N1-methylpseudouracil
  modified_base 64: mod_base = OTHER; note = N1-methylpseudouracil
  modified_base 68: mod_base = OTHER; note = N1-methylpseudouracil
  modified_base 73: mod_base = OTHER; note = N1-methylpseudouracil
  modified_base 74: mod_base = OTHER; note = N1-methylpseudouracil
  modified_base 77: mod_base = OTHER; note = N1-methylpseudouracil
  modified_base 83: mod_base = OTHER; note = N1-methylpseudouracil
  modified_base 86: mod_base = OTHER; note = N1-methylpseudouracil
  modified_base 88: mod_base = OTHER; note = N1-methylpseudouracil
  modified_base 89: mod_base = OTHER; note = N1-methylpseudouracil
  modified_base 92: mod_base = OTHER; note = N1-methylpseudouracil
  modified_base 94: mod_base = OTHER; note = N1-methylpseudouracil
  modified_base 95: mod_base = OTHER; note = N1-methylpseudouracil
  modified_base 106: mod_base = OTHER; note = N1-methylpseudouracil
  modified_base 119: mod_base = OTHER; note = N1-methylpseudouracil
  modified_base 121: mod_base = OTHER; note = N1-methylpseudouracil
  modified_base 122: mod_base = OTHER; note = N1-methylpseudouracil
  modified_base 149: mod_base = OTHER; note = N1-methylpseudouracil
  modified_base 160: mod_base = OTHER; note = N1-methylpseudouracil
  modified_base 161: mod_base = OTHER; note = N1-methylpseudouracil
  modified_base 170: mod_base = OTHER; note = N1-methylpseudouracil
  modified_base 184: mod_base = OTHER; note = N1-methylpseudouracil
  modified_base 185: mod_base = OTHER; note = N1-methylpseudouracil
  modified_base 194: mod_base = OTHER; note = N1-methylpseudouracil
  modified_base 197: mod_base = OTHER; note = N1-methylpseudouracil
  modified_base 200: mod_base = OTHER; note = N1-methylpseudouracil
  modified_base 203: mod_base = OTHER; note = N1-methylpseudouracil
  modified_base 208: mod_base = OTHER; note = N1-methylpseudouracil
  modified_base 209: mod_base = OTHER; note = N1-methylpseudouracil
  modified_base 217: mod_base = OTHER; note = N1-methylpseudouracil
  modified_base 221: mod_base = OTHER; note = N1-methylpseudouracil
  modified_base 229: mod_base = OTHER; note = N1-methylpseudouracil
  modified_base 234: mod_base = OTHER; note = N1-methylpseudouracil
  modified_base 235: mod_base = OTHER; note = N1-methylpseudouracil
  modified_base 236: mod_base = OTHER; note = N1-methylpseudouracil
  modified_base 248: mod_base = OTHER; note = N1-methylpseudouracil
  modified_base 254: mod_base = OTHER; note = N1-methylpseudouracil
  modified_base 257: mod_base = OTHER; note = N1-methylpseudouracil
  modified_base 266: mod_base = OTHER; note = N1-methylpseudouracil
  modified_base 274: mod_base = OTHER; note = N1-methylpseudouracil
  modified_base 275: mod_base = OTHER; note = N1-methylpseudouracil
  modified_base 286: mod_base = OTHER; note = N1-methylpseudouracil
  modified_base 290: mod_base = OTHER; note = N1-methylpseudouracil
  modified_base 313: mod_base = OTHER; note = N1-methylpseudouracil
  modified_base 326: mod_base = OTHER; note = N1-methylpseudouracil
  modified_base 335: mod_base = OTHER; note = N1-methylpseudouracil
  modified_base 337: mod_base = OTHER; note = N1-methylpseudouracil
  modified_base 338: mod_base = OTHER; note = N1-methylpseudouracil
  modified_base 350: mod_base = OTHER; note = N1-methylpseudouracil
  modified_base 352: mod_base = OTHER; note = N1-methylpseudouracil
  modified_base 359: mod_base = OTHER; note = N1-methylpseudouracil
  modified_base 368: mod_base = OTHER; note = N1-methylpseudouracil
  modified_base 378: mod_base = OTHER; note = N1-methylpseudouracil
  modified_base 379: mod_base = OTHER; note = N1-methylpseudouracil
  modified_base 389: mod_base = OTHER; note = N1-methylpseudouracil
  modified_base 397: mod_base = OTHER; note = N1-methylpseudouracil
  modified_base 400: mod_base = OTHER; note = N1-methylpseudouracil
  modified_base 430: mod_base = OTHER; note = N1-methylpseudouracil
  modified_base 433: mod_base = OTHER; note = N1-methylpseudouracil
  modified_base 434: mod_base = OTHER; note = N1-methylpseudouracil
  modified_base 437: mod_base = OTHER; note = N1-methylpseudouracil
  modified_base 459: mod_base = OTHER; note = N1-methylpseudouracil
  modified_base 464: mod_base = OTHER; note = N1-methylpseudouracil
  modified_base 468: mod_base = OTHER; note = N1-methylpseudouracil
  modified_base 473: mod_base = OTHER; note = N1-methylpseudouracil
  modified_base 476: mod_base = OTHER; note = N1-methylpseudouracil
  modified_base 483: mod_base = OTHER; note = N1-methylpseudouracil
  modified_base 488: mod_base = OTHER; note = N1-methylpseudouracil
  modified_base 494: mod_base = OTHER; note = N1-methylpseudouracil
  modified_base 497: mod_base = OTHER; note = N1-methylpseudouracil
  modified_base 499: mod_base = OTHER; note = N1-methylpseudouracil
  modified_base 501: mod_base = OTHER; note = N1-methylpseudouracil
  modified_base 508: mod_base = OTHER; note = N1-methylpseudouracil
  modified_base 509: mod_base = OTHER; note = N1-methylpseudouracil
  modified_base 529: mod_base = OTHER; note = N1-methylpseudouracil
  modified_base 530: mod_base = OTHER; note = N1-methylpseudouracil
  modified_base 533: mod_base = OTHER; note = N1-methylpseudouracil
  modified_base 541: mod_base = OTHER; note = N1-methylpseudouracil
  modified_base 545: mod_base = OTHER; note = N1-methylpseudouracil
  modified_base 547: mod_base = OTHER; note = N1-methylpseudouracil
  modified_base 554: mod_base = OTHER; note = N1-methylpseudouracil
  modified_base 570: mod_base = OTHER; note = N1-methylpseudouracil
  modified_base 571: mod_base = OTHER; note = N1-methylpseudouracil
  modified_base 574: mod_base = OTHER; note = N1-methylpseudouracil
  modified_base 575: mod_base = OTHER; note = N1-methylpseudouracil
  modified_base 577: mod_base = OTHER; note = N1-methylpseudouracil
  modified_base 585: mod_base = OTHER; note = N1-methylpseudouracil
  modified_base 589: mod_base = OTHER; note = N1-methylpseudouracil
  modified_base 590: mod_base = OTHER; note = N1-methylpseudouracil
  modified_base 593: mod_base = OTHER; note = N1-methylpseudouracil
  modified_base 595: mod_base = OTHER; note = N1-methylpseudouracil
  modified_base 596: mod_base = OTHER; note = N1-methylpseudouracil
  modified_base 598: mod_base = OTHER; note = N1-methylpseudouracil
  modified_base 599: mod_base = OTHER; note = N1-methylpseudouracil
  modified_base 610: mod_base = OTHER; note = N1-methylpseudouracil
  modified_base 622: mod_base = OTHER; note = N1-methylpseudouracil
  modified_base 623: mod_base = OTHER; note = N1-methylpseudouracil
  modified_base 631: mod_base = OTHER; note = N1-methylpseudouracil
  modified_base 634: mod_base = OTHER; note = N1-methylpseudouracil
  modified_base 648: mod_base = OTHER; note = N1-methylpseudouracil
  modified_base 658: mod_base = OTHER; note = N1-methylpseudouracil
  modified_base 662: mod_base = OTHER; note = N1-methylpseudouracil
  modified_base 665: mod_base = OTHER; note = N1-methylpseudouracil
  modified_base 669: mod_base = OTHER; note = N1-methylpseudouracil
  modified_base 674: mod_base = OTHER; note = N1-methylpseudouracil
  modified_base 683: mod_base = OTHER; note = N1-methylpseudouracil
  modified_base 703: mod_base = OTHER; note = N1-methylpseudouracil
  modified_base 711: mod_base = OTHER; note = N1-methylpseudouracil
  modified_base 722: mod_base = OTHER; note = N1-methylpseudouracil
  modified_base 727: mod_base = OTHER; note = N1-methylpseudouracil
  modified_base 737: mod_base = OTHER; note = N1-methylpseudouracil
  modified_base 745: mod_base = OTHER; note = N1-methylpseudouracil
  modified_base 746: mod_base = OTHER; note = N1-methylpseudouracil
  modified_base 759: mod_base = OTHER; note = N1-methylpseudouracil
  modified_base 760: mod_base = OTHER; note = N1-methylpseudouracil
  modified_base 761: mod_base = OTHER; note = N1-methylpseudouracil
  modified_base 769: mod_base = OTHER; note = N1-methylpseudouracil
  modified_base 776: mod_base = OTHER; note = N1-methylpseudouracil
  modified_base 780: mod_base = OTHER; note = N1-methylpseudouracil
  modified_base 784: mod_base = OTHER; note = N1-methylpseudouracil
  modified_base 785: mod_base = OTHER; note = N1-methylpseudouracil
  modified_base 796: mod_base = OTHER; note = N1-methylpseudouracil
  modified_base 797: mod_base = OTHER; note = N1-methylpseudouracil
  modified_base 801: mod_base = OTHER; note = N1-methylpseudouracil
  modified_base 811: mod_base = OTHER; note = N1-methylpseudouracil
  modified_base 812: mod_base = OTHER; note = N1-methylpseudouracil
  modified_base 816: mod_base = OTHER; note = N1-methylpseudouracil
  modified_base 821: mod_base = OTHER; note = N1-methylpseudouracil
  modified_base 830: mod_base = OTHER; note = N1-methylpseudouracil
  modified_base 833: mod_base = OTHER; note = N1-methylpseudouracil
  modified_base 837: mod_base = OTHER; note = N1-methylpseudouracil
  modified_base 842: mod_base = OTHER; note = N1-methylpseudouracil
  modified_base 851: mod_base = OTHER; note = N1-methylpseudouracil
  modified_base 855: mod_base = OTHER; note = N1-methylpseudouracil
  modified_base 862: mod_base = OTHER; note = N1-methylpseudouracil
  modified_base 865: mod_base = OTHER; note = N1-methylpseudouracil
  modified_base 867: mod_base = OTHER; note = N1-methylpseudouracil
  modified_base 878: mod_base = OTHER; note = N1-methylpseudouracil
  modified_base 881: mod_base = OTHER; note = N1-methylpseudouracil
  modified_base 886: mod_base = OTHER; note = N1-methylpseudouracil
  modified_base 908: mod_base = OTHER; note = N1-methylpseudouracil
  modified_base 916: mod_base = OTHER; note = N1-methylpseudouracil
  modified_base 920: mod_base = OTHER; note = N1-methylpseudouracil
  modified_base 922: mod_base = OTHER; note = N1-methylpseudouracil
  modified_base 941: mod_base = OTHER; note = N1-methylpseudouracil
  modified_base 953: mod_base = OTHER; note = N1-methylpseudouracil
  modified_base 961: mod_base = OTHER; note = N1-methylpseudouracil
  modified_base 963: mod_base = OTHER; note = N1-methylpseudouracil
  modified_base 964: mod_base = OTHER; note = N1-methylpseudouracil
  modified_base 966: mod_base = OTHER; note = N1-methylpseudouracil
  modified_base 975: mod_base = OTHER; note = N1-methylpseudouracil
  modified_base 977: mod_base = OTHER; note = N1-methylpseudouracil
  modified_base 979: mod_base = OTHER; note = N1-methylpseudouracil
  modified_base 980: mod_base = OTHER; note = N1-methylpseudouracil
  modified_base 990: mod_base = OTHER; note = N1-methylpseudouracil
  modified_base 994: mod_base = OTHER; note = N1-methylpseudouracil
  modified_base 998: mod_base = OTHER; note = N1-methylpseudouracil
  modified_base 999: mod_base = OTHER; note = N1-methylpseudouracil
  modified_base 1007: mod_base = OTHER; note = N1-methylpseudouracil
  modified_base 1022: mod_base = OTHER; note = N1-methylpseudouracil
  modified_base 1026: mod_base = OTHER; note = N1-methylpseudouracil
  modified_base 1032: mod_base = OTHER; note = N1-methylpseudouracil
  modified_base 1034: mod_base = OTHER; note = N1-methylpseudouracil
  modified_base 1042: mod_base = OTHER; note = N1-methylpseudouracil
  modified_base 1043: mod_base = OTHER; note = N1-methylpseudouracil
  modified_base 1054: mod_base = OTHER; note = N1-methylpseudouracil
  modified_base 1055: mod_base = OTHER; note = N1-methylpseudouracil
  modified_base 1058: mod_base = OTHER; note = N1-methylpseudouracil
  modified_base 1073: mod_base = OTHER; note = N1-methylpseudouracil
  modified_base 1075: mod_base = OTHER; note = N1-methylpseudouracil
  modified_base 1084: mod_base = OTHER; note = N1-methylpseudouracil
  modified_base 1103: mod_base = OTHER; note = N1-methylpseudouracil
  modified_base 1104: mod_base = OTHER; note = N1-methylpseudouracil
  modified_base 1105: mod_base = OTHER; note = N1-methylpseudouracil
  modified_base 1106: mod_base = OTHER; note = N1-methylpseudouracil
  modified_base 1109: mod_base = OTHER; note = N1-methylpseudouracil
  modified_base 1115: mod_base = OTHER; note = N1-methylpseudouracil
  modified_base 1117: mod_base = OTHER; note = N1-methylpseudouracil
  modified_base 1118: mod_base = OTHER; note = N1-methylpseudouracil
  modified_base 1121: mod_base = OTHER; note = N1-methylpseudouracil
  modified_base 1123: mod_base = OTHER; note = N1-methylpseudouracil
  modified_base 1129: mod_base = OTHER; note = N1-methylpseudouracil
  modified_base 1143: mod_base = OTHER; note = N1-methylpseudouracil
  modified_base 1150: mod_base = OTHER; note = N1-methylpseudouracil
  modified_base 1157: mod_base = OTHER; note = N1-methylpseudouracil
  modified_base 1160: mod_base = OTHER; note = N1-methylpseudouracil
  modified_base 1163: mod_base = OTHER; note = N1-methylpseudouracil
  modified_base 1166: mod_base = OTHER; note = N1-methylpseudouracil
  modified_base 1169: mod_base = OTHER; note = N1-methylpseudouracil
  modified_base 1175: mod_base = OTHER; note = N1-methylpseudouracil
  modified_base 1179: mod_base = OTHER; note = N1-methylpseudouracil
  modified_base 1186: mod_base = OTHER; note = N1-methylpseudouracil
  modified_base 1194: mod_base = OTHER; note = N1-methylpseudouracil
  modified_base 1199: mod_base = OTHER; note = N1-methylpseudouracil
  modified_base 1207: mod_base = OTHER; note = N1-methylpseudouracil
  modified_base 1210: mod_base = OTHER; note = N1-methylpseudouracil
  modified_base 1227: mod_base = OTHER; note = N1-methylpseudouracil
  modified_base 1234: mod_base = OTHER; note = N1-methylpseudouracil
  modified_base 1236: mod_base = OTHER; note = N1-methylpseudouracil
  modified_base 1237: mod_base = OTHER; note = N1-methylpseudouracil
  modified_base 1247: mod_base = OTHER; note = N1-methylpseudouracil
  modified_base 1249: mod_base = OTHER; note = N1-methylpseudouracil
  modified_base 1250: mod_base = OTHER; note = N1-methylpseudouracil
  modified_base 1258: mod_base = OTHER; note = N1-methylpseudouracil
  modified_base 1259: mod_base = OTHER; note = N1-methylpseudouracil
  modified_base 1266: mod_base = OTHER; note = N1-methylpseudouracil
  modified_base 1268: mod_base = OTHER; note = N1-methylpseudouracil
  modified_base 1271: mod_base = OTHER; note = N1-methylpseudouracil
  modified_base 1281: mod_base = OTHER; note = N1-methylpseudouracil
  modified_base 1289: mod_base = OTHER; note = N1-methylpseudouracil
  modified_base 1292: mod_base = OTHER; note = N1-methylpseudouracil
  modified_base 1303: mod_base = OTHER; note = N1-methylpseudouracil
  modified_base 1305: mod_base = OTHER; note = N1-methylpseudouracil
  modified_base 1310: mod_base = OTHER; note = N1-methylpseudouracil
  modified_base 1312: mod_base = OTHER; note = N1-methylpseudouracil
  modified_base 1313: mod_base = OTHER; note = N1-methylpseudouracil
  modified_base 1322: mod_base = OTHER; note = N1-methylpseudouracil
  modified_base 1330: mod_base = OTHER; note = N1-methylpseudouracil
  modified_base 1336: mod_base = OTHER; note = N1-methylpseudouracil
  modified_base 1337: mod_base = OTHER; note = N1-methylpseudouracil
  modified_base 1349: mod_base = OTHER; note = N1-methylpseudouracil
  modified_base 1351: mod_base = OTHER; note = N1-methylpseudouracil
  modified_base 1352: mod_base = OTHER; note = N1-methylpseudouracil
  modified_base 1355: mod_base = OTHER; note = N1-methylpseudouracil
  modified_base 1360: mod_base = OTHER; note = N1-methylpseudouracil
  modified_base 1373: mod_base = OTHER; note = N1-methylpseudouracil
  modified_base 1377: mod_base = OTHER; note = N1-methylpseudouracil
  modified_base 1382: mod_base = OTHER; note = N1-methylpseudouracil
  modified_base 1386: mod_base = OTHER; note = N1-methylpseudouracil
  modified_base 1392: mod_base = OTHER; note = N1-methylpseudouracil
  modified_base 1397: mod_base = OTHER; note = N1-methylpseudouracil
  modified_base 1400: mod_base = OTHER; note = N1-methylpseudouracil
  modified_base 1406: mod_base = OTHER; note = N1-methylpseudouracil
  modified_base 1407: mod_base = OTHER; note = N1-methylpseudouracil
  modified_base 1410: mod_base = OTHER; note = N1-methylpseudouracil
  modified_base 1418: mod_base = OTHER; note = N1-methylpseudouracil
  modified_base 1427: mod_base = OTHER; note = N1-methylpseudouracil
  modified_base 1438: mod_base = OTHER; note = N1-methylpseudouracil
  modified_base 1441: mod_base = OTHER; note = N1-methylpseudouracil
  modified_base 1443: mod_base = OTHER; note = N1-methylpseudouracil
  modified_base 1458: mod_base = OTHER; note = N1-methylpseudouracil
  modified_base 1466: mod_base = OTHER; note = N1-methylpseudouracil
  modified_base 1470: mod_base = OTHER; note = N1-methylpseudouracil
  modified_base 1471: mod_base = OTHER; note = N1-methylpseudouracil
  modified_base 1483: mod_base = OTHER; note = N1-methylpseudouracil
  modified_base 1487: mod_base = OTHER; note = N1-methylpseudouracil
  modified_base 1489: mod_base = OTHER; note = N1-methylpseudouracil
  modified_base 1493: mod_base = OTHER; note = N1-methylpseudouracil
  modified_base 1494: mod_base = OTHER; note = N1-methylpseudouracil
  modified_base 1496: mod_base = OTHER; note = N1-methylpseudouracil
  modified_base 1499: mod_base = OTHER; note = N1-methylpseudouracil
  modified_base 1508: mod_base = OTHER; note = N1-methylpseudouracil
  modified_base 1510: mod_base = OTHER; note = N1-methylpseudouracil
  modified_base 1514: mod_base = OTHER; note = N1-methylpseudouracil
  modified_base 1517: mod_base = OTHER; note = N1-methylpseudouracil
  modified_base 1533: mod_base = OTHER; note = N1-methylpseudouracil
  modified_base 1535: mod_base = OTHER; note = N1-methylpseudouracil
  modified_base 1547: mod_base = OTHER; note = N1-methylpseudouracil
  modified_base 1555: mod_base = OTHER; note = N1-methylpseudouracil
  modified_base 1557: mod_base = OTHER; note = N1-methylpseudouracil
  modified_base 1558: mod_base = OTHER; note = N1-methylpseudouracil
  modified_base 1570: mod_base = OTHER; note = N1-methylpseudouracil
  modified_base 1571: mod_base = OTHER; note = N1-methylpseudouracil
  modified_base 1574: mod_base = OTHER; note = N1-methylpseudouracil
  modified_base 1578: mod_base = OTHER; note = N1-methylpseudouracil
  modified_base 1587: mod_base = OTHER; note = N1-methylpseudouracil
  modified_base 1588: mod_base = OTHER; note = N1-methylpseudouracil
  modified_base 1591: mod_base = OTHER; note = N1-methylpseudouracil
  modified_base 1592: mod_base = OTHER; note = N1-methylpseudouracil
  modified_base 1601: mod_base = OTHER; note = N1-methylpseudouracil
  modified_base 1607: mod_base = OTHER; note = N1-methylpseudouracil
  modified_base 1608: mod_base = OTHER; note = N1-methylpseudouracil
  modified_base 1617: mod_base = OTHER; note = N1-methylpseudouracil
  modified_base 1618: mod_base = OTHER; note = N1-methylpseudouracil
  modified_base 1622: mod_base = OTHER; note = N1-methylpseudouracil
  modified_base 1626: mod_base = OTHER; note = N1-methylpseudouracil
  modified_base 1633: mod_base = OTHER; note = N1-methylpseudouracil
  modified_base 1634: mod_base = OTHER; note = N1-methylpseudouracil
  modified_base 1647: mod_base = OTHER; note = N1-methylpseudouracil
  modified_base 1648: mod_base = OTHER; note = N1-methylpseudouracil
  modified_base 1649: mod_base = OTHER; note = N1-methylpseudouracil
  modified_base 1654: mod_base = OTHER; note = N1-methylpseudouracil
  modified_base 1655: mod_base = OTHER; note = N1-methylpseudouracil
  modified_base 1667: mod_base = OTHER; note = N1-methylpseudouracil
  modified_base 1668: mod_base = OTHER; note = N1-methylpseudouracil
  modified_base 1669: mod_base = OTHER; note = N1-methylpseudouracil
  modified_base 1674: mod_base = OTHER; note = N1-methylpseudouracil
  modified_base 1676: mod_base = OTHER; note = N1-methylpseudouracil
  modified_base 1697: mod_base = OTHER; note = N1-methylpseudouracil
  modified_base 1715: mod_base = OTHER; note = N1-methylpseudouracil
  modified_base 1718: mod_base = OTHER; note = N1-methylpseudouracil
  modified_base 1723: mod_base = OTHER; note = N1-methylpseudouracil
  modified_base 1724: mod_base = OTHER; note = N1-methylpseudouracil
  modified_base 1727: mod_base = OTHER; note = N1-methylpseudouracil
  modified_base 1760: mod_base = OTHER; note = N1-methylpseudouracil
  modified_base 1761: mod_base = OTHER; note = N1-methylpseudouracil
  modified_base 1769: mod_base = OTHER; note = N1-methylpseudouracil
  modified_base 1772: mod_base = OTHER; note = N1-methylpseudouracil
  modified_base 1780: mod_base = OTHER; note = N1-methylpseudouracil
  modified_base 1781: mod_base = OTHER; note = N1-methylpseudouracil
  modified_base 1789: mod_base = OTHER; note = N1-methylpseudouracil
  modified_base 1790: mod_base = OTHER; note = N1-methylpseudouracil
  modified_base 1793: mod_base = OTHER; note = N1-methylpseudouracil
  modified_base 1801: mod_base = OTHER; note = N1-methylpseudouracil
  modified_base 1803: mod_base = OTHER; note = N1-methylpseudouracil
  modified_base 1804: mod_base = OTHER; note = N1-methylpseudouracil
  modified_base 1806: mod_base = OTHER; note = N1-methylpseudouracil
  modified_base 1827: mod_base = OTHER; note = N1-methylpseudouracil
  modified_base 1828: mod_base = OTHER; note = N1-methylpseudouracil
  modified_base 1831: mod_base = OTHER; note = N1-methylpseudouracil
  modified_base 1832: mod_base = OTHER; note = N1-methylpseudouracil
  modified_base 1852: mod_base = OTHER; note = N1-methylpseudouracil
  modified_base 1853: mod_base = OTHER; note = N1-methylpseudouracil
  modified_base 1856: mod_base = OTHER; note = N1-methylpseudouracil
  modified_base 1864: mod_base = OTHER; note = N1-methylpseudouracil
  modified_base 1866: mod_base = OTHER; note = N1-methylpseudouracil
  modified_base 1872: mod_base = OTHER; note = N1-methylpseudouracil
  modified_base 1876: mod_base = OTHER; note = N1-methylpseudouracil
  modified_base 1877: mod_base = OTHER; note = N1-methylpseudouracil
  modified_base 1883: mod_base = OTHER; note = N1-methylpseudouracil
  modified_base 1887: mod_base = OTHER; note = N1-methylpseudouracil
  modified_base 1902: mod_base = OTHER; note = N1-methylpseudouracil
  modified_base 1912: mod_base = OTHER; note = N1-methylpseudouracil
  modified_base 1931: mod_base = OTHER; note = N1-methylpseudouracil
  modified_base 1937: mod_base = OTHER; note = N1-methylpseudouracil
  modified_base 1946: mod_base = OTHER; note = N1-methylpseudouracil
  modified_base 1949: mod_base = OTHER; note = N1-methylpseudouracil
  modified_base 1952: mod_base = OTHER; note = N1-methylpseudouracil
  modified_base 1957: mod_base = OTHER; note = N1-methylpseudouracil
  modified_base 1958: mod_base = OTHER; note = N1-methylpseudouracil
  modified_base 1964: mod_base = OTHER; note = N1-methylpseudouracil
  modified_base 1967: mod_base = OTHER; note = N1-methylpseudouracil
  modified_base 1970: mod_base = OTHER; note = N1-methylpseudouracil
  modified_base 1979: mod_base = OTHER; note = N1-methylpseudouracil
  modified_base 1987: mod_base = OTHER; note = N1-methylpseudouracil
  modified_base 2003: mod_base = OTHER; note = N1-methylpseudouracil
  modified_base 2012: mod_base = OTHER; note = N1-methylpseudouracil
  modified_base 2015: mod_base = OTHER; note = N1-methylpseudouracil
  modified_base 2020: mod_base = OTHER; note = N1-methylpseudouracil
  modified_base 2021: mod_base = OTHER; note = N1-methylpseudouracil
  modified_base 2026: mod_base = OTHER; note = N1-methylpseudouracil
  modified_base 2027: mod_base = OTHER; note = N1-methylpseudouracil
  modified_base 2029: mod_base = OTHER; note = N1-methylpseudouracil
  modified_base 2033: mod_base = OTHER; note = N1-methylpseudouracil
  modified_base 2037: mod_base = OTHER; note = N1-methylpseudouracil
  modified_base 2049: mod_base = OTHER; note = N1-methylpseudouracil
  modified_base 2054: mod_base = OTHER; note = N1-methylpseudouracil
  modified_base 2063: mod_base = OTHER; note = N1-methylpseudouracil
  modified_base 2064: mod_base = OTHER; note = N1-methylpseudouracil
  modified_base 2068: mod_base = OTHER; note = N1-methylpseudouracil
  modified_base 2072: mod_base = OTHER; note = N1-methylpseudouracil
  modified_base 2084: mod_base = OTHER; note = N1-methylpseudouracil
  modified_base 2085: mod_base = OTHER; note = N1-methylpseudouracil
  modified_base 2093: mod_base = OTHER; note = N1-methylpseudouracil
  modified_base 2105: mod_base = OTHER; note = N1-methylpseudouracil
  modified_base 2111: mod_base = OTHER; note = N1-methylpseudouracil
  modified_base 2118: mod_base = OTHER; note = N1-methylpseudouracil
  modified_base 2121: mod_base = OTHER; note = N1-methylpseudouracil
  modified_base 2127: mod_base = OTHER; note = N1-methylpseudouracil
  modified_base 2128: mod_base = OTHER; note = N1-methylpseudouracil
  modified_base 2129: mod_base = OTHER; note = N1-methylpseudouracil
  modified_base 2135: mod_base = OTHER; note = N1-methylpseudouracil
  modified_base 2142: mod_base = OTHER; note = N1-methylpseudouracil
  modified_base 2150: mod_base = OTHER; note = N1-methylpseudouracil
  modified_base 2151: mod_base = OTHER; note = N1-methylpseudouracil
  modified_base 2153: mod_base = OTHER; note = N1-methylpseudouracil
  modified_base 2154: mod_base = OTHER; note = N1-methylpseudouracil
  modified_base 2162: mod_base = OTHER; note = N1-methylpseudouracil
  modified_base 2168: mod_base = OTHER; note = N1-methylpseudouracil
  modified_base 2171: mod_base = OTHER; note = N1-methylpseudouracil
  modified_base 2174: mod_base = OTHER; note = N1-methylpseudouracil
  modified_base 2177: mod_base = OTHER; note = N1-methylpseudouracil
  modified_base 2182: mod_base = OTHER; note = N1-methylpseudouracil
  modified_base 2183: mod_base = OTHER; note = N1-methylpseudouracil
  modified_base 2191: mod_base = OTHER; note = N1-methylpseudouracil
  modified_base 2203: mod_base = OTHER; note = N1-methylpseudouracil
  modified_base 2204: mod_base = OTHER; note = N1-methylpseudouracil
  modified_base 2207: mod_base = OTHER; note = N1-methylpseudouracil
  modified_base 2209: mod_base = OTHER; note = N1-methylpseudouracil
  modified_base 2215: mod_base = OTHER; note = N1-methylpseudouracil
  modified_base 2237: mod_base = OTHER; note = N1-methylpseudouracil
  modified_base 2240: mod_base = OTHER; note = N1-methylpseudouracil
  modified_base 2245: mod_base = OTHER; note = N1-methylpseudouracil
  modified_base 2246: mod_base = OTHER; note = N1-methylpseudouracil
  modified_base 2249: mod_base = OTHER; note = N1-methylpseudouracil
  modified_base 2257: mod_base = OTHER; note = N1-methylpseudouracil
  modified_base 2261: mod_base = OTHER; note = N1-methylpseudouracil
  modified_base 2266: mod_base = OTHER; note = N1-methylpseudouracil
  modified_base 2267: mod_base = OTHER; note = N1-methylpseudouracil
  modified_base 2269: mod_base = OTHER; note = N1-methylpseudouracil
  modified_base 2279: mod_base = OTHER; note = N1-methylpseudouracil
  modified_base 2282: mod_base = OTHER; note = N1-methylpseudouracil
  modified_base 2284: mod_base = OTHER; note = N1-methylpseudouracil
  modified_base 2286: mod_base = OTHER; note = N1-methylpseudouracil
  modified_base 2289: mod_base = OTHER; note = N1-methylpseudouracil
  modified_base 2291: mod_base = OTHER; note = N1-methylpseudouracil
  modified_base 2296: mod_base = OTHER; note = N1-methylpseudouracil
  modified_base 2299: mod_base = OTHER; note = N1-methylpseudouracil
  modified_base 2302: mod_base = OTHER; note = N1-methylpseudouracil
  modified_base 2307: mod_base = OTHER; note = N1-methylpseudouracil
  modified_base 2314: mod_base = OTHER; note = N1-methylpseudouracil
  modified_base 2318: mod_base = OTHER; note = N1-methylpseudouracil
  modified_base 2323: mod_base = OTHER; note = N1-methylpseudouracil
  modified_base 2327: mod_base = OTHER; note = N1-methylpseudouracil
  modified_base 2328: mod_base = OTHER; note = N1-methylpseudouracil
  modified_base 2330: mod_base = OTHER; note = N1-methylpseudouracil
  modified_base 2331: mod_base = OTHER; note = N1-methylpseudouracil
  modified_base 2337: mod_base = OTHER; note = N1-methylpseudouracil
  modified_base 2338: mod_base = OTHER; note = N1-methylpseudouracil
  modified_base 2344: mod_base = OTHER; note = N1-methylpseudouracil
  modified_base 2357: mod_base = OTHER; note = N1-methylpseudouracil
  modified_base 2360: mod_base = OTHER; note = N1-methylpseudouracil
  modified_base 2365: mod_base = OTHER; note = N1-methylpseudouracil
  modified_base 2366: mod_base = OTHER; note = N1-methylpseudouracil
  modified_base 2369: mod_base = OTHER; note = N1-methylpseudouracil
  modified_base 2377: mod_base = OTHER; note = N1-methylpseudouracil
  modified_base 2385: mod_base = OTHER; note = N1-methylpseudouracil
  modified_base 2388: mod_base = OTHER; note = N1-methylpseudouracil
  modified_base 2390: mod_base = OTHER; note = N1-methylpseudouracil
  modified_base 2391: mod_base = OTHER; note = N1-methylpseudouracil
  modified_base 2392: mod_base = OTHER; note = N1-methylpseudouracil
  modified_base 2396: mod_base = OTHER; note = N1-methylpseudouracil
  modified_base 2401: mod_base = OTHER; note = N1-methylpseudouracil
  modified_base 2403: mod_base = OTHER; note = N1-methylpseudouracil
  modified_base 2407: mod_base = OTHER; note = N1-methylpseudouracil
gggaaanaagagagaaaagaagagnaagaagaaananaagaccccggcgccgccaccangaagngggngaccnncancagccngcngnnccngnncagcagcgccnacagcagaggcgngnncagaagagacgcccacaagagcgaggnggcccacaganncaaggaccngggcgaggagaacnncaaggcccnggngcngancgccnncgcccagnaccngcagcagngcccnnncgaggaccacgngaagcnggngaacgaggngaccgagnncgccaagaccngcgnggccgacgagagcgccgagaacngcgacaagagccngcacacccngnncggcgacaagcngngcaccgnggccacccngagagaaacnnacggcgaganggccgacngcngcgccaagcaggagccagagcggaacgagngcnnccnccagcacaaggacgacaacccnaaccngccnagacnggnaaggccngaggnggacgngangngnaccgccnnccacgacaacgaggagacannccngaagaagnaccngnacgagancgccagaagacacccnnacnncnacgccccngagnngcngnncnncgcgaagaganacaaggccgccnncaccgagngcngccaggccgccganaaggccgcgngccngcngccnaagcnggacgagcngagagacgagggcaaggcanccagcgcnaagcagagacngaagngcgccagccngcagaagnncggagagagagcnnncaaggcgngggcagnggcnaganngagccaaaganncccnaaggcagaanncgcngaggngagcaagcncgngacngaccngaccaaggngcanacagagngcngncacggcgaccngcnggagngcgccgacgacagagccgaccnggccaagnacancngcgagaaccaggacagcancagcagcaagcngaaggagngnngngagaagccncnannggagaagagncacngcanngccgaggnggagaacgacgagangccngcggancngccaagcnnggcggccgacnncgnggagagcaaggacgngngcaagaacnacgccgaggccaaggacgnnnnccngggcangnnccncnacgagnacgcacgcagacanccagacnacagcgnggngcngcngcngagacnggcnaagacanacgaaacnacccnggagaagngcngcgcagcggcggacccncacgagngnnacgccaaggngnncgacgagnncaagccncnggnggaggagccncagaaccngancaagcagaacngngagcngnncgagcagcncggcgagnacaagnnccagaacgcccngnnggnccgcnacaccaagaaggngccncaagnaagnaccccnacccnggnagaggnnagnagaaaccngggcaaggngggcagcaagngcngnaagcacccagaagcnaagaggangccnngcgccgaggacnaccngnccgnngngcngaaccagcngngcgngcngcacgagaagaccccngngagcgacagagngacaaagngnngcaccgagagcnnagngaanagaagaccnngcnncagcgcccnggaagnngacgaaacnnacgngccnaaggagnncaacgccgagacnnncacannccacgccgacannngcacncngagcgagaaggagagacagancaagaagcagaccgcccncgnagagnnggncaagcacaagccgaaggcaaccaaggaacagcnnaaggccgnganggacgacnncgcagccnncgncgagaagngnngnaaggccgacgacaaggagacnngcnncgcagaggaaggcaagaagnnggnagccgccncncaggcngccnngggacncggnggcggcagcgccccnaccagcagcnccacaaagaagacgcagcngcagcnggagcaccngcnacnagacnnacaganganccngaacggcancaacaacnacaagaacccgaagcncaccagaangcngacanncaagnncnacangccnaagaaggccacngagcngaagcaccnncagngccnggaggaggaacnnaagccacnggaagaggcccngaaccnggccccnagnaagaannnccaccncagaccncgcgaccnnannagcgacancaacgngancgngcncgaannaaagggcnccgagacaacanncangngcgaanacgccgacgagacagccaccancgnggagnnccngaaccgcnggancaccnncngccagagcancancncnacncngaccnganaanaggcnggagccncggnggccnagcnncnngccccnngggccnccccccagccccnccnccccnnccngcacccgnacccccgnggncnnngaanaaagncngagngggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 38 - RNA, 1410 nt, synthetic construct
  source 1..1410: mol_type = other RNA
atggaatttggactgtcttgggtttttctggtagctctgtttcgtggagtgcaatgcgaggttcagcttgtcgagagcggcggcggtctcgtgcaacccggacggagtctgagactcagctgtgctgcttcaggatttaccttcgacgattatgcgatgcattgggtgcggcaggctcctggcaaaggattagaatgggtgagcgcaattacatggaactctggccacatcgactacgccgattcagtggagggcaggttcaccattagcagagataatgctaagaatagcttatacttgcagatgaacagcctgcgcgccgaggatactgcggtgtactattgcgccaaggtcagttatctgtccaccgccagctccctagattattgggggcagggaaccttggtgacagtcagttccgcaagcacgaaagggcccagcgttttccctctcgcaccctcttcaaaatccacttcaggggggacagctgcactgggatgtcttgtcaaggattacttccccgaacctgtgaccgtctcatggaattccggtgccttgacctcaggggtgcataccttccccgcagtccttcagagttctggactctactccctgagttctgtggtaacagtcccttctagctccctcgggacacagacctacatttgcaatgtgaaccataaaccgagtaacactaaagtggacaaaaaagtagaacccaaaagctgcgataagacccacacgtgccccccgtgtccggcccccgagcttctgggcggcccttcggtgttcctgtttccacctaagcccaaagacacactgatgatctctcgcactcctgaagtaacatgcgtggtggtcgacgtgagccacgaagatccagaagttaagttcaattggtatgttgacggggtggaggtgcacaacgccaagactaagccaagggaggagcaatacaattccacataccgagttgtcagcgtacttacggttctgcaccaagactggctaaatggtaaagagtacaagtgtaaggtgtctaacaaagccctgccagcgccgatagaaaagactatctcaaaagcaaagggccagcccagagaacctcaggtgtacaccttgccaccatcaagggacgagctcaccaagaatcaggttagtctcacctgtctggtgaaggggttctatccatccgacatcgccgtggagtgggagagtaacggccagcccgaaaacaattacaagacgacaccaccagtcttggatagcgacggttctttctttctatattcgaagctcactgtcgacaagtcgcggtggcagcagggcaacgtatttagctgctccgtcatgcatgaggccctgcacaaccactatactcaaaaatccctgtccttatctcctggcaag

SEQ ID NO: 39 - RNA, 705 nt, synthetic construct
  source 1..705: mol_type = other RNA
atgaagtatctactgcctaccgctgcagctgggctgttgcttctggctgctcaaccagccatggctcagatcgtgctctcccagtcccccgcgattttgagcgcatccccaggcgagaaggtaacaatgacgtgccgcgcgagcagttcagtgtcttacatccactggttccagcaaaagccaggctctagccccaagccctggatttacgcaaccagcaatcttgccagtggcgtcccggtgaggttctcggggtctggaagcggtacatcttatagtctcaccataagcagggtagaggccgaggacgccgctacttactattgccagcagtggacctctaacccacccaccttcggcggtggaaccaagctggaaatcaaacgaacagtcgccgccccgtccgtgtttatcttccctccttctgacgagcagctgaaatccgggaccgcatccgtcgtttgtctactgaacaacttttacccccgggaagcaaaagtccagtggaaggttgataacgccctccaaagtggaaatagccaggaatcagtgactgagcaagattccaaagatagtacttacagcttatcaagcacgctcactctgtccaaagccgactatgaaaaacacaaggtgtacgcctgtgaggtgacacatcaggggctgtcatcacctgttacaaagtcgtttaatagaggcgaatgc

SEQ ID NO: 40 - RNA, 858 nt, synthetic construct
  source 1..858: mol_type = other RNA
atgaaatgggttacgttcatctccttgctgttcctgttctcttccgcttactccgataaaacacatacctgtcccccatgtcccgcccccgagctcctcggcggtcccagtgtgtttctgtttccaccaaaacccaaggacactctcatgataagccgcacgcccgaggttacatgcgtcgtcgtcgacgtctcccacgaggatccggaggtgaaatttaactggtatgtagatggcgtggaagtgcacaacgcaaaaaccaagccccgtgaagaacaatacaactccacatacagggtcgtgagcgtgctgactgtactccatcaggattggttaaatggcaaggagtataagtgtaaggtgagtaataaggccctacctgcacctattgaaaagacaattagcaaagccaagggccagccgagggagccacaggtgtatacactgccacccagccgggatgagttaacaaagaatcaggtttcactcacctgccttgtaaaaggtttttacccatcagacatcgctgtggaatgggagtctaacggacagcctgaaaacaactataaaaccactcctcctgtgctggactctgacggatcgttcttcttgtactcaaaactaaccgtggacaagagtagatggcagcagggaaatgttttctcatgcagcgtcatgcacgaggccctgcataatcactacacccagaagagtttgtctctgagcccgggtaagggaggcggcgggggcatcgaagggcctactctgagacaatggctggcagcccgggctggcgggggtggaggggggggaggcatcgaggggccaacccttcgccaatggcttgcggcgcgagcc

SEQ ID NO: 41 - RNA, 432 nt, synthetic construct
  source 1..432: mol_type = other RNA
atgtggctgcaatccctgctgctgcttggcacagtggcatgctccatcagcgctcctgcccgatctccctcacccagcacacaaccctgggagcatgtcaatgcgatacaggaggccctccggctcttaaatctctcgcgcgatactgcagctgagatgaacgaaaccgtggaagtgatcagtgaaatgttcgatctgcaggaacccacctgtctgcagaccaggctagagttgtacaaacagggcttgagaggatcactaactaaacttaaggggcctctgacgatgatggcctcccactataagcagcactgtccaccaactccagaaacaagttgcgccacacagatcattacctttgagagctttaaggagaacttaaaagacttcctcctggttattcctttcgactgctgggagccggtacaggag

SEQ ID NO: 42 - RNA, 1425 nt, synthetic construct
  source 1..1425: mol_type = other RNA
atgggtgtccctacccagctcctgggactgctcctgctgtggatcaccgacgccatctgcgacatccagatgacccagagccctgccagcctgtctaccagcctgggcgagacagtgaccatccagtgtcaggccagcgaggacatctactctggcctggcttggtatcagcagaagcccggcaagagccctcagctgctgatctacggcgccagcgacctgcaggacggcgtgcctagcagattcagcggcagcggctccggaacccagtacagcctgaagatcaccagcatgcagaccgaggacgagggcgtgtacttctgccagcaaggcctgacctaccctagaaccttcggaggaggcaccaagctggaactgaagggcggaggcggaagtggaggcggaggatctggcggcggaggctctgaagtgcagctgcagcagtctggcgctgaactggtccggcctggcactagcgtgaagctgtcctgcaaggtgtccggcgacaccatcaccttctactacatgcacttcgtgaagcagaggccaggacagggcctggaatggatcggcagaatcgaccctgaggacgagagcaccaagtacagcgagaagttcaagaacaaggccaccctgaccgccgacaccagcagcaacaccgcctacctgaagctgtctagcctgacctccgaggacaccgccacctacttttgcatctacggcggctactacttcgactactggggccagggcgtgatggtcaccgtgtccagcatcgagttcatgtacccccctccctacctggacaacgagagaagcaacggcaccatcatccacatcaaagaaaagcacctgtgccacacccagagcagccccaagctgttctgggccctggtggtggtggccggcgtgctgttctgttacggcctgctggtcacagtggccctgtgcgtgatctggaccaacagcagaagaaacagaggcggccagagcgactacatgaacatgacccccagaaggccaggcctgaccagaaagccctaccagccctacgcccctgccagagacttcgccgcctacagacccagagccaagttcagcagatccgccgagacagccgccaacctgcaggatcccaaccagctgttcaacgagctgaacctgggcagacgggaggaattcgacgtgctggaaaagaagagagccagggaccccgagatgggcggcaagcagcagagaagaagaaaccctcaggaaggcgtctacaacgccctgcagaaagacaagatggccgaggcctacagcgagatcggcaccaagggcgagagaagaaggggcaagggccacgatggcctgttccagggcctgtccaccgccaccaaggacaccttcgacgccctgcacatgcagaccctggcccccaga

SEQ ID NO: 43 - protein, 162 aa, synthetic construct; identical to SEQ ID NO: 14

SEQ ID NO: 44 - RNA, 30 nt, synthetic construct
  source 1..30: mol_type = other RNA
aaaaaaaaaaaaaaaaaaaaaaaaaaaaaa

SEQ ID NO: 45 - RNA, 70 nt, synthetic construct
  source 1..70: mol_type = other RNA
aaaaaaaaaaaaaaaaaaaaaaaaaaaaaaaaaaaaaaaaaaaaaaaaaaaaaaaaaaaaaaaaaaaaaa

SEQ ID NO: 46 - RNA, 200 nt, synthetic construct
  source 1..200: mol_type = other RNA
  misc_difference 1..200: note = This sequence may encompass 100-200 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 47 - RNA, 11 nt, synthetic construct
  source 1..11: mol_type = other RNA
gggatcctacc

SEQ ID NO: 48 - RNA, 582 nt, synthetic construct
  source 1..582: mol_type = other RNA
atgggcgtgcacgagtgccccgcctggctgtggctgctgctgagcctgctgagcctgcccctgggcctgcccgtgctgggcgcccccccccggctgatctgcgacagccgggtgctggagcggtacctgctggaggccaaggaggccgagaacatcaccaccggctgcgccgagcactgcagcctgaacgagaacatcaccgtgcccgacaccaaggtgaacttctacgcctggaagcggatggaggtgggccagcaggccgtggaggtgtggcagggcctggccctgctgagcgaggccgtgctgcggggccaggccctgctggtgaacagcagccagccctgggagcccctgcagctgcacgtggacaaggccgtgagcggcctgcggagcctgaccaccctgctgcgggccctgggcgcccagaaggaggccatcagcccccccgacgccgccagcgccgcccccctgcggaccatcaccgccgacaccttccggaagctgttccgggtgtacagcaacttcctgcggggcaagctgaagctgtacaccggcgaggcctgccggaccggcgaccggtga

SEQ ID NO: 49 - protein, 153 aa, synthetic construct; identical to SEQ ID NO: 30

SEQ ID NO: 50 - DNA, 1431 bp, synthetic construct
  source 1..1431: mol_type = other DNA
atggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccgctacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtaa

SEQ ID NO: 51 - DNA, 1491 bp, synthetic construct
  source 1..1491: mol_type = other DNA
atgtggtggcgcctgtggtggctgctgctgctgctgctgctgctgtggcccatggtgtgggccgtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccgctacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtaa

SEQ ID NO: 52 - DNA, 1473 bp, synthetic construct
  source 1..1473: mol_type = other DNA
atgggctgcgtgtgcagcagcaaccccgagaacaacaacaacatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccgctacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtaa

SEQ ID NO: 53 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 54 - DNA, 462 bp, synthetic construct
  source 1..462: mol_type = other DNA
atgtacaggatgcaactcctgtcttgcattgcactaagtcttgcacttgtcacaaacagtgcacccaccagcagcagcaccaagaagacccagctgcagctggagcacctgctgctggacctgcagatgatcctgaacggcatcaacaactacaagaaccccaagctgaccagaatgctgaccttcaagttctacatgcccaagaaggccaccgagctgaagcacctgcagtgcctggaggaggagctgaagcccctggaggaggtgctgaacctggcccagagcaagaacttccacctgagacccagagacctgatcagcaacatcaacgtgatcgtgctggagctgaagggcagcgagaccaccttcatgtgcgagtacgccgacgagaccgccaccatcgtggagttcctgaacagatggatcaccttcagccagagcatcatcagcaccctgacctga

SEQ ID NO: 55 - DNA, 462 bp, synthetic construct
  source 1..462: mol_type = other DNA
atgtacaggatgcaactcctgtcttgcattgcactaagtcttgcacttgtcacaaacagtgcacccaccagcagcagcaccaagaagacccagctgcagctggagcacctgctgctggacctgcagatgatcctgaacggcatcaacaactacaagaaccccaagctgaccagaatgctgaccgccaagttctacatgcccaagaaggccaccgagctgaagcacctgcagtgcctggaggaggagctgaagcccctggaggaggtgctgaacctggcccagagcaagaacttccacttcgaccccagagacgtggtgagcaacatcaacgtgttcgtgctggagctgaagggcagcgagaccaccttcatgtgcgagtacgccgacgagaccgccaccatcgtggagttcctgaacagatggatcaccttcagccagagcatcatcagcaccctgacctga

SEQ ID NO: 56 - DNA, 972 bp, synthetic construct
  source 1..972: mol_type = other DNA
atgtacaggatgcaactcctgtcttgcattgcactaagtcttgcacttgtcacaaacagtagcaagaacttccacctgagacccagagacctgatcagcaacatcaacgtgatcgtgctggagctgaagggcagcgagaccaccttcatgtgcgagtacgccgacgagaccgccaccatcgtggagttcctgaacagatggatcaccttcagccagagcatcatcagcaccctgaccggcggcagcagcagcaccaagaagacccagctgcagctggagcacctgctgctggacctgcagatgatcctgaacggcatcaacaactacaagaaccccaagctgaccagaatgctgaccttcaagttctacatgcccaagaaggccaccgagctgaagcacctgcagtgcctggaggaggagctgaagcccctggaggaggtgctgaacctggcccagggcagcggcggcggcagcgagctgtgcgacgacgacccccccgagatcccccacgccaccttcaaggccatggcctacaaggagggcaccatgctgaactgcgagtgcaagagaggcttcagaagaatcaagagcggcagcctgtacatgctgtgcaccggcaacagcagccacagcagctgggacaaccagtgccagtgcaccagcagcgccaccagaaacaccaccaagcaggtgaccccccagcccgaggagcagaaggagagaaagaccaccgagatgcagagccccatgcagcccgtggaccaggccagcctgcccggccactgcagagagccccccccctgggagaacgaggccaccgagagaatctaccacttcgtggtgggccagatggtgtactaccagtgcgtgcagggctacagagccctgcacagaggccccgccgagagcgtgtgcaagatgacccacggcaagaccagatggacccagccccagctgatctgcaccggctga

SEQ ID NO: 57 - DNA, 2286 bp, synthetic construct
  source 1..2286: mol_type = other DNA
atgctgctgctggtgaccagcctgctgctgtgcgagctgccccaccccgccttccttttgattccagaggtgcaattacagcaaagcggtgcagaactcgtgaagccaggcgcttcagtaaaaatgtcctgcaaagcttccggctacacattcactagctacaacatgcattgggttaaacaaacacctggccagggattggagtggatcggggctatatatccgggtaacggtgacactagttacaatcagaaattcaagggaaaggccacgcttacagcagacaagagtagcagtaccgcctatatgcaactcagcagcttgaccagcgaagacagcgcagattattactgtgccagatccaactattacggctcaagctactggttctttgacgtgtggggcgccggaaccactgttactgtcagcagcggaggcggtgggagtggcggcggtggttccggcgggggcgggtcagatattgtgttgacgcaatcccccgcaatcctgtccgcgtccccaggcgaaaaagtgacaatgacatgccgggccagctcaagcgtgaattacatggattggtatcagaagaaacccgggagcagccccaagccgtggatctatgcgacgagtaacctggcttccggcgtccccgcccgattcagcgggtccggcagcggcactagttacagtttaaccatcagcagggtggaggctgaagacgcggcaacctattattgccagcagtggtcatttaatcctcccacgtttggcggcggcaccaagctggagataaagggcggggggggcagtggaggcggaggtagcggcggaggaggatcaggaggggggggtagcggaggagggggttcagacatacagatgacacagacaacctcgagcctgagcgcatccctgggggaccgagtcaccatctcatgtagagcatcgcaggacatcagcaaatatctcaactggtaccagcaaaaacccgatggtacagtgaaactcttaatttaccacacatcccggttacatagcggagtcccaagccgctttagcggatccggaagcgggaccgactactccctcacgatcagcaacctcgaacaggaggatatagccacttatttctgccagcagggtaacaccctgccgtatacatttggcgggggcacaaaactagaaatcacaggcagtacaagtggtagtgggaagcccggtagcggagaaggtagcacgaaaggcgaggttaaacttcaggaatcaggcccaggcctagttgccccttcacaaagtctaagcgtcacgtgtaccgtgtccggcgtaagcctgccagattatggagtcagctggattaggcagcctccacggaagggactggagtggctgggcgtgatttgggggtcggagaccacctattataacagcgcgctgaaatcgcggctgactattattaaggataatagcaagtcacaggtgtttcttaagatgaatagcctgcagactgatgacactgcgatatactactgtgccaagcactactactacggagggtcatatgctatggattactgggggcagggaacctccgtgactgtaagttccgccgccgctactacaacccctgccccacgtcccccgaccccagcacctactatcgcatcccagcccctttcccttcgccctgaggcctgcaggcctgcagctggaggagccgttcacaccaggggcctcgactttgcatgcgatatttacatctgggccccactggctggcacctgcggcgtcttgctgctgagcctcgtgatcaccctctattgtaagagggggcgaaaaaagctcctgtacattttcaaacagcctttcatgagacctgtgcagacaacacaggaagaggacggttgctcctgtaggtttccggaggaagaagagggagggtgtgaattgcgtgtaaagttctccagatcagctgatgccccagcctatcaacaaggacagaatcagctgtacaacgagcttaatttggggcggagagaggaatacgatgtcttggacaagagacgggggcgcgaccccgagatgggcgggaagcctagaaggaagaaccctcaggaggggctgtataatgagctccaaaaggataagatggccgaagcttacagcgagatcgggatgaaaggagagcgccgccgcggaaaaggccatgatgggttataccagggccttagcaccgccactaaggacacttacgacgctctacacatgcaggctctcccaccccgatga

SEQ ID NO: 58 - DNA, 1404 bp, synthetic construct
  source 1..1404: mol_type = other DNA
atgagggcttggatcttctttctgctctgcctggccgggcgcgccttggccgaagttcaactggtagagagtggaggtgggcttgtgcaaccaggcggatccttgcgactgtcctgcgccgcttcaggcttcaacatcaaggacacctacatccattgggtccgccaggcaccaggaaaaggtcttgaatgggtggccagaatctaccctactaacggttacaccagatatgcagactccgttaaggggcgatttaccatttcagcagacacctctaagaacaccgcttacctgcagatgaactcacttcgagctgaggacaccgccgtttactattgcagcagatggggcggtgacggcttctacgctatggattactggggacaggggactttggtaactgtgagtagtgcatctacaaaggggccaagcgtgttcccacttgccccatcttctaaaagcacctcaggagggactgcagccttgggttgcttggttaaagattattttccagagcctgtaactgtatcctggaatagtggggccctcacaagcggagtacatactttccctgcagtattgcagtctagtggactctactctctcagcagtgtagtgaccgtaccttccagttcacttggaacacagacctatatttgcaatgtgaatcataagccatctaatactaaagtggataagaaagtggagcctaaatcttgtgacaagactcatacatgccctccctgccctgcccctgaactgttgggagggccctctgtatttctcttcccccctaaaccaaaggacaccctgatgatcagtcgaactcctgaggtgacttgtgtggttgttgacgtgtcacatgaggatcccgaagtgaaattcaactggtacgtcgatggagtagaggtacacaatgcaaagacaaaacctagggaggaacagtataattctacctatagagtggtgtctgttctcacagttctccatcaagactggttgaacggtaaagaatataaatgcaaagtctccaataaggctttgcccgctcccattgaaaaaacaatcagtaaagccaaaggccagccacgcgaaccacaggtctacacccttccaccatctcgcgaagaaatgactaagaatcaagtgtcactcacatgcctcgtcaagggcttttacccttctgatattgcagttgaatgggagtctaacgggcagcccgagaataattacaagactaccccccccgtccttgactctgatggcagctttttcctgtactcaaaattgactgtggacaagtctcgatggcaacagggtaatgttttctcctgtagcgtaatgcacgaggcccttcataaccattatacccaaaaatctctttcattgtcccctggaaaatga

SEQ ID NO: 59 - DNA, 696 bp, synthetic construct
  source 1..696: mol_type = other DNA
atgagggcttggatcttctttctgctctgcctggccgggcgcgccttggccgatattcagatgactcagagccccagcagcctgtctgctagcgttggggatagagtcactataacatgtcgggcttcccaggatgtgaatactgctgtcgcttggtatcaacagaagcccggcaaggcaccaaaactgctgatatatagtgcctctttcctttactccggggttcccagtcgattctctggaagccgcagcggcactgatttcacacttactataagtagtctgcaacctgaggactttgctacatactactgccagcagcactatacaacacccccaactttcgggcaaggcactaaggtagaaattaaaaggaccgttgctgctccatccgtctttatttttccaccatctgatgaacagttgaagagtggaacagcaagcgtcgtttgtctcctgaacaatttttacccacgagaggcaaaagttcaatggaaggtagacaatgcccttcagagcggcaattcccaggagagcgtaaccgagcaggatagcaaagactctacatactctttgagttcaacccttaccctgagcaaggctgattacgaaaaacacaaggtgtacgcttgcgaggtaacccatcagggattgtcatccccagtcacaaaatcattcaataggggcgagtgctga

SEQ ID NO: 60 - RNA, 1398 nt, synthetic construct
  source 1..1398: mol_type = other RNA
atggaatttggactgtcttgggtttttctggtagctctgtttcgtggagtgcaatgccaggttcagttggtccagtcgggtgtggaggttaaaaagcccggcgcctctgtgaaggttagctgcaaggctagcggatatactttcaccaattactacatgtattgggtgaggcaggctccagggcagggactcgagtggatgggaggcatcaacccgtccaacggcggcaccaacttcaatgagaagttcaaaaatagagtgactctcaccactgatagttccacgacaaccgcatacatggaactgaaaagtctgcaattcgatgacaccgccgtgtattactgcgcccgcagagactaccggtttgacatgggatttgattactggggtcagggcaccacagtgacagtctcatccgcctctaccaaaggaccctcggtttttccactcgcaccttgctcccgaagcacctcagagtccaccgcggcccttgggtgtctggtgaaagattatttccccgaaccagtgaccgtgtcatggaattcaggggcactgacaagtggggtccacacgttcccagcggtgctgcagtcctccgggctgtatagccttagctctgtagtcactgtcccgtccagttccctggggactaagacttacacatgtaacgtagatcacaaacctagtaacaccaaggtggacaagcgcgtggagtcaaagtacggacctccatgtcccccatgtcccgcccccgaatttttgggaggaccttcagtgttcctgttccctccaaaacctaaagacacactgatgataagcaggacacccgaggtcacctgcgtcgtggtcgacgtaagccaggaagatccagaggtacagttcaactggtacgtcgacggtgtagaggtccataatgccaagacaaagcccagggaggagcagtttaactcaacatatcgtgttgtgtctgtgttgacggttctacatcaagactggctaaacggcaaggagtataaatgcaaggttagtaataagggcctccccagctctattgagaaaactatcagcaaagctaagggccagcctcgggaaccacaagtttacactttaccgcccagccaggaggaaatgacgaagaatcaggtgagcctgacatgtcttgtcaaggggttttacccctctgatatcgctgtagaatgggaatccaatggtcaacctgaaaacaactacaagaccaccccgcctgtgctcgactctgacggcagctttttcctgtattcgagacttacagtggataaaagccggtggcaggagggcaatgtgttttcctgcagtgtgatgcacgaagcattacacaaccattatactcaaaaatcactcagtttgtctctggggaag

SEQ ID NO: 61 - RNA, 579 nt, synthetic construct
  source 1..579: mol_type = other RNA
atgggggtccacgaatgcccggcatggctgtggttactgctatcgctgctcagcctgccactgggcctgcccgtacttggcgcccctccacgcctcatctgtgactcccgggtcctggagcggtaccttttggaggccaaagaagcagagaatatcactacaggctgcgctgagcactgtagtttgaacgaaaatataacagtacctgacacaaaggtgaatttttacgcctggaaaagaatggaggtgggccaacaggcagtggaagtgtggcagggtctcgctttacttagcgaggccgtgctgcgaggtcaggcactcctagttaactccagccagccctgggagccactccagttgcatgtggataaggccgtctctggactaagatccctcaccaccctgctgagggcattgggagcgcaaaaggaggctatttcacctcccgatgccgctagtgctgcgccccttcgcactatcaccgccgacacgttccgtaagctgttcagggtttattcaaactttctccgggggaagctgaaactgtatactggggaagcctgcagaaccggagatagg

SEQ ID NO: 62 - RNA, 1455 nt, synthetic construct
  source 1..1455: mol_type = other RNA
atgaaatgggttacgttcatctccttgctgttcctgttctcttccgcttactccctgccagcccaggtcgctttcacgccatacgctcctgaaccaggtagcacttgtcgtctacgggaatactatgaccagactgcacagatgtgctgttccaagtgctctcctggccaacatgccaaagtgttttgcactaagactagcgatacagtgtgcgatagctgcgaggattctacatatacccagctctggaactgggttcccgagtgtctcagctgtggcagccggtgtagtagcgaccaggtggagacccaggcctgcacacgagagcagaaccggatctgcacatgtagacccgggtggtactgtgcgttgagtaaacaggagggatgtagactctgcgcccctcttaggaagtgccgcccagggtttggagtcgcacgcccaggcacagaaaccagtgacgtcgtctgcaagccgtgtgccccgggcacattctccaacactacctcaagtacagatatttgtagacctcatcagatctgcaatgtcgtggcaattcccggtaacgcctcgatggatgccgtgtgcacctctacgtcgcccactaggtccatggccccaggagcagtacacttacctcagcctgttagcacacgctcccagcacacgcaacccacccctgagccttccactgcaccgtccaccagcttcctgctgcccatgggcccaagtcctccagcggagggcagcacaggggacgagccaaagtcctgcgacaaaacccatacttgtcccccttgccccgcacccgaactccttgggggaccatcagtattcttgtttccccccaagccaaaggacacattaatgatttctaggactccggaggtgacttgcgtcgttgttgacgtgagccacgaagacccagaggtgaaattcaactggtatgtggatggagtcgaagttcacaatgcgaaaaccaaacctcgagaggaacagtacaactcaacctatagggtagtgagtgttctgaccgtgctgcaccaggactggctcaatgggaaggaatacaagtgtaaggtatccaataaagccctgcctgcccctatcgaaaagacaataagcaaggctaaggggcaacctcgcgaaccccaggtgtacacgctgccaccaagcagagaggagatgacaaaaaaccaagtgtcactgacctgcctggtgaaaggcttttacccgagcgatatcgctgtggagtgggagtcaaacggacaacccgagaataactataaaaccacgccccccgtgctggattcagacggttctttcttcctttattccaagttgaccgtggacaagtctcggtggcagcagggcaatgtcttttcctgtagtgtcatgcacgaagctctacacaatcattacacccaaaaaagcctgtcactctctcccggtaaa

SEQ ID NO: 63 - RNA, 612 nt, synthetic construct
  source 1..612: mol_type = other RNA
atggcaggcccagctacgcagagcccaatgaaactgatggcactgcagctcctgttatggcacagcgccctgtggaccgtccaggaagcaacacccctcgggcctgcgtccagtctcccccagagctttctacttaaatgtctagagcaggtgcggaagatccaaggagacggggccgcactgcaggagaagctctgcgctacatacaagctttgtcacccagaggaactggtgctgttaggccattcactgggaatcccttgggcccctttgtctagttgcccgtcccaggctttacagttggccggctgcctgagccagctgcactccggactatttctgtatcagggacttttgcaggccctggagggtatttcccctgagctcgggccgactctggatacactgcagcttgatgtcgccgactttgctactaccatatggcaacaaatggaggaactcggtatggccccagcgctgcaacccacccagggggccatgcccgctttcgcgtctgccttccagcgcagagcaggcggcgtgttggtggcctcacatctccaatcgttcctggaagtttcttaccgagtactcaggcacttggcacagccc

(19) United States
(12) Patent Application Publication
(10) Pub. No.: US 2026/0263575 A1
(43) Pub. Date: Sep. 10, 2026

(54) MRNA THERAPEUTIC COMPOSITIONS

(71) Applicant: Sail Biomedicines, Inc., Cambridge, MA (US)

(72) Inventors: Munir MOSAHEB, Cambridge, MA (US); Siddharth PATEL, Cambridge, MA (US); Stacie CLARK, Cambridge, MA (US); Emad ARAFA, Cambridge, MA (US); Roman BOGORAD, Cambridge, MA (US)

(21) Appl. No.: 18/721,328

(22) PCT Filed: Dec. 20, 2022
(86) PCT No.: PCT/US2022/053492

§ 371 Date: Jun. 18, 2024

Related U.S. Application Data
(60) Provisional application No. 63/291,686, filed on Dec. 20, 2021.
(60) Provisional application No. 63/320,664, filed on Mar. 16, 2022.
(60) Provisional application No. 63/401,214, filed on Aug. 26, 2022.

(51) Int. Cl. A61K 39/00; A61K 9/00; A61K 9/1271; A61K 9/51; A61K 38/20; A61K 45/06; A61K 47/18; A61K 47/22; A61P 35/00; A61P 37/04

(52) U.S. Cl. CPC A61K 39/00114; A61K 9/0019; A61K 9/1271; A61K 9/5123; A61K 9/5176; A61K 38/2013; A61K 45/06; A61K 47/18; A61K 47/22; A61P 35/00; A61P 37/04; A61K 2039/53; A61K 2039/54; A61K 2039/545; A61K 2039/55555

(57) ABSTRACT

Disclosed herein are mRNA therapeutic compositions including one or more polynucleotides encoding one or more tumor antigenic, immunogenic, or signaling polypeptides, formulated within a lipid reconstructed plant messenger packs (LPMPs) comprising natural lipids and an ionizable lipid. The disclosure also includes a method for making a mRNA therapeutic composition, comprising reconstituting a film comprising purified PMP lipids in the presence of an ionizable lipid to produce a LPMP comprising the ionizable lipid, and loading into the LPMPs with one or more polynucleotides encoding one or more tumor antigenic or immunogenic polypeptides.

### CROSS-REFERENCE TO RELATED APPLICATIONS

This application claims benefit of priority to U.S. Provisional Application No. 63/291,686, filed Dec. 20, 2021; U.S. Provisional Application No. 63/320,664, filed Mar. 16, 2022; and U.S. Provisional Application No. 63/401,214, filed Aug. 26, 2022; all of which are herein incorporated by reference in their entirety.

### BACKGROUND

Commercial or developing vaccines (e.g., cancer vaccines) are typically based on whole microorganisms, protein antigens, peptides, polysaccharides or deoxyribonucleic acid (DNA) vaccines and their combinations. The use of RNA polynucleotides as therapeutics is a new and emerging field.

A need therefore exists for developing an enhanced RNA delivery system for a more effective, easily scalable, and stable delivery of RNA therapeutics (such as cancer vaccines).

### SUMMARY OF THE INVENTION

In one aspect, provided herein is a mRNA therapeutic composition, comprising one or more polynucleotides encoding one or more antigenic (e.g., tumor antigenic) or signaling polypeptides. The one or more polynucleotides are formulated within a lipid reconstructed plant messenger packs (LPMPs) comprising natural lipids and an ionizable lipid. The ionizable lipid has two or more of the characteristics listed below:
(i) at least 2 ionizable amines;
(ii) at least 3 lipid tails, wherein each of the lipid tails is at least 6 carbon atoms in length;
(iii) a pKa of about 4.5 to about 7.5;
(iv) an ionizable amine and a heteroorganic group separated by a chain of at least two atoms; and
(v) an N:P ratio of at least 10.

In another aspect, provided herein is a method for making a mRNA therapeutic composition. The method comprises reconstituting a film comprising purified PMP lipids in the presence of an ionizable lipid to produce a lipid reconstructed plant messenger packs (LPMP) comprising the ionizable lipid. The ionizable lipid has two or more of the characteristics listed below:
(i) at least 2 ionizable amines;
(ii) at least 3 lipid tails; wherein each of the lipid tails is at least 6 carbon atoms in length;
(iii) a pKa of about 4.5 to about 7.5;
(iv) an ionizable amine and a heteroorganic group separated by a chain of at least two atoms; and
(v) an N:P ratio of at least 10.

The method further comprises loading into the LPMPs with one or more polynucleotides encoding one or more antigenic (e.g., tumor antigenic) or signaling polypeptides.

In some embodiments, the polynucleotides are polynucleotide constructs, which encode one or more wild type or engineered antigens (or an antibody to an antigen). The antigen may be derived from a tumor, e.g., a tumor specific antigen, a tumor associated antigen, a tumor neoantigen, or a combination thereof.

In some embodiments, the polypeptide encoded by the polynucleotide is an antigenic (e.g., tumor antigenic) or signaling polypeptide comprising p53, ART-4, BAGE, ss-catenin/m, Bcr-abL CAMEL, CAP-1, CASP-8, CDC27/m, CDK4/m, CEA, CLAUDIN-12, c-MYC, CT, Cyp-B, DAM, ELF2M, ETV6-AML1, G250, GAGE, GnT-V, Gap 100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A10, MAGE-A11, or MAGE-A12, MAGE-B, MAGE-C, MART-1/Melan-A, MC1R, Myosin/m, MUC1, MUM-1, -2, -3, NA88-A, NF1, NY-ESO-1, NY-BR-1, pI90 minor BCR-abL, Plac-1, Pml/RARa, PRAME, proteinase 3, PSA, PSM, RAGE, RU1 or RU2, SAGE, SART-1 or S ART-3, SCGB3A2, SCP1, SCP2, SCP3, SSX, SURVIVIN, TEL/AML1, TPI/m, TRP-1, TRP-2, TRP-2/INT2, TPTE, WT, WT-1, or a combination thereof.

In some embodiments, the polypeptide encoded by the polynucleotide is a antigenic (e.g., tumor antigenic) or signaling polypeptide comprising CD2, CD3, CD4, CD8, CD11b, CD14, CD16, CD19, CD20, CD22, CD25, CD27, CD33, CD37, CD38, CD40, CD44, CD45, CD47, CD52, CD56, CD70, CD79, CD137, 4-IBB, 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbBI, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gplOO, gpA33, GPNMB, HLA, HLA-DR, ICOS, IGF1R, Integrin av, Integrin αvβ, LAG-3, Lewis Y, Mesothelin, c-MET, MN Carbonic anhydrase IX, MUC1, MUC16, Nectin-4, KGD2, NOTCH, OX40, OX40L, PD-1, PDL1, PSCA, PSMA, RANKL, ROR1, ROR2, SLC44A4, Syndecan-1, TACI, TAG-72, Tenascin, TIM3, TRAILR1, TRAILR2, VEGFR-1, VEGFR-2, VEGFR-3, and variants thereof.

In some embodiments, the polypeptide encoded by the polynucleotide is a antigenic (e.g., tumor antigenic) or signaling polypeptide comprising IL-1 α, IL-1 β, IL-1ra, IL-2, IL-3, IL-4, IL-5, IL-6, IL-7, IL-8, IL-9, IL-10, IL-11, IL-12, IL-13, IL-14, IL-15, IL-16, IL-17A, IL-17B, IL-17C, IL-17D, IL-17E, IL-17F, IL-18, IL-19, IL-20, IL-21, IL-22, IL-23, IL-24, IL-25, IL-26, IL-27, IL-28A/B, IL-29, IL-30, IL-31, IL-32, IL-33, IL-35, TGF-β, GM-CSF, M-CSF, G-CSF, TNF-α, TNF-β, LAF, TCGF, BCGF, TRF, BAF, BDG, MP, LIF, OSM, TMF, PDGF, IFN-α, IFN-β, IFN-γ, Uteroglobins, Foxp3, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CCL1, CCL2, CCL3, CCL4, CCL5, CCL6, CCL7, CCL8, CCL9/10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, XCL1, XCL2, CX3CL1 and variants thereof.

In some embodiments, the polypeptide encoded by the polynucleotide is IL-2 peptide, IL-2-Ra, tdTomato, Cre recombinase, GFP, eGFP, Anti-CD19, CD20, CAR-T, Anti-HER2, Etanercept (Enbrel), Humira, erythropoietin, Epogen, Filgrastim, Keytruda, Rituximab, Romiplostim, Sargramostim, or a fragment or subunit thereof. In one embodiment, the polypeptide is IL-2 peptide, or a fragment or subunit thereof. In one embodiment, the polypeptide is erythropoietin or Epogen, or a fragment or subunit thereof.

In some embodiments, the polypeptide encoded by the polynucleotide is IL-15 peptide, IL-15-Ra, or a fragment or subunit thereof. In one embodiment, the polypeptide is IL-15 peptide, or a fragment or subunit thereof.

In some embodiments, the tumor antigenic polypeptide comprises a tumor antigen selected from the group consisting of a carcinoma, a sarcoma, a melanoma, a lymphoma, a leukemia, and a combination thereof. In one embodiment, the tumor antigenic polypeptide comprises a lung cancer antigen.

In some embodiments, the polynucleotide may be a mRNA, an siRNA or siRNA precursor, a microRNA (miRNA) or miRNA precursor, a plasmid, a Dicer substrate small interfering RNA (dsiRNA), a short hairpin RNA (shRNA), an asymmetric interfering RNA (aiRNA), a peptide nucleic acid (PNA), a morpholino, a locked nucleic acid (LNA), a piwi-interacting RNA (piRNA), a ribozyme, a deoxyribozyme (DNAzyme), an aptamer, a circular RNA (circRNA), a guide RNA (gRNA), or a DNA molecule encoding any of these RNAs. In one embodiment, the polynucleotide is an mRNA.

In one embodiment, the polynucleotide is an mRNA which encodes an IL-2 molecule comprising an amino acid sequence having at least 85%, 90%, 95%, 96%, 97%, 98%, 99% or 100% identity to the amino acid sequence of an IL-2 molecule provided in any one of Tables I-III.

In one embodiment, the polynucleotide is an mRNA which encodes an IL-15 or IL-15RA molecule comprising an amino acid sequence having at least 85%, 90%, 95%, 96%, 97%, 98%, 99% or 100% identity to the amino acid sequence of an IL-15 molecule provided in Tables IV. In one embodiment, the polynucleotide is an mRNA which encodes an IL-15 molecule comprising a nucleic acid sequence having at least 85%, 90%, 95%, 96%, 97%, 98%, 99% or 100% identity to the nucleic acid sequence of an IL-15 or IL-15RA molecule provided in Tables IV.

In some embodiments, the mRNA is derived from (a) a DNA molecule, or (b) an RNA molecule. In the mRNA, T is optionally substituted with U.

In some embodiments, the mRNA is derived from a DNA molecule. The DNA molecule can further comprise a promoter. In some embodiments, the promoter is a T7 promoter, a T3 promoter, or an SP6 promoter. In some embodiments, the promoter is located at the 5′ UTR.

In some embodiments, the mRNA is derived from an RNA molecule. The RNA molecule may be a self-replicating RNA molecule.

In some embodiments, the mRNA is an RNA molecule. The RNA molecule may further comprise a 5′ cap. The 5′ cap can have a Cap 1 structure, a Cap 1 (m6A) structure, a Cap 2 structure, a Cap 3 structure, a Cap 0 structure, or any combination thereof.

In some embodiments, the polynucleotide is an mRNA which encodes an IL-2 molecule. In one embodiment, the IL-2 molecule comprises a naturally occurring IL-2 molecule, a fragment of a naturally occurring IL-2 molecule, or a variant thereof. In one embodiment, the IL-2 molecule comprises a variant of a naturally occurring IL-2 molecule (e.g., an IL-2 variant, e.g., as described herein), or a fragment thereof.

In some embodiments, the polynucleotide is an mRNA which encodes an IL-15 molecule. In one embodiment, the IL-15 molecule comprises a naturally occurring IL-15 molecule, a fragment of a naturally occurring IL-15 molecule, or a variant thereof. In one embodiment, the IL-15 molecule comprises a variant of a naturally occurring IL-15 molecule (e.g., an IL-15 variant, e.g., as described herein), or a fragment thereof. In one embodiment, the polynucleotide is an mRNA which encodes an IL-15 superagonist, IL-15 molecule, IL-15RA molecule, or a combination thereof.

In some embodiments, the mRNA comprises a 5′ untranslated region (UTR) and/or a 3′ UTR.

In some embodiments, the mRNA comprises a 5′ UTR. The 5′ UTR may comprise a Kozak sequence.

In some embodiments, the mRNA comprises a 3′ UTR. In some embodiments, the 3′ UTR comprises one or more sequences derived from an amino-terminal enhancer of split (AES). In some embodiments, the 3′ UTR comprises a sequence derived from mitochondrially encoded 12S rRNA (mtRNRI).

In some embodiments, the mRNA comprises a poly(A) sequence. In one embodiment, the poly(A) sequence is a 110-nucleotide sequence consisting of a sequence of 30 adenosine residues, a 10-nucleotide linker sequence, and a sequence of 70 adenosine residues.

In some embodiments, the polynucleotide is encapsulated by the lipid reconstructed plant messenger packs (LPMPs). In some embodiments, the polynucleotide is embedded on the surface of the LPMPs. In some embodiments, the polynucleotide is conjugated to the surface of the LPMPs.

In some embodiments, the LPMP is produced by a method comprising lipid extrusion. In some embodiments, the LPMP is produced by a method comprising processing a solution comprising a lipid extract of the PMPs in a microfluidics device comprising an aqueous phase, thereby producing the LPMPs. In some embodiments, the aqueous phase comprises the polynucleotides.

In some embodiments, the natural lipids of the LPMPs are extracted from lemon or algae.

In some embodiments, the ionizable lipid of the LPMPs is selected from the group consisting of 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), MD1 (cKK-E12), OF2, EPC, ZA3-Ep10, TT3, LP01, 5A2-SC8, Lipid 5, SM-102 (Lipid H), and ALC-315.

In one embodiment, the ionizable lipid is C12-200.

In some embodiments, the ionizable lipid is

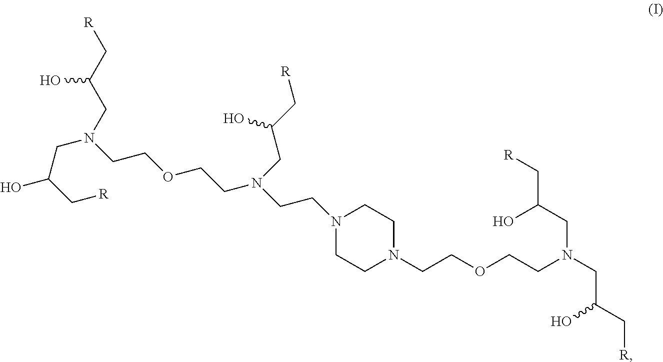

wherein R is C8-C14 alkyl group.

In some embodiments, the reconstitution is performed in the presence of a sterol, thereby producing a LPMP that comprises natural lipids, an ionizable lipid, and a sterol. In some embodiments, the sterol is cholesterol or sitosterol.

In some embodiments, the reconstitution is performed in the presence of a PEGylated lipid (or a PEG-lipid conjugate), thereby producing a LPMP that comprises natural lipids, a ionizable lipid, and a PEG-lipid conjugate. In some embodiments, the PEG-lipid conjugate is C14-PEG2k, C18-PEG2k, or DMPE-PEG2k. In some embodiments, the PEG-lipid conjugate is PEG-DMG or PEG-PE. In some embodiments, the PEG-DMG is PEG2000-DMG or PEG2000-PE.

In some embodiments, the LPMPs further comprise a sterol and a polyethylene glycol (PEG)-lipid conjugate.

In some embodiments, the LPMP comprises:
about 20 mol % to about 50 mol % of the ionizable lipid,
about 20 mol % to about 60 mol % of the natural lipids,
about 7 mol % to about 20 mol % of the sterol, and
about 0.5 mol % to about 3 mol % of the polyethylene glycol (PEG)-lipid conjugate.

In some embodiments, the LPMP comprises:
about 35 mol % of the ionizable lipid,
about 50 mol % of the natural lipids,
about 12.5 mol % of the sterol, and
about 2.5 mol % the polyethylene glycol (PEG)-lipid conjugate.

In one embodiment, the LPMPs comprise the ionizable lipid:natural lipids:sterol:PEG-lipid at a molar ratio of about 35:50:12.5:2.5. In one embodiment, the LPMPs comprise the ionizable lipid:natural lipids:sterol:PEG-lipid at a molar ratio of about 35:20:42.5:2.5.

In some embodiments, the LPMPs comprise:
natural lipids extracted from lemon or algae,
C12-200,
cholesterol, and
DMPE-PEG2k.

In one embodiment, the LPMPs comprise:
natural lipids extracted from lemon,
C12-200,
cholesterol, and
DMPE-PEG2k. The LPMPs may comprise C12-200:lemon lipids:cholesterol:DMPE-PEG2k at a molar ratio of about 35:50:12.5:2.5.

In one embodiment, the LPMPs comprise:
natural lipids extracted from algae,
C12-200,
cholesterol, and
DMPE-PEG2k. The LPMPs may comprise C12-200:algae lipids:cholesterol:DMPE-PEG2k at a molar ratio of about 35:20:42.5:2.5.

In some embodiments, the LPMP is a lipophilic moiety selected from the group consisting of a lipoplex, a liposome, a lipid nanoparticle, a polymer-based carrier, an exosome, a lamellar body, a micelle, and an emulsion. In one embodiment, the LPMP is a liposome selected from the group consisting of a cationic liposome, a nanoliposome, a proteoliposome, a unilamellar liposome, a multilamellar liposome, a ceramide-containing nanoliposome, and a multivesicular liposome. In one embodiment, the LPMP is a lipid nanoparticle.

In some embodiments, the LPMP has a size of less than about 200 nm. In one embodiment, the LPMP has a size of less than about 150 nm. In one embodiment, the LPMP has a size of less than about 100 nm. In one embodiment, the LPMP has a size of about 55 nm to about 80 nm.

In some embodiments, the mRNA therapeutic composition has a total lipid:polynucleotide weight ratio of about 50:1 to about 10:1. In one embodiment, the mRNA therapeutic composition has a total lipid:polynucleotide weight ratio of about 44:1 to about 24:1. In one embodiment, the mRNA therapeutic composition has a total lipid:polynucleotide weight ratio of about 40:1 to about 28:1. In one embodiment, the mRNA therapeutic composition has a total lipid:polynucleotide weight ratio of about 38:1 to about 30:1. In one embodiment, the mRNA therapeutic composition has a total lipid:polynucleotide weight ratio of about 37:1 to about 33:1.

In some embodiments, the mRNA therapeutic composition, e.g., the aqueous phase, further comprises a HEPES or TRIS buffer. The HEPES or TRIS buffer may have a pH of about 7.0 to about 8.5. The HEPES or TRIS buffer can be at a concentration of about 7 mg/mL to about 15 mg/mL. The aqueous phase may further comprise about 2.0 mg/mL to about 4.0 mg/mL of NaCl.

In some embodiments, the mRNA therapeutic composition, e.g., the aqueous phase comprises water, PBS, or a citrate buffer. In one embodiment, the aqueous phase comprises a citrate buffer having a pH of about 3.2.

In some embodiments, the aqueous phase and the lipid solution are mixed at a 3:1 volumetric ratio.

In some embodiments, the mRNA therapeutic composition further comprises one or more cryoprotectants. The one or more cryoprotectants may be sucrose, glycerol, or a combination thereof. In one embodiment, the mRNA therapeutic composition comprises a combination of sucrose at a concentration of about 70 mg/mL to about 110 mg/mL and glycerol at a concentration of about 50 mg/mL to about 70 mg/mL.

In some embodiments, the mRNA therapeutic composition is a lyophilized composition. The lyophilized mRNA therapeutic composition may comprise one or more lyoprotectants. The lyophilized mRNA therapeutic composition may comprise a poloxamer, potassium sorbate, sucrose, or any combination thereof. In one embodiment, the lyophilized mRNA therapeutic composition comprises a poloxamer, e.g., poloxamer 188.

In some embodiments, the mRNA therapeutic composition is a lyophilized composition. In one embodiment, the lyophilized mRNA therapeutic composition comprises about 0.01 to about 1.0% w/w of the polynucleotides. In one embodiment, the lyophilized mRNA therapeutic composition comprises about 1.0 to about 5.0% w/w lipids. In one embodiment, the lyophilized mRNA therapeutic composition comprises about 0.5 to about 2.5% w/w of TRIS buffer. In one embodiment, the lyophilized mRNA therapeutic composition comprises about 0.75 to about 2.75% w/w of NaCl. In one embodiment, the lyophilized mRNA therapeutic composition comprises about 85 to about 95% w/w of a sugar, e.g., sucrose. In one embodiment, the lyophilized mRNA therapeutic composition comprises about 0.01 to about 1.0% w/w of a poloxamer, e.g., poloxamer 188. In one embodiment, the lyophilized mRNA therapeutic composition comprises about 1.0 to about 5.0% w/w of potassium sorbate.

In another aspect, provided herein is a method of delivering an mRNA therapeutic in a subject, comprising administering to the subject the mRNA therapeutic composition discussed in the above aspects of the invention.

In another aspect, provided herein is a method of inducing an immune response in a subject, comprising administering to the subject the mRNA therapeutic composition discussed in the above aspects of the invention.

In another aspect, provided herein is a method of treating or preventing a cancer in a subject, comprising administering to the subject the mRNA therapeutic composition discussed in the above aspects of the invention.

In these aspects of the invention regarding a method of delivering an mRNA therapeutic, a method of inducing an immune reponse, and a method of treating or preventing a cencer, the mRNA therapeutic composition may be administered by oral, intravenous, intradermal, intramuscular, intranasal, intraocular, or rectal, and/or subcutaneous administration. In certain embodiments, the mRNA therapeutic composition is administered by oral, intravenous, intramuscular, and/or subcutaneous administration.

In some embodiments, the mRNA therapeutic composition is administered at a dosage level sufficient to deliver about 0.001 mg/kg to about 0.5 mg/kg (e.g., about 0.005 mg/kg to about 0.5 mg, about 0.006 mg/kg to about 0.5 mg/kg, or 0.01 mg/kg to about 0.4 mg/kg) of the polynucleotide (e.g., mRNA) to the subject. In some embodiments, the mRNA therapeutic composition is administered at a dosage level sufficient to deliver about 0.006 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, or about 0.4 mg/kg of the polynucleotide (e.g., mRNA) to the subject. In some embodiments, the mRNA therapeutic composition is administered at a dosage level sufficient to deliver about 0.0001 mg/kg to about 0.0005 mg/kg (e.g., about 0.0003 mg/kg to about 0.002 mg/kg) of the polynucleotide (e.g., mRNA) to the subject.

In some embodiments, the mRNA therapeutic composition is administered to the subject once, twice, three times, four times, or more. In some embodiments, the mRNA therapeutic composition is administered to the subject once or twice. In some embodiments, the mRNA therapeutic composition is administered to the subject four times.

In some embodiments, the method further comprises administering an additional therapeutic agent to the subject.

In some embodiments, the additional therapeutic agent is an anti-cancer therapeutic agent.

In some embodiments, the additional therapeutic agent is an immunogenic therapeutic agent.

In some embodiments, the additional therapeutic agent is a signaling therapeutic agent.

In some embodiments, the additional therapeutic agent is a therapeutic agent that treats and/or prevents a chronic pain. In one embodiment, the additional therapeutic agent is an opioid analgesic such as buprenorphine, a non-steroidal anti-inflammatory drugs (NSAIDs) such as meloxicam SR, or combinations thereof.

In some embodiments, the additional therapeutic agent is administered prior to, concurrent with, or after the administration of the mRNA therapeutic composition.

### Definitions

As used herein, the term “effective amount,” “effective concentration,” or “concentration effective to” refers to an amount of a LPMP, or nucleic acid composition, sufficient to effect the recited result or to reach a target level (e.g., a predetermined or threshold level) in or on a target organism.

As used herein, the term “therapeutic agent” refers to an agent that can act on an animal, e.g., a mammal (e.g., a human), an animal pathogen, or a pathogen vector, such as an antifungal agent, an antibacterial agent, a virucidal agent, an anti-viral agent, an insecticidal agent, a nematicidal agent, an antiparasitic agent, or an insect repellent.

As defined herein, the term “nucleic acid” and “polynucleotide” are interchangeable and refer to RNA or DNA that is linear or branched, single or double stranded, or a hybrid thereof, regardless of length (e.g., at least 2, 3, 4, 5, 6, 7, 8, 9, 10, 20, 30, 40, 50, 100, 150, 200, 250, 500, 1000, or more nucleic acids). The term also encompasses RNA/DNA hybrids. Nucleotides are typically linked in a nucleic acid by phosphodiester bonds, although the term “nucleic acid” also encompasses nucleic acid analogs having other types of linkages or backbones (e.g., phosphoramide, phosphorothioate, phosphorodithioate, O-methylphosphoroamidate, morpholino, locked nucleic acid (LNA), glycerol nucleic acid (GNA), threose nucleic acid (TNA), and peptide nucleic acid (PNA) linkages or backbones, among others). The nucleic acids may be single-stranded, double-stranded, or contain portions of both single-stranded and double-stranded sequence. A nucleic acid can contain any combination of deoxyribonucleotides and ribonucleotides, as well as any combination of bases, including, for example, adenine, thymine, cytosine, guanine, uracil, and modified or non-canonical bases (including, e.g., hypoxanthine, xanthine, 7-methylguanine, 5,6-dihydrouracil, 5-methylcytosine, and 5 hydroxymethylcytosine).

As used herein, the term “peptide,” “protein,” or “polypeptide” encompasses any chain of naturally or non-naturally occurring amino acids (either D- or L-amino acids), regardless of length (e.g., at least 2, 3, 4, 5, 6, 7, 10, 12, 14, 16, 18, 20, 25, 30, 40, 50, 100, or more amino acids), the presence or absence of post-translational modifications (e.g., glycosylation or phosphorylation), or the presence of, e.g., one or more non-amino acyl groups (for example, sugar, lipid, etc.) covalently linked to the peptide, and includes, for example, natural proteins, synthetic, or recombinant polypeptides and peptides, hybrid molecules, peptoids, or peptidomimetics.

As used herein, “percent identity” between two sequences is determined by the BLAST 2.0 algorithm, which is described in Altschul et al., (1990) J. Mol. Biol. 215:403-410. Software for performing BLAST analyses is publicly available through the National Center for Biotechnology Information.

As used herein, the term “plant” refers to whole plants, plant organs, plant tissues, seeds, plant cells, seeds, and progeny of the same. Plant cells include, without limitation, cells from seeds, suspension cultures, embryos, meristematic regions, callus tissue, leaves, roots, shoots, gametophytes, sporophytes, pollen, and microspores. Plant parts include differentiated and undifferentiated tissues including, but not limited to the following: roots, stems, shoots, leaves, pollen, seeds, fruit, harvested produce, tumor tissue, sap (e.g., xylem sap and phloem sap), and various forms of cells and culture (e.g., single cells, protoplasts, embryos, and callus tissue). The plant tissue may be in a plant or in a plant organ, tissue, or cell culture.

As used herein, the term “modified PMPs” or “modified LPMPs” refers to a composition including a plurality of PMPs or LPMPs that include one or more heterologous agents (e.g., one or more exogenous lipids, such as a ionizable lipids, e.g., a PMP or LPMP comprising an ionizable lipid and a sterol and/or a PEGylated lipid) capable of increasing cell uptake (e.g., animal cell uptake, plant cell uptake, bacterial cell uptake, or fungal cell uptake) of the PMP or LPMP, or a portion or component thereof, relative to an unmodified PMP or LPMP; capable of enabling or increasing delivery of a heterologous functional agent (e.g., an agricultural or therapeutic agent) by the PMP or LPMP to a cell, and/or capable of enabling or increasing loading (e.g., loading efficiency or loading capacity) of a heterologous functional agent (e.g., an agricultural or therapeutic agent). The PMPs or LPMPs may be modified in vitro or in vivo.

As used herein, the term “unmodified PMPs” or “unmodified LPMPs” refers to a composition including a plurality of PMPs or LPMPs that lack a heterologous cell uptake agent capable of increasing cell uptake (e.g., animal cell uptake, plant cell uptake, bacterial cell uptake, or fungal cell uptake) of the PMP.

As used herein, the term “cell uptake” refers to uptake of a PMP or LPMP or a portion or component thereof (e.g., a polynucleotide carried by the PMP or LPMP) by a cell, such as an animal cell, a plant cell, bacterial cell, or fungal cell. For example, uptake can involve transfer of the PMP (e.g., LPMP) or a portion of component thereof from the extracellular environment into or across the cell membrane, the cell wall, the extracellular matrix, or into the intracellular environment of the cell). Cell uptake of PMPs (e.g., LPMPs) may occur via active or passive cellular mechanisms. Cell uptake includes aspects in which the entire PMP (e.g., LPMP) is taken up by a cell, e.g., taken up by endocytosis. In some embodiments, one or more polynucleotides are exposed to the cytoplasm of the target cell following endocytosis and endosomal escape. In some embodiments, a modified LPMP (e.g., a LPMP comprising an ionizable lipid, e.g., a LPMP comprising an ionizable lipid and a sterol and/or a PEGylated lipid) has an increased rate of endosomal escape relative to an unmodified LPMP. Cell uptake also includes aspects in which the PMP (e.g., LPMP) fuses with the membrane of the target cell. In some embodiments, one or more polynucleotides are exposed to the cytoplasm of the target cell following membrane fusion. In some embodiments, a LPMPs has an increased rate of fusion with the membrane of the target cell (e.g., is more fusogenic) relative to an unmodified LPMP.

As used herein, the term “cell-penetrating agent” refers to agents that alter properties (e.g., permeability) of the cell wall, extracellular matrix, or cell membrane of a cell (e.g., an animal cell, a plant cell, a bacterial cell, or a fungal cell) in a manner that promotes increased cell uptake relative to a cell that has not been contacted with the agent.

As used herein, the term “plant extracellular vesicle”, “plant EV”, or “EV” refers to an enclosed lipid-bilayer structure naturally occurring in a plant. Optionally, the plant EV includes one or more plant EV markers. As used herein, the term “plant EV marker” refers to a component that is naturally associated with a plant, such as a plant protein, a plant nucleic acid, a plant small molecule, a plant lipid, or a combination thereof, including but not limited to any of the plant EV markers listed in the Appendix. In some instances, the plant EV marker is an identifying marker of a plant EV but is not a pesticidal agent. In some instances, the plant EV marker is an identifying marker of a plant EV and also a pesticidal agent (e.g., either associated with or encapsulated by the plurality of PMPs or LPMPs, or not directly associated with or encapsulated by the plurality of PMPs or LPMPs).

As used herein, the term “plant messenger pack” or “PMP” refers to a lipid structure (e.g., a lipid bilayer, unilamellar, multilamellar structure; e.g., a vesicular lipid structure), that is about 5-2000 nm (e.g., at least 5-1000 nm, at least 5-500 nm, at least 400-500 nm, at least 25-250 nm, at least 50-150 nm, or at least 70-120 nm) in diameter that is derived from (e.g., enriched, isolated or purified from) a plant source or segment, portion, or extract thereof, including lipid or non-lipid components (e.g., peptides, nucleic acids, or small molecules) associated therewith and that has been enriched, isolated or purified from a plant, a plant part, or a plant cell, the enrichment or isolation removing one or more contaminants or undesired components from the source plant. PMPs may be highly purified preparations of naturally occurring EVs. Preferably, at least 1% of contaminants or undesired components from the source plant are removed (e.g., at least 2%, 5%, 10%, 15%, 20%, 25%, 30%, 40%, 45%, 50%, 55%, 60%, 70%, 80%, 90%, 95%, 96%, 98%, 99%, or 100%) of one or more contaminants or undesired components from the source plant, e.g., plant cell wall components; pectin; plant organelles (e.g., mitochondria; plastids such as chloroplasts, leucoplasts or amyloplasts; and nuclei); plant chromatin (e.g., a plant chromosome); or plant molecular aggregates (e.g., protein aggregates, protein-nucleic acid aggregates, lipoprotein aggregates, or lipido-proteic structures). Preferably, a PMP is at least 30% pure (e.g., at least 40% pure, at least 50% pure, at least 60% pure, at least 70% pure, at least 80% pure, at least 90% pure, at least 99% pure, or 100% pure) relative to the one or more contaminants or undesired components from the source plant as measured by weight (w/w), spectral imaging (% transmittance), or conductivity (S/m).

A lipid reconstructed PMP (LPMP) is used herein. The terms “lipid reconstructed PMP” and “LPMP” refer to a PMP that has been derived from a lipid structure (e.g., a lipid bilayer, unilamellar, multilamellar structure; e.g., a vesicular lipid structure) derived from (e.g., enriched, isolated or purified from) a plant source, wherein the lipid structure is disrupted (e.g., disrupted by lipid extraction) and reassembled or reconstituted in a liquid phase (e.g., a liquid phase containing a cargo) using standard methods, e.g., reconstituted by a method comprising lipid film hydration and/or solvent injection, to produce the LPMP, as is described herein. The method may, if desired, further comprise sonication, freeze/thaw treatment, and/or lipid extrusion, e.g., to reduce the size of the reconstituted LPMPs. Alternatively, LPMPs may be produced using a microfluidic device (such as a NanoAssemblr® IGNITE™ microfluidic instrument (Precision NanoSystems)).

As used herein, the term“cationic lipid” refers to an amphiphilic molecule (e.g., a lipid or a lipidoid) that is positively charged, containing a cationic group (e.g., a cationic head group).

As used herein, the term “ionizable lipid” refers to an amphiphilic molecule (e.g., a lipid or a lipidoid, e.g., a synthetic lipid or lipidoid) containing a group (e.g., a head group) that can be ionized, e.g., dissociated to produce one or more electrically charged species, under a given condition (e.g., pH). For instance, an ionizable lipid may carry a net positive charge at a selected pH, such as physiological pH (e.g., pH of about 7.0). Under this scenario, those molecules that contain a group that is charged (e.g., a positively charged lipid, i.e., a cationic lipid) may be considered as the ionizable lipid.

It has been surprisingly found that ionizable lipids comprising alkyl chains with multiple sites of unsaturation, e.g., at least two or three sites of unsaturation, are particularly useful for forming lipid particles with increased membrane fluidity. A number of ionizable lipids and related analogs, suitable for use herein, have been described in U.S. Patent Publication Nos. 20060083780 and 20060240554; U.S. Pat. Nos. 5,208,036; 5,264,618; 5,279,833; 5,283,185; 5,753,613; and 5,785,992; and PCT Publication No. WO 96/10390, the disclosures of which are herein incorporated by reference in their entirety for all purposes.

In some embodiments, ionizable lipids are ionizable such that they can dissociate to exist in a positively charged form depending on pH. The ionization of an ionizable lipid affects the surface charge of a lipid nanoparticle comprising the ionizable lipid under different pH conditions. The surface charge of the lipid nanoparticle in turn can influence its plasma protein absorption, blood clearance, and tissue distribution (Semple, S. C., et al., Adv. Drug Deliv Rev 32:3-17 (1998)) as well as its ability to form endosomolytic non-bilayer structures (Hafez, I. M., et al., Gene Ther 8: 1188-1196 (2001)) that can influence the intracellular delivery of nucleic acids.

In some embodiments, ionizable lipids are those that are generally neutral, e.g., at physiological pH (e.g., pH about 7), but can carry net charge(s) at an acidic pH or basic pH. In one embodiment, ionizable lipids are those that are generally neutral at pH about 7, but can carry net charge(s) at an acidic pH. In one embodiment, ionizable lipids are those that are generally neutral at pH about 7, but can carry net charge(s) at a basic pH.

In some embodiments, ionizable lipids do not include those cationic lipids or anionic lipids that generally carry net charge(s) at physiological pH (e.g., pH about 7).

As used herein, the term “lipidoid” refers to a molecule having one or more characteristics of a lipid.

As used herein, the term “stable LPMP formulation” refers to a LPMP composition that over a period of time (e.g., at least 24 hours, at least 48 hours, at least 1 week, at least 2 weeks, at least 3 weeks, at least 4 weeks, at least 30 days, at least 60 days, or at least 90 days) retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of the initial number of LPMPs (e.g., LPMPs per mL of solution) relative to the number of LPMPs in the LPMP formulation (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)); or retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of its activity (e.g., cell wall penetrating activity and/or activity of the mRNA formulated within the LPMP) relative to the initial activity of the LPMP (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)).

### BRIEF DESCRIPTION OF THE DRAWINGS

FIG. 1 is a photograph and a bar graph showing the level of erythropoietin (EPO) in pg/mL in serum from Ai9 (tomato red loxP) mice treated orally with reconstructed LPMPs (recPMPs) derived from lemon and comprising Cre recombinase mRNA. The photograph shows fluorescence of a marker in the mouse.

FIG. 2 is a bar graph showing the percentage of in vivo immune cells (CD-4 cells, CD-8 cells, or B cells) transfected from the spleens of Ai9 (tomato red loxP) mice treated with recLPMPs derived from lemon and comprising CRE recombinase mRNA; a lipid nanoparticle (LNP) control; or a phosphate-buffered saline (PBS) control.

FIG. 3A is a bar graph showing the size (in nm) and polydispersity index (PDI) of the lipid nanoparticles (LNPs) and reconstructed LPMPs (recPMPs) of Example 2. FIG. 3B is a bar graph showing the percent encapsulation efficiency of an RNA cargo of the LNPs and recPMPs of Example 2.

FIG. 4 is a plot showing expression of erythropoietin (EPO) in serum following repeat doses of reconstructed lemon LPMPs comprising EPO mRNA in cynomolgus macaques. The reconstructed lemon LPMPs were administered intramuscularly (IM) at doses of 0.1 mg/kg on Day 1 and 0.05 mg/kg on Day 8. The concentration of human erythropoietin (hEPO) is shown pre-dose and at 6, 12, and 24 hours post-dose.

FIG. 5 is a plot showing expression of EPO in serum following repeat doses of reconstructed lemon LPMPs comprising EPO mRNA in cynomolgus macaques. The reconstructed lemon LPMPs were administered IM at doses of 0.1 mg/kg on Day 1; 0.05 mg/kg on Day 8; and 0.05 mg/kg on Day 15. The concentration of hEPO is shown pre-dose and at 6, 12, and 24 hours post-dose. 50% of animals were pre-medicated with Buprenorphine and Meloxicam SR at 0.2 mg/kg (IV injection) preceding doses 2 and 3.

FIG. 6 is a plot showing the expression of EPO in serum following repeat doses of reconstructed lemon LPMPs comprising EPO mRNA in cynomolgus macaques. The reconstructed lemon LPMPs were administered IM at doses of 0.1 mg/kg on Day 1; 0.05 mg/kg on Day 8; and 0.01 mg/kg on Day 15. The concentration of hEPO is shown pre-dose and at 6, 12, and 24 hours post-dose. 50% of animals were pre-medicated with Buprenorphine and Meloxicam SR at 0.2 mg/kg (IV injection) preceding dose 2, and 90% of animals were pre-medicated preceding dose 3.

FIG. 7 is a plot showing the expression of EPO in serum following repeat doses of reconstructed lemon LPMPs comprising EPO mRNA in cynomolgus macaques. The reconstructed lemon LPMPs were administered subcutaneously (SubQ) at doses of 0.1 mg/kg on Day 1; 0.05 mg/kg on Day 8; and 0.01 mg/kg on Day 15. The concentration of hEPO is shown pre-dose and at 6, 12, and 24 hours post-dose. 50% of animals were pre-medicated with Buprenorphine and Meloxicam SR at 0.2 mg/kg (IV injection) preceding dose 2, and 90% of animals were pre-medicated preceding dose 3.

FIG. 8 is a plot showing expression of EPO in serum following repeat doses of reconstructed lemon LPMPs comprising EPO mRNA in cynomolgus macaques. The reconstructed lemon LPMPs were administered SubQ at doses of 0.1 mg/kg on Day 1; 0.05 mg/kg on Day 8; and 0.05 mg/kg on Day 15. The concentration of hEPO is shown pre-dose and at 6, 12, and 24 hours post-dose. 50% of animals were pre-medicated with Buprenorphine and Meloxicam SR at 0.2 mg/kg (IV injection) preceding doses 2 and 3.

FIG. 9 is a bar graph showing the expression of EPO in serum following oral delivery of reconstructed lemon LPMPs comprising EPO mRNA to mice.

FIG. 10 is a set of bar graphs showing the frequency of tomato red-positive cells (immune cells transfected in vivo) in parent populations of splenocytes, lung cells, and bone marrow cells (circulating immune cells and progenitor cells) in tomato red loxP mice cells treated with intravenously administered (IV) reconstructed lemon LPMPs comprising Cre recombinase.

FIG. 11 is a pair of bar graphs showing the percentage of GFP-positive immune cells (CD4 T cells, CD8 T cells, and B cells) in cynomolgus macaques treated with intramuscularly administered (IM; top panel) or subcutaneously administered (SubQ; bottom panel) reconstructed lemon LPMPs comprising GFP mRNA.

FIG. 12A shows the tumor volume measured at days post implantation of MC38 tumor cells in the mouse after the first and second peritumoral doses of the recLemon LPMP/IL-2 mRNA formulation at 5 μg. FIG. 12B shows the tumor volume measured at days post implantation of MC38 tumor cells in the mouse after all four peritumoral doses of the recLemon LPMP/IL-2 mRNA formulation. FIG. 12C shows the survival rate of the mice at days post implantation of MC38 tumor cells after all four peritumoral doses of the recLemon LPMP/IL-2 mRNA formulation, with decreased survival in mice receiving buffer.

FIG. 13A shows the level of IL-2 in the serum of the mice 4 hours post the 4^{th} peritumoral dose of the recLemon LPMP/IL-2 mRNA formulation dose at 5 μg. FIGS. 13B-13G show the level of IL4 (FIG. 13B), IL5 (FIG. 13C), IFNγ (FIG. 13D), TNFα (FIG. 13E), IL6 (FIG. 13F), and CXCL1 (KC) (FIG. 13G) in the serum of the mice 4 hours post the 4^{th} peritumoral dose of the recLemon LPMP/IL-2 mRNA formulation dose at 5 μg.

FIGS. 14A-14C show the level of IL6 (FIG. 14A), IFNγ (FIG. 14B), and TNFα (FIG. 14C) in the serum of the mice 48 hours post the 2^{nd} peritumoral dose of the recLemon LPMP/IL-2 mRNA formulation dose at 5 μg.

FIG. 15 shows the circulating human IL-2 levels 6 hours post-dose. Intramuscular injection; recLemon LPMP/mRNA=0.4 mg/kg (equal to 10 μg of IL-2); N=9 mice per group for 6 hours post dosing and 6 mice per group for 2 days post dosing.

FIGS. 16A-16B show the levels of circulating anti-tumor cytokines 6 hours post-dose (FIG. 16A), and the levels of circulating anti-tumor cytokines 2 days post-dose (FIG. 16B). Intramuscular injection; recLemon LPMP/mRNA=0.4 mg/kg (equal to 10 μg of IL-2); N=9 mice per group for 6 hours post dosing and 6 mice per group for 2 days post dosing.

FIG. 17 shows the T cell profiles 6 days post-dose. The graphs show that a single dose of the recLemon LPMP/IL-2 mRNA formulation was potent and induced durable T cell effects lasting 6 days post dosing. Intramuscular injection; recLemon LPMP/mRNA at 0.4 mg/kg (equal to 10 μg of IL-2); N=3 mice per group.

FIG. 18 shows the IL-15 serum concentration post-dose. The graph shows a systemic effect: a single intramuscular dose of the recLemon LPMP/IL-15 mRNA formulation increased systemic protein level up to 72 hours post-dose. Intramuscular injection; recLemon LPMP/mRNA=0.4 mg/kg (equal to 10 μg of IL-15); N=3 mice per group.

FIG. 19 shows the cytokines and T cell profiles in the blood 6 days post-dose. The graphs show the cytokine effect: a single intramuscular dose of the recLemon LPMP/IL-15 mRNA formulation resulted in higher frequency of T cells producing IFNγ in the blood 6 days post dose. Intramuscular injection; recLemon LPMP/mRNA=0.4 mg/kg (equal to 10 μg of IL-15); N=3 mice per group.

FIG. 20 shows the T cell profiles in the spleen 6 days post-dose. The graphs show the cellular effect: a single intramuscular dose of the recLemon LPMP/IL-15 mRNA formulation resulted in an increased number of T cells and NK cells in the spleen 6 days post dose. Intramuscular injection; recLemon LPMP/mRNA=0.4 mg/kg (equal to 10 μg of IL-15); N=3 mice per group.

FIGS. 21A-21B show the cytokines/chemokines in the blood 4, 6, 24, 48, and 72 hours post-dose. The graphs show the cytokine effect: a single intramuscular dose of the recLemon LPMP/IL-15 mRNA formulation resulted in higher frequency of pro-inflammatory cytokines and chemokines (IL-6 and IP-10) in the blood of mice within 24 hours post dose. Intramuscular injection; recLemon LPMP/mRNA=0.4 mg/kg (equal to 10 μg of IL-15); N=3 mice per group.

FIGS. 22A-22B show that a single intramuscular dose of recLemon LPMP/mRNA formulation (IL-15) in mice was able to increase IL-2Ra expression on NK cells (FIG. 22A) and IFNγ+NK cells (FIG. 22B) in the spleen of mice 10 days post-dose. Intramuscular injection; recLemon LPMP/mRNA=0.4 mg/kg (equal to 10 μg of IL-15); N=3 mice per group.

FIG. 23 shows that a single intramuscular dose of recLemon LPMP/mRNA formulation (IL-15) in NHPs was able to increase IL-2Ra (CD25) expression on NK cells and T cells in the blood of NHPs 24 hours (Day 1) post-dose. Intramuscular injection; recLemon LPMP/mRNA=0.02 mg/kg IL-15; 0.006 mg/kg IL-15; 0.006 mg/kg IL-2; N=3 NHPs per group, except control (N=1).

FIG. 24 shows that a single intramuscular dose of recLemon LPMP/mRNA formulation (IL-15) was able to increase Granzyme B/perforin and IFNγ expression in T cells (FIG. 24A) and NK cells (FIG. 24B) in the blood of NHPs 24 hours (Day 1) post-dose. Intramuscular injection; recLemon LPMP/mRNA=0.02 mg/kg IL-15; 0.006 mg/kg IL-15; 0.006 mg/kg IL-2; N=3 NHPs per group, except control (N=1).

FIG. 25 shows that a single intramuscular dose of recLemon LPMP/mRNA formulation (IL-15 and IL-2) resulted in T cell and NK cell proliferation in the blood of NHPs 4 days post-dose. Intramuscular injection; recLemon LPMP/mRNA=0.02 mg/kg IL-15; 0.006 mg/kg IL-15; 0.006 mg/kg IL-2; N=3 NHPs per group, except control (N=1).

FIG. 26 shows that a single intramuscular dose of recLemon LPMP/mRNA formulation (IL-15 and IL-2) resulted in increased levels of Granzyme B/perforin and IFNγ expression in T cells (FIG. 26A) and NK cells (FIG. 26B) in the blood of NHPs 4 days post-dose. Intramuscular injection; recLemon LPMP/mRNA=0.02 mg/kg IL-15; 0.006 mg/kg IL-15; 0.006 mg/kg IL-2; N=3 NHPs per group, except control (N=1).

FIG. 27 shows that a single intramuscular dose of recLemon LPMP/mRNA formulation (IL-15) resulted in increased IL-2Ra (CD25) expression on NK cells and T cells in the blood of NHPs 4 days post-dose. Intramuscular injection; recLemon LPMP/mRNA=0.02 mg/kg IL-15; 0.006 mg/kg IL-15; 0.006 mg/kg IL-2; N=3 NHPs per group, except control (N=1).

FIGS. 28A-B show that a single intramuscular dose of recLemon LPMP/mRNA formulation (IL-15) resulted in increased levels of Granzyme B/perforin and IFNγ expression in T cells (FIG. 28A) and NK cells (FIG. 28B) in the blood of NHPs 8 days post-dose. Intramuscular injection; recLemon LPMP/mRNA=0.02 mg/kg IL-15; 0.006 mg/kg IL-15; 0.006 mg/kg IL-2; N=3 NHPs per group, except control (N=1).

FIG. 29 shows that a single intramuscular dose of recLemon LPMP/mRNA formulation (IL-15 and IL-2) resulted in increased levels of IL-2Ra (CD25) on NK cells in the blood of NHPs 8 days post-dose. Intramuscular injection; recLemon LPMP/mRNA=0.02 mg/kg IL-15; 0.006 mg/kg IL-15; 0.006 mg/kg IL-2; N=3 NHPs per group, except control (N=1).

FIG. 30 shows that a single intramuscular dose of recLemon LPMP/mRNA formulation (IL-15) resulted in T cell and NK cell proliferation in the blood of NHPs 8 days post-dose. Intramuscular injection; recLemon LPMP/mRNA=0.02 mg/kg IL-15; 0.006 mg/kg IL-15; 0.006 mg/kg IL-2; N=3 NHPs per group, except control (N=1).

FIG. 31 shows that a single intramuscular dose of recLemon LPMP/mRNA formulation (IL-15 and IL-2) resulted in T cell and NK cell proliferation in the blood of NHPs 13 days post-dose. Intramuscular injection; recLemon LPMP/mRNA=0.02 mg/kg IL-15; 0.006 mg/kg IL-15; 0.006 mg/kg IL-2; N=3 NHPs per group, except control (N=1).

FIG. 32 shows that a single intramuscular dose of recLemon LPMP/mRNA formulation (IL-15) resulted in increased levels of IL-2Ra (CD25) on T cells and NK cells in the blood of NHPs 13 days post-dose. Intramuscular injection; recLemon LPMP/mRNA=0.02 mg/kg IL-15; 0.006 mg/kg IL-15; 0.006 mg/kg IL-2; N=3 NHPs per group, except control (N=1).

FIGS. 33A-B are line graphs depicting blood cytokine levels of IL-15 (FIG. 33A) and IL-2 (FIG. 33B) measured at 4 hours, 6 hours, 24 hours, 4 days (96 hours), 8 days (192 hours), and 13 days (312 hours) after a single intramuscular dose of a recLemon LPMP/mRNA formulation. Intramuscular injection; recLemon LPMP/mRNA=0.02 mg/kg IL-15; 0.006 mg/kg IL-15; 0.006 mg/kg IL-2; N=3 NHPs per group, except control (N=1).

FIG. 34 is a line graph depicting the expansion of CD56+NK cells compared to a naïve control after a single intramuscular dose of recLemon LPMP/mRNA formulation (IL-15 and IL-2) in the blood of NHPs over the course of 13 days post-dose. Intramuscular injection; recLemon LPMP/mRNA=0.02 mg/kg IL-15; 0.006 mg/kg IL-15; 0.006 mg/kg IL-2; N=3 NHPs per group, except control (N=1).

FIGS. 35A-C are line graphs depicting blood cytokine levels of pro-inflammatory cytokines, including IP-10 (FIG. 35A), IFNγ (FIG. 35B), and IL-6 (FIG. 35C), respectively, measured at 4 hours, 6 hours, 24 hours, 4 days (96 hours), 8 days (192 hours), and 13 days (312 hours) post-dose. Intramuscular injection; recLemon LPMP/mRNA=0.02 mg/kg IL-15; 0.006 mg/kg IL-15; 0.006 mg/kg IL-2; N=3 NHPs per group, except control (N=1).

FIGS. 36A-B are comparisons of dosage and animal models through the measure of systemic IL-15 production 6 hours post dose between mice (FIG. 36A) and non-human primates (FIG. 36B). Both bar graphs show comparable IL-15 production between varying animal models based on the dosages given. Intramuscular injection; recLemon LPMP/mRNA=0.5 mg/kg IL-15; 0.02 mg/kg IL-15; 0.006 mg/kg IL-15; N=3 NHPs or mice per group.

### DETAILED DESCRIPTION

Featured herein are mRNA therapeutic compositions (e.g., nucleic acid cancer vaccine of an RNA, e.g., mRNA), that can safely direct the body's cellular machinery to produce nearly any cancer protein or fragment thereof of interest. The mRNA therapeutic compositions may be used to induce a balanced immune response against cancers, comprising both cellular and humoral immunity, without risking the possibility of insertional mutagenesis, for example. These mRNA therapeutic compositions include one or more polynucleotides (e.g., RNA such as messenger RNA (mRNA)) encoding one or more antigenic (e.g., tumor antigenic) or signaling polypeptides, formulated within a lipid reconstructed plant messenger packs (LPMPs) comprising natural lipids and an ionizable lipid. PMPs are lipid assemblies produced wholly or in part from plant extracellular vesicles (EVs), or segments, portions, or extracts thereof. LPMPs are PMPs derived from a lipid structure wherein the lipid structure is disrupted and reassembled or reconstituted in a liquid phase.

The disclosure also includes a method for making a mRNA therapeutic composition, comprising reconstituting a film comprising purified PMP lipids in the presence of an ionizable lipid to produce a LPMP comprising the ionizable lipid, and loading into the LPMPs with one or more polynucleotides encoding one or more antigenic (e.g., tumor antigenic) or signaling polypeptides.

### Lipid Reconstructed Plant Messenger Packs (LPMPs)

### Plant Messenger Pack (PMPs)

A PMP is a lipid (e.g., lipid bilayer, unilamellar, or multilamellar structure) structure that includes a plant EV, or segment, portion, or extract (e.g., lipid extract) thereof. Plant EVs refer to an enclosed lipid-bilayer structure that naturally occurs in a plant and that is about 5-2000 nm in diameter. Plant EVs can originate from a variety of plant biogenesis pathways. In nature, plant EVs can be found in the intracellular and extracellular compartments of plants, such as the plant apoplast, the compartment located outside the plasma membrane and formed by a continuum of cell walls and the extracellular space. Alternatively, PMPs can be enriched plant EVs found in cell culture media upon secretion from plant cells. Plant EVs can be separated from plants, thereby providing PMPs, by a variety of methods further described herein. Further, the PMPs can optionally include a therapeutic agent, which can be introduced in vivo or in vitro.

PMPs can include plant EVs, or segments, portions, or extracts, thereof. Optionally, PMPs can also include exogenous lipids (e.g., sterols (e.g., cholesterol or sitosterol), ionizable lipids, and/or PEGylated lipids) in addition to lipids derived from plant EVs. In some embodiments, the plant EVs are about 5-1000 nm in diameter. For example, the PMP can include a plant EV, or segment, portion, or extract thereof, that has a mean diameter of about 5-50 nm, about 50-100 nm, about 100-150 nm, about 150-200 nm, about 200-250 nm, about 250-300 nm, about 300-350 nm, about 350-400 nm, about 400-450 nm, about 450-500 nm, about 500-550 nm, about 550-600 nm, about 600-650 nm, about 650-700 nm, about 700-750 nm, about 750-800 nm, about 800-850 nm, about 850-900 nm, about 900-950 nm, about 950-1000 nm, about 1000-1250 nm, about 1250-1500 nm, about 1500-1750 nm, or about 1750-2000 nm. In some instances, the PMP includes a plant EV, or segment, portion, or extract thereof, that has a mean diameter of about 5-950 nm, about 5-900 nm, about 5-850 nm, about 5-800 nm, about 5-750 nm, about 5-700 nm, about 5-650 nm, about 5-600 nm, about 5-550 nm, about 5-500 nm, about 5-450 nm, about 5-400 nm, about 5-350 nm, about 5-300 nm, about 5-250 nm, about 5-200 nm, about 5-150 nm, about 5-100 nm, about 5-50 nm, or about 5-25 nm. In certain instances, the plant EV, or segment, portion, or extract thereof, has a mean diameter of about 50-200 nm. In certain instances, the plant EV, or segment, portion, or extract thereof, has a mean diameter of about 50-300 nm. In certain instances, the plant EV, or segment, portion, or extract thereof, has a mean diameter of about 200-500 nm. In certain instances, the plant EV, or segment, portion, or extract thereof, has a mean diameter of about 30-150 nm.

In some instances, the PMP may include a plant EV, or segment, portion, or extract thereof, that has a mean diameter of at least 5 nm, at least 50 nm, at least 100 nm, at least 150 nm, at least 200 nm, at least 250 nm, at least 300 nm, at least 350 nm, at least 400 nm, at least 450 nm, at least 500 nm, at least 550 nm, at least 600 nm, at least 650 nm, at least 700 nm, at least 750 nm, at least 800 nm, at least 850 nm, at least 900 nm, at least 950 nm, or at least 1000 nm. In some instances, the PMP includes a plant EV, or segment, portion, or extract thereof, that has a mean diameter less than 1000 nm, less than 950 nm, less than 900 nm, less than 850 nm, less than 800 nm, less than 750 nm, less than 700 nm, less than 650 nm, less than 600 nm, less than 550 nm, less than 500 nm, less than 450 nm, less than 400 nm, less than 350 nm, less than 300 nm, less than 250 nm, less than 200 nm, less than 150 nm, less than 100 nm, or less than 50 nm. A variety of methods (e.g., a dynamic light scattering method) standard in the art can be used to measure the particle diameter of the plant EV, or segment, portion, or extract thereof.

In some instances, the PMP may include a plant EV, or segment, portion, or extract thereof, that has a mean surface area of 77 nm² to 3.2×10⁶ nm² (e.g., 77-100 nm², 100-1000 nm², 1000-1×10⁴ nm², 1×10⁴-1×10⁵ nm², 1×10⁵-1×10⁶ nm², or 1×10⁶-3.2×10⁶ nm²). In some instances, the PMP may include a plant EV, or segment, portion, or extract thereof, that has a mean volume of 65 nm³ to 5.3×10⁸ nm³ (e.g., 65-100 nm³, 100-1000 nm³, 1000-1×10⁴ nm³, 1×10⁴-1×10⁵ nm³, 1×10⁵-1×10⁶ nm³, 1×10⁶-1×10⁷ nm³, 1×10⁷-1×10⁸ nm³, 1×10⁸-5.3×10⁸ nm³). In some instances, the PMP may include a plant EV, or segment, portion, or extract thereof, that has a mean surface area of at least 77 nm², (e.g., at least 77 nm², at least 100 nm², at least 1000 nm², at least 1×10⁴ nm², at least 1×10⁵ nm², at least 1×10⁶ nm², or at least 2×10⁶ nm²). In some instances, the PMP may include a plant EV, or segment, portion, or extract thereof, that has a mean volume of at least 65 nm³ (e.g., at least 65 nm³, at least 100 nm³, at least 1000 nm³, at least 1×10⁴ nm³, at least 1×10⁵ nm³, at least 1×10⁶ nm³, at least 1×10⁷ nm³, at least 1×10⁸ nm³, at least 2×10⁸ nm³, at least 3×10⁸ nm³, at least 4×10⁸ nm³, or at least 5×10⁸ nm³.

In some instances, the PMP can have the same size as the plant EV or segment, extract, or portion thereof. Alternatively, the PMP may have a different size than the initial plant EV from which the PMP is produced. For example, the PMP may have a diameter of about 5-2000 nm in diameter. For example, the PMP can have a mean diameter of about 5-50 nm, about 50-100 nm, about 100-150 nm, about 150-200 nm, about 200-250 nm, about 250-300 nm, about 300-350 nm, about 350-400 nm, about 400-450 nm, about 450-500 nm, about 500-550 nm, about 550-600 nm, about 600-650 nm, about 650-700 nm, about 700-750 nm, about 750-800 nm, about 800-850 nm, about 850-900 nm, about 900-950 nm, about 950-1000 nm, about 1000-1200 nm, about 1200-1400 nm, about 1400-1600 nm, about 1600-1800 nm, or about 1800-2000 nm. In some instances, the PMP may have a mean diameter of at least 5 nm, at least 50 nm, at least 100 nm, at least 150 nm, at least 200 nm, at least 250 nm, at least 300 nm, at least 350 nm, at least 400 nm, at least 450 nm, at least 500 nm, at least 550 nm, at least 600 nm, at least 650 nm, at least 700 nm, at least 750 nm, at least 800 nm, at least 850 nm, at least 900 nm, at least 950 nm, at least 1000 nm, at least 1200 nm, at least 1400 nm, at least 1600 nm, at least 1800 nm, or about 2000 nm. A variety of methods (e.g., a dynamic light scattering method) standard in the art can be used to measure the particle diameter of the PMPs. In some instances, the size of the PMP is determined following loading of a therapeutic agent, or following other modifications to the PMPs.

In some instances, the PMP may have a mean surface area of 77 nm² to 1.3×10⁷ nm² (e.g., 77-100 nm², 100-1000 nm², 1000-1×10⁴ nm², 1×10⁴-1×10⁵ nm², 1×10⁵-1×10⁶ nm², or 1×10⁶-1.3×10⁷ nm²). In some instances, the PMP may have a mean volume of 65 nm³ to 4.2×10⁹ nm³ (e.g., 65-100 nm³, 100-1000 nm³, 1000-1×10⁴ nm³, 1×10⁴-1×10⁵ nm³, 1×10⁵-1×10⁶ nm³, 1×10⁶-1×10⁷ nm³, 1×10⁷-1×10⁸ nm³, 1×10⁸-1×10⁹ nm³, or 1×10⁹-4.2×10⁹ nm³). In some instances, the PMP has a mean surface area of at least 77 nm², (e.g., at least 77 nm², at least 100 nm², at least 1000 nm², at least 1×10⁴ nm², at least 1×10⁵ nm², at least 1×10⁶ nm², or at least 1×10⁷ nm²). In some instances, the PMP has a mean volume of at least 65 nm³ (e.g., at least 65 nm³, at least 100 nm³, at least 1000 nm³, at least 1×10⁴ nm³, at least 1×10⁵ nm³, at least 1×10⁶ nm³, at least 1×10⁷ nm³, at least 1×10⁸ nm³, at least 1×10⁹ nm³, at least 2×10⁹ nm³, at least 3×10⁹ nm³, or at least 4×10⁹ nm³).

In some instances, the PMP may include an intact plant EV. Alternatively, the PMP may include a segment, portion, or extract of the full surface area of the vesicle (e.g., a segment, portion, or extract including less than 100% (e.g., less than 90%, less than 80%, less than 70%, less than 60%, less than 50%, less than 40%, less than 30%, less than 20%, less than 10%, less than 10%, less than 5%, or less than 1%) of the full surface area of the vesicle) of a plant EV. The segment, portion, or extract may be any shape, such as a circumferential segment, spherical segment (e.g., hemisphere), curvilinear segment, linear segment, or flat segment. In instances where the segment is a spherical segment of the vesicle, the spherical segment may represent one that arises from the splitting of a spherical vesicle along a pair of parallel lines, or one that arises from the splitting of a spherical vesicle along a pair of non-parallel lines. Accordingly, the plurality of PMPs can include a plurality of intact plant EVs, a plurality of plant EV segments, portions, or extracts, or a mixture of intact and segments of plant EVs. One skilled in the art will appreciate that the ratio of intact to segmented plant EVs will depend on the particular isolation method used. For example, grinding or blending a plant, or part thereof, may produce PMPs that contain a higher percentage of plant EV segments, portions, or extracts than a non-destructive extraction method, such as vacuum-infiltration.

In instances where, the PMP includes a segment, portion, or extract of a plant EV, the EV segment, portion, or extract may have a mean surface area less than that of an intact vesicle, (e.g., a mean surface area less than 77 nm², 100 nm², 1000 nm², 1×10⁴ nm², 1×10⁵ nm², 1×10⁶ nm², or 3.2×10⁶ nm²). In some instances, the EV segment, portion, or extract has a surface area of less than 70 nm², 60 nm², 50 nm², 40 nm², 30 nm², 20 nm², or 10 nm². In some instances, the PMP may include a plant EV, or segment, portion, or extract thereof, that has a mean volume less than that of an intact vesicle, (e.g., a mean volume of less than 65 nm³, 100 nm³, 1000 nm³, 1×10⁴ nm³, 1×10⁵ nm³, 1×10⁶ nm³, 1×10⁷ nm³, 1×10⁸ nm³, or 5.3×10⁸ nm³).

In instances where the PMP includes an extract of a plant EV, e.g., in instances where the PMP includes lipids extracted (e.g., with chloroform) from a plant EV, the PMP may include at least 1%, 2%, 5%, 10%, 20%, 30%, 40%, 50%, 60%, 70%, 80%, 90%, 95%, 99%, or more than 99%, of lipids extracted (e.g., with chloroform) from a plant EV. The PMPs in the plurality may include plant EV segments and/or plant EV-extracted lipids or a mixture thereof.

### Production of PMPs

PMPs may be produced from plant EVs, or a segment, portion or extract (e.g., lipid extract) thereof, that occur naturally in plants, or parts thereof, including plant tissues or plant cells. An exemplary method for producing PMPs includes (a) providing an initial sample from a plant, or a part thereof, wherein the plant or part thereof comprises EVs; and (b) isolating a crude PMP fraction from the initial sample, wherein the crude PMP fraction has a decreased level of at least one contaminant or undesired component from the plant or part thereof relative to the level in the initial sample. The method can further include an additional step (c) comprising purifying the crude PMP fraction, thereby producing a plurality of pure PMPs, wherein the plurality of pure PMPs have a decreased level of at least one contaminant or undesired component from the plant or part thereof relative to the level in the crude EV fraction. Each production step is discussed in further detail, below. Exemplary methods regarding the isolation and purification of PMPs is found, for example, in Rutter and Innes, Plant Physiol. 173(1): 728-741, 2017; Rutter et al, Bio. Protoc. 7(17): e2533, 2017; Regente et al, J of Exp. Biol. 68(20): 5485-5496, 2017; Mu et al, Mol. Nutr. Food Res., 58, 1561-1573, 2014, and Regente et al, FEBS Letters. 583: 3363-3366, 2009, each of which is herein incorporated by reference.

In some instances, a plurality of PMPs may be isolated from a plant by a process which includes the steps of: (a) providing an initial sample from a plant, or a part thereof, wherein the plant or part thereof comprises EVs; (b) isolating a crude PMP fraction from the initial sample, wherein the crude PMP fraction has a decreased level of at least one contaminant or undesired component from the plant or part thereof relative to the level in the initial sample (e.g., a level that is decreased by at least 1%, 2%, 5%, 10%, 15%, 20%, 25%, 30%, 40%, 45%, 50%, 55%, 60%, 70%, 80%, 90%, 95%, 96%, 98%, 99%, or 100%); and (c) purifying the crude PMP fraction, thereby producing a plurality of pure PMPs, wherein the plurality of pure PMPs have a decreased level of at least one contaminant or undesired component from the plant or part thereof relative to the level in the crude EV fraction (e.g., a level that is decreased by at least 1%, 2%, 5%, 10%, 15%, 20%, 25%, 30%, 40%, 45%, 50%, 55%, 60%, 70%, 80%, 90%, 95%, 96%, 98%, 99%, or 100%).

The PMPs can include a plant EV, or segment, portion, or extract thereof, produced from a variety of plants. PMPs may be produced from any genera of plants (vascular or nonvascular), including but not limited to angiosperms (monocotyledonous and dicotyledonous plants), gymnosperms, ferns, selaginellas, horsetails, psilophytes, lycophytes, algae (e.g., unicellular or multicellular, e.g., archaeplastida), or bryophytes. In certain instances, PMPs can be produced using a vascular plant, for example monocotyledons or dicotyledons or gymnosperms. For example, PMPs can be produced using alfalfa, apple, Arabidopsis, banana, barley, a Brassica species (e.g., Arabidopsis thaliana or Brassica napus), canola, castor bean, chicory, chrysanthemum, clover, cocoa, coffee, cotton, cottonseed, corn, crambe, cranberry, cucumber, dendrobium, dioscorea, eucalyptus, fescue, flax, gladiolus, liliacea, linseed, millet, muskmelon, mustard, oat, oil palm, oilseed rape, papaya, peanut, pineapple, ornamental plants, Phaseolus, potato, rapeseed, rice, rye, ryegrass, safflower, sesame, sorghum, soybean, sugarbeet, sugarcane, sunflower, strawberry, tobacco, tomato, turfgrass, wheat or vegetable crops such as lettuce, celery, broccoli, cauliflower, cucurbits; fruit and nut trees, such as apple, pear, peach, orange, grapefruit, lemon, lime, almond, pecan, walnut, hazel; vines, such as grapes, kiwi, hops; fruit shrubs and brambles, such as raspberry, blackberry, gooseberry; forest trees, such as ash, pine, fir, maple, oak, chestnut, popular; with alfalfa, canola, castor bean, corn, cotton, crambe, flax, linseed, mustard, oil palm, oilseed rape, peanut, potato, rice, safflower, sesame, soybean, sugarbeet, sunflower, tobacco, tomato, or wheat.

PMPs may be produced using a whole plant (e.g., a whole rosettes or seedlings) or alternatively from one or more plant parts (e.g., leaf, seed, root, fruit, vegetable, pollen, phloem sap, or xylem sap). For example, PMPs can be produced using shoot vegetative organs/structures (e.g., leaves, stems, or tubers), roots, flowers and floral organs/structures (e.g., pollen, bracts, sepals, petals, stamens, carpels, anthers, or ovules), seed (including embryo, endosperm, or seed coat), fruit (the mature ovary), sap (e.g., phloem or xylem sap), plant tissue (e.g., vascular tissue, ground tissue, tumor tissue, or the like), and cells (e.g., single cells, protoplasts, embryos, callus tissue, guard cells, egg cells, or the like), or progeny of same. For instance, the isolation step may involve (a) providing a plant, or a part thereof. In some examples, the plant part is an Arabidopsis leaf. The plant may be at any stage of development. For example, the PMPs can be produced using seedlings, e.g., 1 week, 2 week, 3 week, 4 week, 5 week, 6 week, 7 week, or 8 week old seedlings (e.g., Arabidopsis seedlings). Other exemplary PMPs can include PMPs produced using roots (e.g., ginger roots), fruit juice (e.g., grapefruit juice), vegetables (e.g., broccoli), pollen (e.g., olive pollen), phloem sap (e.g., Arabidopsis phloem sap), or xylem sap (e.g., tomato plant xylem sap).

In some embodiments, the PMPs are produced from algae or lemon.

PMPs can be produced using a plant, or part thereof, by a variety of methods. Any method that allows release of the EV-containing apoplastic fraction of a plant, or an otherwise extracellular fraction that contains PMPs comprising secreted EVs (e.g., cell culture media) is suitable in the present methods. EVs can be separated from the plant or plant part by either destructive (e.g., grinding or blending of a plant, or any plant part) or non-destructive (washing or vacuum infiltration of a plant or any plant part) methods. For instance, the plant, or part thereof, can be vacuum-infiltrated, ground, blended, or a combination thereof to isolate EVs from the plant or plant part, thereby producing PMPs. For instance, the isolating step may involve vacuum infiltrating the plant (e.g., with a vesicle isolation buffer) to release and collect the apoplastic fraction. Alternatively, the isolating step may involve grinding or blending the plant to release the EVs, thereby producing PMPs.

Upon isolating the plant EVs, thereby producing PMPs, the PMPs can be separated or collected into a crude PMP fraction (e.g., an apoplastic fraction). For instance, the separating step may involve separating the plurality of PMPs into a crude PMP fraction using centrifugation (e.g., differential centrifugation or ultracentrifugation) and/or filtration to separate the plant PMP-containing fraction from large contaminants, including plant tissue debris or plant cells. As such, the crude PMP fraction will have a decreased number of large contaminants, including plant tissue debris or plant cells, as compared to the initial sample from the plant or plant part. Depending on the method used, the crude PMP fraction may additionally comprise a decreased level of plant cell organelles (e.g., nuclei, mitochondria or chloroplasts), as compared to the initial sample from the plant or plant part.

In some instances, the isolating step may involve separating the plurality of PMPs into a crude PMP fraction using centrifugation (e.g., differential centrifugation or ultracentrifugation) and/or filtration to separate the PMP-containing fraction from plant cells or cellular debris. In such instances, the crude PMP fraction will have a decreased number of plant cells or cellular debris, as compared to the initial sample from the source plant or plant part.

The crude PMP fraction can be further purified by additional purification methods to produce a plurality of pure PMPs. For example, the crude PMP fraction can be separated from other plant components by ultracentrifugation, e.g., using a density gradient (iodixanol or sucrose) and/or use of other approaches to remove aggregated components (e.g., precipitation or size-exclusion chromatography). The resulting pure PMPs may have a decreased level of contaminants or other undesired components from the source plant (e.g., one or more non-PMP components, such as protein aggregates, nucleic acid aggregates, protein-nucleic acid aggregates, free lipoproteins, lipido-proteic structures), nuclei, cell wall components, cell organelles, or a combination thereof) relative to one or more fractions generated during the earlier separation steps, or relative to a pre-established threshold level, e.g., a commercial release specification. For example, the pure PMPs may have a decreased level (e.g., by about 5%, 10%, 15%, 20%, 30%, 40%, 50%, 60%, 70%, 80%, 90%, 100%, or more than 100%; or by about 2× fold, 4× fold, 5× fold, 10× fold, 20× fold, 25× fold, 50× fold, 75× fold, 100× fold, or more than 100× fold) of plant organelles or cell wall components relative to the level in the initial sample. In some instances, the pure PMPs are substantially free (e.g., have undetectable levels) of one or more non-PMP components, such as protein aggregates, nucleic acid aggregates, protein-nucleic acid aggregates, free lipoproteins, lipido-proteic structures), nuclei, cell wall components, cell organelles, or a combination thereof. The PMPs may be at a concentration of, e.g., 1×10⁹, 5×10⁹, 1×10¹⁰, 5×10¹⁰, 5×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, or more than 1×10¹³ PMPs/mL.

For example, protein aggregates may be removed from PMPs. For example, the PMPs can be taken through a range of pHs (e.g., as measured using a pH probe) to precipitate out protein aggregates in solution. The pH can be adjusted to, e.g., pH 3, pH 5, pH 7, pH 9, or pH 11 with the addition of, e.g., sodium hydroxide or hydrochloric acid. Once the solution is at the specified pH, it can be filtered to remove particulates. Alternatively, the PMPs can be flocculated using the addition of charged polymers, such as Polymin-P or Praestol 2640. Briefly, Polymin-P or Praestol 2640 is added to the solution and mixed with an impeller. The solution can then be filtered to remove particulates. Alternatively, aggregates can be solubilized by increasing salt concentration. For example, NaCl can be added to the PMPs until it is at, e.g., 1 mol/L. The solution can then be filtered to isolate the PMPs. Alternatively, aggregates are solubilized by increasing the temperature. For example, the PMPs can be heated under mixing until the solution has reached a uniform temperature of, e.g., 50° C. for 5 minutes. The PMP mixture can then be filtered to isolate the PMPs. Alternatively, soluble contaminants from PMP solutions can be separated by size-exclusion chromatography column according to standard procedures, where PMPs elute in the first fractions, whereas proteins and ribonucleoproteins and some lipoproteins are eluted later. The efficiency of protein aggregate removal can be determined by measuring and comparing the protein concentration before and after removal of protein aggregates via BCA/Bradford protein quantification.

Any of the production methods described herein can be supplemented with any quantitative or qualitative methods known in the art to characterize or identify the PMPs at any step of the production process. PMPs may be characterized by a variety of analysis methods to estimate PMP yield, PMP concentration, PMP purity, PMP composition, or PMP sizes. PMPs can be evaluated by a number of methods known in the art that enable visualization, quantitation, or qualitative characterization (e.g., identification of the composition) of the PMPs, such as microscopy (e.g., transmission electron microscopy), dynamic light scattering, nanoparticle tracking, spectroscopy (e.g., Fourier transform infrared analysis), or mass spectrometry (protein and lipid analysis). In certain instances, methods (e.g., mass spectroscopy) may be used to identify plant EV markers present on the PMP, such as markers disclosed in the Appendix. To aid in analysis and characterization, of the PMP fraction, the PMPs can additionally be labelled or stained. For example, the PMPs can be stained with 3,3′-dihexyloxacarbocyanine iodide (DIOC₆), a fluorescent lipophilic dye, PKH67 (Sigma Aldrich); Alexa Fluor®488 (Thermo Fisher Scientific), or DyLight™ 800 (Thermo Fisher). In the absence of sophisticated forms of nanoparticle tracking, this relatively simple approach quantifies the total membrane content and can be used to indirectly measure the concentration of PMPs (Rutter and Innes, Plant Physiol. 173(1): 728-741, 2017; Rutter et al, Bio. Protoc. 7(17): e2533, 2017). For more precise measurements, and to assess the size distributions of PMPs, nanoparticle tracking can be used.

During the production process, the PMPs can optionally be prepared such that the PMPs are at an increased concentration (e.g., by about 5%, 10%, 15%, 20%, 30%, 40%, 50%, 60%, 70%, 80%, 90%, 100%, or more than 100%; or by about 2× fold, 4× fold, 5× fold, 10× fold, 20× fold, 25× fold, 50× fold, 75× fold, 100× fold, or more than 100× fold) relative to the EV level in a control or initial sample. The PMPs may make up about 0.1% to about 100% of the PMP composition, such as any one of about 0.01% to about 100%, about 1% to about 99.9%, about 0.1% to about 10%, about 1% to about 25%, about 10% to about 50%, about 50% to about 99%, or about 75% to about 100%. In some instances, the composition includes at least any of 0.1%, 0.5%, 1%, 5%, 10%, 20%, 30%, 40%, 50%, 60%, 70%, 80%, 90%, 95%, or more PMPs, e.g., as measured by wt/vol, percent PMP protein composition, and/or percent lipid composition (e.g., by measuring fluorescently labelled lipids). In some instances, the concentrated agents are used as commercial products, e.g., the final user may use diluted agents, which have a substantially lower concentration of active ingredient. In some embodiments, the composition is formulated as an agricultural concentrate formulation, e.g., an ultra-low-volume concentrate formulation.

### Lipid Reconstructed Plant Messenger Packs (LPMPs)

A lipid reconstructed PMP (LPMP) is used herein. LPMP refers to a PMP that has been derived from a lipid structure (e.g., a lipid bilayer, unilamellar, multilamellar structure; e.g., a vesicular lipid structure) derived from (e.g., enriched, isolated or purified from) a plant source, wherein the lipid structure is disrupted (e.g., disrupted by lipid extraction) and reassembled or reconstituted in a liquid phase (e.g., a liquid phase containing a cargo) using standard methods, e.g., reconstituted by a method comprising lipid film hydration and/or solvent injection, to produce the LPMP, as is described herein. The method may, if desired, further comprise sonication, freeze/thaw treatment, and/or lipid extrusion, e.g., to reduce the size of the reconstituted LPMPs. Alternatively, LPMPs may be produced using a microfluidic device (such as a NanoAssemblr® IGNITE™ microfluidic instrument (Precision NanoSystems)).

In some embodiments, the LPMPs are produced by a process which comprises the steps of (a) providing a plurality of purified PMPs (e.g., PMPs purified as described in Section IA herein); (b) processing the plurality of PMPs to produce a lipid film; (c) reconstituting the lipid film in an organic solvent or solvent combination, thereby producing a lipid solution; and (d) processing the lipid solution of step (c) in a microfluidics device comprising an aqueous phase, thereby producing the LPMPs.

In some instances, processing the plurality of PMPs to produce a lipid film includes extracting lipids from the plurality of PMPs, e.g., extracting lipids using the Bligh-Dyer method (Bligh and Dyer, J Biolchem Physiol, 37: 911-917, 1959). The extracted lipids may be provided as a stock solution, e.g., a solution in chloroform:methanol. Producing the lipid film may comprise, e.g., evaporation of the solvent with a stream of inert gas (e.g., nitrogen).

### Natural Lipids

A LPMP may comprise between 10% and 100% lipids derived from the lipid structure from the plant source (e.g., lemon or algae), e.g., may contain at least 10%, at least 20%, at least 30%, at least 40%, at least 50%, at least 60%, at least 70%, at least 80%, at least 90%, at least 95%, or 100% lipids derived from the lipid structure from the plant source. A LPMP may comprise all or a fraction of the lipid species present in the lipid structure from the plant source (e.g., lemon or algae), e.g., it may contain at least 10%, at least 20%, at least 30%, at least 40%, at least 50%, at least 60%, at least 70%, at least 80%, at least 90%, or 100% of the lipid species present in the lipid structure from the plant source. A LPMP may comprise none, a fraction, or all of the protein species present in the lipid structure from the plant source (e.g., lemon or algae), e.g., may contain 0%, less than 1%, less than 5%, less than 10%, less than 15%, less than 20%, less than 30%, less than 40%, less than 50%, less than 60%, less than 70%, less than 80%, less than 90%, less than 100%, or 100% of the protein species present in the lipid structure from the plant source (e.g., lemon or algae). In some instances, the lipid bilayer of the LPMP does not contain proteins. In some instances, the lipid structure of the LPMP contains a reduced amount of proteins relative to the lipid structure from the plant source.

In some embodiments, the natural lipids of the LPMPs are extracted from lemon or algae.

### Exogenous Lipids

The LPMPs may be modified to contain a heterologous agent (e.g., a cell-penetrating agent) that is capable of increasing cell uptake (e.g., animal cell uptake (e.g., mammalian cell uptake, e.g., human cell uptake), plant cell uptake, bacterial cell uptake, or fungal cell uptake) relative to an unmodified LPMP. For example, the modified LPMPs may include (e.g., be loaded with, e.g., encapsulate or be conjugated to) or be formulated with (e.g., be suspended or resuspended in a solution comprising) a plant cell-penetrating agent, such as an ionizable lipid. Each of the modified LPMPs may comprise at least 1%, 5%, 10%, 20%, 30%, 40%, 50%, 60%, 70%, 80%, 90%, or more than 90% ionizable lipid.

LPMPs may include one or more exogenous lipids, e.g., lipids that are exogenous to the plant (e.g., originating from a source that is not the plant or plant part from which the LPMP is produced). The lipid composition of the LPMP may include 0%, less than 1%, or at least 1%, 2%, 5%, 10%, 15%, 20%, 25%, 30%, 40%, 50%, 60%, 70%, 80%, 90%, 95%, or more than 95% exogenous lipid. In some examples, the exogenous lipid (e.g., ionizable lipid) is added to amount to 25% or 40% (w/w) of total lipids in the preparation. In some examples, the exogenous lipid is added to the preparation prior to step (b), e.g., mixed with extracted PMP lipids prior to step (b).

Exemplary exogenous lipids include ionizable lipids.

Exogenous lipids may also include cationic lipids.

In some instances, the exogenous lipid may be an ionizable lipid or cationic lipid chosen from 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), DLin-MC3-DMA (MC3), dioleoyl-3-trimethylammonium propane (DODAP), DC-cholesterol, DOTAP, Ethyl PC, GL67, DLin-KC2-DMA (KC2), MD1 (cKK-E12), OF2, EPC, ZA3-Ep10, TT3, LP01, 5A2-SC8, Lipid 5 (Moderna), a cationic sulfonamide amino lipid, an amphiphilic zwitterionic amino lipid, DODAC, DOBAQ, YSK05, DOBAT, DOBAQ, DOPAT, DOMPAQ, DOAAQ, DMAP-BLP, DLinDMA, DODMA, DOTMA, DSDMA, DOSPA, DODAC, DOBAQ, DMRIE, DOTAP-cholesterol, GL67A, and 98N12-5 or a combination thereof.

In some embodiments, the exogenous lipid may be an ionizable lipid or cationic lipid chosen from C12-200, MC3, DODAP, DC-cholesterol, DOTAP, Ethyl PC, GL67, KC2, MD1, OF2, EPC, ZA3-Ep10, TT3, LP01, 5A2-SC8, Lipid 5 (Moderna), a cationic sulfonamide amino lipid, and an amphiphilic zwitterionic amino lipid or a combination thereof. In some embodiments, the ionizable lipid is chosen from C12-200, MC3, DODAP, and DC-cholesterol or combinations thereof. In some instances, the ionizable lipid is an ionizable lipid. In some embodiments, the ionizable lipid is 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200) or (6Z,9Z,28Z,31Z)-Heptatriaconta-6,9,28,31-tetraen-19-yl 4-(dimethylamino)butanoate, DLin-MC3-DMA (MC3). In some instances, the exogenous lipid is a cationic lipid. In some embodiments, the cationic lipid is DC-cholesterol or dioleoyl-3-trimethylammonium propane (DOTAP).

In some instances, the LPMPs comprise at least 1%, 5%, 10%, 20%, 30%, 40%, 50%, 60%, 70%, 80%, 90%, or more than 90% ionizable lipid.

In some instances, the LPMPs comprise a molar ratio of least 0.1%, 1%, 5%, 10%, 15%, 20%, 25%, 30%, 35%, 40%, 45%, 50%, 55%, 60%, 65%, 70%, 75% 80%, 85%, 90%, or more than 90% ionizable lipid, e.g., 1%-10%, 10%-20%, 20%-30%, 30%-40%, 40%-50%, 50%-60%, 60%-70%, 70%-80%, or 80%-90% ionizable lipid, e.g., about 30%-75% ionizable lipid (e.g., about 30%-75% ionizable lipid). In some embodiments, the LPMP comprises 25% C12-200. In some embodiments, the LPMP comprises a molar ratio of 35% C12-200. In some embodiments, the LPMP comprises a molar ratio of 50% C12-200. In some embodiments, the LPMP comprises 40% MC3. In some embodiments, the LPMP comprises a molar ratio of 50% C12-200. In some embodiments, the LPMP comprises 20% or 40% DC-cholesterol. In some embodiments, the LPMP comprises 25% or 40% DOTAP.

The agent may increase uptake of the LPMP as a whole or may increase uptake of a portion or component of the LPMP (e.g., the mRNA therapeutic) carried by the LPMP. The degree to which cell uptake is increased may vary depending on the plant or plant part to which the composition is delivered, the LPMP formulation, and other modifications made to the LPMP, For example, the modified LPMPs may have an increased cell uptake (e.g., animal cell uptake, plant cell uptake, bacterial cell uptake, or fungal cell uptake) of at least 1%, 2%, 5%, 10%, 15%, 20%, 30%, 40%, 50%, 60%, 70%, 80%, 90%, or 100% relative to an unmodified LPMP. In some instances, the increased cell uptake is an increased cell uptake of at least 2×-fold, 4×-fold, 5×-fold, 10×-fold, 100×-fold, or 1000×-fold relative to an unmodified LPMP.

In some embodiments, a LPMP that has been modified with a ionizable lipid more efficiently encapsulates a negatively charged a polynucleotide than a LPMP that has not been modified with an ionizable lipid. In some aspects, a LPMP that has been modified with an ionizable lipid has altered biodistribution relative to a LPMP that has not been modified with an ionizable lipid. In some aspects, a LPMP that has been modified with an ionizable lipid has altered (e.g., increased) fusion with an endosomal membrane of a target cell relative to a LPMP that has not been modified with an ionizable lipid.

### Ionizable Lipids

In some embodiments, the ionizable lipid has at least one (e.g., one, two, three, four or all five) of the characteristics listed below:
(i) at least 2 ionizable amines (e.g., at least 2, at least 3, at least 4, at least 5, at least 6, or more than 6 ionizable amines, e.g., 2, 3, 4, 5, 6, 7, 8, 9, 10, 11, 12, or more than 12 ionizable amines);
(ii) at least 3 lipid tails (e.g., at least 3, at least 4, at least 5, at least 6, or more than 6 lipid tails, e.g., 2, 3, 4, 5, 6, 7, 8, 9, 10, 11, 12, or more than 12 lipid tails), wherein each of the lipid tails is independently at least 6 carbon atoms in length (e.g., at least 6, at least 7, at least 8, at least 9, at least 10, at least 11, at least 12, at least 13, at least 14, at least 15, at least 16, at least 17, at least 18, or more than 18 carbon atoms in length, e.g., 6, 7, 8, 9, 10, 11, 12, 13, 14, 15, 16, 17, 18, 19, 20, 21, 22, 23, 24, 25, or more than 25 carbon atoms in length);
(iii) an acid dissociation constant (pKa) of from about 4.5 to about 7.5 (e.g., a pKa of about 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5 (e.g., a pKa of from about 6.5 and about 7.5 (e.g., a pKa of about 6.5, 6.6, 6.7, 6.8, 6.9, 7.0, 7.1, 7.2, 7.3, 7.4, or 7.5));
(iv) an ionizable amine and a heteroorganic group; and
(v) an N:P (amines of ionizable lipid:phosphates of mRNA) ratio of at least 10;

In some embodiments, the ionizable lipid is not selected from 1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), MD1 (cKK-E12), OF2, EPC, ZA3-Ep10, TT3, LP01, 5A2-SC8, Lipid 5 (Moderna), and 98N12-5.

In some embodiments, the ionizable lipid is selected from the group consisting of 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), MD1 (cKK-E12), OF2, EPC, ZA3-Ep10, TT3, LP01, 5A2-SC8, Lipid 5, SM-102 (Lipid H), and ALC-315.

In some embodiments, the ionizable lipid is an ionizable amine and a heteroorganic group. In some embodiments, the heteroorganic group is hydroxyl. In some embodiments, the heteroorganic group comprises a hydrogen bond donor. In some embodiments, the heteroorganic group comprises a hydrogen bond acceptor. In some embodiments, the heteroorganic group is —OH, —SH, —(CO)H, —CO₂H, —NH₂, —CONH₂, optionally substituted C₁-C₆ alkoxy, or fluorine.

In some embodiments, the ionizable lipid is an ionizable amine and a heteroorganic group separated by a chain of at least two atoms

In some embodiments, the ionizable lipid is represented by the following formula I:

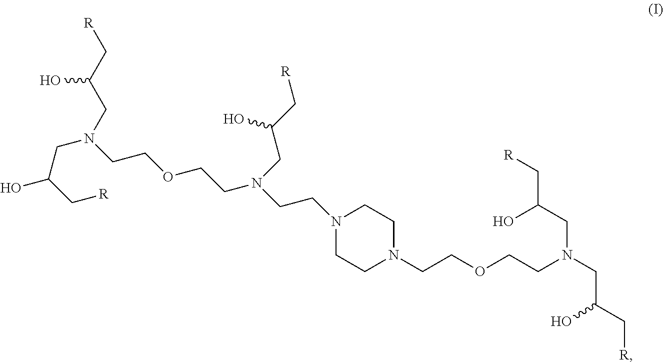

wherein R is C8-C14 alkyl group.

In some embodiments, a lipid membrane of the LPMPs comprises at least 35% of the lipid of formula I, e.g., at least 40%, 45%, 50%, 55%, 60%, 65%, 70%, 75% 80%, 85%, 90%, or more than 90% of the lipid of formula I, e.g., 35%-40%, 40%-50%, 50%-60%, 60%-70%, 70%-80%, or 80%-90% of the lipid of formula I.

In some instances, the LPMPs comprise at least 1%, 5%, 10%, 20%, 30%, 40%, 50%, 60%, 70%, 80%, 90%, or more than 90% ionizable lipid.

In some instances, the LPMPs comprise a molar ratio of at least 0.1%, 1%, 5%, 10%, 15%, 20%, 25%, 30%, 35%, 40%, 45%, 50%, 55%, 60%, 65%, 70%, 75% 80%, 85%, 90%, or more than 90% ionizable lipid, e.g., 1%-10%, 10%-20%, 20%-30%, 30%-40%, 40%-50%, 50%-60%, 60%-70%, 70%-80%, or 80%-90% ionizable lipid, e.g., about 25%-75% ionizable lipid (e.g., about 25%-75% ionizable lipid).

The ionizable lipid described herein may include an amine core described herein substituted with one or more (e.g., 1, 2, 3, 4, 5, or 6) lipid tails. In some embodiments, the ionizable lipid described herein include at least 3 lipid tails. A lipid tail may be a C₈-C₁₈ hydrocarbon (e.g., C₆-C₁₈ alkyl or C₆-C₁₈ alkanoyl). An amine core may be substituted with one or more lipid tails at a nitrogen atom (e.g., one hydrogen atom attached to the nitrogen atom may be replaced with a lipid tail).

In some embodiments, the amine core has a structure of:

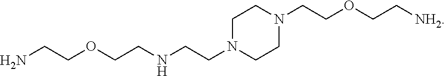 [SMILES: NCCOCCNCCN1CCN(CCOCCN)CC1]

In some embodiments, the amine core has a structure of:

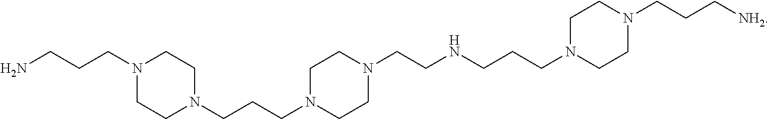 [SMILES: NCCCN1CCN(CCCNCCN2CCN(CCCN3CCN(CCCN)CC3)CC2)CC1]

In some embodiments, the amine core has a structure of:

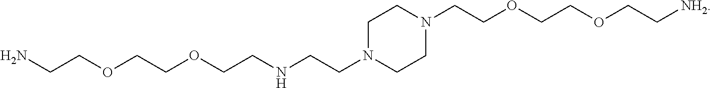 [SMILES: NCCOCCOCCNCCN1CCN(CCOCCOCCN)CC1]

In some embodiments, the amine core has a structure of:

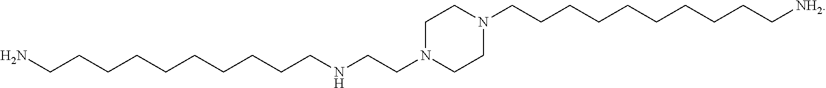 [SMILES: NCCCCCCCCCCNCCN1CCN(CCCCCCCCCCN)CC1]

In some embodiments, the amine core has a structure of:

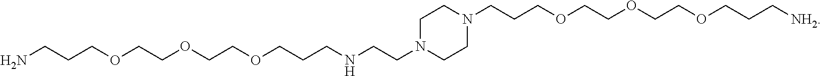 [SMILES: NCCCOCCOCCOCCCNCCN1CCN(CCCOCCOCCOCCCN)CC1]

In some embodiments, the amine core has a structure of:

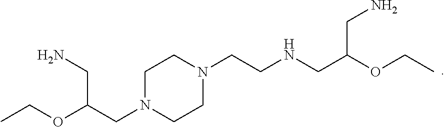 [SMILES: CCOC(CN)CNCCN1CCN(CC(CN)OCC)CC1]

In some embodiments, the amine core has a structure of:

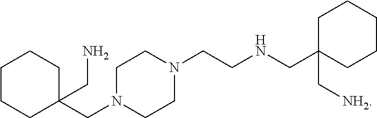 [SMILES: NCC1(CNCCN2CCN(CC3(CN)CCCCC3)CC2)CCCCC1]

In some embodiments, the amine core has a structure of:

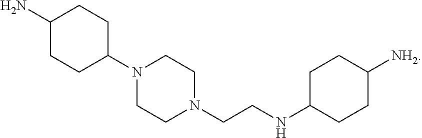 [SMILES: NC1CCC(NCCN2CCN(C3CCC(N)CC3)CC2)CC1]

Other suitable lipids for use in the mRNA therapeutic composition and methods for making and using thereof include the lipids as described in International Patent Publication WO 2016/118725, which is incorporated herein by reference in its entirety.

In certain embodiments, the mRNA therapeutic composition and methods for making and using thereof include a lipid having a compound structure of:

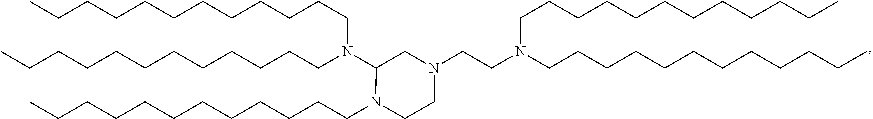 [SMILES: CCCCCCCCCCCCN(CCCCCCCCCCCC)CCN1CCN(CCCCCCCCCCCC)C(N(CCCCCCCCCCCC)CCCCCCCCCCCC)C1]

and pharmaceutically acceptable salts thereof.

Other suitable lipids for use in the mRNA therapeutic composition and methods for making and using thereof include the lipids as described in International Patent Publication WO 2016/118724, which is incorporated herein by reference in its entirety.

In certain embodiments, the mRNA therapeutic composition and methods for making and using thereof include a lipid having a compound structure of:

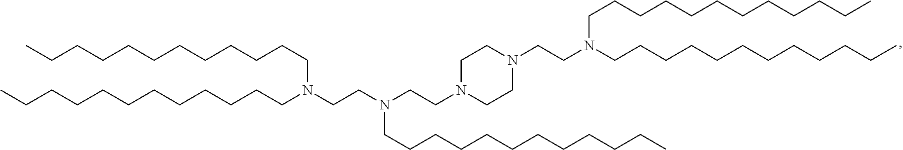 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1]

and pharmaceutically acceptable salts thereof.

Other suitable lipids for use in the mRNA therapeutic composition and methods for making and using thereof include a lipid having the formula of 14,25-ditridecyl 15,18,21,24-tetraaza-octatriacontane, and pharmaceutically acceptable salts thereof.

Other suitable lipids for use in the mRNA therapeutic composition and methods for making and using thereof include the lipids as described in International Patent Publications WO 2013/063468 and WO 2016/205691, each of which is incorporated herein by reference in its entirety.

In some embodiments, the mRNA therapeutic composition and methods for making and using thereof include a lipid of the following formula:

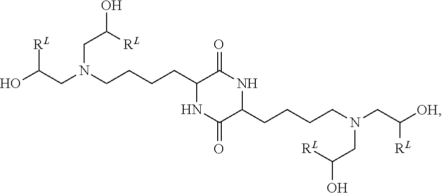

or pharmaceutically acceptable salts thereof, wherein each instance of R^{L} is independently optionally substituted C6-C40 alkenyl.

In certain embodiments, the mRNA therapeutic composition and methods for making and using thereof include a lipid having a compound structure of:

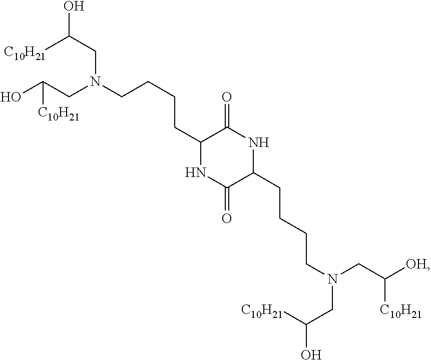

and pharmaceutically acceptable salts thereof.

In certain embodiments, the mRNA therapeutic composition and methods for making and using thereof include a lipid having a compound structure of:

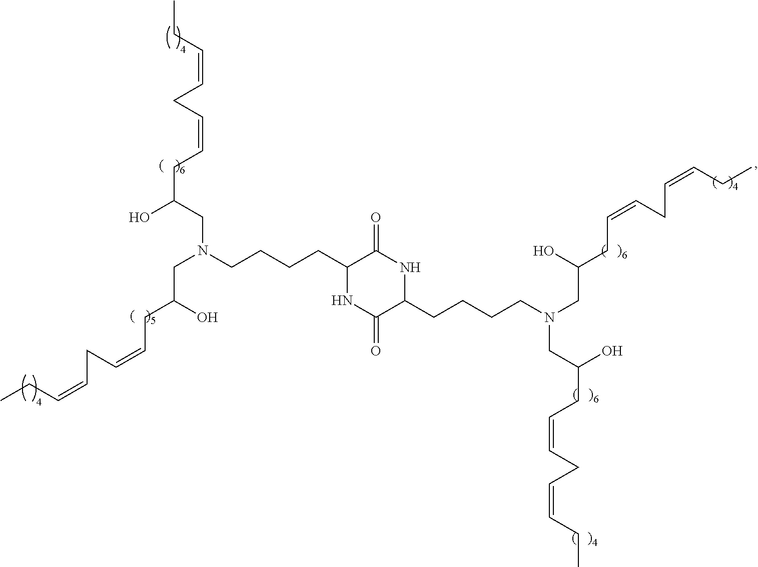

and pharmaceutically acceptable salts thereof.

In certain embodiments, the mRNA therapeutic composition and methods for making and using thereof include a lipid having a compound structure of:

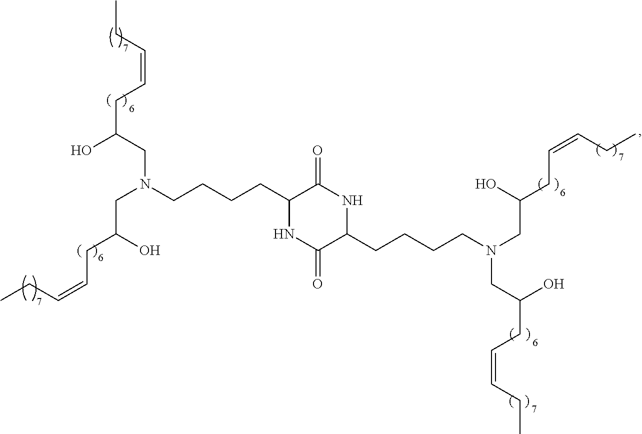

and pharmaceutically acceptable salts thereof.

In certain embodiments, the mRNA therapeutic composition and methods for making and using thereof include a lipid having a compound structure of:

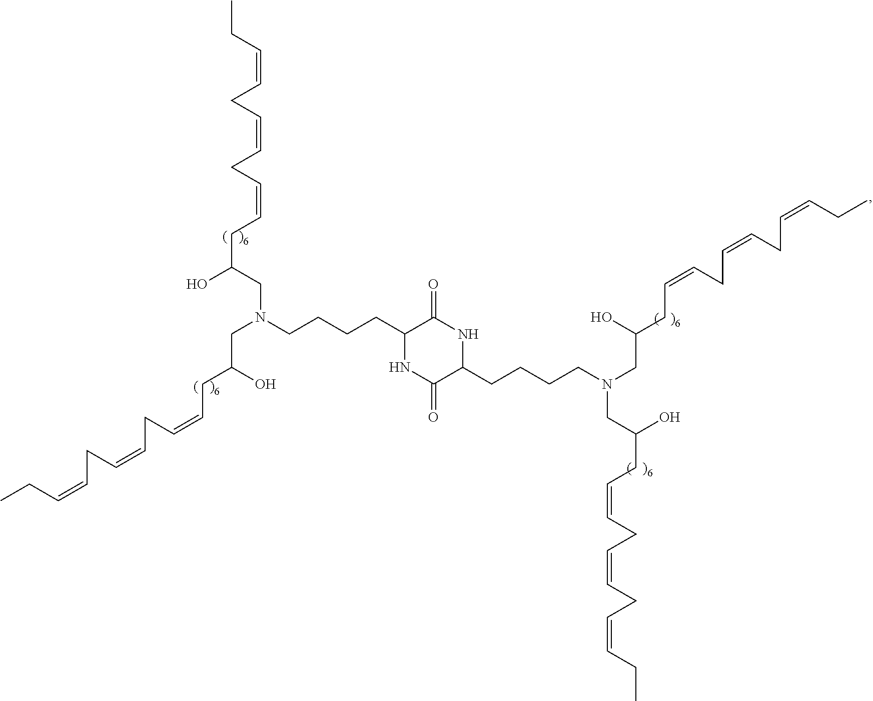

and pharmaceutically acceptable salts thereof.

Other suitable lipids for use in the mRNA therapeutic composition and methods for making and using thereof include the lipids as described in International Patent Publication WO 2015/184256, which is incorporated herein by reference in its entirety. In some embodiments, the mRNA therapeutic composition and methods for making and using thereof include a lipid of the following formula:

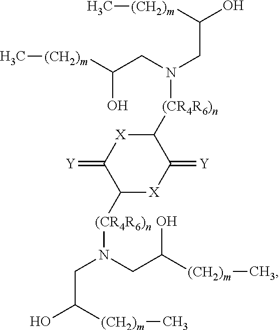

or a pharmaceutically acceptable salt thereof, wherein each X independently is O or S; each Y independently is O or S; each m independently is 0 to 20; each n independently is 1 to 6; each R_{A} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen; and each RB is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen.

In certain embodiments, the mRNA therapeutic composition and methods for making and using thereof include a lipid, “Target 23”, having a compound structure of:

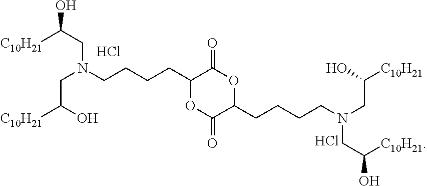

(Target 23) and pharmaceutically acceptable salts thereof.

Other suitable lipids for use in the mRNA therapeutic composition and methods for making and using thereof include the lipids as described in International Patent Publication WO 2016/004202, which is incorporated herein by reference in its entirety.

In some embodiments, the mRNA therapeutic composition and methods for making and using thereof include a lipid having the compound structure:

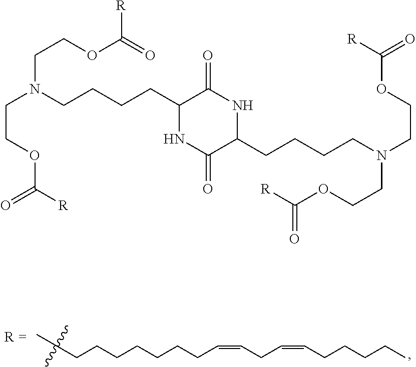

or a pharmaceutically acceptable salt thereof.

In some embodiments, the mRNA therapeutic composition and methods for making and using thereof include a lipid having the compound structure:

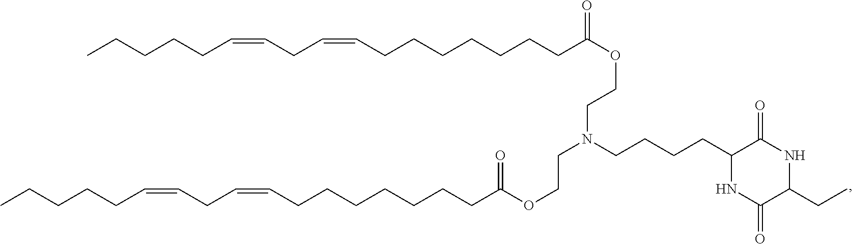 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC]

or a pharmaceutically acceptable salt thereof.

In some embodiments, the mRNA therapeutic composition and methods for making and using thereof include a lipid having the compound structure:

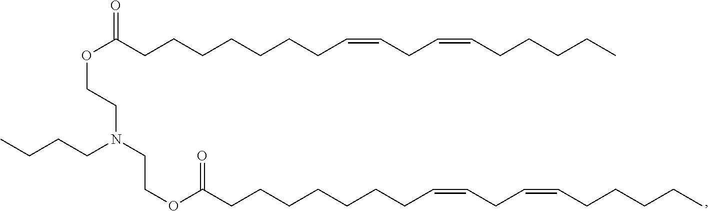 [SMILES: CCCCC/C=C\C/C=C\CCCCCCCC(=O)OCCN(CCCC)CCOC(=O)CCCCCCC/C=C\C/C=C\CCCCC]

or a pharmaceutically acceptable salt thereof.

Other suitable lipids for use in the mRNA therapeutic composition and methods for making and using thereof include lipids as described in U.S. Provisional Patent Application Ser. No. 62/758,179, which is incorporated herein by reference in its entirety.

In some embodiments, the mRNA therapeutic composition and methods for making and using thereof include a lipid of the following formula:

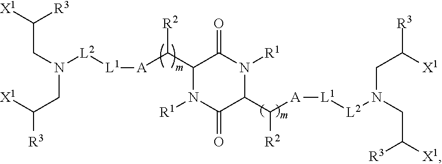

or a pharmaceutically acceptable salt thereof, wherein each R¹ and R² is independently H or C1-C6 aliphatic; each m is independently an integer having a value of 1 to 4; each A is independently a covalent bond or arylene; each L¹ is independently an ester, thioester, disulfide, or anhydride group; each L² is independently C2-C10 aliphatic; each X¹ is independently H or OH; and each R³ is independently C6-C20 aliphatic.

In some embodiments, the mRNA therapeutic composition and methods for making and using thereof include a lipid of the following formula:

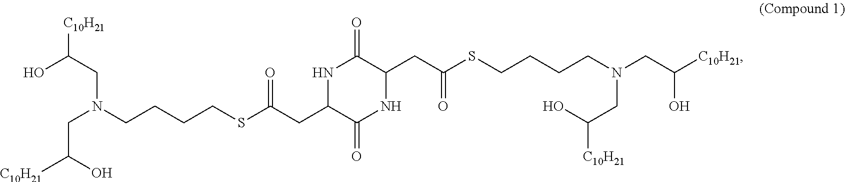

or a pharmaceutically acceptable salt thereof.

In some embodiments, the mRNA therapeutic composition and methods for making and using thereof include a lipid of the following formula:

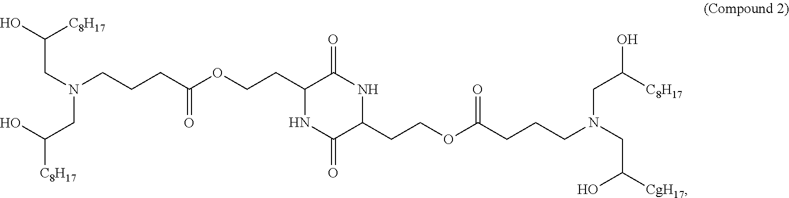

or a pharmaceutically acceptable salt thereof.

In some embodiments, the mRNA therapeutic composition and methods for making and using thereof include a lipid of the following formula:

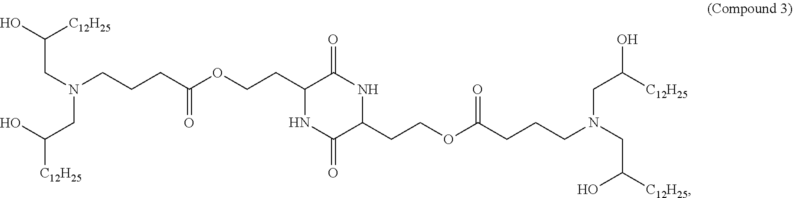

or a pharmaceutically acceptable salt thereof.

Other suitable lipids for use in the mRNA therapeutic composition and methods for making and using thereof include the lipids as described in J. McClellan, M. C. King, Cell 2010, 141, 210-217 and in Whitehead et al., Nature Communications (2014) 5:4277, which is incorporated herein by reference in its entirety.

In certain embodiments, the lipids of the mRNA therapeutic composition and methods for making and using thereof include a lipid having a compound structure of:

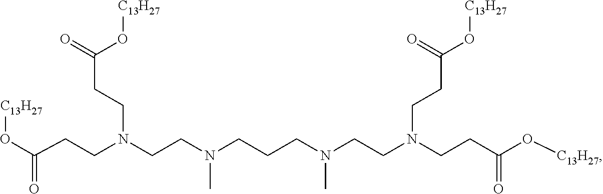

and pharmaceutically acceptable salts thereof.

Other suitable lipids for use in the mRNA therapeutic composition and methods for making and using thereof include the lipids as described in International Patent Publication WO 2015/199952, which is incorporated herein by reference in its entirety.

In some embodiments, the mRNA therapeutic composition and methods for making and using thereof include a lipid having the compound structure:

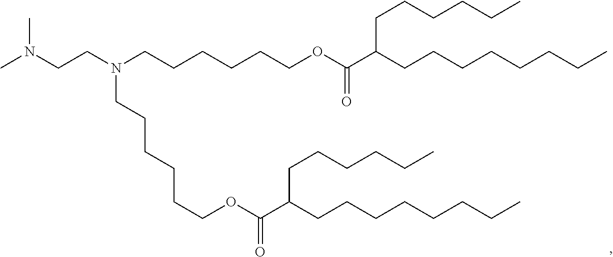 [SMILES: CCCCCCCCC(CCCCCC)C(=O)OCCCCCCN(CCCCCCOC(=O)C(CCCCCC)CCCCCCCC)CCN(C)C]

and pharmaceutically acceptable salts thereof.

In some embodiments, the mRNA therapeutic composition and methods for making and using thereof include a lipid having the compound structure:

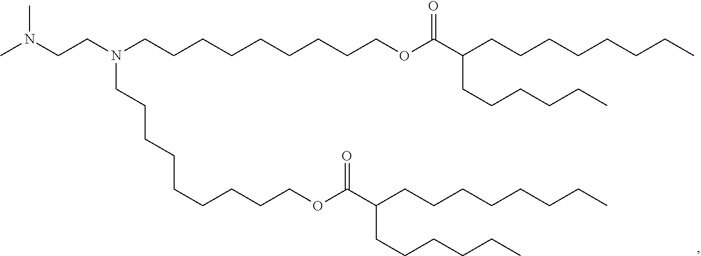 [SMILES: CCCCCCCCC(CCCCCC)C(=O)OCCCCCCCCCN(CCCCCCCCCOC(=O)C(CCCCCC)CCCCCCCC)CCN(C)C]

and pharmaceutically acceptable salts thereof.

In some embodiments, the mRNA therapeutic composition and methods for making and using thereof include a lipid having the compound structure:

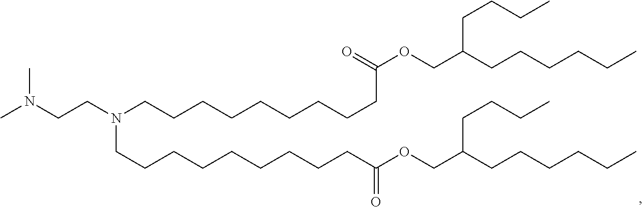 [SMILES: CCCCCCC(CCCC)COC(=O)CCCCCCCCCN(CCCCCCCCCC(=O)OCC(CCCC)CCCCCC)CCN(C)C]

and pharmaceutically acceptable salts thereof.

In some embodiments, the mRNA therapeutic composition and methods for making and using thereof include a lipid having the compound structure:

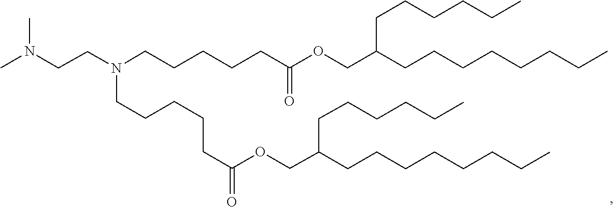 [SMILES: CCCCCCCCC(CCCCCC)COC(=O)CCCCCN(CCCCCC(=O)OCC(CCCCCC)CCCCCCCC)CCN(C)C]

and pharmaceutically acceptable salts thereof.

In some embodiments, the mRNA therapeutic composition and methods for making and using thereof include a lipid having the compound structure:

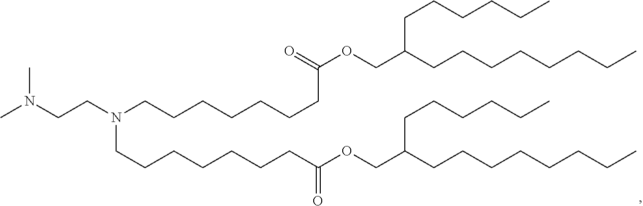 [SMILES: CCCCCCCCC(CCCCCC)COC(=O)CCCCCCCN(CCCCCCCC(=O)OCC(CCCCCC)CCCCCCCC)CCN(C)C]

and pharmaceutically acceptable salts thereof.

In some embodiments, the mRNA therapeutic composition and methods for making and using thereof include a lipid having the compound structure:

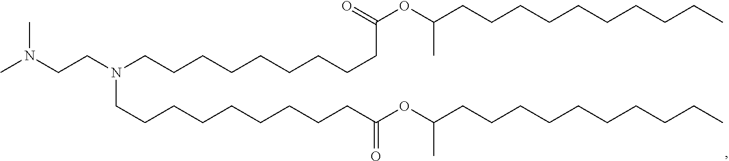 [SMILES: CCCCCCCCCCC(C)OC(=O)CCCCCCCCCN(CCCCCCCCCC(=O)OC(C)CCCCCCCCCC)CCN(C)C]

and pharmaceutically acceptable salts thereof.

In some embodiments, the mRNA therapeutic composition and methods for making and using thereof include a lipid having the compound structure:

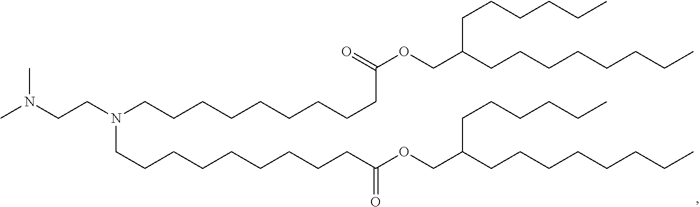 [SMILES: CCCCCCCCC(CCCCCC)COC(=O)CCCCCCCCCN(CCCCCCCCCC(=O)OCC(CCCCCC)CCCCCCCC)CCN(C)C]

and pharmaceutically acceptable salts thereof.

In some embodiments, the mRNA therapeutic composition and methods for making and using thereof include a lipid having the compound structure:

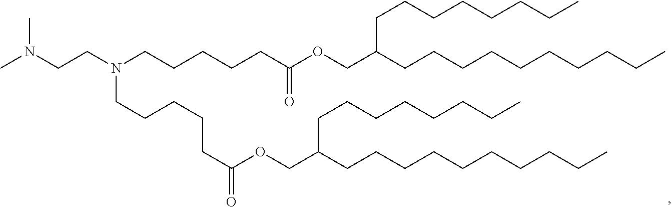 [SMILES: CCCCCCCCCCC(CCCCCCCC)COC(=O)CCCCCN(CCCCCC(=O)OCC(CCCCCCCC)CCCCCCCCCC)CCN(C)C]

and pharmaceutically acceptable salts thereof.

In some embodiments, the mRNA therapeutic composition and methods for making and using thereof include a lipid having the compound structure:

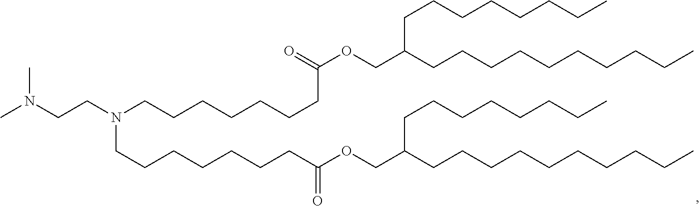 [SMILES: CCCCCCCCCCC(CCCCCCCC)COC(=O)CCCCCCCN(CCCCCCCC(=O)OCC(CCCCCCCC)CCCCCCCCCC)CCN(C)C]

and pharmaceutically acceptable salts thereof.

In some embodiments, the mRNA therapeutic composition and methods for making and using thereof include a lipid having the compound structure:

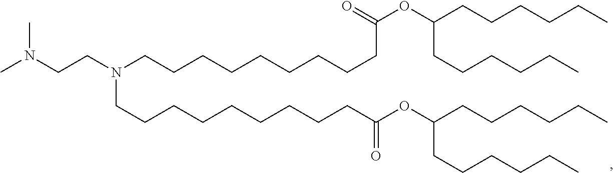 [SMILES: CCCCCCC(CCCCCC)OC(=O)CCCCCCCCCN(CCCCCCCCCC(=O)OC(CCCCCC)CCCCCC)CCN(C)C]

and pharmaceutically acceptable salts thereof.

In some embodiments, the mRNA therapeutic composition and methods for making and using thereof include a lipid having the compound structure:

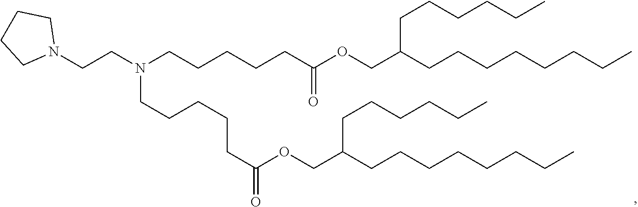 [SMILES: CCCCCCCCC(CCCCCC)COC(=O)CCCCCN(CCCCCC(=O)OCC(CCCCCC)CCCCCCCC)CCN1CCCC1]

and pharmaceutically acceptable salts thereof.

In some embodiments, the mRNA therapeutic composition and methods for making and using thereof include a lipid having the compound structure:

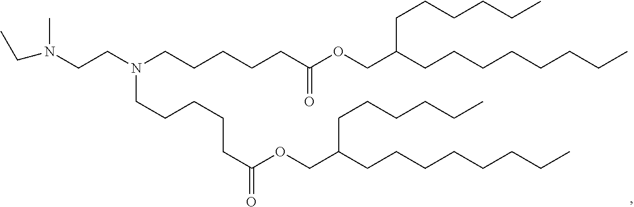 [SMILES: CCCCCCCCC(CCCCCC)COC(=O)CCCCCN(CCCCCC(=O)OCC(CCCCCC)CCCCCCCC)CCN(C)CC]

and pharmaceutically acceptable salts thereof.

In some embodiments, the mRNA therapeutic composition and methods for making and using thereof include a lipid having the compound structure:

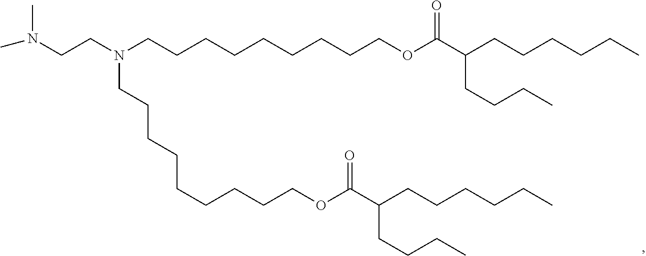 [SMILES: CCCCCCC(CCCC)C(=O)OCCCCCCCCCN(CCCCCCCCCOC(=O)C(CCCC)CCCCCC)CCN(C)C]

and pharmaceutically acceptable salts thereof.

Other suitable lipids for use in the mRNA therapeutic composition and methods for making and using thereof include the lipids as described in International Patent Publication WO 2017/004143, which is incorporated herein by reference in its entirety.

In some embodiments, the mRNA therapeutic composition and methods for making and using thereof include a lipid having the compound structure:

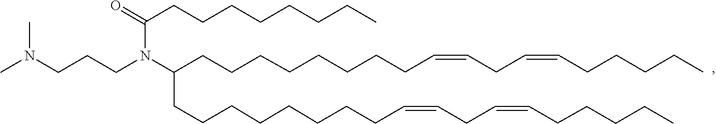 [SMILES: CCCCC/C=C\C/C=C\CCCCCCCCC(CCCCCCCC/C=C\C/C=C\CCCCC)N(CCCN(C)C)C(=O)CCCCCCCC]

and pharmaceutically acceptable salts thereof.

In some embodiments, the mRNA therapeutic composition and methods for making and using thereof include a lipid having the compound structure:

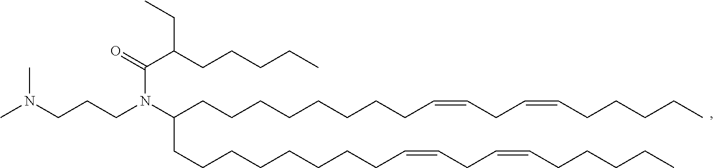 [SMILES: CCCCC/C=C\C/C=C\CCCCCCCCC(CCCCCCCC/C=C\C/C=C\CCCCC)N(CCCN(C)C)C(=O)C(CC)CCCCC]

and pharmaceutically acceptable salts thereof.

In some embodiments, the mRNA therapeutic composition and methods for making and using thereof include a lipid having the compound structure:

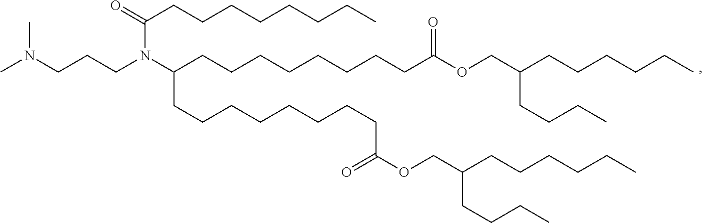 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

and pharmaceutically acceptable salts thereof.

In some embodiments, the mRNA therapeutic composition and methods for making and using thereof include a lipid having the compound structure:

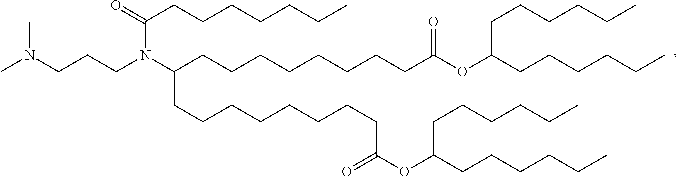 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof.

In some embodiments, the mRNA therapeutic composition and methods for making and using thereof include a lipid having the compound structure:

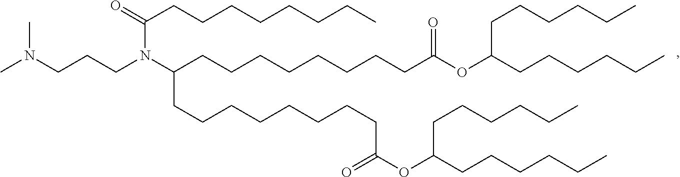 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof.

In some embodiments, the mRNA therapeutic composition and methods for making and using thereof include a lipid having the compound structure:

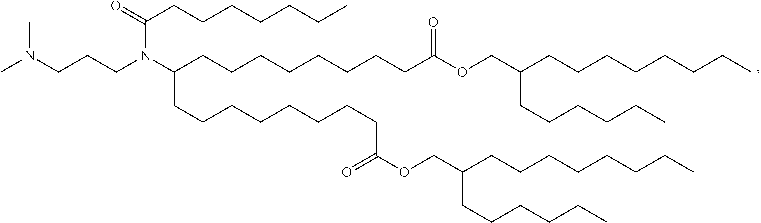 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

and pharmaceutically acceptable salts thereof.

In some embodiments, the mRNA therapeutic composition and methods for making and using thereof include a lipid having the compound structure:

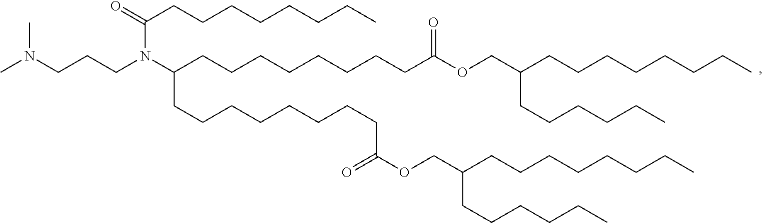 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC]

and pharmaceutically acceptable salts thereof.

In some embodiments, the mRNA therapeutic composition and methods for making and using thereof include a lipid having the compound structure:

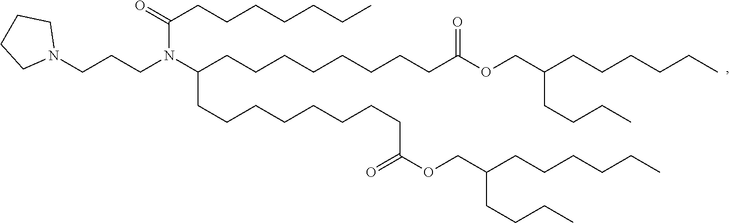 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof.

In some embodiments, the mRNA therapeutic composition and methods for making and using thereof include a lipid having the compound structure:

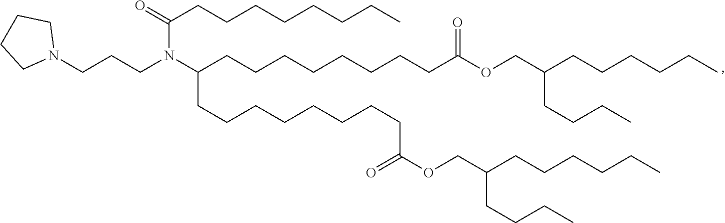 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof.

In some embodiments, the mRNA therapeutic composition and methods for making and using thereof include a lipid having the compound structure:

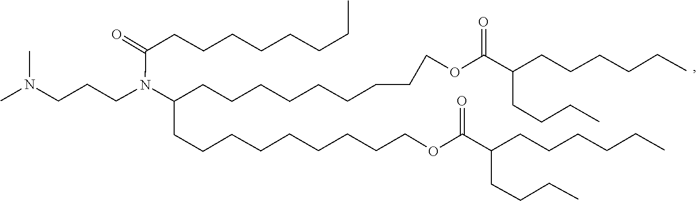 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC]

and pharmaceutically acceptable salts thereof.

In some embodiments, the mRNA therapeutic composition and methods for making and using thereof include a lipid having the compound structure:

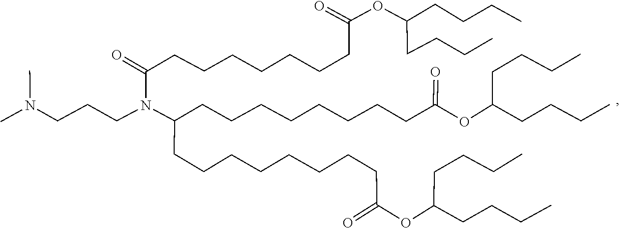 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC]

and pharmaceutically acceptable salts thereof.

In some embodiments, the mRNA therapeutic composition and methods for making and using thereof include a lipid having the compound structure:

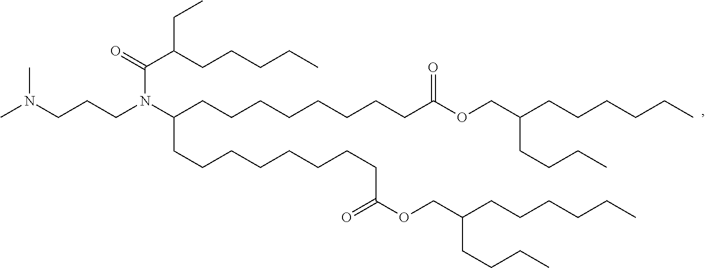 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC]

and pharmaceutically acceptable salts thereof.

In some embodiments, the mRNA therapeutic composition and methods for making and using thereof include a lipid having the compound structure:

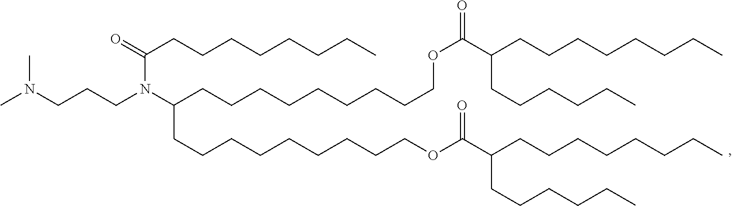 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC]

and pharmaceutically acceptable salts thereof.

In some embodiments, the mRNA therapeutic composition and methods for making and using thereof include a lipid having the compound structure:

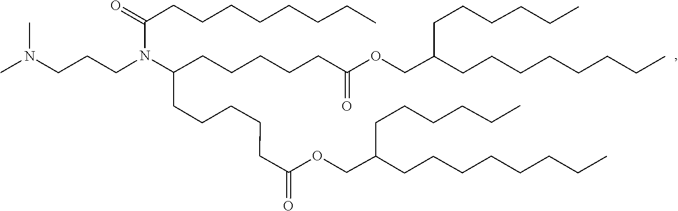 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof.

In some embodiments, the mRNA therapeutic composition and methods for making and using thereof include a lipid having the compound structure:

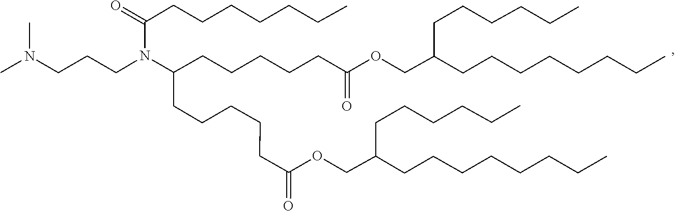 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC]

and pharmaceutically acceptable salts thereof.

In some embodiments, the mRNA therapeutic composition and methods for making and using thereof include a lipid having the compound structure:

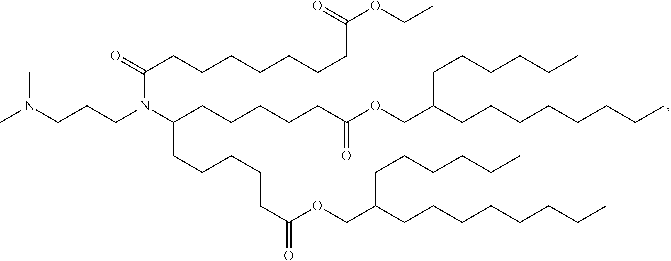 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC]

and pharmaceutically acceptable salts thereof.

In some embodiments, the mRNA therapeutic composition and methods for making and using thereof include a lipid having the compound structure:

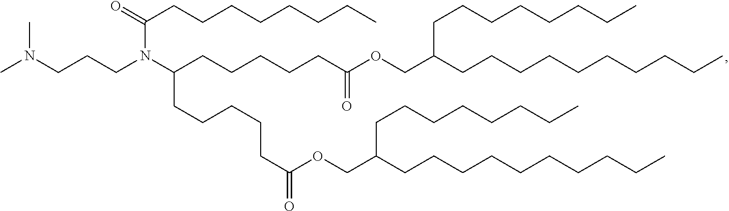 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC]

and pharmaceutically acceptable salts thereof.

Other suitable lipids for use in the mRNA therapeutic composition and methods for making and using thereof include the lipids as described in International Patent Publication WO 2017/075531, which is incorporated herein by reference in its entirety.

In some embodiments, the mRNA therapeutic composition and methods for making and using thereof include a lipid of the following formula:

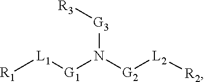

or a pharmaceutically acceptable salt thereof, wherein one of L¹ or L² is —O(C═O)—, —(C═O)O—, —C(═O)—, —O—, —S(O)x, —S—S—, —C(═O)S—, —SC(═O)—, —NR^{a}C(═O)—, —C(═O)NR^{a}—, NR^{a}C(═O)NR^{a}—, —OC(═O)NR^{a}—, or —NR^{a}C(═O)O—; and the other of L¹ or L² is —O(C═O)—, —(C═O)O—, —C(═O)—, —O—, —S(O)ₓ, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond; G¹ and G² are each independently unsubstituted C₁-C₁₂alkylene or C₁-C₁₂ alkenylene; G³ is C₁-C₂₄ alkylene, C₁-C₂₄ alkenylene, C₃-C₈ cycloalkylene, C₃-C₈ cycloalkenylene; R^{a} is H or C₁-C₁₂ alkyl; R¹ and R² are each independently C₆-C₂₄ alkyl or C₆ -C₂₄ alkenyl; R³ is H, OR⁵, CN, —C(═O)OR⁴, —OC(═O)R⁴ or —NR⁵C(═O)R⁴; R⁴ is C₁-C₁₂ alkyl; R⁵ is H or C₁-C₆ alkyl; and x is 0, 1 or 2.

Other suitable lipids for use in the mRNA therapeutic composition and methods for making and using thereof include the lipids as described in International Patent Publication WO 2017/117528, which is incorporated herein by reference in its entirety. In some embodiments, the mRNA therapeutic composition and methods for making and using thereof include a lipid having the compound structure:

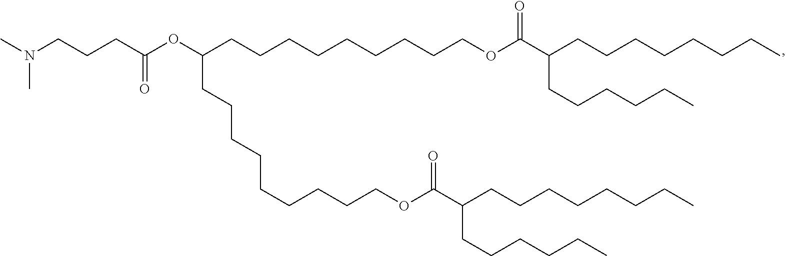 [SMILES: CCCCCCCCC(CCCCCC)C(=O)OCCCCCCCCCC(CCCCCCCCCOC(=O)C(CCCCCC)CCCCCCCC)OC(=O)CCCN(C)C]

and pharmaceutically acceptable salts thereof.

In some embodiments, the mRNA therapeutic composition and methods for making and using thereof include a lipid having the compound structure:

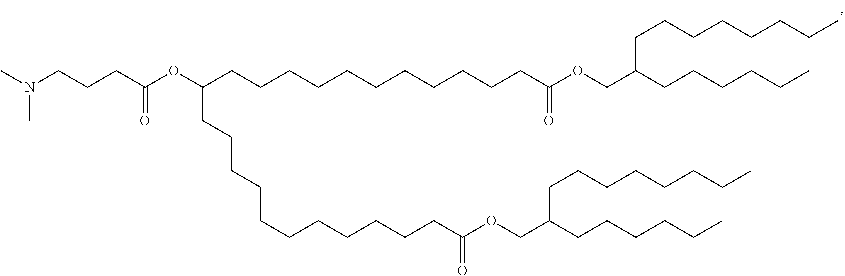 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C]

and pharmaceutically acceptable salts thereof.

In some embodiments, the mRNA therapeutic composition and methods for making and using thereof include a lipid having the compound structure:

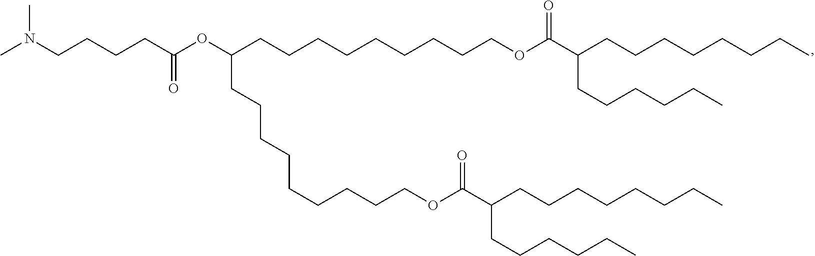 [SMILES: CCCCCCCCC(CCCCCC)C(=O)OCCCCCCCCCC(CCCCCCCCCOC(=O)C(CCCCCC)CCCCCCCC)OC(=O)CCCCN(C)C]

and pharmaceutically acceptable salts thereof.

Other suitable lipids for use in the mRNA therapeutic composition and methods for making and using thereof include the lipids as described in International Patent Publication WO 2017/049245, which is incorporated herein by reference in its entirety.

In some embodiments, the lipids of the mRNA therapeutic composition and methods for making and using thereof include a compound of one of the following formulas:

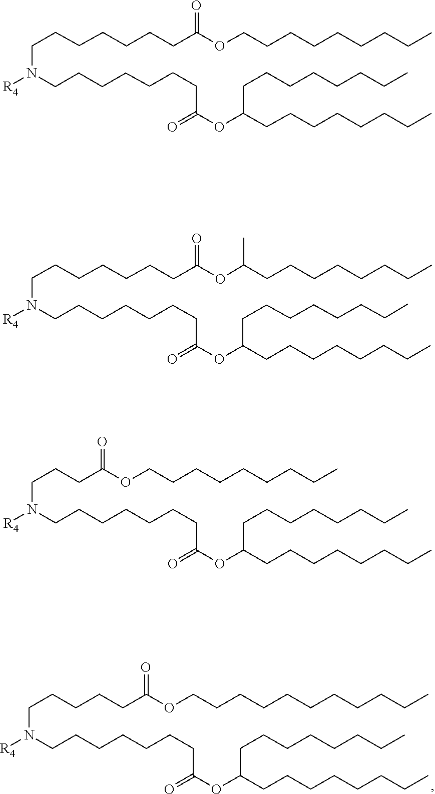

and pharmaceutically acceptable salts thereof. For any one of these four formulas, R₄ is independently selected from —(CH₂)ₙQ and —(CH₂)ₙCHQR; Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle; and n is 1, 2, or 3.

In certain embodiments, the mRNA therapeutic composition and methods for making and using thereof include a lipid having a compound structure of:

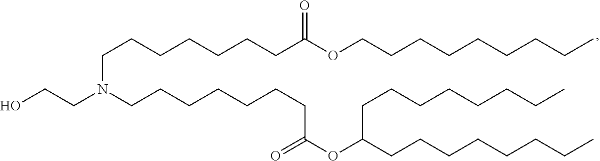 [SMILES: CCCCCCCCCOC(=O)CCCCCCCN(CCO)CCCCCCCC(=O)OC(CCCCCCCC)CCCCCCCC]

and pharmaceutically acceptable salts thereof.

In certain embodiments, the mRNA therapeutic composition and methods for making and using thereof include a lipid having a compound structure of:

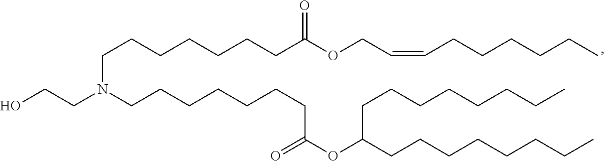 [SMILES: CCCCCC/C=C\COC(=O)CCCCCCCN(CCO)CCCCCCCC(=O)OC(CCCCCCCC)CCCCCCCC]

and pharmaceutically acceptable salts thereof.

In certain embodiments, the mRNA therapeutic composition and methods for making and using thereof include a lipid having a compound structure of:

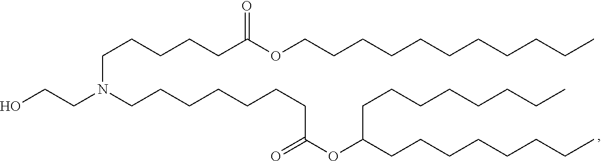 [SMILES: CCCCCCCCCCCOC(=O)CCCCCN(CCO)CCCCCCCC(=O)OC(CCCCCCCC)CCCCCCCC ; ","]

and pharmaceutically acceptable salts thereof.

In certain embodiments, the mRNA therapeutic composition and methods for making and using thereof include a lipid having a compound structure of:

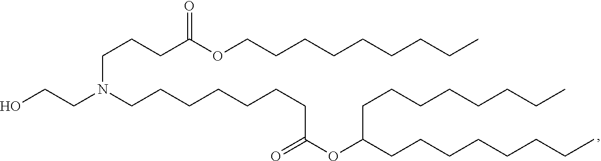 [SMILES: CCCCCCCCCOC(=O)CCCN(CCO)CCCCCCCC(=O)OC(CCCCCCCC)CCCCCCCC ; ","]

and pharmaceutically acceptable salts thereof.

Other suitable lipids for use in the mRNA therapeutic composition and methods for making and using thereof include the lipids as described in International Patent Publication WO 2017/173054 and WO 2015/095340, each of which is incorporated herein by reference in its entirety. In certain embodiments, the mRNA therapeutic composition and methods for making and using thereof include a lipid having a compound structure of:

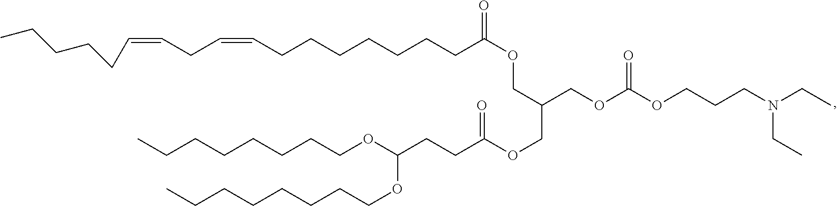 [SMILES: CCCCC/C=C\C/C=C\CCCCCCCC(=O)OCC(COC(=O)CCC(OCCCCCCCC)OCCCCCCCC)COC(=O)OCCCN(CC)CC]

and pharmaceutically acceptable salts thereof.

In certain embodiments, the mRNA therapeutic composition and methods for making and using thereof include a lipid having a compound structure of:

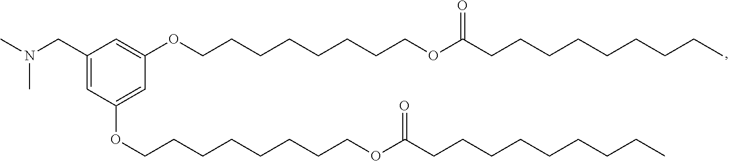 [SMILES: CCCCCCCCCC(=O)OCCCCCCCCOc1cc(CN(C)C)cc(OCCCCCCCCOC(=O)CCCCCCCCC)c1]

and pharmaceutically acceptable salts thereof.

In certain embodiments, the mRNA therapeutic composition and methods for making and using thereof include a lipid having a compound structure of:

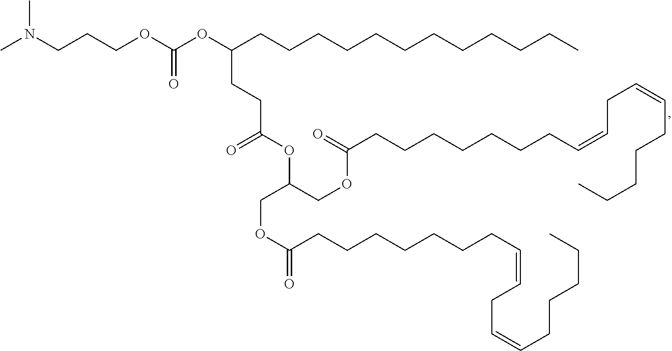 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

and pharmaceutically acceptable salts thereof.

In certain embodiments, the mRNA therapeutic composition and methods for making and using thereof include a lipid having a compound structure of:

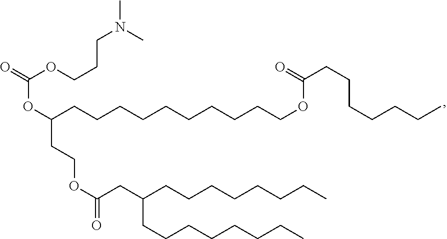 [SMILES: CCCCCCCCC(CCCCCCCC)CC(=O)OCCC(CCCCCCCCCCOC(=O)CCCCCCC)OC(=O)OCCCN(C)C]

and pharmaceutically acceptable salts thereof.

In some embodiments, the LPMPs described herein may include a ionizable lipid as described in, may be formulated as described in, or may comprise or be comprised by a composition as described in WO2016118724, WO2016118725, WO2016187531, WO2017176974, WO2018078053, WO2019027999, WO2019036030, WO2019089828, WO2019099501, WO2020072605, WO2020081938, WO2020118041, WO2020146805, or WO2020219876, each of which is incorporated by reference herein in its entirety.

### Other Lipids and Other Agents

The exogenous lipid may be a cell-penetrating agent, may be capable of increasing delivery of a polypeptide by the LPMP to a cell, and/or may be capable of increasing loading (e.g., loading efficiency or loading capacity) of a polypeptide. Further exemplary exogenous lipids include sterols and PEGylated lipids.

The LPMPs can be modified with other components (e.g., lipids, e.g., sterols, e.g., cholesterol; or small molecules) to further alter the functional and structural characteristics of the LPMP. For example, the LPMPs can be further modified with stabilizing molecules that increase the stability of the LPMPs (e.g., for at least one day at room temperature, and/or stable for at least one week at 4° C.).

In some embodiments, the LPMP is modified with a sterol, e.g., sitosterol, sitostanol, ß-sitosterol, 7α-hydroxycholesterol, pregnenolone, cholesterol (e.g., ovine cholesterol or cholesterol isolated from plants), stigmasterol, campesterol, fucosterol, or an analog (e.g., a glycoside, ester, or peptide) of any sterol. In some examples, the exogenous sterol is added to the preparation prior to step (b), e.g., mixed with extracted PMP lipids prior to step (b). The exogenous sterol may be added to amount to, e.g., 1%, 5%, 10%, 20%, 30%, 40%, 50%, 60%, 70%, 80%, 90%, or more than 90% (w/w) of total lipids and sterols in the preparation.

In some embodiments, the sterol is cholesterol or sitosterol. In some instances, the LPMPs comprise a molar ratio of least 1%, 5%, 10%, 15%, 20%, 25%, 30%, 35%, 40%, 45%, 50%, 55%, 60%, or more than 60% sterol (e.g., cholesterol or sitosterol), e.g., 1%-10%, 10%-20%, 20%-30%, 30%-40%, 40%-50%, or 50%-60% sterol. In some embodiments, the LPMP comprises a molar ratio of about 35%-50% sterol (e.g., cholesterol or sitosterol), e.g., about 36%, 38.5%, 42.5%, or 46.5% sterol. In some embodiments, the LPMP comprises a molar ratio of about 20%-40% sterol.

In some embodiments, a LPMP that has been modified with a sterol has altered stability (e.g., increased stability) relative to a LPMP that has not been modified with a sterol. In some aspects, a LPMP that has been modified with a sterol has a greater rate of fusion with a membrane of a target cell relative to a LPMP that has not been modified with a sterol.

In some instances, the LPMPs comprise an exogenous lipid and an exogenous sterol.

In some embodiments, the LPMP is modified with a PEGylated lipid. Polyethylene glycol (PEG) length can vary from 1 kDa to 10 kDa; in some aspects, PEG having a length of 2 kDa is used.

In some embodiments, the PEGylated lipid is C14-PEG2k, C18-PEG2k, or DMPE-PEG2k. In some instances, the LPMPs comprise a molar ratio of at least 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.5%, 4%, 4.5%, 5%, 10%, 20%, 30%, 40%, 50%, or more than 50% PEGylated lipid (e.g., C14-PEG2k, C18-PEG2k, or DMPE-PEG2k), e.g., 0.1%-0.5%, 0.5%-1%, 1%-1.5%, 1.5%-2.5%, 2.5%-3.5%, 3.5%-5%, 5%-10%, 10%-20%, 20%-30%, 30%-40%, or 30%-50% PEGylated lipid. In some embodiments, the LPMP comprises a molar ratio of about about 0.1%-10% PEGylated lipid (e.g., C14-PEG2k, C18-PEG2k, or DMPE-PEG2k), e.g., about 1%-3% PEGylated lipid, e.g., about 1.5% or about 2.5% PEGylated lipid. In some embodiments, a LPMP that has been modified with a PEGylated lipid has altered stability (e.g., increased stability) relative to a LPMP that has not been modified with a PEGylated lipid. In some embodiments, a LPMP that has been modified with a PEGylated lipid has altered particle size relative to a LPMP that has not been modified with a PEGylated lipid. In some embodiments, a LPMP that has been modified with a PEGylated lipid is less likely to be phagocytosed than a LPMP that has not been modified with a PEGylated lipid. The addition of PEGylated lipids can also affect stability in GI tract and enhance particle migration through mucus. PEG may be used as a method to attach targeting moieties.

In some embodiments, the LPMPs are modified with an ionizable lipid (e.g., C12-200 or MC3) and one or both of a sterol (e.g., cholesterol or sitosterol) and a PEGylated lipid (e.g., C14-PEG2k, C18-PEG2k, or DMPE-PEG2k).

In some embodiments, the modified LPMPs comprise a molar ratio of about 5%-50% LPMP lipids (e.g., about 10%-20% LPMP lipids, e.g., about 10%, 12.5%, 16%, or 20% LPMP lipids); about 30%-75% ionizable lipids (e.g., about 35% or about 50% ionizable lipids); about 35%-50% sterol (e.g., about 36%, 38.5%, 42.5%, or 46.5% sterol); and about 0.1%-10% PEGylated lipid (e.g., about 1%-3% PEGylated lipid, e.g., about 1.5% or about 2.5% PEGylated lipid).

In some embodiments, the modified LPMPs comprise a molar ratio of about 5%-60% LPMP lipids (e.g., about 10%-20%, 20%-30%, 30%-40%, 40%-50%, or 50%-60% LPMP lipids, e.g., about 10%, 12.5%, 16%, 20%, 30%, 40%, 50%, or 60% LPMP lipids); about 25%-75% ionizable lipids (e.g., about 35% or about 50% ionizable lipids); about 10%-50% sterol (e.g., about 10%, 12.5%, 14%, 16%, 18%, 20%, 36%, 38.5%, 42.5%, or 46.5% sterol); and about 0.1%-10% PEGylated lipid (e.g., about 0.5%-5% PEGylated lipid, e.g., about 1%-3% PEGylated lipid, or about 1.5% or about 2.5% PEGylated lipid).

In some embodiments, the ionizable lipids, LPMP lipids, sterol, and PEGylated lipid comprise about 25%-75%, about 20%-60%, about 10%-45%, and about 0.5%-5%, respectively, of the lipids in the modified PMP.

In some embodiments, the ionizable lipids, LPMP lipids, sterol, and PEGylated lipid comprise about 30%-75%, about 20%-50%, about 10%-45%, and about 1%-5%, respectively, of the lipids in the modified PMP.

In some embodiments, the ionizable lipids, LPMP lipids, sterol, and PEGylated lipid comprise about 35%-75%, about 20%-50%, about 10%-45%, and about 1%-5%, respectively, of the lipids in the modified PMP.

In some embodiments, the ionizable lipids, LPMP lipids, sterol, and PEGylated lipid are formulated at a molar ratio of about 35:50:12.5:2.5.

In some embodiments, the ionizable lipids, LPMP lipids, sterol, and PEGylated lipid are formulated at a molar ratio of about 35:50:11.5:3.5.

In some embodiments, the ionizable lipids, LPMP lipids, sterol, and PEGylated lipid are formulated at a molar ratio of about 35:20:42.5:2.5.

In some embodiments, a LPMP that has been modified with an ionizable lipid (and/or cationic lipid) and a sterol and/or a PEGylated lipid more efficiently encapsulates a negatively charged cargo (e.g., a nucleic acid) than a LPMP that has not been modified with an ionizable lipid (and/or cationic lipid) and a sterol and/or a PEGylated lipid. The modified LPMP may have an encapsulation efficiency for the cargo (e.g., nucleic acid, e.g., RNA or DNA) that is at least 5%, 10%, 15%, 20%, 30%, 40%, 50%, 60%, 70%, 80%, 90%, 95%, 98%, 99%, or more than 99%, e.g., may have an encapsulation efficiency of 5%-30%, 30%-50%, 50%-70%, 70%-80%, 80%-90%, 90%-95%, or 95%-100%.

Cell uptake of the modified LPMPs can be measured by a variety of methods known in the art. For example, the LPMPs, or a component thereof, can be labelled with a marker (e.g., a fluorescent marker) that can be detected in isolated cells to confirm uptake.

In some embodiments, a LPMP formulation provided herein comprises two or more different modified LPMPs, e.g., comprises modified LPMPs derived from different unmodified LPMPs (e.g., unmodified LPMPs from two or more different plant sources) and/or comprises modified LPMPs comprising different species and/or different ratios of ionizable lipids, sterols, and/or PEGylated lipids.

In some instances, the organic solvent in which the lipid film is dissolved is dimethylformamide:methanol (DMF:MeOH). Alternatively, the organic solvent or solvent combination may be, e.g., acetonitrile, acetone, ethanol, methanol, dimethylformamide, tetrahydrofuran, 1-buthanol, dimethyl sulfoxide, acetonitrile:ethanol, acetonitrile:methanol, acetone:methanol, methyl tert-butyl ether:propanol, tetrahydrofuran:methanol, dimethyl sulfoxide:methanol, or dimethylformamide:methanol.

The aqueous phase may be any suitable solution, e.g., a citrate buffer (e.g., a citrate buffer having a pH of about 3.2), water, or phosphate-buffered saline (PBS). The aqueous phase may further comprise a nucleic acid (e.g., an siRNA or siRNA precursor (e.g., dsRNA), miRNA or miRNA precursor, mRNA, or plasmid (pDNA)) or a small molecule.

The lipid solution and the aqueous phase may be mixed in the microfluidics device at any suitable ratio. In some examples, aqueous phase and the lipid solution are mixed at a 3:1 volumetric ratio.

LPMPs may optionally include additional agents, e.g., cell-penetrating agents, therapeutic agents, polynucleotides, polypeptides, or small molecules. The LPMPs can carry or associate with additional agents in a variety of ways to enable delivery of the agent to a target plant, e.g., by encapsulating the agent, incorporation of the agent in the lipid bilayer structure, or association of the agent (e.g., by conjugation) with the surface of the lipid bilayer structure. Nucleic acid molecules can be incorporated into the LPMPs either in vivo (e.g., in planta) or in vitro (e.g., in tissue culture, in cell culture, or synthetically incorporated).

### Zeta Potential

The LPMPs comprising an ionizable lipid (e.g., C12-200 or MC3), and optionally a cationic lipid (e.g., DC-cholesterol or DOTAP) may have, e.g., a zeta potential of greater than −30 mV when in the absence of cargo, greater than −20 mV, greater than −5 mV, greater than 0 mV, or about 30 my when in the absence of cargo. In some examples, the LPMP has a negative zeta potential, e.g., a zeta potential of less than 0 mV, less than −10 mV, less than −20 mV, less than −30 mV, less than −40 mV, or less than −50 mV when in the absence of cargo. In some examples, the LPMP has a positive zeta potential, e.g., a zeta potential of greater than 0 mV, greater than 10 mV, greater than 20 mV, greater than 30 mV, greater than 40 mV, or greater than 50 mV when in the absence of cargo. In some examples, the LPMP has a zeta potential of about 0.

The zeta potential of the LPMP may be measured using any method known in the art. Zeta potentials are generally measured indirectly, e.g., calculated using theoretical models from the data obtained using methods and techniques known in the art, e.g., electrophoretic mobility or dynamic electrophoretic mobility. Electrophoretic mobility is typically measured using microelectrophoresis, electrophoretic light scattering, or tunable resistive pulse sensing. Electrophoretic light scattering is based on dynamic light scattering. Typically, zeta potentials are accessible from dynamic light scattering (DLS) measurements, also known as photon correlation spectroscopy or quasi-elastic light scattering.

### Plant EV-Markers

The LPMPs in the mRNA therapeutic composition and methods of making and using thereof may have a range of markers that identify the LPMPs as being produced using a plant EV, and/or including a segment, portion, or extract thereof. As used herein, the term “plant EV-marker” refers to a component that is naturally associated with a plant and incorporated into or onto the plant EV in planta, such as a plant protein, a plant nucleic acid, a plant small molecule, a plant lipid, or a combination thereof. Examples of plant EV-markers can be found, for example, in Rutter and Innes, Plant Physiol. 173(1): 728-741, 2017; Raimondo et al., Oncotarget. 6(23): 19514, 2015; Ju et al., Mol. Therapy. 21(7):1345-1357, 2013; Wang et al., Molecular Therapy. 22(3): 522-534, 2014; and Regente et al, J of Exp. Biol. 68(20): 5485-5496, 2017; each of which is incorporated herein by reference.

Additional examples of the suitable plant EV-markers include those described and listed in International Patent Application Publication No. WO 2021/041301, which is incorporated herein by reference in its entirety.

### Loading of Agents (e.g., Nucleic Acids)

The LPMPs are modified to include a therapeutic agent (e.g., a nucleic acid molecule) to form the mRNA therapeutic composition. The LPMPs can carry or associate with such agents by a variety of means to enable delivery of the agent to a target organism (e.g., a target animal), e.g., by encapsulating the agent, incorporation of the component in the lipid bilayer structure, or association of the component (e.g., by conjugation) with the surface of the lipid bilayer structure of the LPMP. In some instances, the agent is included in the LPMP formulation, as described herein.

The agent can be incorporated or loaded into or onto the LPMPs by any methods known in the art that allow association, directly or indirectly, between the LPMPs and agent. The agents can be incorporated into the LPMPs by an in vivo method (e.g., in planta, e.g., through production of LPMPs from a transgenic plant that comprises the agent), or in vitro (e.g., in tissue culture, or in cell culture), or both in vivo and in vitro methods.

In some instances, the LPMPs are loaded in vitro. The substance may be loaded onto or into (e.g., may be encapsulated by) the LPMPs using, but not limited to, physical, chemical, and/or biological methods (e.g., in tissue culture or in cell culture). For example, the agent may be introduced into LPMPs by one or more of electroporation, sonication, passive diffusion, stirring, lipid extraction, or extrusion. In some instances, the agent is incorporated into the LPMP using a microfluidic device, e.g., using a method in which LPMP lipids are provided in an organic phase, the heterologous functional agent is provided in an aqueous phase, and the organic and aqueous phases are combined in the microfluidics device to produce a LPMP comprising the heterologous functional agent. Loaded LPMPs can be assessed to confirm the presence or level of the loaded agent using a variety of methods, such as HPLC (e.g., to assess small molecules), immunoblotting (e.g., to assess proteins); and/or quantitative PCR (e.g., to assess nucleotides). However, it should be appreciated by those skilled in the art that the loading of a substance of interest into LPMPs is not limited to the above-illustrated methods.

In some instances, the agent can be conjugated to the LPMP, in which the agent is connected or joined, indirectly or directly, to the LPMP. For instance, one or more agents can be chemically linked to a LPMP, such that the one or more agents are joined (e.g., by covalent or ionic bonds) directly to the lipid bilayer of the LPMP. In some instances, the conjugation of various agents to the LPMPs can be achieved by first mixing the one or more agents with an appropriate cross-linking agent (e.g., N-ethylcarbo-diimide (“EDC”), which is generally utilized as a carboxyl activating agent for amide bonding with primary amines and also reacts with phosphate groups) in a suitable solvent. After a period of incubation sufficient to allow the agent to attach to the cross-linking agent, the cross-linking agent/agent mixture can then be combined with the LPMPs and, after another period of incubation, subjected to a sucrose gradient (e.g., and 8, 30, 45, and 60% sucrose gradient) to separate the free agent and free LPMPs from the agent conjugated to the LPMPs. As part of combining the mixture with a sucrose gradient, and an accompanying centrifugation step, the LPMPs conjugated to the agent are then seen as a band in the sucrose gradient, such that the conjugated LPMPs can then be collected, washed, and dissolved in a suitable solution for use as described herein.

In some instances, the LPMPs are stably associated with the agent prior to and following delivery of the LPMP, e.g., to a plant. In other instances, the LPMPs are associated with the agent such that the agent becomes dissociated from the LPMPs following delivery of the LPMP, e.g., to a plant.

The LPMPs can be loaded or the LPMP can be formulated with various concentrations of the agent, depending on the particular agent or use. For example, in some instances, the LPMPs are loaded or the LPMP is formulated such that the LPMP formulation disclosed herein includes about 0.001, 0.01, 0.1, 1.0, 2, 3, 4, 5, 6, 7, 8, 9, 10, 15, 20, 30, 40, 50, 60, 70, 80, 90, or 95 (or any range between about 0.001 and 95) or more wt % of an agent. In some instances, the LPMPs are loaded or the LPMP is formulated such that the LPMP formulation includes about 95, 90, 80, 70, 60, 50, 40, 30, 20, 15, 10, 9, 8, 7, 6, 5, 4, 3, 2, 1.0, 0.1, 0.01, 0.001 (or any range between about 95 and 0.001) or less wt % of an agent. For example, the LPMP formulation can include about 0.001 to about 0.01 wt %, about 0.01 to about 0.1 wt %, about 0.1 to about 1 wt %, about 1 to about 5 wt %, or about 5 to about 10 wt %, about 10 to about 20 wt % of the agent. In some instances, the LPMP can be loaded or the LPMP is formulated with about 1, 5, 10, 50, 100, 200, or 500, 1,000, 2,000 (or any range between about 1 and 2,000) or more μg/ml of an agent. A LPMP of the invention can be loaded or a LPMP can be formulated with about 2,000, 1,000, 500, 200, 100, 50, 10, 5, 1 (or any range between about 2,000 and 1) or less μg/ml of an agent.

In some instances, the LPMPs are loaded or the LPMP is formulated such that the LPMP formulation disclosed herein includes at least 0.001 wt %, at least 0.01 wt %, at least 0.1 wt %, at least 1.0 wt %, at least 2 wt %, at least 3 wt %, at least 4 wt %, at least 5 wt %, at least 6 wt %, at least 7 wt %, at least 8 wt %, at least 9 wt %, at least 10 wt %, at least 15 wt %, at least 20 wt %, at least 30 wt %, at least 40 wt %, at least 50 wt %, at least 60 wt %, at least 70 wt %, at least 80 wt %, at least 90 wt %, or at least 95 wt % of an agent. In some instances, the LPMP can be loaded or the LPMP can be formulated with at least 1 μg/ml, at least 5 μg/ml, at least 10 μg/ml, at least 50 μg/ml, at least 100 μg/ml, at least 200 μg/ml, at least 500 μg/ml, at least 1,000 μg/ml, at least 2,000 μg/ml of an agent.

In some instances, the LPMP is formulated with the agent by suspending the LPMPs in a solution comprising or consisting of the agent, e.g., suspending or resuspending the LPMPs by vigorous mixing. The agent (e.g., cell-penetrating agent, e.g., nucleic acids, enzyme, detergent, ionic, fluorous, or zwitterionic liquid, or ionizable lipid may comprise, e.g., less than 1% or at least 1%, 5%, 10%, 20%, 30%, 40%, 50%, 60%, 70%, 80%, 90%, or 100% of the solution.

### Pharmaceutical Formulations

The modified LPMPs are formulated into pharmaceutical compositions (i.e., a mRNA therapeutic composition), e.g., for administration to an animal (e.g., a human). The pharmaceutical composition may be administered to an animal (e.g., human) with a pharmaceutically acceptable diluent, carrier, and/or excipient. Depending on the mode of administration and the dosage, the pharmaceutical composition of the methods described herein will be formulated into suitable pharmaceutical compositions to permit facile delivery. The single dose may be in a unit dose form as needed.

The LPMP/mRNA therapeutic composition may be formulated for e.g., oral administration, intravenous administration (e.g., injection or infusion), intramuscular, or subcutaneous administration to an animal. For injectable formulations, various effective pharmaceutical carriers are known in the art (See, e.g., Remington: The Science and Practice of Pharmacy, 22^{nd} ed., (2012) and ASHP Handbook on Injectable Drugs, 18^{th} ed., (2014)).

Suitable pharmaceutically acceptable carriers and excipients are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol. The LPMP/mRNA therapeutic composition may be formulated according to conventional pharmaceutical practice. The concentration of the compound in the formulation will vary depending upon a number of factors, including the dosage of the active agent (e.g., LPMPs and nucleic acids) to be administered, and the route of administration.

For oral administration to an animal, the LPMP/mRNA therapeutic composition can be prepared in the form of an oral formulation. Formulations for oral use can include tablets, caplets, capsules, syrups, or oral liquid dosage forms containing the active ingredient(s) in a mixture with nontoxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like. Formulations for oral use may also be provided in unit dosage form as chewable tablets, non-chewable tablets, caplets, capsules (e.g., as hard gelatin capsules wherein the active ingredient is mixed with an inert solid diluent, or as soft gelatin capsules wherein the active ingredient is mixed with water or an oil medium). The compositions disclosed herein may also further include an immediate-release, extended release or delayed-release formulation.

For parenteral administration to an animal, the LPMP/mRNA therapeutic compositions may be formulated in the form of liquid solutions or suspensions and administered by a parenteral route (e.g., subcutaneous, intravenous, or intramuscular). The pharmaceutical composition can be formulated for injection or infusion. Pharmaceutical compositions for parenteral administration can be formulated using a sterile solution or any pharmaceutically acceptable liquid as a vehicle. Pharmaceutically acceptable vehicles include, but are not limited to, sterile water, physiological saline, or cell culture media (e.g., Dulbecco's Modified Eagle Medium (DMEM), α-Modified Eagles Medium (α-MEM), and F-12 medium). Formulation methods are known in the art, see e.g., Gibson (ed.) Pharmaceutical Preformulation and Formulation (2nd ed.) Taylor & Francis Group, CRC Press (2009).

### Polynucleotides

The LPMP/mRNA therapeutic composition includes one or more nucleic acid molecules, e.g., polynucleotides, which encode one or more wild type or engineered proteins, peptides, or polypeptides. Exemplary polynucleotides, e.g., polynucleotide constructs, include antigen-encoding RNA polynucleotides, e.g., mRNAs.

Examples of polypeptides that can be used herein can include an enzyme (e.g., a metabolic recombinase, a helicase, an integrase, a RNAse, a DNAse, or an ubiquitination protein), a pore-forming protein, a signaling ligand, a cell penetrating peptide, a transcription factor, a receptor, an antibody, a nanobody, a gene editing protein (e.g., CRISPR-Cas system, TALEN, or zinc finger), riboprotein, a protein aptamer, or a chaperone.

Polypeptides included herein may include naturally occurring polypeptides or recombinantly produced variants. In some instances, the polypeptide may be a functional fragments or variants thereof (e.g., an enzymatically active fragment or variant thereof). For example, the polypeptide may be a functionally active variant of any of the polypeptides described herein with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a polypeptide described herein or a naturally occurring polypeptide. In some instances, the polypeptide may have at least 50% (e.g., at least 50%, 60%, 70%, 80%, 90%, 95%, 97%, 99%, or greater) identity to a protein of interest.

The LPMP/mRNA therapeutic composition may include any number or type (e.g., classes) of polypeptides, such as at least about any one of 1 polypeptide, 2, 3, 4, 5, 10, 15, 20, or more polypeptides. A suitable concentration of each polypeptide in the LPMP/mRNA therapeutic composition depends on factors such as efficacy, stability of the polypeptide, number of distinct polypeptides in the formulation, and methods of application of the formulation. In some instances, each polypeptide in a liquid formulation is from about 0.1 ng/mL to about 100 mg/mL. In some instances, each polypeptide in a solid formulation is from about 0.1 ng/g to about 100 mg/g.

### Nucleic Acids Encoding Peptides

In some instances, the LPMP/mRNA therapeutic composition include a heterologous nucleic acid encoding a polypeptide. Nucleic acids encoding a polypeptide may have a length from about 10 to about 50,000 nucleotides (nts), about 25 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, about 5000 to about 6000 nts, about 6000 to about 7000 nts, about 7000 to about 8000 nts, about 8000 to about 9000 nts, about 9000 to about 10,000 nts, about 10,000 to about 15,000 nts, about 10,000 to about 20,000 nts, about 10,000 to about 25,000 nts, about 10,000 to about 30,000 nts, about 10,000 to about 40,000 nts, about 10,000 to about 45,000 nts, about 10,000 to about 50,000 nts, or any range therebetween.

The LPMP/mRNA therapeutic composition may also include active variants of a nucleic acid sequence of interest. In some instances, the variant of the nucleic acids has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a nucleic acid of interest. In some instances, the invention includes an active polypeptide encoded by a nucleic acid variant as described herein. In some instances, the active polypeptide encoded by the nucleic acid variant has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire amino acid sequence, to a sequence of a polypeptide of interest or the naturally derived polypeptide sequence.

Certain methods for expressing a nucleic acid encoding a protein may involve expression in cells, including insect, yeast, plant, bacteria, or other cells under the control of appropriate promoters. Expression vectors may include nontranscribed elements, such as an origin of replication, a suitable promoter and enhancer, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, enhancer, splice, and polyadenylation sites may be used to provide the other genetic elements required for expression of a heterologous DNA sequence. Appropriate cloning and expression vectors for use with bacterial, fungal, yeast, and mammalian cellular hosts are described in Green et al., Molecular Cloning: A Laboratory Manual, Fourth Edition, Cold Spring Harbor Laboratory Press, 2012.

Genetic modification using recombinant methods is generally known in the art. A nucleic acid sequence coding for a desired gene can be obtained using recombinant methods known in the art, such as, for example by screening libraries from cells expressing the gene, by deriving the gene from a vector known to include the same, or by isolating directly from cells and tissues containing the same, using standard techniques. Alternatively, a gene of interest can be produced synthetically, rather than cloned.

Expression of natural or synthetic nucleic acids is typically achieved by operably linking a nucleic acid encoding the gene of interest to a promoter, and incorporating the construct into an expression vector. Expression vectors can be suitable for replication and expression in bacteria. Expression vectors can also be suitable for replication and integration in eukaryotes. Typical cloning vectors contain transcription and translation terminators, initiation sequences, and promoters useful for expression of the desired nucleic acid sequence.

Additional promoter elements, e.g., enhancers, regulate the frequency of transcriptional initiation. Typically, these are located in the region 30-110 basepairs (bp) upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Alternatively, the promoter may be an inducible promoter. The use of an inducible promoter provides a molecular switch capable of turning on expression of the polynucleotide sequence to which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes may be used for identifying potentially transformed cells and for evaluating the functionality of regulatory sequences. In general, a reporter gene is a gene that is not present in or expressed by the recipient source and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., FEBS Letters 479:79-82, 2000). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some instances, an organism may be genetically modified to alter expression of one or more proteins. Expression of the one or more proteins may be modified for a specific time, e.g., development or differentiation state of the organism. In one instance, provided is a composition to alter expression of one or more proteins, e.g., proteins that affect activity, structure, or function. Expression of the one or more proteins may be restricted to a specific location(s) or widespread throughout the organism.

mRNA

The LPMP/mRNA therapeutic composition may include a mRNA molecule, e.g., a mRNA molecule encoding a polypeptide. The mRNA molecule can be synthetic and modified (e.g., chemically). The mRNA molecule can be chemically synthesized or transcribed in vitro. The mRNA molecule can be disposed on a plasmid, e.g., a viral vector, bacterial vector, or eukaryotic expression vector. In some examples, the mRNA molecule can be delivered to cells by transfection, electroporation, or transduction (e.g., adenoviral or lentiviral transduction).

In some instances, the modified RNA agent of interest described herein has modified nucleosides or nucleotides. Such modifications are known and are described, e.g., in WO 2012/019168. Additional modifications are described, e.g., in WO 2015/038892; WO 2015/038892; WO 2015/089511; WO 2015/196130; WO 2015/196118 and WO 2015/196128 A2, which are herein incorporated by reference in their entirety.

In some instances, the modified RNA encoding a polypeptide of interest has one or more terminal modification, e.g., a 5′ cap structure and/or a poly-A tail (e.g., of between 100-200 nucleotides in length). The 5′ cap structure may be selected from the group consisting of CapO, CapI, ARCA, inosine, NI-methyl-guanosine, 2′fluoro-guanosine, 7-deaza-guanosine, 8-oxo-guanosine, 2-amino-guanosine, LNA-guanosine, and 2-azido-guanosine. In some cases, the modified RNAs also contain a 5′ UTR including at least one Kozak sequence, and a 3′ UTR. Such modifications are known and are described, e.g., in WO 2012/135805 and WO 2013/052523, which are incorporated herein by reference in their entirety. Additional terminal modifications are described, e.g., in WO 2014/164253 and WO 2016/011306, WO 2012/045075, and WO 2014/093924, which are incorporated herein by reference in their entirety. Chimeric enzymes for synthesizing capped RNA molecules (e.g., modified mRNA) which may include at least one chemical modification are described in WO 2014/028429, which is incorporated herein by reference in its entirety.

In some instances, a modified mRNA (mmRNA) may be cyclized, or concatemerized, to generate a translation competent molecule to assist interactions between poly-A binding proteins and 5′-end binding proteins. The mechanism of cyclization or concatemerization may occur through at least 3 different routes: 1) chemical, 2) enzymatic, and 3) ribozyme catalyzed. The newly formed 5′-/3′-linkage may be intramolecular or intermolecular. Such modifications are described, e.g., in WO 2013/151736.

Methods of making and purifying modified RNAs are known and disclosed in the art. For example, modified RNAs are made using only in vitro transcription (IVT) enzymatic synthesis. Methods of making IVT polynucleotides are known in the art and are described in WO 2013/151666, WO 2013/151668, WO 2013/151663, WO 2013/151669, WO 2013/151670, WO 2013/151664, WO 2013/151665, WO 2013/151671, WO 2013/151672, WO 2013/151667 and WO 2013/151736, which are incorporated herein by reference in their entirety. Methods of purification include purifying an RNA transcript including a polyA tail by contacting the sample with a surface linked to a plurality of thymidines or derivatives thereof and/or a plurality of uracils or derivatives thereof (polyT/U) under conditions such that the RNA transcript binds to the surface and eluting the purified RNA transcript from the surface (WO 2014/152031); using ion (e.g., anion) exchange chromatography that allows for separation of longer RNAs up to 10,000 nucleotides in length via a scalable method (WO 2014/144767); and subjecting a modified mRNA sample to DNAse treatment (WO 2014/152030).

Formulations of modified RNAs are known and are described, e.g., in WO 2013/090648. For example, the formulation may be, but is not limited to, nanoparticles, poly(lactic-co-glycolic acid)(PLGA) microspheres, lipidoids, lipoplex, liposome, polymers, carbohydrates (including simple sugars), cationic lipids, fibrin gel, fibrin hydrogel, fibrin glue, fibrin sealant, fibrinogen, thrombin, rapidly eliminated lipid nanoparticles (reLNPs) and combinations thereof.

Modified RNAs encoding polypeptides in the fields of human disease, antibodies, viruses, and a variety of in vivo settings are known and are disclosed in for example, Table 6 of International Publication Nos. WO 2013/151666, WO 2013/151668, WO 2013/151663, WO 2013/151669, WO 2013/151670, WO 2013/151664, WO 2013/151665, WO 2013/151736; Tables 6 and 7 International Publication No. WO 2013/151672; Tables 6, 178 and 179 of International Publication No. WO 2013/151671; Tables 6, 185 and 186 of International Publication No WO 2013/151667; which are incorporated herein by reference in their entirety. Any of the foregoing may be synthesized as an IVT polynucleotide, chimeric polynucleotide or a circular polynucleotide, and each may include one or more modified nucleotides or terminal modifications.

### Inhibitory RNA

In some instances, the LPMP/mRNA therapeutic composition includes an inhibitory RNA molecule, e.g., that acts via the RNA interference (RNAi) pathway. In some instances, the inhibitory RNA molecule decreases the level of gene expression in a plant and/or decreases the level of a protein in the plant. In some instances, the inhibitory RNA molecule inhibits expression of a plant gene. For example, an inhibitory RNA molecule may include a short interfering RNA or its precursor, short hairpin RNA, and/or a microRNA or its precursor that targets a gene in the plant. Certain RNA molecules can inhibit gene expression through the biological process of RNA interference (RNAi). RNAi molecules include RNA or RNA-like structures typically containing 15-50 base pairs (such as about 18-25 base pairs) and having a nucleobase sequence identical (or complementary) or nearly identical (or substantially complementary) to a coding sequence in an expressed target gene within the cell. RNAi molecules include, but are not limited to short interfering RNAs (siRNAs), double-strand RNAs (dsRNA), short hairpin RNAs (shRNA), meroduplexes, dicer substrates, and multivalent RNA interference (U.S. Pat. Nos. 8,084,599 8,349,809, 8,513,207 and 9,200,276, which are incorporated herein by reference in their entirety). The inhibitory RNA molecule can be chemically synthesized or transcribed in vitro.

Additional examples of the inhibitory RNA molecules include those described in details in International Patent Application Publication No. WO 2021/041301, which is incorporated herein by reference in its entirety.

### Gene Editing

In some instances, the LPMP/mRNA therapeutic compositions may include a component of a gene editing system. For example, the agent may introduce an alteration (e.g., insertion, deletion (e.g., knockout), translocation, inversion, single point mutation, or other mutation) in a gene in the plant. Exemplary gene editing systems include the zinc finger nucleases (ZFNs), Transcription Activator-Like Effector-based Nucleases (TALEN), and the clustered regulatory interspaced short palindromic repeat (CRISPR) system. ZFNs, TALENs, and CRISPR-based methods are described, e.g., in Gaj et al., Trends Biotechnol. 31(7):397-405, 2013.

Additional descriptions about the component and process of the gene editing system may be found in International Patent Application Publication No. WO 2021/041301, which is incorporated herein by reference in its entirety.

mRNA Therapeutics

The LPMP/mRNA therapeutic composition comprises one or more polynucleotides (e.g., mRNA) encoding one or more antigenic or signaling polypeptides for therapeutic purpose, such as to combat cancer. The one or more polynucleotides (e.g., mRNA) encode one or more antigenic (e.g., tumor antigenic) or signaling polypeptides.

### Tumor Antigens and Signaling Therapeutics

Cancers or tumors include but are not limited to neoplasms, malignant tumors, metastases, or any disease or disorder characterized by uncontrolled cell growth such that it would be considered cancerous. The cancer may be a primary or metastatic cancer. Specific cancers that can be treated according to the present invention include, but are not limited to, those listed below (for a review of such disorders, see Fishman et al, 1985, Medicine, 2d Ed., J.B. Lippincott Co., Philadelphia). Cancers include, but are not limited to, biliary tract cancer; bladder cancer; brain cancer including glioblastomas and medulloblastomas; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma, teratomas, choriocarcinomas; stromal tumors and germ cell tumors; thyroid cancer including thyroid adenocarcinoma and medullar carcinoma; and renal cancer including adenocarcinoma and Wilms' tumor. Commonly encountered cancers include breast, prostate, lung, ovarian, colorectal, and brain cancer.

In some embodiments, the cancer is selected from the group consisting of non-small cell lung cancer (NSCLC), small cell lung cancer, melanoma, bladder urothelial carcinoma, HPV-negative head and neck squamous cell carcinoma (HNSCC), and a solid malignancy that is microsatellite high (MSI H)/mismatch repair (MMR) deficient. In some embodiments, the NSCLC lacks an EGFR sensitizing mutation and/or an ALK translocation. In some embodiments, the solid malignancy that is microsatellite high (MSI H)/mismatch repair (MMR) deficient is selected from the group consisting of colorectal cancer, stomach adenocarcinoma, esophageal adenocarcinoma, and endometrial cancer.

In some embodiments, the cancer is selected from cancer of the pancreas, peritoneum, large intestine, small intestine, biliary tract, lung, endometrium, ovary, genital tract, gastrointestinal tract, cervix, stomach, urinary tract, colon, rectum, and hematopoietic and lymphoid tissues. In some embodiments, the cancer is colorectal cancer.

In some embodiments, the antigenic (e.g., tumor antigenic) or signaling polypeptide comprises p53, ART-4, BAGE, ss-catenin/m, Bcr-abL CAMEL, CAP-1, CASP-8, CDC27/m, CDK4/m, CEA, CLAUDIN-12, c-MYC, CT, Cyp-B, DAM, ELF2M, ETV6-AML1, G250, GAGE, GnT-V, Gap 100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A10, MAGE-A11, or MAGE-A12, MAGE-B, MAGE-C, MART-1/Melan-A, MC1R, Myosin/m, MUC1, MUM-1, -2, -3, NA88-A, NF1, NY-ESO-1, NY-BR-1, pI90 minor BCR-abL, Plac-1, Pml/RARa, PRAME, proteinase 3, PSA, PSM, RAGE, RU1 or RU2, SAGE, SART-1 or S ART-3, SCGB3A2, SCP1, SCP2, SCP3, SSX, SURVIVIN, TEL/AML1, TPI/m, TRP-1, TRP-2, TRP-2/INT2, TPTE, WT, WT-1, or a combination thereof.

In some embodiments, the tumor antigen is one of the following antigens: CD2, CD3, CD4, CD8, CD11b, CD14, CD16, CD19, CD20, CD22, CD25, CD27, CD33, CD37, CD38, CD40, CD44, CD45, CD47, CD52, CD56, CD70, CD79, CD137, 4-IBB, 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbBI, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gplOO, gpA33, GPNMB, HLA, HLA-DR, ICOS, IGF1R, Integrin av, Integrin αvβ, LAG-3, Lewis Y, Mesothelin, c-MET, MN Carbonic anhydrase IX, MUC1, MUC16, Nectin-4, KGD2, NOTCH, OX40, OX40L, PD-1, PDL1, PSCA, PSMA, RANKL, ROR1, ROR2, SLC44A4, Syndecan-1, TACI, TAG-72, Tenascin, TIM3, TRAILR1, TRAILR2, VEGFR-1, VEGFR-2, VEGFR-3, and variants thereof.

In some embodiments, the antigenic, tumor antigenic, or signaling polypeptide is IL-2 peptide, IL-2-Ra, tdTomato, Cre recombinase, GFP, eGFP, Anti-CD19, CD20, CAR-T, Anti-HER2, Etanercept (Enbrel), Humira, erythropoietin, Epogen, Filgrastim, Keytruda, Rituximab, Romiplostim, Sargramostim, or a fragment or subunit thereof. In one embodiment, the polypeptide is IL-2 peptide, or a fragment or subunit thereof. In one embodiment, the polypeptide is erythropoietin or Epogen, or a fragment or subunit thereof.

In some embodiments, the polypeptide encoded by the polynucleotide is a antigenic (e.g., tumor antigenic) or signaling polypeptide comprising IL-1 α, IL-1 β, IL-1ra, IL-2, IL-3, IL-4, IL-5, IL-6, IL-7, IL-8, IL-9, IL-10, IL-11, IL-12, IL-13, IL-14, IL-15, IL-16, IL-17A, IL-17B, IL-17C, IL-17D, IL-17E, IL-17F, IL-18, IL-19, IL-20, IL-21, IL-22, IL-23, IL-24, IL-25, IL-26, IL-27, IL-28A/B, IL-29, IL- 30, IL-31, IL-32, IL-33, IL-35, TGF-β, GM-CSF, M-CSF, G-CSF, TNF-α, TNF-β, LAF, TCGF, BCGF, TRF, BAF, BDG, MP, LIF, OSM, TMF, PDGF, IFN-α, IFN-β, IFN-γ, Uteroglobins, Foxp3, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CCL1, CCL2, CCL3, CCL4, CCL5, CCL6, CCL7, CCL8, CCL9/10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, XCL1, XCL2, CX3CL1 or a fragment, subunit, or variant thereof.

In some embodiments, the polypeptide encoded by the polynucleotide is IL-15 peptide, IL-15-R^{a}, or a fragment or subunit thereof. In one embodiment, the polypeptide is IL-15 peptide, or a fragment or subunit thereof.

In some embodiments, the antigenic, tumor antigenic, or signaling polypeptide comprises a tumor antigen selected from the group consisting of a carcinoma, a sarcoma, a melanoma, a lymphoma, a leukemia, and a combination thereof.

In one embodiment, the tumor antigenic polypeptide comprises a melanoma tumor antigen. In one embodiment, the tumor antigenic polypeptide comprises a prostate cancer antigen. In one embodiment, the tumor antigenic polypeptide comprises a HPV16 positive head and neck cancer antigen. In one embodiment, the tumor antigenic polypeptide comprises a breast cancer antigen. In one embodiment, the tumor antigenic polypeptide comprises an ovarian cancer antigen. In one embodiment, the tumor antigenic polypeptide comprises a lung cancer antigen. In one embodiment, the tumor antigenic polypeptide comprises an NSCLC antigen.

In some embodiments, the antigen is a self-antigenic polypeptide or an immunogenic variant or an immunogenic fragment thereof. In some embodiments, a self-antigenic polypeptide comprises an antigen that is typically expressed on cells and is recognized as a self-antigen by an immune system. In some embodiments, the self-antigenic polypeptide comprises: a multiple sclerosis antigenic polypeptide, a Rheumatoid arthritis antigenic polypeptide, a lupus antigenic polypeptide, a celiac disease antigenic polypeptide, a Sjogren's syndrome antigenic polypeptide, or an ankylosing spondylitis antigenic polypeptide, or a combination thereof.

Immune Potentiator mRNAs

In some embodiments, the one or more polynucleotides comprise mRNAs that encode a polypeptide that stimulates or enhances an immune response against one or more cancer antigens of interest. Such mRNAs that enhance immune responses to the cancer antigen(s) of interest are referred to herein as immune potentiator mRNA constructs or immune potentiator mRNAs, including chemically modified mRNAs (mmRNAs). An immune potentiator enhances an immune response to an antigen of interest in a subject. The enhanced immune response can be a cellular response, a humoral response or both. As used herein, a “cellular” immune response is intended to encompass immune responses that involve or are mediated by T cells, whereas a “humoral” immune response is intended to encompass immune responses that involve or are mediated by B cells. An immune potentiator may enhance an immune response by, for example,
(i) stimulating Type I interferon pathway signaling;
(ii) stimulating NFkB pathway signaling;
(iii) stimulating an inflammatory response;
(iv) stimulating cytokine production; or
(v) stimulating dendritic cell development, activity or mobilization; and
(vi) a combination of any of (i)-(vi).

As used herein, “stimulating Type I interferon pathway signaling” is intended to encompass activating one or more components of the Type I interferon signaling pathway (e.g., modifying phosphorylation, dimerization or the like of such components to thereby activate the pathway), stimulating transcription from an interferon-sensitive response element (ISRE) and/or stimulating production or secretion of Type I interferon (e.g., IFN-a, IFN-β, IFN-ε, IFN-K and/or IFN-co). As used herein, “stimulating NFkB pathway signaling” is intended to encompass activating one or more components of the NFkB signaling pathway (e.g., modifying phosphorylation, dimerization or the like of such components to thereby activate the pathway), stimulating transcription from an NFkB site and/or stimulating production of a gene product whose expression is regulated by NFkB. As used herein, “stimulating an inflammatory response” is intended to encompass stimulating the production of inflammatory cytokines (including but not limited to Type I interferons, IL-6 and/or TNFa). As used herein, “stimulating dendritic cell development, activity or mobilization” is intended to encompass directly or indirectly stimulating dendritic cell maturation, proliferation and/or functional activity.

In some embodiments, the mRNA encodes a polypeptide that stimulates or enhances an immune response in a subject in need thereof (e.g., potentiates an immune response in the subject) by, for example, inducing adaptive immunity (e.g., by stimulating Type I interferon production), stimulating an inflammatory response, stimulating FkB signaling and/or stimulating dendritic cell (DC) development, activity or mobilization in the subject. In some embodiments, administration of an immune potentiator mRNA to a subject in need thereof enhances cellular immunity (e.g., T cell-mediated immunity), humoral immunity (e.g., B cell-mediated immunity) or both cellular and humoral immunity in the subject. In some embodiments, administration of an immune potentiator mRNA stimulates cytokine production (e.g., inflammatory cytokine production), stimulates cancer antigen-specific CD8⁺ effector cell responses, stimulates antigen-specific CD4⁺ helper cell responses, increases the effector memory CD62L¹⁰ T cell population, stimulates B cell activity or stimulates antigen-specific antibody production, including combinations of the foregoing responses. In some embodiments, administration of an immune potentiator mRNA stimulates cytokine production (e.g., inflammatory cytokine production) and stimulates antigen-specific CD8⁺ effector cell responses. In some embodiments, administration of an immune potentiator mRNA stimulates cytokine production (e.g., inflammatory cytokine production), and stimulates antigen-specific CD4⁺ helper cell responses. In some embodiments, administration of an immune potentiator mRNA stimulates cytokine production (e.g., inflammatory cytokine production), and increases the effector memory CD62L¹⁰ T cell population. In some embodiments, administration of an immune potentiator mRNA stimulates cytokine production (e.g., inflammatory cytokine production), and stimulates B cell activity or stimulates antigen-specific antibody production.

In one embodiment, an immune potentiator increases cancer antigen-specific CD8⁺ effector cell responses (cellular immunity). For example, an immune potentiator can increase one or more indicators of antigen-specific CD8⁺ effector cell activity, including but not limited to CD8+ T cell proliferation and CD8+ T cell cytokine production. For example, in one embodiment, an immune potentiator increases production of IFN-γ, TNFα and/or IL-2 by antigen-specific CD8+ T cells. In various embodiments, an immune potentiator can increase CD8+ T cell cytokine production (e.g., IFN-γ, TNFα and/or IL-2 production) in response to an antigen (as compared to CD8+ T cell cytokine production in the absence of the immune potentiator) by at least 5% or at least 10% or at least 15% or at least 20% or at least 25% or at least 30%) or at least 35% or at least 40% or at least 45% or at least 50%. For example, T cells obtained from a treated subject can be stimulated in vitro with the cancer antigens and CD8+ T cell cytokine production can be assessed in vitro. CD8+ T cell cytokine production can be determined by standard methods known in the art, including but not limited to measurement of secreted levels of cytokine production (e.g., by ELISA or other suitable method known in the art for determining the amount of a cytokine in supernatant) and/or determination of the percentage of CD8+ T cells that are positive for intracellular staining (ICS) for the cytokine. For example, intracellular staining (ICS) of CD8+ T cells for expression of IFN-γ, TNFa and/or JL-2 can be carried out by methods known in the art. In one embodiment, an immune potentiator increases the percentage of CD8+ T cells that are positive by ICS for one or more cytokines (e.g., IFN-γ, TNFα and/or IL-2) in response to an antigen (as compared to the percentage of CD8+ T cells that are positive by ICS for the cytokine(s) in the absence of the immune potentiator) by at least 5% or at least 10% or at least 15%> or at least 20% or at least 25% or at least 30%> or at least 35% or at least 40% or at least 45% or at least 50%.

In one embodiment, an immune potentiator increases the percentage of CD8+ T cells among the total T cell population (e.g., splenic T cells and/or PBMCs), as compared to the percentage of CD8+ T cells in the absence of the immune potentiator. For example, an immune potentiator can increase the percentage of CD8+ T cells among the total T cell population by at least 5% or at least 10% or at least 15% or at least 20% or at least 25% or at least 30%) or at least 35% or at least 40% or at least 45% or at least 50%, as compared to the percentage of CD8+ T cells in the absence of the immune potentiator. The total percentage of CD8+ T cells among the total T cell population can be determined by standard methods known in the art, including but not limited to fluorescent activated cell sorting (FACS) or magnetic activated cell sorting (MACS).

In one embodiment, an immune potentiator increases a tumor-specific immune cell response, as determined by a decrease in tumor volume in vivo in the presence of the immune potentiator as compared to tumor volume in the absence of the immune potentiator. For example, an immune potentiator can decrease tumor volume by at least 5% or at least 10% or at least 15% or at least 20% or at least 25% or at least 30% or at least 35% or at least 40% or at least 45% or at least 50%, as compared to tumor volume in the absence of the immune potentiator. Measurement of tumor volume can be determined by methods well established in the art. In another embodiment, an immune potentiator increases B cell activity (humoral immune response), for example by increasing the amount of antigen-specific antibody production, as compared to antigen-specific antibody production in the absence of the immune potentiator. For example, an immune potentiator can increase antigen-specific antibody production by at least 5% or at least 10% or at least 15% or at least 20% or at least 25%) or at least 30%> or at least 35% or at least 40% or at least 45% or at least 50%, as compared to antigen-specific antibody production in the absence of the immune potentiator. In one embodiment, antigen-specific IgG production is evaluated. Antigen-specific antibody production can be evaluated by methods well established in the art, including but not limited to ELISA, RIA and the like that measure the level of antigen-specific antibody (e.g., IgG) in a sample (e.g., a serum sample).

In one embodiment, an immune potentiator increases the effector memory CD62L¹⁰ T cell population. For example, an immune potentiator can increase the total % of CD62L¹⁰ T cells among CD8+ T cells. Among other functions, the effector memory CD62L¹⁰ T cell population has been shown to have an important function in lymphocyte trafficking (see e.g., Schenkel, J. M. and Masopust, D. (2014) Immunity 41:886-897). In various embodiments, an immune potentiator can increase the total percentage of effector memory CD62L¹⁰ T cells among the CD8+ T cells in response to an antigen (as compared to the total percentage of CD62L¹⁰ T cells among the CD8+ T cells population in the absence of the immune potentiator) by at least 5% or at least 10% or at least 15% or at least 20% or at least 25% or at least 30% or at least 35% or at least 40% or at least 45% or at least 50%. The total percentage of effector memory CD62L¹⁰ T cells among the CD8+ T cells can be determined by standard methods known in the art, including but not limited to fluorescent activated cell sorting (FACS) or magnetic activated cell sorting (MACS).

The ability of an immune potentiator mRNA to enhance an immune response to a cancer antigen can be evaluated in mouse model systems known in the art. In one embodiment, an immune competent mouse model system is used. In one embodiment, the mouse model system comprises C57/B16 mice. In another embodiment, the mouse model system comprises BalbC mice or CD1 mice {e.g., to evaluate B cell responses, such an antigen-specific antibody responses). In one embodiment, an immune potentiator polypeptide functions downstream of at least one Toll-like receptor (TLR) to thereby enhance an immune response. Accordingly, in one embodiment, the immune potentiator is not a TLR but is a molecule within a TLR signaling pathway downstream from the receptor itself.

In one embodiment, the mRNA encoding an immune potentiator can comprises one or more modified nucleobases. Suitable modifications are discussed further below.

In one embodiment, the mRNA encoding an immune potentiator is formulated into the LPMP formulation. In one embodiment, the mRNA encodes a cancer antigen. In one embodiment, the LPMP/mRNA formulation is administered to a subject to enhance an immune response against a cancer antigen in the subject.

Immune Potentiator mRNAs that Stimulate Type I Interferon

In some embodiments, the disclosure provides an immune potentiator mRNA encoding a polypeptide that stimulates or enhances an immune response against an antigen of interest by stimulating or enhancing Type I interferon pathway signaling, thereby stimulating or enhancing Type I interferon (IFN) production.

A number of components involved in Type I IFN pathway signaling have been established, including STING, Interferon Regulatory Factors, such as IRF1, IRF3, IRF5, IRF7, IRF8, and IRF9, TBK1, IKKi, MyD88 and TRAM. Additional components involved in Type I IFN pathway signaling include TRAF3, TRAF6, IRAK-1, IRAK-4, TRIF, IPS-1, TLR-3, TLR-4, TLR-7, TLR-8, TLR-9, RIG-1, DAI, and IFI16.

Accordingly, in one embodiment, an immune potentiator mRNA encodes any of the foregoing components involved in Type I IFN pathway signaling.

### Agents for Promotion of Antigen Presenting Cells

In some embodiments, LPMP/mRNA therapeutic composition can be combined with agents for promoting the production of antigen presenting cells (APCs), for instance, by converting non-APCs into pseudo-APCs. Antigen presentation is a key step in the initiation, amplification and duration of an immune response. In this process fragments of antigens are presented through the Major Histocompatibility Complex (MHC) or Human Leukocyte Antigens (HLA) to T cells driving an antigen-specific immune response. For immune prophylaxis and therapy, enhancing this response is important for improved efficacy. The LPMP/mRNA therapeutic composition may be designed or enhanced to drive efficient antigen presentation. One method for enhancing APC processing and presentation, is to provide better targeting of the LPMP/mRNA therapeutic composition to antigen presenting cells (APC). Another approach involves activating the APC cells with immune-stimulatory formulations and/or components. Alternatively, methods for reprograming non-APC into becoming APC may be used with the LPMP/mRNA therapeutic composition. Importantly, most cells that take up mRNA formulations and are targets of their therapeutic actions are not APC. Therefore, designing a way to convert these cells into APC would be beneficial for efficacy. Methods and approaches for delivering the LPMP/mRNA therapeutic composition, e.g., mRNA vaccines to cells while also promoting the shift of a non-APC to an APC are provided herein. In some embodiments, a mRNA encoding an APC reprograming molecule is included in the LPMP/mRNA therapeutic composition or coadministered with the LPMP/mRNA therapeutic composition.

An APC reprograming molecule, as used herein, is a molecule that promotes a transition in a non APC cell to an APC-like phenotype. An APC-like phenotype is property that enables MHC class II processing. Thus, an APC cell having an APC-like phenotype is a cell having one or more exogenous molecules (APC reprograming molecule) which has enhanced MHC class II processing capabilities in comparison to the same cell not having the one or more exogenous molecules. In some embodiments, an APC reprograming molecule is a CUT A (a central regulator of MHC Class II expression); a chaperone protein such as CLIP, HLA-DO, HLA-DM etc. (enhancers of loading of antigen fragments into MHC Class II) and/or a costimulatory molecule like CD40, CD80, CD86 etc. (enhancers of T cell antigen recognition and T cell activation).

A CIITA protein is a transactivator that enhances activation of transcription of MHC Class II genes (Steimle et aL, 1993, Cell 75: 135-146) by interacting with a conserved set of DNA binding proteins that associate with the class II promoter region. The transcriptional activation function of CIITA has been mapped to an amino terminal acidic domain (amino acids 26-137). A nucleic acid molecule encoding a protein that interacts with CIITA, termed CIITA-interacting protein 104 (also referred to herein as CIP104). Both CITTA and CIP104 have been shown to enhance transcription from MHC class II promoters and thus are useful as APC reprograming molecule of the invention. In some embodiments the APC reprograming molecule are full length CIITA, CIP 104 or other related molecules or active fragments thereof, such as amino acids 26-137 of CIITA, or amino acids having at least 80% sequence identity thereto and maintaining the ability to enhance activation of transcription of MHC Class II genes.

In some embodiments, the LPMP/mRNA therapeutic composition may include a recall antigen, also sometimes referred to as a memory antigen. A recall antigen is an antigen that has previously been encountered by an individual and for which there are pre-existent memory lymphocytes. In some embodiments, the recall antigen may be an infectious disease antigen that the individual has likely encountered such as an influenza antigen. The recall antigen helps promote a more robust immune response.

The antigens or neoepitopes selected for inclusion in the LPMP/mRNA therapeutic composition typically will be high affinity binding peptides. In some embodiments, the antigens or neoepitopes binds an HLA protein with greater affinity than a wild-type peptide. The antigen or neoepitope has an IC50 of at least less than 5000 nM, at least less than 500 nM, at least less than 250 nM, at least less than 200 nM, at least less than 150 nM, at least less than 100 nM, at least less than 50 nM or less in some embodiments. Typically, peptides with predicted IC50<50 nM, are generally considered medium to high affinity binding peptides and will be selected for testing their affinity empirically using biochemical assays of HLA-binding.

The cancer antigens can be personalized cancer antigens. The LPMP/mRNA therapeutic composition may include RNA encoding for one or more known cancer antigens specific for the tumor or cancer antigens specific for each subject, referred to as neoepitopes or subject specific epitopes or antigens. A “subject specific cancer antigen” is an antigen that has been identified as being expressed in a tumor of a particular patient. The subject specific cancer antigen may or may not be typically present in tumor samples generally. Tumor associated antigens that are not expressed or rarely expressed in non-cancerous cells, or whose expression in non-cancerous cells is sufficiently reduced in comparison to that in cancerous cells and that induce an immune response induced upon vaccination, are referred to as neoepitopes. Neoepitopes, like tumor associated antigens, are completely foreign to the body and thus would not produce an immune response against healthy tissue or be masked by the protective components of the immune system. In some embodiments, the LPMP/mRNA therapeutic compositions based on neoepitopes are desirable because such vaccine formulations will maximize specificity against a patient's specific tumor. Mutation-derived neoepitopes can arise from point mutations, non-synonymous mutations leading to different amino acids in the protein; read-through mutations in which a stop codon is modified or deleted, leading to translation of a longer protein with a novel tumor-specific sequence at the C-terminus; splice site mutations that lead to the inclusion of an intron in the mature mRNA and thus a unique tumor-specific protein sequence; chromosomal rearrangements that give rise to a chimeric protein with tumor-specific sequences at the junction of 2 proteins (i.e., gene fusion); frameshift mutations or deletions that lead to a new open reading frame with a novel tumor-specific protein sequence; and translocations.

Thus, in some embodiments, the LPMP/mRNA therapeutic compositions include at least 1 cancer antigens including mutations selected from the group consisting of frame-shift mutations and recombinations or any of the other mutations described herein. In some embodiments, the LPMP/mRNA therapeutic compositions include at least 1 immunogenic polypeptide including mutations selected from the group consisting of frame-shift mutations and recombinations or any of the other mutations described herein. In some embodiments, the LPMP/mRNA therapeutic compositions include at least 1 signaling polypeptide including mutations selected from the group consisting of frame-shift mutations and recombinations or any of the other mutations described herein.

### Nucleic Acid Sequences

In some embodiments, the polynucleotides are polynucleotide constructs, which encode one or more wild type or engineered antigens (or an antibody to an antigen). In some embodiments, the polynucleotide comprises an antigenic polypeptide or an immunogenic variant or an immunogenic fragment thereof. The antigen may be derived from a tumor, e.g., a tumor specific antigen, a tumor associated antigen, a tumor neoantigen, or a combination thereof. In some embodiments, the polynucleotide comprises a signaling or anti-cancer protein, such as a secreted cytokine, cytokine receptor complex, hormone, or immune potentiator.

In some embodiments, the polynucleotide may be a mRNA, an siRNA or siRNA precursor, a microRNA (miRNA) or miRNA precursor, a plasmid, a Dicer substrate small interfering RNA (dsiRNA), a short hairpin RNA (shRNA), an asymmetric interfering RNA (aiRNA), a peptide nucleic acid (PNA), a morpholino, a locked nucleic acid (LNA), a piwi-interacting RNA (piRNA), a ribozyme, a deoxyribozyme (DNAzyme), an aptamer, a circular RNA (circRNA), a guide RNA (gRNA), or a DNA molecule encoding any of these RNAs. In one embodiment, the polynucleotide is an mRNA.

In some embodiments, the polynucleotide encodes a polypeptide comprising p53, ART-4, BAGE, ss-catenin/m, Bcr-abL CAMEL, CAP-1, CASP-8, CDC27/m, CDK4/m, CEA, CLAUDIN-12, c-MYC, CT, Cyp-B, DAM, ELF2M, ETV6-AML1, G250, GAGE, GnT-V, Gap 100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A10, MAGE-A11, or MAGE-A12, MAGE-B, MAGE-C, MART-1/Melan-A, MC1R, Myosin/m, MUC1, MUM-1, -2, -3, NA88-A, NF1, NY-ESO-1, NY-BR-1, pI90 minor BCR-abL, Plac-1, Pml/RARa, PRAME, proteinase 3, PSA, PSM, RAGE, RU1 or RU2, SAGE, SART-1 or S ART-3, SCGB3A2, SCP1, SCP2, SCP3, SSX, SURVIVIN, TEL/AML1, TPI/m, TRP-1, TRP-2, TRP-2/INT2, TPTE, WT, WT-1, or a combination thereof.

In some embodiments, the polynucleotide encodes an IL-2 peptide, IL-2-Ra, tdTomato, Cre recombinase, GFP, eGFP, Anti-CD19, CD20, CAR-T, Anti-HER2, Etanercept (Enbrel), Humira, erythropoietin, Epogen, Filgrastim, Keytruda, Rituximab, Romiplostim, Sargramostim, or a variant, fragment or subunit thereof. Exemplary sequences include those shown in Table 9.

In some embodiments, the polynucleotide encodes an IL-15 peptide, IL-15-Ra, or a fragment or subunit thereof. In one embodiment, the polypeptide is IL-15 peptide, or a fragment or subunit thereof. Exemplary sequences include those shown in Table 9.

In some embodiments, the polynucleotide encodes IL-1 α, IL-1 β, IL-1ra, IL-2, IL-3, IL-4, IL-5, IL-6, IL-7, IL-8, IL-9, IL-10, IL-11, IL-12, IL-13, IL-14, IL-15, IL-16, IL-17A, IL-17B, IL-17C, IL-17D, IL-17E, IL-17F, IL-18, IL-19, IL-20, IL-21, IL-22, IL-23, IL-24, IL-25, IL-26, IL-27, IL-28A/B, IL-29, IL-30, IL-31, IL-32, IL-33, IL-35, TGF-β, GM-CSF, M-CSF, G-CSF, TNF-α, TNF-β, LAF, TCGF, BCGF, TRF, BAF, BDG, MP, LIF, OSM, TMF, PDGF, IFN-α, IFN-β, IFN-γ, Uteroglobins, Foxp3, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CCL1, CCL2, CCL3, CCL4, CCL5, CCL6, CCL7, CCL8, CCL9/10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, XCL1, XCL2, CX3CL1, or a fragment, subunit, or variant thereof.

Antigen variants or other polypeptide variants refers to molecules that differ in their amino acid sequence from a wild-type, native, or reference sequence. The polypeptide variants may possess substitutions, deletions, and/or insertions at certain positions within the amino acid sequence, as compared to a native or reference sequence. Ordinarily, variants possess at least 50% identity to a wild-type, native or reference sequence. In some embodiments, variants share at least 80%, or at least 90% identity with a wild-type, native, or reference sequence.

Variant polypeptides encoded by nucleic acids of the disclosure may contain amino acid changes that confer any of a number of desirable properties, e.g., that enhance their immunogenicity, enhance their expression, and/or improve their stability or PK/PD properties in a subject. Variant antigens/polypeptides can be made using routine mutagenesis techniques and assayed as appropriate to determine whether they possess the desired property. Assays to determine expression levels and immunogenicity are well known in the art and exemplary such assays are set forth in the Examples section. Similarly, PK/PD properties of a protein variant can be measured using art recognized techniques, e.g., by determining expression of antigens in a vaccinated subject overtime and/or by looking at the durability of the induced immune response. The stability of protein(s) encoded by a variant nucleic acid may be measured by assaying thermal stability or stability upon urea denaturation or may be measured using in silico prediction. Methods for such experiments and in silico determinations are known in the art.

The term “identity” refers to a relationship between the sequences of two or more polypeptides (e.g. antigens, anti-cancer proteins or signaling proteins) or polynucleotides (nucleic acids), as determined by comparing the sequences. Identity also refers to the degree of sequence relatedness between or among sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related antigens, proteins, or nucleic acids can be readily calculated by known methods. “Percent (%) identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide (e.g., antigen, anti-cancer protein, or signaling protein) have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. &Wunsch, C. D. (1970) “A general method applicable to the search for similarities in the amino acid sequences of two proteins.” J. Mol. Biol. 48:443-453). More recently, a Fast Optimal Global Sequence Alignment Algorithm (FOGSAA) has been developed that purportedly produces global alignment of nucleotide and protein sequences faster than other optimal global alignment methods, including the Needleman-Wunsch algorithm.

As such, polynucleotides encoding peptides or polypeptides containing substitutions, insertions and/or additions, deletions, and covalent modifications with respect to reference sequences, in particular the polypeptide (e.g., antigen or signaling protein) sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification, or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence that is soluble or linked to a solid support. In some embodiments, sequences for (or encoding) signal sequences, termination sequences, transmembrane domains, linkers, multimerization domains (such as, e.g., foldon regions) and the like may be substituted with alternative sequences that achieve the same or a similar function. In some embodiments, cavities in the core of proteins can be filled to improve stability, e.g., by introducing larger amino acids. In other embodiments, buried hydrogen bond networks may be replaced with hydrophobic resides to improve stability. In yet other embodiments, glycosylation sites may be removed and replaced with appropriate residues. Such sequences are readily identifiable to one of skill in the art. It should also be understood that some of the sequences provided herein contain sequence tags or terminal peptide sequences (e.g., at the N-terminal or C-terminal ends) that may be deleted, for example, prior to use in the preparation of an RNA (e.g., mRNA) vaccine.

As recognized by those skilled in the art, protein fragments, functional protein domains, and homologous proteins are also considered to be within the scope of coronavirus antigens of interest. For example, provided herein is any protein fragment (meaning a polypeptide sequence at least one amino acid residue shorter than a reference antigen sequence but otherwise identical) of a reference protein, provided that the fragment is immunogenic and confers a protective immune response to the coronavirus. In addition to variants that are identical to the reference protein but are truncated, in some embodiments, an antigen includes 2, 3, 4, 5, 6, 7, 8, 9, 10, or more mutations, as shown in any of the sequences provided or referenced herein. Antigens/antigenic polypeptides can range in length from about 4, 6, or 8 amino acids to full length proteins.

In some embodiments, the polynucleotide is an mRNA. Messenger RNA (mRNA) is any RNA that encodes a (at least one) protein (a naturally-occurring, non-naturally-occurring, or modified polymer of amino acids) and can be translated to produce the encoded protein in vitro, in vivo, in situ, or ex vivo. The skilled artisan will appreciate that, except where otherwise noted, nucleic acid sequences set forth in the instant application may recite “T”s in a representative DNA sequence but where the sequence represents RNA (e.g., mRNA), the “T”s would be substituted for “U”s. Thus, any of the DNAs disclosed and identified by a particular sequence identification number herein also disclose the corresponding RNA (e.g., mRNA) sequence complementary to the DNA, where each “T” of the DNA sequence is substituted with “U.”

In some embodiments, the polynucleotide (e.g., mRNA) having an open reading frame (ORF) encoding an antigen, tumor antigen, or signaling polypeptide. An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. The sequences may further comprise additional elements, e.g., 5′ and 3′ UTRs.

In some embodiments, the RNA (e.g., mRNA) further comprises a 5′ UTR, 3′ UTR, a poly(A) tail and/or a 5′ cap analog.

In some embodiments, the mRNA comprises a 5′ untranslated region (UTR) and/or a 3′ UTR.

In some embodiments, the mRNA is derived from (a) a DNA molecule; or (b) an RNA molecule. In the mRNA, T is optionally substituted with U.

In some embodiments, the mRNA is derived from a DNA molecule. The DNA molecule can further comprise a promoter. In some embodiments, the promoter is a T7 promoter, a T3 promoter, or an SP6 promoter. In some embodiments, the promoter is located at the 5′ UTR.

In some embodiments, the mRNA is an RNA molecule. The RNA molecule may be a self-replicating RNA molecule.

In some embodiments, the mRNA is an RNA molecule. The RNA molecule may further comprise a 5′ cap. The 5′ cap can have a Cap 1 structure, a Cap 1 (m6A) structure, a Cap 2 structure, a Cap 3 structure, a Cap 0 structure, or any combination thereof.

In some embodiments, the polynucleotide is an mRNA which encodes an IL-2 molecule. In one embodiment, the IL-2 molecule comprises a naturally occurring IL-2 molecule, a fragment of a naturally occurring IL-2 molecule, or a variant thereof. In one embodiment, the IL-2 molecule comprises a variant of a naturally occurring IL-2 molecule (e.g., an IL-2 variant, e.g., as described herein), or a fragment thereof.

In one embodiment, the polynucleotide is an mRNA which encodes an IL-2 molecule comprising an amino acid sequence having at least 85%, 90%, 95%, 96%, 97%, 98%, 99% or 100% identity to the amino acid sequence of an IL-2 molecule provided in any one of Tables I-III.

In some embodiments, the polynucleotide is an mRNA which encodes an IL-15 molecule. In one embodiment, the IL-15 molecule comprises a naturally occurring IL-15 molecule, a fragment of a naturally occurring IL-15 molecule, or a variant thereof. In one embodiment, the IL-15 molecule comprises a variant of a naturally occurring IL-15 molecule (e.g., an IL-15 variant, e.g., as described herein), or a fragment thereof.

In one embodiment, the polynucleotide is an mRNA which encodes an IL-15 molecule comprising an amino acid sequence having at least 85%, 90%, 95%, 96%, 97%, 98%, 99% or 100% identity to the amino acid sequence of an IL-15 molecule provided in Table IV.

In some embodiments, the mRNA comprises a 5′ untranslated region (UTR) and/or a 3′ UTR.

In some embodiments, the mRNA comprises a 5′ UTR. The 5′ UTR may comprise a Kozak sequence.

In some embodiments, the mRNA comprises a 3′ UTR. In some embodiments, the 3′ UTR comprises one or more sequences derived from an amino-terminal enhancer of split (AES). In some embodiments, the 3′ UTR comprises a sequence derived from mitochondrially encoded 12S rRNA (mtRNRI).

In some embodiments, the mRNA comprises a poly(A) sequence. In one embodiment, the poly(A) sequence is a 110-nucleotide sequence consisting of a sequence of 30 adenosine residues, a 10-nucleotide linker sequence, and a sequence of 70 adenosine residues.

TABLE I
Exemplary IL-2 sequences, human serum albumin (HAS) sequences, and HAS-IL-2
sequences

| SEQ ID | Sequence | |
|---|---|---|
| NO | information | Sequence |
| 1 | Human IL-2 | APTSSSTKKTQLQLEHLLLDLQMILNGINNYKNPKLTRMLTFKF |
| | | YMPKKATELKHLQCLEEELKPLEEVLNLAQSKNFHLRPRDLIS |
| | | NINVIVLELKGSETTFMCEYADETATIVEFLNRWITFCQSIISTLT |
| 30 | Human IL-2 | MYRMQLLSCIALSLALVTNSAPTSSSTKKTQLQLEHLLLDLQMI |
| | | LNGINNYKNPKLTRMLTFKFYMPKKATELKHLQCLEEELKPLE |
| | | EVLNLAQSKNFHLRPRDLISNINVIVLELKGSETTFMCEYADET |
| | | ATIVEFLNRWITFCQSIISTLT |
| 2 | Human IL-2 | APTSSSTKKTQLQLEHLLLDLQMILNGINNYKNPKLTRMLTFKF |
| | N88D | YMPKKATELKHLQCLEEELKPLEEVLNLAQSKNFHLRPRDLIS |
| | | DINVIVLELKGSETTFMCEYADETATIVEFLNRWITFCQSIISTLT |
| 31 | Human IL-2 | MYRMQLLSCIALSLALVTNSAPTSSSTKKTQLQLEHLLLDLQMI |
| | N88D | LNGINNYKNPKLTRMLTFKFYMPKKATELKHLQCLEEELKPLE |
| | | EVLNLAQSKNFHLRPRDLISDINVIVLELKGSETTFMCEYADET |
| | | ATIVEFLNRWITFCQSIISTLT |
| 3 | Human IL-2 | APTSSSTKKTQLQLEHLLLDLQMILNGINNYKNPKLIRMLTFKF |
| | V69A, Q74P, | YMPKKATELKHLQCLEEELKPLEEALNLAPSKNFHLRPRDLIS |
| | N88D | DINVIVLELKGSETTFMCEYADETATIVEFLNRWITFCQSIISTLT |
| 32 | Human IL-2 | MYRMQLLSCIALSLALVTNSAPTSSSTKKTQLQLEHLLLDLQMI |
| | V69A, Q74P, | LNGINNYKNPKLTRMLTFKFYMPKKATELKHLQCLEEELKPLE |
| | N88D | EALNLAPSKNFHLRPRDLISDINVIVLELKGSETTFMCEYADET |
| | | ATIVEFLNRWITFCQSIISTLT |
| 4 | Human IL-2, | APTSSSTKKTQLQLEHLLLDLQMILNGINNYKNPKLTRMLTFKF |
| | V91K | YMPKKATELKHLQCLEEELKPLEEVLNLAQSKNFHLRPRDLIS |
| | | NINKIVLELKGSETTFMCEYADETATIVEFLNRWITFCQSHSTLT |
| 33 | Human IL-2, | MYRMQLLSCIALSLALVTNSAPTSSSTKKTQLQLEHLLLDLQMI |
| | V91K | LNGINNYKNPKLTRMLTFKFYMPKKATELKHLQCLEEELKPLE |
| | | EVLNLAQSKNFHLRPRDLISNINKIVLELKGSETTFMCEYADET |
| | | ATIVEFLNRWITFCQSIISTLT |
| 5 | Human IL-2 | APTSSSTKKTQLQLEHLLLDLQMILNGINNYKNPKLTRMLTFKF |
| | V69A, Q74P, | YMPKKATELKHLQCLEEELKPLEEALNLAPSKNFHLRPRDLIS |
| | V9IK | NINKIVLELKGSETTFMCEYADETATIVEFLNRWITFCQSHSTLT |
| 34 | Human IL-2 | MYRMQLLSCIALSLALVTNSAPTSSSTKKTQLQLEHLLLDLQMI |
| | V69A, Q74P, | INGINNYKNPKLTRMLTFKFYMPKKATELKHLQCLEEELKPLE |
| | V91K | EALNLAPSKNFHLRPRDLISNINKIVLELKGSETTFMCEYADET |
| | | ATIVEFLNRWITFCQSIISTLT |
| 6 | Human IL-2 | APTSSSTKKTQLQLEHLLLTLQMILNGINNYKNPKLTRMLTEKF |
| | D20T | YMPKKATELKHLQCLEEELKPLEEVLNLAQSKNFHLRPRDLIS |
| | | NINVIVLELKGSETTFMCEYADETATIVEFLNRWITFCQSIISTLT |
| 35 | Human IL-2 | MYRMQLLSCIALSLALVTNSAPTSSSTKKTQLQLEHLLLTLQMI |
| | D20T | LNGINNYKNPKLTRMLTFKFYMPKKATELKHLQCLEEELKPLE |
| | | EVLNLAQSKNFHLRPRDLISNINVIVLELKGSETTFMCEYADET |
| | | ATIVEFLNRWITFCQSIISTLT |
| 7 | Human IL-2 | AGUUCCCUAU CACUCUCUUU AAUCACUACU |
| | mRNA | CACAGUAACC UCAACUCCUG CCACAAUGUA |
| | sequence | CAGGAUGCAA CUCCUGUCUU GCAUUGCACU |
| | | AAGUCUUGCA CUUGUCACAA ACAGUGCACC |
| | | UACUUCAAGU UCUACAAAGA AAACACAGCU |
| | | ACAACUGGAG CAUUUACUGC UGGAUUUACA |
| | | GAUGAUUUUG AAUGGAAUUA AUAAUUACAA |
| | | GAAUCCCAAA CUCACCAGGA UGCUCACAUU |
| | | UAAGUUUUAC AUGCCCAAGA AGGCCACAGA |
| | | ACUGAAACAU CUUCAGUGUC UAGAAGAAGA |
| | | ACUCAAACCU CUGGAGGAAG UGCUAAAUUU |
| | | AGCUCAAAGC AAAAACUUUC ACUUAAGACC |
| | | CAGGGACUUA AUCAGCAAUA UCAACGUAAU |
| | | AGUUCUGGAA CUAAAGGGAU CUGAAACAAC |
| | | AUUCAUGUGU GAAUAUGCUG AUGAGACAGC |
| | | AACCAUUGUA GAAUUUCUGA ACAGAUGGAU |
| | | UACCUUUUGU CAAAGCAUCA UCUCAACACU |
| | | GACUUGAUAA UUAAGUGCUU CCCACUUAAA |
| | | ACAUAUCAGG CCUUCUAUUU AUUUAAAUAU |
| | | UUAAAUUUUA UAUUUAUUGU UGAAUGUAUG |
| | | GUUUGCUACC UAUUGUAACU AUUAUUCUUA |
| | | AUCUUAAAAC UAUAAAUAUG GAUCUUUUAU |
| | | GAUUCUUUUU GUAAGCCCUA GGGGCUCUAA |
| | | AAUGGUUUCA CUUAUUUAUC CCAAAAUAUU |
| | | UAUUAUUAUG UUGAAUGUUA AAUAUAGUAU |
| | | CUAUGUAGAU UGGUUAGUAA AACUAUUUAA |
| | | UAAAUUUGAU AAAUAUAAAA AAAAAAAAAA |
| | | AAAAAAAAAA AA |
| 8 | Human | AHKSEVAHRFKDLGEENFKALVLIAFAQYLQQCPFEDHVKLVN |
| | serum | EVTEFAKTCVADESAENCDKSLHTLFGDKLCTVATLRETYGE |
| | albumin | MADCCAKQEPERNECFLQHKDDNPNLPRLVRPEVDVMCTAFH |
| | (HSA) | DNEETFLKKYLYEIARRHPYFYAPELLFFAKRYKAAFTECCQA |
| | | ADKAACLLPKLDELRDEGKASSAKQRLKCASLQKFGERAFKA |
| | | WAVARLSQRFPKAEFAEVSKLVTDLTKVHTECCHGDLLECAD |
| | | DRADLAKYICENQDSISSKLKECCEKPLLEKSHCIAEVENDEMP |
| | | ADLPSLAADFVESKDVCKNYAEAKDVFLGMFLYEYARRHPDY |
| | | SVVLLLRLAKTYETTLEKCCAAADPHECYAKVFDEFKPLVEEP |
| | | QNLIKQNCELFEQLGEYKFQNALLVRYTKKVPQVSTPTLVEVS |
| | | RNLGKVGSKCCKHPEAKRMPCAEDYLSVVLNQLCVLHEKTPV |
| | | SDRVTKCCTESLVNRRPCFSALEVDETYVPKEFNAETFTFHADI |
| | | CTLSEKERQIKKQTALVELVKHKPKATKEQLKAVMDDFAAFV |
| | | EKCCKADDKETCFAEEGKKLVAASQAALGL |
| 9 | Leader- | MKWVTFISLLFLESSAYSRGVFRRDAHKSEVAHRFKDLGEEN |
| | HSA-(G3S)- | FKALVLIAFAQYLQQCPFEDHVKLVNEVTEFAKTCVADESAEN |
| | (Human | CDKSIHTLFGDKLCTVATLRETYGEMADCCAKQEPERNECFL |
| | IL-2) | QHKDDNPNLPRLVRPEVDVMCTAFHDNEETFLKKYLYEIARR |
| | | HPYFYAPELLFFAKRYKAAFTECCQAADKAACLLPKLDELRDE |
| | | GKASSAKQRLKCASLQKFGERAFKAWAVARLSQRFPKAEFAE |
| | | VSKLVTDLTKVHTECCHGDLLECADDRADLAKYICENQDSISS |
| | | KLKECCEKPLLEKSHCIAEVENDEMPADLPSLAADFVESKDVC |
| | | KNYAEAKDVFLGMFLYEYARRHPDYSVVLLLRLAKTYETTLE |
| | | KCCAAADPHECYAKVFDEFKPLVEEPQNLIKQNCELFEQLGEY |
| | | KFQNALLVRYTKKVPQVSTPTLVEVSRNLGKVGSKCCKHPEA |
| | | KRMPCAEDYLSVVLNQLCVLHEKTPVSDRVTKCCTESLVNRR |
| | | PCFSALEVDETYVPKEFNAETFTFHADICTLSEKERQIKKQTAL |
| | | VELVKHKPKATKEQLKAVMDDFAAFVEKCCKADDKETCFAE |
| | | EGKKLVAASQAALGLGGGSAPTSSSTKKTQLQLEHLLLDLQMI |
| | | LNGINNYKNPKLIRMLTEKEYMPKKATELKHLQCLEEELKPLE |
| | | EVINLAQSKNFHLRPRDLISNINVIVLELKGSETTFMCEYADET |
| | | ATIVEFLNRWITFCQSIISTLT |
| 10 | Leader- | MKWVTFISLLFLFSSAYSRGVFRRDAHKSEVAHRFKDLGEE |
| | HSA-(G3S)- | NFKALVLIAFAQYLQQCPFEDHVKLVNEVTEFAKTCVADESAE |
| | Human | NCDKSLHTLFGDKLCTVATLRETYGEMADCCAKQEPERNECF |
| | IL-2 NSSD) | LQHKDDNPNLPRLVRPEVDVMCTAFHDNEETELKKYLYELAR |
| | | RHPYFYAPELLFFAKRYKAAFTECCQAADKAACLLPKLDELR |
| | | DEGKASSAKQRLKCASLQKFGERAFKAWAVARLSQRFPKAEF |
| | | AEVSKLVTDLIKVATECCHGDLLECADDRADLAKYICENQDSI |
| | | SSKLKECCEKPLLEKSHCIAEVENDEMPADLPSLAADFVESKD |
| | | VCKNYAEAKDVFLGMFLYEYARRHPDYSVVLLLRLAKTYETT |
| | | LEKCCAAADPHECYAKVFDEFKPLVEEPQNLIKQNCELFEQLG |
| | | EYKFQNALLYRYTKKVPQVSTPTLVEVSRNLGKVGSKCCKHP |
| | | EAKRMPCAEDYLSVVLNQLCVLHEKTPVSDRVTKCCTESLVN |
| | | RRPCFSALEVDETYVPKEFNAETFTFHADICTLSEKERQIKKQT |
| | | ALVELVKHKPKATKEQLKAVMDDFAAFVEKCCKADDKETCF |
| | | AEEGKKLVAASQAALGLGGGSAPTSSSIKKTQLQLEHLLLDLQ |
| | | MILNGINNYKNPKLTRMLTEKFYMPKKATELKHLQCLEEELKP |
| | | LEEVLNLAQSKNEHLRPRDLISDINVIVLELKGSEITFMCEYAD |
| | | ETATIVEFLNRWITFCQSIISTLT |
| 11 | Leader- | MKWVTFISLLFLFSSAYSRGVFRRDAHKSEVAHRFKDLGEEN |
| | HSA-(G3S)- | FKALVLIAFAQYLQQCPFEDHVKLVNEVTEFAKTCVADESAEN |
| | (Human | CDKSLHTLFGDKLCTVATLRETYGEMADCCAKQEPERNECFL |
| | IL-2 V69A, | QHKDDNPNLPRLVRPEVDVMCTAFHDNEETFLKKYLYELARR |
| | Q74P, | HPYFYAPELLFFAKRYKAAFTECCQAADKAACLIPKLDELRDE |
| | N88D) | GKASSAKQRLKCASLQKFGERAFKAWAVARLSQRFPKAEFAE |
| | | VSKLVTDLTKVHTECCHGDLLECADDRADLAKYICENQDSISS |
| | | KLKECCEKPLLEKSHCIAEVENDEMPADLPSLAADFVESKDVC |
| | | KNYAEAKDVFLGMFLYEYARRHPDYSVVLLLRLAKTYETTLE |
| | | KCCAAADPHECYAKVFDEFKPLVEEPQNLIKQNCELFEQLGEY |
| | | KFQNALLVRYTKKVPQVSTPTLVEVSRNLGKVGSKCCKHPEA |
| | | KRMPCAEDYLSVVLNQLCVLHEKTPVSDRVTKCCTESLVNRR |
| | | PCFSALEVDETYVPKEFNAETFTFHADICTLSEKERQIKKQTAL |
| | | VELVKHKPKATKEQLKAVMDDFAAFVEKCCKADDKETCFAE |
| | | EGKKLVAASQAALGLGGGSAPTSSSTKKTQLQLEHLLLDLQMI |
| | | LNGINNYKNPKLTRMLTFKFYMPKKATELKHLQCLEEELKPLE |
| | | EALNLAPSKNEHLRPRDLISDINVIVLELKGSETTFMCEYADET |
| | | ATIVEFLNRWITFCQSIISTLT |
| 36 | Leader- | AUGAAGUGGGUGACCUUCAUCAGCCUGCUGUUCCUGUUCA |
| | HSA-(G3S)- | GCAGCGCCUACAGCAGAGGCGUGUUCAGAAGAGACGCCCA |
| | (Human | CAAGAGCGAGGUGGCCCACAGAUUCAAGGACCUGGGCGAG |
| | IL-2 V69A, | GAGAACUUCAAGGCCCUGGUGCUGAUCGCCUUCGCCCAGU |
| | Q74P, | ACCUGCAGCAGUGCCCUUUCGAGGACCACGUGAAGCUGGU |
| | N88D) ORF | GAACGAGGUGACCGAGUUCGCCAAGACCUGCGUGGCCGAC |
| | mRNA | GAGAGCGCCGAGAACUGCGACAAGAGCCUGCACACCCUGU |
| | sequence | UCGGCGACAAGCUGUGCACCGUGGCCACCCUGAGAGAAAC |
| | | UUACGGCGAGAUGGCCGACUGCUGCGCCAAGCAGGAGCCA |
| | | GAGCGGAACGAGUGCUUCCUCCAGCACAAGGACGACAACC |
| | | CUAACCUGCCUAGACUGGUAAGGCCUGAGGUGGACGUGAU |
| | | GUGUACCGGCUUCCACGACAACGAGGAGACAUUCCUGAAG |
| | | AAGUACCUGUACGAGAUCGCCAGAAGACACCCUUACUUCU |
| | | ACGCCCCUGAGUUGCUGUUCUUCGCGAAGAGAUACAAGGC |
| | | CGCCUUCACCGAGUGCUGCCAGGCCGCCGAUAAGGCCGCG |
| | | UGCCUGCUGCCUAAGCUGGACGAGCUGAGAGACGAGGGCA |
| | | AGGCAUCCAGCGCUAAGCAGAGACUGAAGUGCGCCAGCCU |
| | | GCAGAAGUUCGGAGAGAGAGCUUUCAAGGCGUGCGCAGU |
| | | GGCUAGAUUGAGCCAAAGAUUCCCUAAGGCAGAAUUCGCU |
| | | GAGGUGAGCAAGCUCGUGACUGACCUGACCAAGGUGCAUA |
| | | CAGAGUGCUGUCACGGCGACCUGCUGGAGUGCGCCGACGA |
| | | CAGAGCCGACCUGGCCAAGUACAUCUGCGAGAACCAGGAC |
| | | AGCAUCAGCAGCAAGCUGAAGGAGUGUUGUGAGAAGCCU |
| | | CUAUUGGAGAAGAGUCACUGCAUUGCCGAGGUGGAGAAC |
| | | GACGAGAUGCCUGCGGAUCUGCCAAGCUUGGCGGCCGACU |
| | | UCGUGGAGAGCAAGGACGUGUGCAAGAACUACGCCGAGGC |
| | | CAAGGACGUUUUCCUGGGCAUGUUCCUCUACGAGUACOCA |
| | | CGCAGACAUCCAGACUACAGCGUGGUGCUGCUGCUGAGAC |
| | | UGGCUAAGACAUACGAAACUACCCUGGAGAAGUGCUGCGC |
| | | AGCGGCGGACCCUCACGAGUGUUACGCCAAGGUGUUCGAC |
| | | GAGUUCAAGOCUCUGGUGGAGGAGCCUCAGAACCUGAUCA |
| | | AGCAGAACUGUGAGCUGUUCGAGCAGCUCGOCGAGUACAA |
| | | GUUCCAGAACGCCCUGUUGGUCCOCUACACCAAGAAGGUG |
| | | CCUCAAGUAAGUACCCCUACCCUGGUAGAGGUUAGUAGAA |
| | | ACCUGGGCAAGGUGGGCAGCAAGUGCUGUAAGCACCCAGA |
| | | AGCUAAGAGGAUGCCUUGCGCCGAGGACUACCUGUCCGUU |
| | | GUGCUGAACCAGCUGUGCGUGCUGCACGAGAAGACCCCUG |
| | | UGAGCGACAGAGUGACAAAGUGUUGCACCGAGAGCUUAG |
| | | UGAAUAGAAGACCUUGCUUCAGCGOCCUGGAAGUUGACGA |
| | | AACUUACGUGCCUAAGGAGUUCAACGCCGAGACUUUCACA |
| | | UUCCACGCCGACAUUUGCACUCUGAGCGAGAAGGAGAGAC |
| | | AGAUCAAGAAGCAGACCGCCCUCGUAGAGUUGGUCAAGCA |
| | | CAAGCCGAAGGCAACCAAGGAACAGCUUAAGGCCGUGAUG |
| | | GACGACUUCGCAGCCUUCGUCGAGAAGUGUUGUAAGGCCG |
| | | ACGACAAGGAGACUUGCUUCGCAGAGGAAGGCAAGAAGU |
| | | UGGUAGCCGCCUCUCAGGCUGCCUUGGGACUCGGUGGCGG |
| | | CAGCGCCCCUACCAGCAGCUCCACAAAGAAGACGCAGCUG |
| | | CAGCUGGAGCACCUGCUACUAGACUUACAGAUGAUCCUGA |
| | | ACGGCAUCAACAACUACAAGAACCCGAAGCUCACCAGAAU |
| | | GCUGACAUUCAAGUUCUACAUGCCUAAGAAGGCCACUGAG |
| | | CUGAAGCACCUUCAGUGCCUGGAGGAGGAACUUAAGCCAC |
| | | UGGAAGAGGCCCUGAACCUGGCCCCUAGUAAGAAUUUCCA |
| | | CCUCAGACCUCGCGACCUUAUUAGCGACAUCAACGUGAUC |
| | | GUGCUCGAAUUAAAGGGCUCCGAGACAACAUUCAUGUOCO |
| | | AAUACGCCGACGAGACAGCCACCAUCGUGGAGUUCCUGAA |
| | | CCGCUGGAUCACCUUCUGCCAGAGCAUCAUCUCUACUCUG |
| | | ACC |
| 12 | Leader- | MKWVTFISLLELFSSAYSRGVFRRDAHKSEVAHRFKDLGEE |
| | HSA-(G3S)- | NFKALVLIAFAQYLQQCPFEDHVKLVNEVTEFAKTCVADESAE |
| | (Human | NCDKSLHTLFGDKLCTVATLRETYGEMADCCAKQEPERNECF |
| | IL-2 V91K) | LQHKDDNPNLPRLVRPEVDVMCTAFHDNEETFLKKYLYEIAR |
| | | RHPYFYAPELLFFAKRYKAAFTECCQAADKAACLLPKLDELR |
| | | DEGKASSAKQRLKCASLQKFGERAFKAWAVARLSQRFPKAEF |
| | | AEVSKLVTDLTKVHTECCHGDLLECADDRADLAKYICENQDSI |
| | | SSKLKECCEKPLLEKSHCIAEVENDEMPADLPSLAADFVESKD |
| | | VCKNYAEAKDVFLGMFLYEYARRHPDYSVVLLLRLAKTYETT |
| | | LEKCCAAADPHECYAKVFDEFKPLVEEPQNLIKQNCELFEQLG |
| | | EYKFQNALLYRYTKKYPQVSTPTLVEVSRNLGKVGSKCCKHP |
| | | EAKRMPCAEDYLSVVLNQLCVLHEKTPVSDRVIKCCTESLVN |
| | | RRPCFSALEVDETYVPKEFNAETFTFHADICTLSEKERQIKKQT |
| | | ALVELVKHKPKATKEQLKAVMDDFAAFVEKCCKADDKETCF |
| | | AEEGKKLVAASQAALGLGGGSAPTSSSTKKTQLQLEHLLLDLQ |
| | | MILNGINNYKNPKLTRMLTFKFYMPKKATELKHLQCLEFELKP |
| | | LEEVLNLAQSKNFHLRPRDLISNINKIVLELKGSETTFMCEYAD |
| | | ETATIVEFLNRWITFCQSIISTLT |
| 13 | Leader- | MKWVTFISLLFLFSSAYSRGVFRRDAHKSEVAHRFKDLGEE |
| | HSA-(G3S)- | NFKALVLIAFAQYLQQCPFEDHVKLVNEVTEFAKTCVADESAE |
| | (Human | NCDKSLHTLEGDKLCTVATLRETYGEMADCCAKQEPERNECF |
| | IL-2 V69A, | LQHKDDNPNLPRLVRPEVDVMCTAFHDNEETFLKKYLYEIAR |
| | Q74P, | RHPYFYAPELLFFAKRYKAAFTECCQAADKAACLLPKLDELR |
| | V91K) | DEGKASSAKQRLKCASLQKFGERAFKAWAVARLSQRFPKAEF |
| | | AEVSKLVIDLIKVHTECCHGDLLECADDRADLAKYICENQDSI |
| | | SSKLKECCEKPLLEKSHCIAEVENDEMPADLPSLAADFVESKD |
| | | VCKNYAEAKDVFLGMFLYEYARRHPDYSVVLLLRLAKTYETT |
| | | LEKCCAAADPHECYAKVFDEFKPLVEEPQNLIKQNCELFEQLG |
| | | EYKFQNALLVRYTKKVPQVSTPTLVEVSRNLGKVGSKCCKHP |
| | | EAKRMPCARDYLSVVLNQLCVLHEKTPVSDRVTKCCTESLVN |
| | | RRPCFSALEVDETYVPKEFNAETFTFHADICTLSEKERQIKKQT |
| | | ALVELVKHKPKATKEQLKAVMDDFAAFVEKCCKADDKETCF |
| | | AEEGKKLVAASQAALGLGGGSAPTSSSTKKTQLQLEHLLLDLQ |
| | | MILNGINNYKNPKLTRMLTFKFYMPKKATELKHLQCLEEELKP |
| | | LEEALNLAPSKNFHLRPRDLISNINKIVLELKGSETTFMCEYAD |
| | | ETATIVEFLNRWITECQSIISTLT |

TABLE II
Exemplary CTLA-4 binder sequences and IL-2 CTLA-4 sequences

| SEQ ID | | |
|---|---|---|
| NO | Sequence information | Sequence |
| 17 | CTLA4_03 (Human, heavy | EVQLVQTGGGLSQFGESLRLSCAVSGFNVSNNYM |
| | chain only antibody binder | SWVRQAPGKGLEWVSIIYSGGGTHYADSVKGRFT |
| | to CTLA4, derived from | ISRDNSKNTLFLQMNSLRAEDTAVYYCARAVPVP |
| | HCAB mice) | HGTDIWGQGTMVTVSS |
| 18 | Leader-CTLA4.03- | MPLLLLLPLLWAGALAEVQLVQTGGGLSQFGESL |
| | (G4S)3-HSA-(G3S)- | RLSCAVSGENVSNNYMSWVRQAPGKGLEWVSIIYSGG |
| | (Human IL-2)-V5 tag | GTHYADSVKGRFTISRDNSKNTLFLQMNSLRAEDTAV |
| | | YYCARAVPVPHGTDIWGQGTMVTVSSGGGGSGGGG |
| | | SGGGGSAHKSEVAHRFKDLGEENFKALVLIAFAQ |
| | | YLQQCPFEDHVKLVNEVTEFAKTCVADESAENCD |
| | | KSLHTLFGDKLCTVATLRETYGEMADCCAKQEPE |
| | | RNECFLQHKDDNPNLPRLVRPEVDVMCTAFHDN |
| | | EETFLKKYLYEIARRHPYFYAPELLFFAKRYKAAF |
| | | TECCQAADKAACLLPKLDELRDEGKASSAKQRLK |
| | | CASLQKFGERAFKAWAVARLSQRFPKAEFAEVSK |
| | | LVTDLTKVHTECCHGDLLECADDRADLAKYICEN |
| | | QDSISSKLKECCEKPLLEKSHCIAEVENDEMPADL |
| | | PSLAADFVESKDVCKNYAEAKDVFLGMFLYEYA |
| | | RRHPDYSVVLLLRLAKTYETTLEKCCAAADPHEC |
| | | YAKVFDEFKPLVEEPQNLIKQNCELFEQLGEYKFQ |
| | | NALLVRYTKKVPQVSTPTLVEVSRNLGKVGSKCC |
| | | KHPEAKRMPCAEDYLSVVLNQLCVLHEKTPVSD |
| | | RVTKCCTESLVNRRPCFSALEVDETYVPKEFNAET |
| | | FTFHADICTLSEKERQIKKQTALVELVKHKPKATK |
| | | EQLKAVMDDFAAFVEKCCKADDKETCFAEEGKK |
| | | LVAASQAALGLGGGSAPTSSSTKKTQLQLEHLLL |
| | | DLQMILNGINNYKNPKLTRMLTFKFYMPKKATEL |
| | | KHLQCLEEELKPLEEVLNLAQSKNFHLRPRDLISNI |
| | | NVIVLELKGSETTFMCEYADETATIVEFLNRWITE |
| | | CQSIISTLTGKPIPNPLLGLDST |
| 21 | Leader-CTLA4.03- | AUGCCUCUGUUACUGCUGCUCCCACUUCUGUGGGCC |
| | (G4S)3-HSA-(G3S)- | GGCGCUCUGGCUGAGGUGCAGCUUGUGCAGACCGGCGG |
| | (Human IL-2)-V5 tag | AGGUCUGUCACAGUUCGGCGAAUCCCUAAGGCUCUCUU |
| | | GCGCCGUGUCUGGUUUCAACGUGAGCAACAACUAUAUG |
| | | AGUUGGGUUCGCCAGGCUCCAGGCAAGGGUCUGGAGUG |
| | | GGUGUCCAUUAUCUAUUCCGGCGGUGGCACACAUUACG |
| | | CCGACUCAGUCAAGGGUAGAUUCACUAUCAGUAGAGAC |
| | | AACUCAAAGAACACUCUUUUCCUACAAAUGAACAGCUU |
| | | GCGGGCAGAGGAUACGGCCGUGUAUUAUUGUGCCCGAG |
| | | CUGUCCCAGUGCCUCACGGAACAGAUAUUUGGGGACAA |
| | | GGUACAAUGGUGACCGUCUCUUCCGGCGGAGGAGGCUC |
| | | UGGAGGCGGAGGAUCUGGCGGAGGCGGCAGCGCCCAUA |
| | | AGAGCGAGGUAGCCCAUAGAUUCAAGGACCUUGGCGAA |
| | | GAGAACUUCAAGGCCUUAGUUCUCAUCGCCUUCGCCCA |
| | | AUACUUGCAGCAGUGUCCAUUCGAAGAUCACGUUAAGC |
| | | UUGUGAACGAGGUGACCGAAUUCGCUAAGACGUGCGUG |
| | | GCCGACGAGUCCGCUGAGAAUUGUGAUAAGUCACUGCA |
| | | CACUUUGUUCGGCGACAAGCUGUGCACCGUCGCCACCC |
| | | UGCGCGAAACAUACGGCGAAAUGGCGGACUGUUGUGCU |
| | | AAGCAGGAGCCAGAACGGAACGAGUGUUUCCUGCAGCA |
| | | UAAGGACGACAAUCCAAACCUCCCUAGGUUGGUGCGCC |
| | | CAGAAGUGGACGUCAUGUGUACCGCUUUCCACGAUAAC |
| | | GAGGAGACAUUCCUUAAGAAGUAUCUGUACGAGAUUGC |
| | | ACGGCGGCACCCUUACUUCUACGCCCCUGAGCUCCUGUU |
| | | CUUCGCAAAGCGGUAUAAGGCUGCAUUCACCGAGUGUU |
| | | GCCAGGCGGCAGACAAGGCCGCCUGCUUGCUCCCUAAG |
| | | CUUGACGAACUUAGGGACGAGGGAAAGGCUAGUAGCGC |
| | | CAAGCAGAGACUCAAGUGUGCAUCGCUACAGAAGUUCG |
| | | GAGAGAGAGCUUUCAAGGCGUGGGCUGUCGCGCGCCUG |
| | | UCCCAGAGGUUCCCAAAGGCCGAAUUCGCCGAGGUGAG |
| | | CAAGUUAGUGACAGAUCUCACCAAGGUGCACACUGAGU |
| | | GUUGCCACGGCGACUUAUUGGAGUGUGCAGACGAUCGG |
| | | GCUGACUUGGCAAAGUACAUCUGUGAGAACCAGGACUC |
| | | AAUUUCCAGUAAGCUGAAGGAGUGUUGCGAGAAGCCAC |
| | | UCUUGGAGAAGUCCCAUUGUAUAGCCGAAGUGGAGAAC |
| | | GACGAGAUGCCGGCUGACCUGCCGUCACUGGCAGCUGA |
| | | UUUCGUAGAGUCGAAGGACGUGUGUAAGAACUACGCUG |
| | | AAGCUAAGGACGUAUUCCUGGGAAUGUUCCUUUACGAG |
| | | UACGCCCGACGGCAUCCAGACUACUCAGUAGUGCUGCU |
| | | UCUGAGACUGGCCAAGACUUACGAGACUACCCUGGAGA |
| | | AGUGUUGUGCAGCGGCCGAUCCACACGAGUGUUACGCC |
| | | AAGGUGUUCGACGAAUUCAAGCCUCUGGUGGAGGAGCC |
| | | GCAGAACCUGAUCAAGCAGAAUUGCGAACUGUUCGAGC |
| | | AGUUGGGAGAAUAUAAGUUCCAGAACGCACUGCUAGUG |
| | | CGGUAUACCAAGAAGGUCCCACAGGUAUCCACUCCGAC |
| | | ACUGGUGGAAGUGUCCAGGAACCUCGGCAAGGUGGGAA |
| | | GCAAGUGCUGUAAGCACCCGGAGGCUAAGCGGAUGCCG |
| | | UGCGCCGAAGACUACCUCAGCGUGGUCCUUAAUCAGCU |
| | | GUGCGUGCUGCACGAGAAGACCCCAGUAAGUGACCGGG |
| | | UUACGAAGUGCUGUACUGAAUCACUUGUGAACAGAAGA |
| | | CCUUGUUUCUCCGCCCUCGAGGUGGACGAAACGUACGU |
| | | UCCGAAGGAAUUCAACGCCGAAACAUUCACCUUCCACG |
| | | CUGACAUUUGUACGCUUUCCGAGAAGGAGAGACAAAUC |
| | | AAGAAGCAGACUGCCCUGGUGGAAUUAGUGAAGCACAA |
| | | GCCGAAGGCCACAAAGGAGCAGCUGAAGGCAGUUAUGG |
| | | ACGACUUCGCUGCAUUCGUAGAGAAGUGCUGCAAGGCA |
| | | GACGACAAGGAAACUUGCUUCGCAGAGGAGGGAAAGAA |
| | | GCUCGUUGCUGCGUCUCAGGCGGCUCUGGGACUGGGAG |
| | | GAGGAUCCGCUCCUACAAGCAGUUCCACAAAGAAGACC |
| | | CAGCUCCAGCUUGAACAUCUGCUUCUCGACCUGCAAAU |
| | | GAUCCUCAACGGUAUCAAUAAUUACAAGAAUCCUAAGU |
| | | UGACUCGCAUGCUGACUUUCAAGUUCUAUAUGCCAAAG |
| | | AAGGCCACUGAGUUGAAGCACCUGCAGUGCCUCGAGGA |
| | | AGAACUGAAGCCUCUGGAGGAAGUCUUAAACCUGGCAC |
| | | AAAGCAAGAACUUCCAUCUGAGGCCUAGGGACCUCAUU |
| | | UCCAAUAUCAACGUUAUUGUGCUGGAGCUCAAGGGUAG |
| | | CGAGACGACAUUCAUGUGCGAGUACGCAGACGAGACAG |
| | | CGACUAUCGUCGAGUUCCUAAAUAGGUGGAUAACGUUC |
| | | UGCCAGUCAAUCAUCUCCACUCUGACCGGCAAGCCGAU |
| | | CCCUAACCCGUUGCUUGGAUUGGAUUCUACA |
| 19 | Leader-CTLA4.03- | MPLLLLLPLLWAGALAEVQLVQTGGGLSQFGES |
| | (G4S)3-HSA-(G3S)- | LRLSCAVSGFNVSNNYMSWVRQAPGKGLEWVSIIYSG |
| | (Human IL-2 D20T)-V5 | GGTHYADSVKGRFTISRDNSKNTLFLQMNSLRAEDT |
| | tag | AVYYCARAVPVPHGTDIWGQGTMVTVSSGGGGSGG |
| | | GGSGGGGSAHKSEVAHRFKDLGEENFKALVLIAF |
| | | AQYLQQCPFEDHVKLVNEVTEFAKTCVADESAEN |
| | | CDKSLHTLFGDKLCTVATLRETYGEMADCCAKQ |
| | | EPERNECFLQHKDDNPNLPRLVRPEVDVMCTAFH |
| | | DNEETFLKKYLYEIARRHPYFYAPELLFFAKRYKA |
| | | AFTECCQAADKAACLLPKLDELRDEGKASSAKQR |
| | | LKCASLQKFGERAFKAWAVARLSQRFPKAEFAEV |
| | | SKLVTDLTKVHTECCHGDLLECADDRADLAKYIC |
| | | ENQDSISSKLKECCEKPLLEKSHCIAEVENDEMPA |
| | | DLPSLAADFVESKDVCKNYAEAKDVFLGMFLYE |
| | | YARRHPDYSVVLLLRLAKTYETTLEKCCAAADPH |
| | | ECYAKVFDEFKPLVEEPQNLIKQNCELFEQLGEYK |
| | | FQNALLVRYTKKVPQVSTPTLVEVSRNLGKVGSK |
| | | CCKHPEAKRMPCAEDYLSVVLNQLCVLHEKTPVS |
| | | DRVTKCCTESLVNRRPCFSALEVDETYVPKEFNA |
| | | ETFTFHADICTLSEKERQIKKQTALVELVKHKPKA |
| | | TKEQLKAVMDDFAAFVEKCCKADDKETCFAEEG |
| | | KKLVAASQAALGLGGGSAPTSSSTKKTQLQLEHL |
| | | LLTLQMILNGINNYKNPKLTRMLTFKFYMPKKAT |
| | | ELKHLQCLEEELKPLEEVLNLAQSKNFHLRPRDLI |
| | | SNINVIVLELKGSETTFMCEYADETATIVEFLNRWI |
| | | TFCQSIISTLTGKPIPNPLLGLDST |
| 22 | Leader-CTLA4.03- | AUGCCUCUGCUGCUCUUACUGCCGCUGCUUUGGGCA |
| | (G4S)3-HSA-(G3S)- | GGAGCUCUGGCCGAGGUCCAGCUAGUGCAGACAGGCGG |
| | (Human IL-2 D20T)-V5 | UGGACUCAGCCAGUUCGGUGAGUCACUGCGUCUAUCCU |
| | tag | GCGCGGUUAGUGGCUUCAACGUGUCCAACAAUUAUAUG |
| | | AGUUGGGUGAGACAAGCACCUGGCAAGGGCCUUGAGUG |
| | | GGUGUCCAUUAUAUAUAGCGGUGGCGGCACCCACUACG |
| | | CUGACUCGGUGAAGGGUCGAUUCACAAUUAGCAGAGAC |
| | | AAUUCCAAGAAUACUCUGUUCCUGCAGAUGAACUCUCU |
| | | CAGGGCGGAGGACACCGCCGUGUACUACUGUGCCCGUG |
| | | CGGUGCCGGUCCCUCACGGUACAGACAUCUGGGGCCAG |
| | | GGUACCAUGGUCACCGUGUCAUCCGGAGGAGGCGGUUC |
| | | UGGUGGAGGAGGAAGUGGAGGCGGCGGCAGUGCACAUA |
| | | AGAGCGAGGUGGCCCAUAGAUUCAAGGAUUUAGGAGAG |
| | | GAGAACUUCAAGGCACUGGUCUUGAUCGCCUUCGCACA |
| | | AUAUCUUCAGCAGUGUCCGUUCGAAGAUCACGUGAAGC |
| | | UCGUCAACGAGGUGACUGAGUUCGCCAAGACCUGCGUG |
| | | GCAGACGAGUCGGCUGAGAAUUGUGAUAAGAGUCUGCA |
| | | CACCCUCUUCGGAGAUAAGCUGUGUACCGUGGCCACUC |
| | | UGAGGGAGACAUACGGCGAGAUGGCUGACUGUUGUGCA |
| | | AAGCAGGAGCCAGAGAGGAACGAGUGUUUCUUGCAGCA |
| | | UAAGGACGACAACCCAAAUCUGCCUCGUCUGGUUAGAC |
| | | CUGAAGUGGACGUAAUGUGCACCGCUUUCCACGACAAC |
| | | GAGGAGACGUUCCUGAAGAAGUAUCUGUACGAGAUUGC |
| | | UAGAAGGCACCCAUACUUCUACGCACCAGAGCUGUUGU |
| | | UCUUCGCUAAGAGAUACAAGGCCGCUUUCACCGAGUGC |
| | | UGCCAAGCAGCAGACAAGGCUGCUUGCCUGCUCCCUAA |
| | | GCUGGACGAGCUGAGAGACGAGGGUAAGGCUUCAUCAG |
| | | CCAAGCAAAGAUUGAAGUGUGCAUCACUGCAGAAGUUC |
| | | GGCGAAAGAGCAUUCAAGGCUUGGGCCGUCGCCAGGCU |
| | | GUCUCAGAGGUUCCCGAAGGCCGAGUUCGCUGAAGUGU |
| | | CUAAGUUAGUGACCGAUCUAACAAAGGUGCACACAGAG |
| | | UGUUGCCACGGAGAUCUGCUUGAGUGCGCCGACGAUCG |
| | | AGCCGAUCUUGCAAAGUACAUCUGUGAGAACCAGGAUU |
| | | CCAUCUCCUCUAAGUUGAAGGAGUGCUGUGAGAAGCCA |
| | | CUUCUCGAGAAGUCUCACUGCAUCGCAGAAGUCGAGAA |
| | | CGACGAAAUGCCAGCUGACCUGCCGAGCUUGGCCGCAG |
| | | AUUUCGUCGAGAGUAAGGACGUUUGCAAGAAUUACGCU |
| | | GAGGCUAAGGACGUCUUCCUGGGCAUGUUCUUGUACGA |
| | | AUACGCCAGGAGGCACCCGGAUUACUCAGUGGUACUUU |
| | | UACUGCGACUGGCUAAGACCUACGAAACGACACUGGAG |
| | | AAGUGCUGCGCAGCCGCCGAUCCUCACGAGUGCUACGC |
| | | UAAGGUUUUCGACGAGUUCAAGCCGCUCGUGGAAGAAC |
| | | CUCAGAACUUAAUCAAGCAGAAUUGUGAGCUAUUCGAA |
| | | CAGCUCGGAGAGUAUAAGUUCCAGAACGCCCUCCUGGU |
| | | GCGGUACACAAAGAAGGUUCCACAGGUCAGCACCCCGA |
| | | CCCUCGUUGAGGUGAGCCGCAACCUCGGCAAGGUUGGC |
| | | UCUAAGUGUUGUAAGCAUCCGGAAGCGAAGCGCAUGCC |
| | | UUGUGCAGAAGAUUAUCUUUCAGUUGUGUUGAACCAGC |
| | | UCUGUGUCCUGCACGAGAAGACCCCAGUUUCCGACCGC |
| | | GUUACCAAGUGUUGCACAGAAAGCUUGGUCAACCGGAG |
| | | GCCUUGCUUCAGCGCAUUGGAGGUAGACGAAACCUACG |
| | | UCCCUAAGGAAUUCAACGCCGAAACAUUCACGUUCCAC |
| | | GCGGACAUUUGCACACUCUCCGAGAAGGAACGCCAGAU |
| | | UAAGAAGCAGACUGCCUUGGUUGAGCUGGUAAAGCACA |
| | | AGCCUAAGGCGACUAAGGAGCAGCUAAAGGCAGUUAUG |
| | | GACGACUUCGCUGCAUUCGUUGAGAAGUGCUGUAAGGC |
| | | CGACGAUAAGGAAACCUGCUUCGCCGAGGAAGGAAAGA |
| | | AGCUUGUUGCCGCUAGCCAGGCCGCACUGGGAUUGGGU |
| | | GGAGGCAGCGCACCAACUUCCUCUAGCACCAAGAAGAC |
| | | UCAAUUACAACUGGAACAUCUCUUACUAACACUCCAAA |
| | | UGAUACUGAACGGCAUAAACAACUAUAAGAACCCGAAG |
| | | UUGACCAGAAUGUUAACCUUCAAGUUCUACAUGCCUAA |
| | | GAAGGCUACGGAACUGAAGCACCUUCAGUGCCUAGAGG |
| | | AGGAGCUCAAGCCUCUCGAGGAGGUUCUGAACCUCGCA |
| | | CAGAGUAAGAAUUUCCACCUGCGCCCAAGGGACCUGAU |
| | | UUCCAAUAUUAACGUGAUCGUCCUUGAACUUAAGGGCA |
| | | GCGAAACCACUUUCAUGUGCGAAUACGCGGACGAAACU |
| | | GCUACCAUAGUUGAGUUCCUUAACCGCUGGAUCACAUU |
| | | CUGCCAGUCAAUCAUCUCUACAUUGACCGGAAAGCCGA |
| | | UUCCGAACCCACUCCUGGGACUUGAUAGCACA |
| 20 | Leader-CTLA4.03- | MPLLLLLPLLWAGALAEVQLVQTGGGLSQFGESL |
| | (G4S)3-HSA-(G3S)- | RLSCAVSGFNVSNNYMSWVRQAPGKGLEWVSIIYSGG |
| | (Human IL-2 N88D)-V5 | GTHYADSVKGRFTISRDNSKNTLFLQMNSLRAEDTAV |
| | tag | YYCARAVPVPHGTDIWGQGTMVTVSSGGGGSGGGG |
| | | SGGGGSAHKSEVAHRFKDLGEENFKALVLIAFAQ |
| | | YLQQCPFEDHVKLVNEVTEFAKTCVADESAENCD |
| | | KSLHTLFGDKLCTVATLRETYGEMADCCAKQEPE |
| | | RNECFLQHKDDNPNLPRLVRPEVDVMCTAFHDN |
| | | EETFLKKYLYEIARRHPYFYAPELLFFAKRYKAAF |
| | | TECCQAADKAACLLPKLDELRDEGKASSAKQRLK |
| | | CASLQKFGERAFKAWAVARLSQRFPKAEFAEVSK |
| | | LVTDLTKVHTECCHGDLLECADDRADLAKYICEN |
| | | QDSISSKLKECCEKPLLEKSHCIAEVENDEMPADL |
| | | PSLAADFVESKDVCKNYAEAKDVFLGMFLYEYA |
| | | RRHPDYSVVLLLRLAKTYETTLEKCCAAADPHEC |
| | | YAKVFDEFKPLVEEPQNLIKQNCELFEQLGEYKFQ |
| | | NALLVRYTKKVPQVSTPTLVEVSRNLGKVGSKCC |
| | | KHPEAKRMPCAEDYLSVVLNQLCVLHEKTPVSD |
| | | RVTKCCTESLVNRRPCFSALEVDETYVPKEFNAET |
| | | FTFHADICTLSEKERQIKKQTALVELVKHKPKATK |
| | | EQLKAVMDDFAAFVEKCCKADDKETCFAEEGKK |
| | | LVAASQAALGLGGGSAPTSSSTKKTQLQLEHLLL |
| | | DLQMILNGINNYKNPKLTRMLTFKFYMPKKATEL |
| | | KHLQCLEEELKPLEEVLNLAQSKNFHLRPRDLISDI |
| | | NVIVLELKGSETTFMCEYADETATIVEFLNRWITF |
| | | CQSIISTLIGKPIPNPLLGLDS |
| 23 | Leader-CTLA4.03- | AUGCCUCUGCUCCUGCUUCUUCCUCUGCUCUGGGCU |
| | (G4S)3-HSA-(G3S)- | GGCGCCCUAGCCGAGGUGCAGCUGGUGCAGACCGGCGG |
| | (Human IL-2 N88D)-V5 | CGGCCUGAGCCAGUUCGGCGAGAGCCUGAGACUGAGCU |
| | tag | GCGCCGUGAGCGGCUUCAACGUGAGCAACAACUACAUG |
| | | AGCUGGGUGAGACAGGCCCCUGGCAAGGGCCUGGAGUG |
| | | GGUGAGCAUCAUCUACAGUGGCGGAGGUACCCACUACG |
| | | CCGACAGCGUGAAGGGCAGAUUCACCAUCAGCAGAGAC |
| | | AACAGCAAGAACACCCUGUUCCUGCAGAUGAACAGCCU |
| | | CAGGGCCGAGGACACCGCCGUGUACUACUGCGCCAGAG |
| | | CCGUGCCUGUGCCUCACGGCACCGACAUCUGGGGCCAG |
| | | GGCACCAUGGUGACCGUGAGUAGCGGUGGAGGCGGAAG |
| | | CGGAGGCGGUGGCUCUGGAGGUGGCGGCAGCGCCCACA |
| | | AGAGCGAGGUGGCCCACAGAUUCAAGGACCUGGGCGAG |
| | | GAGAACUUCAAGGCCCUGGUGCUGAUCGCCUUCGCCCA |
| | | GUACCUGCAGCAGUGCCCUUUCGAGGACCACGUGAAGC |
| | | UGGUGAACGAGGUGACCGAGUUCGCCAAGACCUGCGUG |
| | | GCCGACGAGAGCGCCGAGAACUGCGACAAGAGCCUGCA |
| | | CACACUCUUCGGCGACAAGCUGUGCACCGUGGCCACCCU |
| | | GAGAGAGACUUACGGCGAGAUGGCCGACUGCUGCGCCA |
| | | AGCAGGAACCAGAGAGGAACGAGUGCUUCCUUCAGCAC |
| | | AAGGACGACAACCCUAACCUGCCUAGACUGGUUCGGCC |
| | | UGAGGUGGACGUGAUGUGCACUGCCUUCCACGACAACG |
| | | AGGAGACGUUCCUGAAGAAGUACCUGUACGAGAUCGCC |
| | | AGAAGACACCCUUACUUCUACGCCCCUGAGCUUCUGUU |
| | | CUUCGCAAAGAGAUACAAGGCCGCCUUCACCGAGUGCU |
| | | GCCAGGCCGCCGACAAGGCUGCCUGCCUCCUGCCAAAGC |
| | | UGGACGAGCUGAGAGACGAGGGCAAGGCCAGCUCUGCG |
| | | AAGCAGAGGCUGAAGUGCGCCAGCCUGCAGAAGUUCGG |
| | | AGAAAGAGCUUUCAAGGCCUGGGCCGUGGCCCGCUUGU |
| | | CUCAAAGAUUCCCUAAGGCGGAGUUCGCAGAAGUGAGC |
| | | AAGCUGGUCACCGACCUGACCAAGGUGCAUACUGAGUG |
| | | UUGCCACGGCGACCUGCUGGAGUGCGCCGACGACAGAG |
| | | CCGACCUGGCCAAGUACAUCUGCGAGAACCAGGACUCU |
| | | AUUAGUAGCAAGUUGAAGGAGUGUUGCGAGAAGCCGCU |
| | | ACUCGAGAAGAGUCACUGCAUUGCGGAGGUUGAGAACG |
| | | ACGAGAUGCCUGCCGACCUCCCAAGCCUGGCAGCCGAU |
| | | UUCGUGGAGAGCAAGGACGUGUGCAAGAAUUACGCUGA |
| | | GGCUAAGGACGUUUUCCUGGGCAUGUUCUUAUACGAAU |
| | | ACGCUAGACGGCACCCAGACUACAGCGUGGUCCUCCUCC |
| | | UGCGGCUUGCAAAGACGUACGAAACUACCCUGGAGAAG |
| | | UGCUGCGCGGCUGCUGACCCUCACGAGUGCUACGCCAA |
| | | GGUGUUCGACGAGUUCAAGCCUCUCGUGGAGGAGCCUC |
| | | AGAACCUGAUCAAGCAGAAUUGCGAGCUGUUCGAGCAG |
| | | UUAGGUGAGUACAAGUUCCAGAACGCCCUGCUUGUGCG |
| | | CUACACCAAGAAGGUGCCUCAGGUUUCCACCCCUACCCU |
| | | GGUAGAGGUGUCCAGAAACCUGGGCAAGGUGGGCAGCA |
| | | AGUGUUGCAAGCAUCCAGAAGCUAAGAGAAUGCCUUGC |
| | | GCCGAAGAUUACCUGUCUGUCGUGCUGAACCAGCUGUG |
| | | CGUGCUGCACGAGAAGACCCCUGUGAGCGACAGAGUGA |
| | | CCAAGUGCUGUACAGAAUCCCUAGUAAAUAGAAGACCU |
| | | UGCUUCUCCGCCCUAGAGGUGGACGAAACCUACGUGCC |
| | | UAAGGAGUUCAACGCCGAGACUUUCACGUUCCACGCGG |
| | | AUAUUUGUACACUCAGCGAGAAGGAGAGACAGAUCAAG |
| | | AAGCAGACCGCACUUGUGGAGCUCGUCAAGCACAAGCC |
| | | AAAGGCCACCAAGGAGCAACUCAAGGCCGUGAUGGACG |
| | | ACUUCGCAGCCUUCGUCGAGAAGUGUUGUAAGGCUGAC |
| | | GAUAAGGAAACCUGCUUCGCUGAGGAAGGCAAGAAGCU |
| | | UGUGGCCGCCAGUCAGGCUGCUCUUGGUCUUGGCGGCG |
| | | GAUCAGCACCUACCAGCAGCUCUACAAAGAAGACGCAG |
| | | CUGCAACUGGAGCACUUGCUGCUUGAUCUGCAAAUGAU |
| | | CCUGAACGGCAUCAACAAUUACAAGAACCCUAAGCUGA |
| | | CCAGAAUGCUGACCUUCAAGUUCUACAUGCCUAAGAAG |
| | | GCCACAGAGCUGAAGCAUUUACAGUGCCUGGAGGAGGA |
| | | GCUUAAGCCACUUGAAGAGGUGCUCAAUCUGGCCCAGU |
| | | CUAAGAACUUCCACCUCAGGCCUCGCGAUCUGAUUAGC |
| | | GACAUCAACGUAAUCGUGCUGGAACUUAAGGGAUCUGA |
| | | AACUACGUUCAUGUGCGAGUACGCCGACGAAACAGCCA |
| | | CCAUCGUGGAGUUCCUGAACCGAUGGAUCACCUUCUGC |
| | | CAGAGUAUUAUUUCAACCCUCACCGGCAAGCCUAUCCC |
| | | UAACCCACUGCUCGGACUCGACAGCACC |

TABLE III
Exemplary IL-2 construct sequences
Note: ″G5″ indicates that all uracils (U) in the mRNA are replaced by N1-methylpseudouracils.

| | mRNA | ORF Sequence | ORF Sequence | 5′ UTR | 3′ UTR | Construct |
|---|---|---|---|---|---|---|
| | Name | (Amino Acid) | (Nucleotide) | Sequence | Sequence | Sequence |
| SEQ | | | | | | |
| ID | | | | | | |
| NO: | | 11 | 25 | 26 | 27 | 28 |
| | HSA- | MKWVTFISLLFLFSSAYS | AUGAAGUGGGUGACC | GGGAAA | UGAUAA | SEQ ID |
| | hsIL2.V69A. | RGVFRRDAHKSEVAHR | UUCAUCAGCCUGCUG | UAAGAG | UAGGCU | NO: 28 |
| | Q74P.N88D | FKDLGEENFKALVLIAFA | UUCCUGUUCAGCAGC | AGAAAA | GGAGCC | consists |
| | G5 | QYLQQCPFEDHVKLVN | GCCUACAGCAGAGGC | GAAGAG | UCGGUG | from 5′ to |
| | Cap: C1 | EVTEFAKTCVADESAEN | GUGUUCAGAAGAGAC | UAAGAA | GCCUAGC | 3′ end: 5′ |
| | Poly A | CDKSLHTLFGDKLCTVA | GCCCACAAGAGCGAG | GAAAUA | UUCUUG | UTR of |
| | tail: | TLRETYGEMADCCAKQ | GUGGCCCACAGAUUC | UAAGACC | CCCCUUG | SEQ ID |
| | 100 nt | EPERNECFLQHKDDNP | AAGGACCUGGGCGAG | CCGGCGC | GGCCUCC | NO: 26, |
| | (SEQ ID NO: | NLPRLVRPEVDVMCTA | GAGAACUUCAAGGCC | CGCCACC | CCCCAGC | ORF |
| | 29) | FHDNEETFLKKYLYEIAR | CUGGUGCUGAUCGCC | | CCCUCCU | sequence |
| | | RHPYFYAPELLFFAKRYK | UUCGCCCAGUACCUG | | CCCCUUC | of SEQ |
| | | AAFTECCQAADKAACL | CAGCAGUGCCCUUUC | | CUGCACC | ID NO: |
| | | LPKLDELRDEGKASSAK | GAGGACCACGUGAAG | | CGUACCC | 25, 3′ |
| | | QRLKCASLQKFGERAFK | CUGGUGAACGAGGUG | | CCGUGG | UTR of |
| | | AWAVARLSQRFPKAEF | ACCGAGUUCGCCAAG | | UCUUUG | SEQ ID |
| | | AEVSKLVTDLTKVHTEC | ACCUGCGUGGCCGAC | | AAUAAA | NO: 27 |
| | | CHGDLLECADDRADLA | GAGAGCGCCGAGAAC | | GUCUGA | and Poly |
| | | KYICENQDSISSKLKECC | UGCGACAAGAGCCUG | | GUGGGC | A tail of |
| | | EKPLLEKSHCIAEVENDE | CACACCCUGUUCGGC | | GGC | SEQ ID |
| | | MPADLPSLAADFVESK | GACAAGCUGUGCACC | | | NO: 29 |
| | | DVCKNYAEAKDVFLGM | GUGGCCACCCUGAGA | | | |
| | | FLYEYARRHPDYSVVLLL | GAAACUUACGGCGAG | | | |
| | | RLAKTYETTLEKCCAAA | AUGGCCGACUGCUGC | | | |
| | | DPHECYAKVFDEFKPLV | GCCAAGCAGGAGCCA | | | |
| | | EEPQNLIKQNCELFEQL | GAGCGGAACGAGUGC | | | |
| | | GEYKFQNALLVRYTKKV | UUCCUCCAGCACAAG | | | |
| | | PQVSTPTLVEVSRNLGK | GACGACAACCCUAACC | | | |
| | | VGSKCCKHPEAKRMPC | UGCCUAGACUGGUAA | | | |
| | | AEDYLSVVLNQLCVLHE | GGCCUGAGGUGGACG | | | |
| | | KTPVSDRVTKCCTESLV | UGAUGUGUACCGCCU | | | |
| | | NRRPCFSALEVDETYVP | UCCACGACAACGAGG | | | |
| | | KEFNAETFTFHADICTLS | AGACAUUCCUGAAGA | | | |
| | | EKERQIKKQTALVELVK | AGUACCUGUACGAGA | | | |
| | | HKPKATKEQLKAVMDD | UCGCCAGAAGACACC | | | |
| | | FAAFVEKCCKADDKETC | CUUACUUCUACGCCC | | | |
| | | FAEEGKKLVAASQAAL | CUGAGUUGCUGUUC | | | |
| | | GLGGGSAPTSSSTKKTQ | UUCGCGAAGAGAUAC | | | |
| | | LQLEHLLLDLQMILNGI | AAGGCCGCCUUCACC | | | |
| | | NNYKNPKLTRMLTFKFY | GAGUGCUGCCAGGCC | | | |
| | | MPKKATELKHLQCLEEE | GCCGAUAAGGCCGCG | | | |
| | | LKPLEEALNLAPSKNFHL | UGCCUGCUGCCUAAG | | | |
| | | RPRDLISDINVIVLELKG | CUGGACGAGCUGAGA | | | |
| | | SETTFMCEYADETATIV | GACGAGGGCAAGGCA | | | |
| | | EFLNRWITFCQSIISTLT | UCCAGCGCUAAGCAG | | | |
| | | | AGACUGAAGUGCGCC | | | |
| | | | AGCCUGCAGAAGUUC | | | |
| | | | GGAGAGAGAGCUUUC | | | |
| | | | AAGGCGUGGGCAGUG | | | |
| | | | GCUAGAUUGAGCCAA | | | |
| | | | AGAUUCCCUAAGGCA | | | |
| | | | GAAUUCGCUGAGGUG | | | |
| | | | AGCAAGCUCGUGACU | | | |
| | | | GACCUGACCAAGGUG | | | |
| | | | CAUACAGAGUGCUGU | | | |
| | | | CACGGCGACCUGCUG | | | |
| | | | GAGUGCGCCGACGAC | | | |
| | | | AGAGCCGACCUGGCC | | | |
| | | | AAGUACAUCUGCGAG | | | |
| | | | AACCAGGACAGCAUC | | | |
| | | | AGCAGCAAGCUGAAG | | | |
| | | | GAGUGUUGUGAGAA | | | |
| | | | GCCUCUAUUGGAGAA | | | |
| | | | GAGUCACUGCAUUGC | | | |
| | | | CGAGGUGGAGAACGA | | | |
| | | | CGAGAUGCCUGCGGA | | | |
| | | | UCUGCCAAGCUUGGC | | | |
| | | | GGCCGACUUCGUGGA | | | |
| | | | GAGCAAGGACGUGUG | | | |
| | | | CAAGAACUACGCCGA | | | |
| | | | GGCCAAGGACGUUUU | | | |
| | | | CCUGGGCAUGUUCCU | | | |
| | | | CUACGAGUACGCACG | | | |
| | | | CAGACAUCCAGACUA | | | |
| | | | CAGCGUGGUGCUGCU | | | |
| | | | GCUGAGACUGGCUAA | | | |
| | | | GACAUACGAAACUAC | | | |
| | | | CCUGGAGAAGUGCUG | | | |
| | | | CGCAGCGGCGGACCC | | | |
| | | | UCACGAGUGUUACGC | | | |
| | | | CAAGGUGUUCGACGA | | | |
| | | | GUUCAAGCCUCUGGU | | | |
| | | | GGAGGAGCCUCAGAA | | | |
| | | | CCUGAUCAAGCAGAA | | | |
| | | | CUGUGAGCUGUUCGA | | | |
| | | | GCAGCUCGGCGAGUA | | | |
| | | | CAAGUUCCAGAACGC | | | |
| | | | CCUGUUGGUCCGCUA | | | |
| | | | CACCAAGAAGGUGCC | | | |
| | | | UCAAGUAAGUACCCC | | | |
| | | | UACCCUGGUAGAGGU | | | |
| | | | UAGUAGAAACCUGGG | | | |
| | | | CAAGGUGGGCAGCAA | | | |
| | | | GUGCUGUAAGCACCC | | | |
| | | | AGAAGCUAAGAGGAU | | | |
| | | | GCCUUGCGCCGAGGA | | | |
| | | | CUACCUGUCCGUUGU | | | |
| | | | GCUGAACCAGCUGUG | | | |
| | | | CGUGCUGCACGAGAA | | | |
| | | | GACCCCUGUGAGCGA | | | |
| | | | CAGAGUGACAAAGUG | | | |
| | | | UUGCACCGAGAGCUU | | | |
| | | | AGUGAAUAGAAGACC | | | |
| | | | UUGCUUCAGCGCCCU | | | |
| | | | GGAAGUUGACGAAAC | | | |
| | | | UUACGUGCCUAAGGA | | | |
| | | | GUUCAACGCCGAGAC | | | |
| | | | UUUCACAUUCCACGC | | | |
| | | | CGACAUCUGUACUCU | | | |
| | | | GAGCGAGAAGGAGAG | | | |
| | | | ACAGAUCAAGAAGCA | | | |
| | | | GACCGCCCUCGUAGA | | | |
| | | | GUUGGUCAAGCACAA | | | |
| | | | GCCGAAGGCAACCAA | | | |
| | | | GGAACAGCUUAAGGC | | | |
| | | | CGUGAUGGACGACUU | | | |
| | | | CGCAGCCUUCGUCGA | | | |
| | | | GAAGUGUUGUAAGG | | | |
| | | | CCGACGACAAGGAGA | | | |
| | | | CUUGCUUCGCAGAGG | | | |
| | | | AAGGCAAGAAGUUGG | | | |
| | | | UAGCCGCCUCUCAGG | | | |
| | | | CUGCCUUGGGACUCG | | | |
| | | | GUGGCGGCAGCGCCC | | | |
| | | | CUACCAGCAGCUCCAC | | | |
| | | | AAAGAAGACGCAGCU | | | |
| | | | GCAGCUGGAGCACCU | | | |
| | | | GCUACUAGACUUACA | | | |
| | | | GAUGAUCCUGAACGG | | | |
| | | | CAUCAACAACUACAA | | | |
| | | | GAACCCGAAGCUCAC | | | |
| | | | CAGAAUGCUGACAUU | | | |
| | | | CAAGUUCUACAUGCC | | | |
| | | | UAAGAAGGCCACUGA | | | |
| | | | GCUGAAGCACCUUCA | | | |
| | | | GUGCCUGGAGGAGGA | | | |
| | | | ACUUAAGCCACUGGA | | | |
| | | | AGAGGCCCUGAACCU | | | |
| | | | GGCCCCUAGUAAGAA | | | |
| | | | UUUCCACCUCAGACC | | | |
| | | | UCGCGACCUUAUUAG | | | |
| | | | CGACAUCAACGUGAU | | | |
| | | | CGUGCUCGAAUUAAA | | | |
| | | | GGGCUCCGAGACAAC | | | |
| | | | AUUCAUGUGCGAAUA | | | |
| | | | CGCCGACGAGACAGC | | | |
| | | | CACCAUCGUGGAGUU | | | |
| | | | CCUGAACCGCUGGAU | | | |
| | | | CACCUUCUGCCAGAG | | | |
| | | | CAUCAUCUCUACUCU | | | |
| | | | GACC | | | |
| SEQ | | | | | | |
| ID | | | | | | |
| NO: | | 11 | 36 | 26 | 27 | 37 |
| | HSA- | MKWVTFISLLFLFSSAYS | AUGAAGUGGGUGA | GGGAAA | UGAUAA | SEQ ID |
| | hsIL2.V69A. | RGVFRRDAHKSEVAHR | CCUUCAUCAGCCUG | UAAGAG | UAGGCU | NO: 37 |
| | Q74P.N88D | FKDLGEENFKALVLIAFA | CUGUUCCUGUUCA | AGAAAA | GGAGCC | consists |
| | G5 | QYLQQCPFEDHVKLVN | GCAGCGCCUACAGC | GAAGAG | UCGGUG | from 5′ to |
| | Cap: C1 | EVTEFAKTCVADESAEN | AGAGGCGUGUUCA | UAAGAA | GCCUAGC | 3′ end: 5′ |
| | Poly A | CDKSLHTLFGDKLCTVA | GAAGAGACGCCCAC | GAAAUA | UUCUUG | UTR of |
| | tail: | TLRETYGEMADCCAKQ | AAGAGCGAGGUGG | UAAGACC | CCCCUUG | SEQ ID |
| | 100 nt | EPERNECFLQHKDDNP | CCCACAGAUUCAAG | CCGGCGC | GGCCUCC | NO: 26, |
| | (SEQ ID NO: | NLPRLVRPEVDVMCTA | GACCUGGGCGAGGA | CGCCACC | CCCCAGC | ORF |
| | 29) | FHDNEETFLKKYLYEIAR | GAACUUCAAGGCCC | | CCCUCCU | sequence |
| | | RHPYFYAPELLFFAKRYK | UGGUGCUGAUCGC | | CCCCUUC | of SEQ |
| | | AAFTECCQAADKAACL | CUUCGCCCAGUACC | | CUGCACC | ID NO: |
| | | LPKLDELRDEGKASSAK | UGCAGCAGUGCCCU | | CGUACCC | 36, 3′ |
| | | QRLKCASLQKFGERAFK | UUCGAGGACCACGU | | CCGUGG | UTR of |
| | | AWAVARLSQRFPKAEF | GAAGCUGGUGAAC | | UCUUUG | SEQ ID |
| | | AEVSKLVTDLTKVHTEC | GAGGUGACCGAGU | | AAUAAA | NO: 27 |
| | | CHGDLLECADDRADLA | UCGCCAAGACCUGC | | GUCUGA | and Poly |
| | | KYICENQDSISSKLKECC | GUGGCCGACGAGAG | | GUGGGC | A tail of |
| | | EKPLLEKSHCIAEVENDE | CGCCGAGAACUGCG | | GGC | SEQ ID |
| | | MPADLPSLAADFVESK | ACAAGAGCCUGCAC | | | NO: 29 |
| | | DVCKNYAEAKDVFLGM | ACCCUGUUCGGCGA | | | |
| | | FLYEYARRHPDYSVVLLL | CAAGCUGUGCACCG | | | |
| | | RLAKTYETTLEKCCAAA | UGGCCACCCUGAGA | | | |
| | | DPHECYAKVFDEFKPLV | GAAACUUACGGCGA | | | |
| | | EEPQNLIKQNCELFEQL | GAUGGCCGACUGCU | | | |
| | | GEYKFQNALLVRYTKKV | GCGCCAAGCAGGAG | | | |
| | | PQVSTPTLVEVSRNLGK | CCAGAGCGGAACGA | | | |
| | | VGSKCCKHPEAKRMPC | GUGCUUCCUCCAGC | | | |
| | | AEDYLSVVLNQLCVLHE | ACAAGGACGACAAC | | | |
| | | KTPVSDRVTKCCTESLV | CCUAACCUGCCUAG | | | |
| | | NRRPCFSALEVDETYVP | ACUGGUAAGGCCU | | | |
| | | KEFNAETFTFHADICTLS | GAGGUGGACGUGA | | | |
| | | EKERQIKKQTALVELVK | UGUGUACCGCCUUC | | | |
| | | HKPKATKEQLKAVMDD | CACGACAACGAGGA | | | |
| | | FAAFVEKCCKADDKETC | GACAUUCCUGAAGA | | | |
| | | FAEEGKKLVAASQAAL | AGUACCUGUACGAG | | | |
| | | GLGGGSAPTSSSTKKTQ | AUCGCCAGAAGACA | | | |
| | | LQLEHLLLDLQMILNGI | CCCUUACUUCUACG | | | |
| | | NNYKNPKLTRMLTFKFY | CCCCUGAGUUGCUG | | | |
| | | MPKKATELKHLQCLEEE | UUCUUCGCGAAGA | | | |
| | | LKPLEEALNLAPSKNFHL | GAUACAAGGCCGCC | | | |
| | | RPRDLISDINVIVLELKG | UUCACCGAGUGCUG | | | |
| | | SETTFMCEYADETATIV | CCAGGCCGCCGAUA | | | |
| | | EFLNRWITFCQSIISTLT | AGGCCGCGUGCCUG | | | |
| | | | CUGCCUAAGCUGGA | | | |
| | | | CGAGCUGAGAGACG | | | |
| | | | AGGGCAAGGCAUCC | | | |
| | | | AGCGCUAAGCAGAG | | | |
| | | | ACUGAAGUGCGCCA | | | |
| | | | GCCUGCAGAAGUUC | | | |
| | | | GGAGAGAGAGCUU | | | |
| | | | UCAAGGCGUGGGC | | | |
| | | | AGUGGCUAGAUUG | | | |
| | | | AGCCAAAGAUUCCC | | | |
| | | | UAAGGCAGAAUUC | | | |
| | | | GCUGAGGUGAGCA | | | |
| | | | AGCUCGUGACUGAC | | | |
| | | | CUGACCAAGGUGCA | | | |
| | | | UACAGAGUGCUGU | | | |
| | | | CACGGCGACCUGCU | | | |
| | | | GGAGUGCGCCGACG | | | |
| | | | ACAGAGCCGACCUG | | | |
| | | | GCCAAGUACAUCUG | | | |
| | | | CGAGAACCAGGACA | | | |
| | | | GCAUCAGCAGCAAG | | | |
| | | | CUGAAGGAGUGUU | | | |
| | | | GUGAGAAGCCUCUA | | | |
| | | | UUGGAGAAGAGUC | | | |
| | | | ACUGCAUUGCCGAG | | | |
| | | | GUGGAGAACGACGA | | | |
| | | | GAUGCCUGCGGAUC | | | |
| | | | UGCCAAGCUUGGCG | | | |
| | | | GCCGACUUCGUGGA | | | |
| | | | GAGCAAGGACGUG | | | |
| | | | UGCAAGAACUACGC | | | |
| | | | CGAGGCCAAGGACG | | | |
| | | | UUUUCCUGGGCAU | | | |
| | | | GUUCCUCUACGAGU | | | |
| | | | ACGCACGCAGACAU | | | |
| | | | CCAGACUACAGCGU | | | |
| | | | GGUGCUGCUGCUG | | | |
| | | | AGACUGGCUAAGAC | | | |
| | | | AUACGAAACUACCC | | | |
| | | | UGGAGAAGUGCUG | | | |
| | | | CGCAGCGGCGGACC | | | |
| | | | CUCACGAGUGUUAC | | | |
| | | | GCCAAGGUGUUCG | | | |
| | | | ACGAGUUCAAGCCU | | | |
| | | | CUGGUGGAGGAGC | | | |
| | | | CUCAGAACCUGAUC | | | |
| | | | AAGCAGAACUGUGA | | | |
| | | | GCUGUUCGAGCAGC | | | |
| | | | UCGGCGAGUACAAG | | | |
| | | | UUCCAGAACGCCCU | | | |
| | | | GUUGGUCCGCUACA | | | |
| | | | CCAAGAAGGUGCCU | | | |
| | | | CAAGUAAGUACCCC | | | |
| | | | UACCCUGGUAGAG | | | |
| | | | GUUAGUAGAAACC | | | |
| | | | UGGGCAAGGUGGG | | | |
| | | | CAGCAAGUGCUGUA | | | |
| | | | AGCACCCAGAAGCU | | | |
| | | | AAGAGGAUGCCUU | | | |
| | | | GCGCCGAGGACUAC | | | |
| | | | CUGUCCGUUGUGC | | | |
| | | | UGAACCAGCUGUGC | | | |
| | | | GUGCUGCACGAGAA | | | |
| | | | GACCCCUGUGAGCG | | | |
| | | | ACAGAGUGACAAAG | | | |
| | | | UGUUGCACCGAGA | | | |
| | | | GCUUAGUGAAUAG | | | |
| | | | AAGACCUUGCUUCA | | | |
| | | | GCGCCCUGGAAGUU | | | |
| | | | GACGAAACUUACGU | | | |
| | | | GCCUAAGGAGUUCA | | | |
| | | | ACGCCGAGACUUUC | | | |
| | | | ACAUUCCACGCCGA | | | |
| | | | CAUUUGCACUCUGA | | | |
| | | | GCGAGAAGGAGAG | | | |
| | | | ACAGAUCAAGAAGC | | | |
| | | | AGACCGCCCUCGUA | | | |
| | | | GAGUUGGUCAAGC | | | |
| | | | ACAAGCCGAAGGCA | | | |
| | | | ACCAAGGAACAGCU | | | |
| | | | UAAGGCCGUGAUG | | | |
| | | | GACGACUUCGCAGC | | | |
| | | | CUUCGUCGAGAAG | | | |
| | | | UGUUGUAAGGCCG | | | |
| | | | ACGACAAGGAGACU | | | |
| | | | UGCUUCGCAGAGG | | | |
| | | | AAGGCAAGAAGUU | | | |
| | | | GGUAGCCGCCUCUC | | | |
| | | | AGGCUGCCUUGGG | | | |
| | | | ACUCGGUGGCGGCA | | | |
| | | | GCGCCCCUACCAGC | | | |
| | | | AGCUCCACAAAGAA | | | |
| | | | GACGCAGCUGCAGC | | | |
| | | | UGGAGCACCUGCUA | | | |
| | | | CUAGACUUACAGAU | | | |
| | | | GAUCCUGAACGGCA | | | |
| | | | UCAACAACUACAAG | | | |
| | | | AACCCGAAGCUCAC | | | |
| | | | CAGAAUGCUGACAU | | | |
| | | | UCAAGUUCUACAUG | | | |
| | | | CCUAAGAAGGCCAC | | | |
| | | | UGAGCUGAAGCACC | | | |
| | | | UUCAGUGCCUGGA | | | |
| | | | GGAGGAACUUAAG | | | |
| | | | CCACUGGAAGAGGC | | | |
| | | | CCUGAACCUGGCCC | | | |
| | | | CUAGUAAGAAUUU | | | |
| | | | CCACCUCAGACCUC | | | |
| | | | GCGACCUUAUUAGC | | | |
| | | | GACAUCAACGUGAU | | | |
| | | | CGUGCUCGAAUUAA | | | |
| | | | AGGGCUCCGAGACA | | | |
| | | | ACAUUCAUGUGCGA | | | |
| | | | AUACGCCGACGAGA | | | |
| | | | CAGCCACCAUCGUG | | | |
| | | | GAGUUCCUGAACCG | | | |
| | | | CUGGAUCACCUUCU | | | |
| | | | GCCAGAGCAUCAUC | | | |
| | | | UCUACUCUGACC | | | |

TABLE IV
Exemplary IL-15 and IL-15RA sequences

| Sequence ID | Sequence ID | |
|---|---|---|
| No | Info | Sequence |
| 40 | IL-15 | MRISKPHLRS ISIQCYLCLL LNSHFLTEAG IHVFILGCFS |
| | | AGLPKTEANW VNVISDLKKI EDLIQSMHID ATLYTESDVH |
| | | PSCKVTAMKC FLLELQVISL ESGDASIHDT VENLIILANN |
| | | SLSSNGNVTE SGCKECEELE EKNIKEFLQS FVHIVQMFIN TS |
| 41 | IL-15 | ATGAGAATTTCGAAACCACATTTGAGAAGTATTTCCATCCAG |
| | | TGCTACTTGTGTTTACTTCTAAACAGTCATTTTCTAACTGAA |
| | | GCTGGCATTCATGTCTTCATTTTGGGCTGTTTCAGTGCAGG |
| | | GCTTCCTAAAACAGAAGCCAACTGGGTGAATGTAATAAGTG |
| | | ATTTGAAAAAAATTGAAGATCTTATTCAATCTATGCATATTGA |
| | | TGCTACTTTATATACGGAAAGTGATGTTCACCCCAGTTGCA |
| | | AAGTAACAGCAATGAAGTGCTTTCTCTTGGAGTTACAAGTTA |
| | | TTTCACTTGAGTCCGGAGATGCAAGTATTCATGATACAGTA |
| | | GAAAATCTGATCATCCTAGCAAACAACAGTTTGTCTTCTAAT |
| | | GGGAATGTAACAGAATCTGGATGCAAAGAATGTGAGGAACT |
| | | GGAGGAAAAAAATATTAAAGAATTTTTGCAGAGTTTTGTACA |
| | | TATTGTCCAAATGTTCATCAACACTTCTTGA |
| 42 | IL-15RA | MAPRRARGCRTLGLPALLLLLLLRPPATRGITCPPPMSVEHADI |
| | | WVKSYSLYSRERYICNSGFKRKAGTSSLTECVLNKATNVAHW |
| | | TTPSLKCIRDPALVHQRPAPPSTVTTAGVTPQPESLSPSGKEP |
| | | AASSPSSNNTAATTAAIVPGSQLMPSKSPSTGTTEISSHESSH |
| | | GTPSQTTAKNWELTASASHQPPGVYPQGHSDTTVAISTSTVL |
| | | LCGLSAVSLLACYLKSRQTPPLASVEMEAMEALPVTWGTSSR |
| | | DEDLENCSHHL |
| 43 | IL-15RA | ATGGCCCCGCGGCGGGCGCGCGGCTGCCGGACCCTCGGTCT |
| | | CCCGGCGCTGCTACTGCTGCTGCTGCTCCGGCCGCCGGCGAC |
| | | GCGGGGCATCACGTGCCCTCCCCCCATGTCCGTGGAACACG |
| | | CAGACATCTGGGTCAAGAGCTACAGCTTGTACTCCAGGGAG |
| | | CGGTACATTTGTAACTCTGGTTTCAAGCGTAAAGCCGGCACG |
| | | TCCAGCCTGACGGAGTGCGTGTTGAACAAGGCCACGAATGT |
| | | CGCCCACTGGACAACCCCCAGTCTCAAATGCATTAGAGACC |
| | | CTGCCCTGGTTCACCAAAGGCCAGCGCCACCCTCCACAGTA |
| | | ACGACGGCAGGGGTGACCCCACAGCCAGAGAGCCTCTCCCC |
| | | TTCTGGAAAAGAGCCCGCAGCTTCATCTC |
| | | CCAGCTCAAACAACACAGCGGCCACAACAGCAGCTATTGTC |
| | | CCGGGCTCCCAGCTGATGCCTTCAAAATCACCTTCCACAGGA |
| | | ACCACAGAGATAAGCAGTCATGAGTCCTCCCACGGCACCCC |
| | | CTCTCAGACAACAGCCAAGAACTGGGAACTCACAGCATCCG |
| | | CCTCCCACCAGCCGCCAGGTGTGTATCCACAGGGC |
| | | CACAGCGACACCACTGTGGCTATCTCCACGTCCACTGTCCTG |
| | | CTGTGTGGGCTGAGCGCTGTGTCTCTCCTGGCATGCTACCTC |
| | | AAGTCAAGGCAAACTCCCCCGCTGGCCAGCGTTGAAATGGA |
| | | AGCCATGGAGGCTCTGCCGGTGACTTGGGGGACCAGCAGCA |
| | | GAGATGAAGACTTGGAAAACTGCTCTCACCACCT |
| | | ATGA |

### Stabilizing Elements

Naturally occurring eukaryotic mRNA molecules can contain stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

In some embodiments, the polynucleotide has an open reading frame encoding at least one antigenic, tumor antigenic, or signaling polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3-O-Me-m7G(5′)ppp(5′) G [the ARCA cap];G(5′)ppp(5′)A; G(5′)ppp(5′)G; m7G(5′)ppp(5′)A; m7G(5′)ppp(5′)G (New England BioLabs, Ipswich, MA). 5′-capping of modified RNA may be completed post-transcriptionally using a Vaccinia Vims Capping Enzyme to generate the “Cap 0” structure: m7G(5′)ppp(5′)G (New England BioLabs, Ipswich, MA). Cap 1 structure may be generated using both Vaccinia Vims Capping Enzyme and a 2′-0 methyl-transferase to generate m7G(5′)ppp(5′)G-2′-O-methyl. Cap 2 structure may be generated from the Cap 1 stmcture followed by the 2′-O-methylation of the 5′-antepenultimate nucleotide using a 2′-0 methyl-transferase. Cap 3 structure may be generated from the Cap 2 stmcture followed by the 2′-O-methylation of the 5′-preantepenultimate nucleotide using a 2′-0 methyl-transferase. Enzymes may be derived from a recombinant source.

The 3′-poly(A) tail is typically a stretch of adenine nucleotides added to the 3′-end of the transcribed mRNA. It can, in some instances, comprise up to about 400 adenine nucleotides. In some embodiments, the length of the 3′-poly(A) tail may be an essential element with respect to the stability of the individual mRNA.

In some embodiments, the polynucleotide includes a stabilizing element. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, the polynucleotide (e.g., mRNA) includes a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, b-Galactosidase, EGFP), or a marker or selection protein (e.g. alpha-Globin, Galactokinase and Xanthine:guanine phosphoribosyl transferase (GPT)).

In some embodiments, the polynucleotide (e.g., mRNA) includes the combination of a poly(A) sequence or polyadenylation signal and at least one histone stem-loop, even though both represent alternative mechanisms in nature, acts synergistically to increase the protein expression beyond the level observed with either of the individual elements. The synergistic effect of the combination of poly(A) and at least one histone stem-loop does not depend on the order of the elements or the length of the poly(A) sequence. In some embodiments, an RNA (e.g., mRNA) does not include a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. In some embodiments, the nucleic acid does not include an intron.

The polynucleotide (e.g., mRNA) may or may not contain an enhancer and/or promoter sequence, which may be modified or unmodified or which may be activated or inactivated. In some embodiments, the histone stem-loop is generally derived from histone genes and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In some embodiments, the polynucleotide (e.g., mRNA) has one or more AU-rich sequences removed. These sequences, sometimes referred to as AURES are destabilizing sequences found in the 3′UTR. The AURES may be removed from the RNA vaccines. Alternatively, the AURES may remain in the RNA vaccine.

### Signal Peptides

In some embodiments, the polynucleotide (e.g., mRNA) has an ORF that encodes a signal peptide fused to the coronavirus antigen. Signal peptides, comprising the N-terminal 15-60 amino acids of proteins, are typically needed for the translocation across the membrane on the secretory pathway and, thus, universally control the entry of most proteins both in eukaryotes and in prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by an ER-resident signal peptidase of the host cell, or they remain uncleaved and function as a membrane anchor. A signal peptide may also facilitate the targeting of the protein to the cell membrane. A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (which regulate expression of genes other than coronavirus antigens in nature) are known in the art and can be tested for desired properties and then incorporated into a nucleic acid of the disclosure. In some embodiments, the signal peptide may comprise those described in WO 2021/154763, which is incorporated by reference in its entirety.

### Fusion Proteins

In some embodiments, the polynucleotide (e.g., mRNA) encodes an antigenic fusion protein. Thus, the encoded antigen or antigens may include two or more proteins (e.g., protein and/or protein fragment) joined together. Alternatively, the protein to which a protein antigen is fused does not promote a strong immune response to itself, but rather to the coronavirus antigen. Antigenic fusion proteins, in some embodiments, retain the functional property from each original protein.

### Scaffold Moieties

The polynucleotide (e.g., mRNA), in some embodiments, encodes fusion proteins that comprise coronavirus antigens linked to scaffold moieties. In some embodiments, such scaffold moieties impart desired properties to an antigen encoded by a nucleic acid of the disclosure. For example, scaffold proteins may improve the immunogenicity of an antigen, e.g., by altering the structure of the antigen, altering the uptake and processing of the antigen, and/or causing the antigen to bind to a binding partner.

In some embodiments, the scaffold moiety is protein that can self-assemble into protein nanoparticles that are highly symmetric, stable, and structurally organized, with diameters of 10-150 nm, a highly suitable size range for optimal interactions with various cells of the immune system.

In some embodiments, bacterial protein platforms may be used. Non-limiting examples of these self-assembling proteins include ferritin, lumazine and encapsulin.

Ferritin is a protein whose main function is intracellular iron storage. Ferritin is made of 24 subunits, each composed of a four-alpha-helix bundle, that self-assemble in a quaternary structure with octahedral symmetry (Cho K. J. et al. J Mol Biol. 2009; 390:83-98). Several high-resolution structures of ferritin have been determined, confirming that Helicobacter pylori ferritin is made of 24 identical protomers, whereas in animals, there are ferritin light and heavy chains that can assemble alone or combine with different ratios into particles of 24 subunits (Granier T. et al. J Biol Inorg Chem. 2003; 8:105-111; Fawson D. M. et al. Nature. 1991; 349:541-544). Ferritin self-assembles into nanoparticles with robust thermal and chemical stability. Thus, the ferritin nanoparticle is well suited to carry and expose antigens.

Fumazine synthase (FS) is also well suited as a nanoparticle platform for antigen display. FS, which is responsible for the penultimate catalytic step in the biosynthesis of riboflavin, is an enzyme present in a broad variety of organisms, including archaea, bacteria, fungi, plants, and eubacteria (Weber S. E. Flavins and Flavoproteins. Methods and Protocols, Series: Methods in Molecular Biology. 2014). The FS monomer is 150 amino acids long, and consists of beta-sheets along with tandem alpha-helices flanking its sides. A number of different quaternary structures have been reported for FS, illustrating its morphological versatility: from homopentamers up to symmetrical assemblies of 12 pentamers forming capsids of 150 A diameter. Even FS cages of more than 100 subunits have been described (Zhang X. et al. J Mol Biol. 2006; 362:753-770).

Encapsulin, a novel protein cage nanoparticle isolated from thermophile Thermotoga maritima, may also be used as a platform to present antigens on the surface of self-assembling nanoparticles. Encapsulin is assembled from 60 copies of identical 31 kDa monomers having a thin and icosahedral T=1 symmetric cage structure with interior and exterior diameters of 20 and 24 nm, respectively (Sutter M. et al. Nat Struct Mol Biol. 2008, 15: 939-947). Although the exact function of encapsulin in T. maritima is not clearly understood yet, its crystal structure has been recently solved and its function was postulated as a cellular compartment that encapsulates proteins such as DyP (Dye decolorizing peroxidase) and Flp (Ferritin like protein), which are involved in oxidative stress responses (Rahmanpour R. et al. FEBS J. 2013, 280: 2097-2104).

In some embodiments, the polynucleotide encodes a coronavirus antigen (e.g., SARS-CoV-2 S protein) fused to a foldon domain. The foldon domain may be, for example, obtained from bacteriophage T4 fibritin (see, e.g., Tao Y, et al. Structure. 1997 Jun. 15; 5(6):789-98).

### Linkers and Cleavable Peptides

In some embodiments, the polynucleotide (e.g., mRNA) encodes more than one polypeptide, referred to herein as fusion proteins. In some embodiments, the mRNA further encodes a linker located between at least one or each domain of the fusion protein. The linker can be, for example, a cleavable linker or protease-sensitive linker. In some embodiments, the linker is selected from the group consisting of F2A linker, P2A linker, T2A linker, E2A linker, and combinations thereof. This family of self-cleaving peptide linkers, referred to as 2 A peptides, has been described in the art (see for example, Kim, J. H. et al. (2011) PLoS ONE 6:el8556). In some embodiments, the linker is an F2A linker. In some embodiments, the fusion protein contains three domains with intervening linkers, having the structure: domain-linker-domain-linker-domain.

Cleavable linkers known in the art may be used in connection with the disclosure. Exemplary such linkers include F2A linkers, T2A linkers, P2A linkers, E2A linkers (See, e.g., WO2017/127750, which is incorporated herein by reference in its entirety). The skilled artisan will appreciate that other art-recognized linkers may be suitable for use in the constructs of the disclosure (e.g., encoded by the nucleic acids of the disclosure). The skilled artisan will likewise appreciate that other polycistronic constructs (mRNA encoding more than one antigen/polypeptide separately within the same molecule) may be suitable for use as provided herein.

### Sequence Optimization

In some embodiments, an ORF encoding an antigen, tumor antigen, signaling or anti-cancer protein of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence ORF (e.g., a naturally-occurring or wild-type mRNA sequence encoding a coronavirus antigen). In some embodiments, a codon optimized sequence shares less than 90% sequence identity to a naturally occurring or wild-type sequence (e.g., a naturally occurring or wild-type mRNA sequence encoding a coronavirus antigen). In some embodiments, a codon optimized sequence shares less than 85% sequence identity to a naturally occurring or wild-type sequence (e.g., a naturally occurring or wild-type mRNA sequence encoding a coronavirus antigen). In some embodiments, a codon optimized sequence shares less than 80% sequence identity to a naturally occurring or wild-type sequence (e.g., a naturally occurring or wild-type mRNA sequence encoding a coronavirus antigen). In some embodiments, a codon optimized sequence shares less than 75% sequence identity to a naturally occurring or wild-type sequence (e.g., a naturally occurring or wild-type mRNA sequence encoding a coronavirus antigen).

In some embodiments, a codon optimized sequence shares between 65% and 85% (e.g., between about 67% and about 85% or between about 67% and about 80%) sequence identity to a naturally occurring or wild-type sequence (e.g., a naturally occurring or wild-type mRNA sequence encoding a coronavirus antigen). In some embodiments, a codon optimized sequence shares between 65% and 75% or about 80% sequence identity to a naturally occurring or wild-type sequence (e.g., a naturally occurring or wild-type mRNA sequence encoding a coronavirus antigen).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), a coronavirus antigen encoded by a non-codon-optimized sequence. When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments, a codon optimized RNA may be one in which the levels of G/C are enhanced. The G/C-content of nucleic acid molecules (e.g., mRNA) may influence the stability of the RNA. RNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Chemically Unmodified Nucleotides

In some embodiments, the polynucleotide (e.g., mRNA) is not chemically modified and comprises the standard ribonucleotides consisting of adenosine, guanosine, cytosine and uridine. In some embodiments, nucleotides and nucleosides of the polynucleotide (e.g., mRNA) comprise standard nucleoside residues such as those present in transcribed RNA (e.g. A, G, C, or U). In some embodiments, nucleotides and nucleosides of the polynucleotide (e.g., mRNA) comprise standard deoxyribonucleosides such as those present in DNA (e.g. dA, dG, dC, or dT).

### Chemical Modifications

The polynucleotide (e.g., mRNA) comprises, in some embodiments, an RNA having an open reading frame encoding a coronavirus antigen, wherein the nucleic acid comprises nucleotides and/or nucleosides that can be standard (unmodified) or modified as is known in the art. In some embodiments, nucleotides and nucleosides of the polynucleotide (e.g., mRNA) comprise modified nucleotides or nucleosides. Such modified nucleotides and nucleosides can be naturally occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

The nucleic acids of the polynucleotide (e.g., mRNA) can comprise standard nucleotides and nucleosides, naturally-occurring nucleotides and nucleosides, non-naturally-occurring nucleotides and nucleosides, or any combination thereof.

Nucleic acids of the polynucleotide (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, the polynucleotide (e.g., mRNA) comprises uridine at one or more or all uridine positions of the nucleic acid. In some embodiments, mRNAs are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the polynucleotide (e.g., mRNA) may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the poly(A) tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C orA+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The mRNAs may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Untranslated Regions (UTRs)

The polynucleotide (e.g., mRNA) may comprise one or more regions or parts that act or function as an untranslated region. Where mRNAs are designed to encode at least one protein of interest, the polynucleotide may comprise one or more of these untranslated regions (UTRs). Wild-type untranslated regions of a nucleic acid sequence are transcribed but not translated. In mRNA, the 5′ UTR starts at the transcription start site and continues to the start codon but does not include the start codon; whereas, the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′ UTR and 3′ UTR sequences are known and available in the art.

A 5′ UTR is region of an mRNA that is directly upstream (5′) from the start codon (the first codon of an mRNA transcript translated by a ribosome). A 5′ UTR does not encode a protein (is non-coding). Natural 5′ UTRs have features that play roles in translation initiation. They harbor signatures like Kozak sequences, which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′ UTR also have been known to form secondary structures which are involved in elongation factor binding.

In some embodiments, the 5′ UTR is a heterologous UTR, i.e., is a UTR found in nature associated with a different ORF. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (U.S. Pat. Nos. 8,278,063; 9,012,219), human cytochrome b-245 a polypeptide, and hydroxysteroid (17b) dehydrogenase, and Tobacco etch virus (U.S. Pat. Nos. 8,278,063, 9,012,219, which are incorporated herein by reference in their entirety). CMV immediate-early 1 (IE1) gene (US2014/0206753, WO2013/185069, which are incorporated herein by reference in their entirety), the sequence GGGAUCCUACC (WO2014/144196) may also be used. In another embodiment, 5′ UTR of a TOP gene is a 5′ UTR of a TOP gene lacking the 5′ TOP motif (the oligopyrimidine tract) (e.g., WO/2015/101414, WO2015/101415, WO/2015/062738, WO2015/024667, WO2015/024667; 5′ UTR element derived from ribosomal protein Large 32 (L32) gene (WO/2015/101414, WO2015/101415, WO/2015/062738), 5′ UTR element derived from the 5′ UTR of an hydroxysteroid (17-b) dehydrogenase 4 gene (HSD17B4) (WO2015/024667), or a 5′ UTR element derived from the 5′ UTR of ATP5A1 (WO2015/024667) can be used. In some embodiments, an internal ribosome entry site (IRES) is used instead of a 5′ UTR.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-α. Class III ARES are less well defined. These U rich regions do not contain an AUUUA motif. c-Jun and Myogenin are two well-studied examples of this class.

Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of the polynucleotide (e.g., mRNA). When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

3′ UTRs may be heterologous or synthetic.

Those of ordinary skill in the art will understand that 5′ UTRs that are heterologous or synthetic may be used with any desired 3′ UTR sequence. For example, a heterologous 5′ UTR may be used with a synthetic 3′ UTR with a heterologous 3′ UTR.

Non-UTR sequences may also be used as regions or subregions within a nucleic acid. For example, introns or portions of introns sequences may be incorporated into regions of nucleic acid sequences of the disclosure. Incorporation of intronic sequences may increase protein production as well as nucleic acid levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in US Patent Application Publication No. 2010/0293625 and PCT/US2014/069155, which are herein incorporated by reference in their entirety. It should be understood that any UTR from any gene may be incorporated into the regions of a nucleic acid sequence. Furthermore, multiple wild-type UTRs of any known gene may be utilized. It is also within the scope of the present disclosure to provide artificial UTRs that are not variants of wild type regions. These UTRs or portions thereof may be placed in the same orientation as in the transcript from which they were selected or may be altered in orientation or location. Hence, a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US Patent publication 2010/0129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature or property. For example, polypeptides of interest may belong to a family of proteins that are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest that share at least one function, structure, feature, localization, origin, or expression pattern.

The untranslated region may also include translation enhancer elements (TEE). As a non-limiting example, the TEE may include those described in US Application No. 2009/0226470, herein incorporated by reference in its entirety, and those known in the art. In vitro Transcription of RNA cDNA encoding the polynucleotides described herein may be transcribed using an in vitro transcription (IVT) system. In vitro transcription of RNA is known in the art and is described in International Publication WO 2014/152027, which is incorporated by reference herein in its entirety. In some embodiments, the RNA of the present disclosure is prepared in accordance with any one or more of the methods described in WO 2018/053209 and WO 2019/036682, each of which is incorporated by reference herein.

In some embodiments, the RNA transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of a RNA polynucleotide, for example, but not limited to coronavirus mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA, which is then isolated and purified. In some embodiments, the DNA template includes a RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a poly(A) tail. The particular nucleic acid sequence composition and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide. When RNA transcripts are being generated, the 5′ UTR may comprise a promoter sequence. Such promoter sequences are known in the art. It should be understood that such promoter sequences will not be present in a vaccine of the disclosure.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “poly(A) tail” is a region of mRNA that is downstream, e.g., directly downstream (i.e., 3′), from the 3′ UTR that contains multiple, consecutive adenosine monophosphates. A poly(A) tail may contain 10 to 300 adenosine monophosphates. For example, a poly(A) tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a poly(A) tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation.

In some embodiments, a nucleic acid includes 200 to 3,000 nucleotides. For example, a nucleic acid may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

An in vitro transcription system typically comprises a transcription buffer, nucleotide triphosphates (NTPs), an RNase inhibitor and a polymerase.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

In some embodiments, the RNA transcript is capped via enzymatic capping. In some embodiments, the polynucleotide (e.g., mRNA) comprises 5′ terminal cap, for example, 7mG(5′)ppp(5′)NlmpNp.

### Chemical Synthesis

Solid-phase chemical synthesis. The polynucleotide (e.g., mRNA) may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

Liquid Phase Chemical Synthesis. The synthesis of the polynucleotide (e.g., mRNA) by the sequential addition of monomer building blocks may be carried out in a liquid phase.

Combination of Synthetic Methods. The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

### Ligation of Nucleic Acid Regions or Subregions

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase-catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the polynucleotide (e.g., mRNA) may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheo alveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Methods of Treatment

Provided herein are compositions {e.g., pharmaceutical compositions), methods, kits and reagents for prevention and/or treatment of cancer in humans and other mammals. the LPMP/mRNA formulations can be used as therapeutic or prophylactic agents. They may be used in medicine to prevent and/or treat cancer.

In one embodiment, the LPMP/mRNA therapeutic compositions are used to provide prophylactic protection from cancer. Prophylactic protection from cancer can be achieved following administration of a LPMP/mRNA therapeutic composition (as vaccines). Vaccines can be administered once, twice, three times, four times or more but it is likely sufficient to administer the vaccine once (optionally followed by a single booster). It is more desirable, to administer the vaccine to an individual having cancer to achieve a therapeutic response. Dosing may need to be adjusted accordingly.

In some embodiments, the LPMP/mRNA therapeutic composition (as vaccines) is administered on a schedule for up to two months, up to three months, up to four month, up to five months, up to six months, up to seven months, up to eight months, up to nine months, up to ten months, up to eleven months, up to 1 year, up to 1 and % years, up to two years, up to three years, or up to four years. The schedule may be the same or varied. In some embodiments the schedule is weekly for the first 3 weeks and then monthly thereafter.

The LPMP/mRNA therapeutic composition (as vaccines) may be administered by any route. In some embodiments the vaccine is administered by an EVI or IV route.

At any point in the treatment, the patient may be examined to determine whether the mutations in the vaccine are still appropriate. Based on that analysis the vaccine may be adjusted or reconfigured to include one or more different mutations or to remove one or more mutations.

### Therapeutic and Prophylactic Compositions

Provided herein are compositions (e.g., pharmaceutical compositions), methods, kits and reagents for prevention, treatment or diagnosis of cancer in humans and other mammals,

In some embodiments, LPMP/mRNA therapeutic composition (as vaccines) can be used in the priming of immune effector cells, for example, to activate peripheral blood mononuclear cells (PBMCs) ex vivo, which are then infused (re-infused) into a subject.

In exemplary embodiments, the LPMP/mRNA therapeutic composition can be administered to a subject (e.g., a mammalian subject, such as a human subject), and the RNA polynucleotides are translated in vivo to produce an antigenic polypeptide.

The LPMP/mRNA therapeutic composition may be induced for translation of a polypeptide {e.g., antigen, tumor antigen, or signaling protein) in a cell, tissue or organism. In exemplary embodiments, such translation occurs in vivo, although there can be envisioned embodiments where such translation occurs ex vivo, in culture or in vitro. In exemplary embodiments, the cell, tissue or organism is contacted with an effective amount of a composition containing a LPMP/mRNA therapeutic composition that contains a polynucleotide that has at least one a translatable region encoding an antigenic (e.g., tumor antigenic) or signaling polypeptide.

An “effective amount” of a LPMP/mRNA therapeutic composition is provided based, at least in part, on the target tissue, target cell type, means of administration, physical characteristics of the polynucleotide (e.g., size, and extent of modified nucleosides) and other components of LPMP/mRNA therapeutic composition, and other determinants. In general, an effective amount of LPMP/mRNA therapeutic composition provides an induced or boosted immune response as a function of antigen or polypeptide production in the cell, preferably more efficient than a composition containing a corresponding unmodified polynucleotide encoding the same antigen or polypeptide. Increased antigen production may be demonstrated by increased cell transfection (the percentage of cells transfected with the LPMP/mRNA therapeutic composition), increased protein translation from the polynucleotide, decreased nucleic acid degradation (as demonstrated, for example, by increased duration of protein translation from a modified polynucleotide), or altered therapeutic or antigen specific immune response of the host cell.

In some embodiments, the LPMP/mRNA therapeutic composition may be used for treatment of cancer.

The LPMP/mRNA therapeutic composition may be administered prophylactically or therapeutically as part of an active immunization scheme to healthy individuals or early in cancer or during active cancer after onset of symptoms. In some embodiments, the amount of the LPMP/mRNA therapeutic composition provided to a cell, a tissue or a subject may be an amount effective for immune prophylaxis.

The LPMP/mRNA therapeutic composition may be administered with other prophylactic or therapeutic compounds. As a non-limiting example, a prophylactic or therapeutic compound may be an immune potentiator, adjuvant, or booster. As used herein, when referring to a composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In exemplary embodiments, the time of administration between the initial administration of the prophylactic composition and the booster may be, but is not limited to, 1 week, 2 weeks, 3 weeks, 1 month, 2 months, 3 months, 6 months or 1 year.

In one embodiment, the LPMP/mRNA therapeutic composition may be administered intramuscularly or intradermally similarly to the administration of vaccines known in the art.

The LPMP/mRNA therapeutic composition may be utilized in various settings depending on the severity of the cancer or the degree or level of unmet medical need. As a non-limiting example, the LPMP/mRNA therapeutic composition may be utilized to treat any stage of cancer. The LPMP/mRNA therapeutic composition have superior properties in that they produce much larger antibody titers, T cell responses and produce responses early than commercially available anti-cancer vaccines. While not wishing to be bound by theory, the inventors hypothesize that the LPMP/mRNA therapeutic composition, as mRNAs, are better designed to produce the appropriate protein conformation on translation as the LPMP/mRNA therapeutic composition co-opt natural cellular machinery. Unlike traditional vaccines which are manufactured ex vivo and may trigger unwanted cellular responses, the LPMP/mRNA therapeutic composition are presented to the cellular system in a more native fashion.

A non-limiting list of cancers that the LPMP/mRNA therapeutic composition may treat is presented below. Peptide epitopes or antigens may be derived from any antigen of these cancers or tumors. Such epitopes are referred to as cancer or tumor antigens. Cancer cells may differentially express cell surface molecules during different phases of tumor progression. For example, a cancer cell may express a cell surface antigen in a benign state, yet down-regulate that particular cell surface antigen upon metastasis. As such, it is envisioned that the tumor or cancer antigen may encompass antigens produced during any stage of cancer progression. The methods of the invention may be adjusted to accommodate for these changes. For instance, several different LPMP/mRNA therapeutic compositions may be generated for a particular patient. For instance, a first vaccine may be used at the start of the treatment. At a later time point, a new LPMP/mRNA therapeutic composition may be generated and administered to the patient to account for different antigens being expressed.

Provided herein are pharmaceutical compositions including the LPMP/mRNA therapeutic composition optionally in combination with one or more pharmaceutically acceptable excipients.

The LPMP/mRNA therapeutic composition may be formulated or administered alone or in conjunction with one or more other components. For instance, the LPMP/mRNA therapeutic composition may comprise other components including, but not limited to, immune potentiators (e.g., adjuvants). In some embodiments, the LPMP/mRNA therapeutic composition do not include an immune potentiator or adjuvant (i.e., they are immune potentiator or adjuvant free).

In other embodiments, the LPMP/mRNA therapeutic composition may be combined with any other therapy useful for treating the patient. For instance, a patient may be treated with the LPMP/mRNA therapeutic composition and an anti-cancer therapeutic agent. Thus, in one embodiment, the methods of the invention can be used in conjunction with one or more cancer therapeutics, for example, in conjunction with an anti-cancer therapeutic agent, a traditional cancer vaccine, chemotherapy, radiotherapy, etc. (e.g., simultaneously, or as part of an overall treatment procedure). Parameters of cancer treatment that may vary include, but are not limited to, dosages, timing of administration or duration or therapy; and the cancer treatment can vary in dosage, timing, or duration. Another treatment for cancer is surgery, which can be utilized either alone or in combination with any of the previous treatment methods. Any agent or therapy (e.g., traditional cancer vaccines, chemotherapies, radiation therapies, surgery, hormonal therapies, and/or biological therapies/immunotherapies) which is known to be useful, or which has been used or is currently being used for the prevention or treatment of cancer can be used in combination with a composition of the invention in accordance with the invention described herein. One of ordinary skill in the medical arts can determine an appropriate treatment for a subject.

Examples of such agents (i.e., anti-cancer therapeutic agents) include, but are not limited to, DNA-interactive agents including, but not limited to, the alkylating agents (e.g., nitrogen mustards, e.g. Chlorambucil, Cyclophosphamide, Isofamide, Mechlorethamine, Melphalan, Uracil mustard; Aziridine such as Thiotepa; methanesulphonate esters such as Busulfan; nitroso ureas, such as Carmustine, Lomustine, Streptozocin; platinum complexes, such as Cisplatin, Carboplatin; bioreductive alkylator, such as Mitomycin, and Procarbazine, Dacarbazine and Altretamine); the DNA strand-breakage agents, e.g., Bleomycin; the intercalating topoisomerase II inhibitors, e.g., Intercalators, such as Amsacrine, Dactinomycin, Daunorubicin, Doxorubicin, Idarubicin, Mitoxantrone, and nonintercalators, such as Etoposide and Teniposide; the nonintercalating topoisomerase II inhibitors, e.g., Etoposide and Teniposde; and the DNA minor groove binder, e.g., Plicamydin; the antimetabolites including, but not limited to, folate antagonists such as Methotrexate and trimetrexate; pyrimidine antagonists, such as Fluorouracil, Fluorodeoxyuridine, CB3717, Azacitidine and Floxuridine; purine antagonists such as Mercaptopurine, 6-Thioguanine, Pentostatin; sugar modified analogs such as Cytarabine and Fludarabine; and ribonucleotide reductase inhibitors such as hydroxyurea; tubulin Interactive agents including, but not limited to, colchicine, Vincristine and Vinblastine, both alkaloids and Paclitaxel and Cytoxan; hormonal agents including, but not limited to, estrogens, conjugated estrogens and Ethinyl Estradiol and Diethylstilbesterol, Chlortrianisen and Idenestrol; progestins such as Hydroxyprogesterone caproate, Medroxyprogesterone, and Megestrol; and androgens such as testosterone, testosterone propionate; fluoxymesterone, methyltestosterone; adrenal corticosteroid, e.g., Prednisone, Dexamethasone, Methylprednisolone, and Prednisolone; leutinizing hormone releasing hormone agents or gonadotropin-releasing hormone antagonists, e.g., leuprolide acetate and goserelin acetate; antihormonal antigens including, but not limited to, antiestrogenic agents such as Tamoxifen, antiandrogen agents such as Flutamide; and antiadrenal agents such as Mitotane and Aminoglutethimide; cytokines including, but not limited to, IL-1. alpha., IL-1 β, IL-2, IL-3, IL-4, IL-5, IL-6, IL-7, IL-8, IL-9, IL-10, IL-11, IL-12, IL-13, IL-18, TGF-β, GM-CSF, M-CSF, G-CSF, TNF-a, TNF-β, LAF, TCGF, BCGF, TRF, BAF, BDG, MP, LIF, OSM, TMF, PDGF, IFN-a, IFN-β, IFN-γ, and Uteroglobins (U.S. Pat. No. 5,696,092); anti-angiogenics including, but not limited to, agents that inhibit VEGF (e.g., other neutralizing antibodies), soluble receptor constructs, tyrosine kinase inhibitors, antisense strategies, RNA aptamers and ribozymes against VEGF or VEGF receptors, Immunotoxins and coagulants, tumor vaccines, and antibodies. Another example of such agents (i.e., anti-cancer therapeutic agents) include cytokines including, but not limited to, IL-15 or recombinant IL-15.

Specific examples of anti-cancer therapeutic agents include, but not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interieukin II, or rIL-2), interferon alpha-2a; interferon alpha-2b; interferon alpha-nl; interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; angiogenesis inhibitors; anti-dorsalizing morphogenetic protein-1; ara-CDP-DL-PTBA; BCR/ABL antagonists; CaRest M3; CARN 700; casein kinase inhibitors (ICOS); clotrimazole; collismycin A; collismycin B; combretastatin A4; crambescidin 816; cryptophycin 8; curacin A; dehydrodidemnin B; didemnin B; dihydro-5-azacytidine; dihydrotaxol, duocarmycin SA; kahalalide F; lamellarin-N triacetate; leuprolide+estrogen+progesterone; lissoclinamide 7; monophosphoryl lipid A+myobacterium cell wall sk; N-acetyldinaline; N-substituted benzamides; 06-benzylguanine; placetin A; placetin B; platinum complex; platinum compounds; platinum-triamine complex; rhenium Re 186 etidronate; RII retinamide; rubiginone B 1; SarCNU; sarcophytol A; sargramostim; senescence derived inhibitor 1; spicamycin D; tallimustine; 5-fluorouracil; thrombopoietin; thymotrinan; thyroid stimulating hormone; variolin B; thalidomide; velaresol; veramine; verdins; verteporfin; vinorelbine; vinxaltine; vitaxin; zanoterone; zeniplatin; and zilascorb.

The invention also encompasses administration of a composition comprising a LPMP/mRNA therapeutic composition in combination with radiation therapy comprising the use of x-rays, gamma rays and other sources of radiation to destroy the cancer cells. In some embodiments, the radiation treatment is administered as external beam radiation or teletherapy wherein the radiation is directed from a remote source. In other embodiments, the radiation treatment is administered as internal therapy or brachytherapy wherein a radioactive source is placed inside the body close to cancer cells or a tumor mass.

In specific embodiments, an appropriate anti-cancer regimen is selected depending on the type of cancer. For instance, a patient with ovarian cancer may be administered a prophylactically or therapeutically effective amount of a composition comprising a LPMP/mRNA therapeutic composition in combination with a prophylactically or therapeutically effective amount of one or more other agents useful for ovarian cancer therapy, including but not limited to, intraperitoneal radiation therapy, such as P32 therapy, total abdominal and pelvic radiation therapy, cisplatin, the combination of paclitaxel (Taxol) or docetaxel (Taxotere) and cisplatin or carboplatin, the combination of cyclophosphamide and cisplatin, the combination of cyclophosphamide and carboplatin, the combination of 5-FU and leucovorin, etoposide, liposomal doxorubicin, gemcitabine or topotecan. Cancer therapies and their dosages, routes of administration and recommended usage are known in the art and have been described in such literature as the Physician's Desk Reference (56th ed., 2002).

In some embodiments, the LPMP/mRNA therapeutic compositions are administered with a T cell activator such as be an immune checkpoint modulator. Immune checkpoint modulators include both stimulatory checkpoint molecules and inhibitory checkpoint molecules, i.e., an anti-CTLA4 and anti-PD1 antibody. Stimulatory checkpoint inhibitors function by promoting the checkpoint process. Several stimulatory checkpoint molecules are members of the tumor necrosis factor (T F) receptor superfamily—CD27, CD40, OX40, GITR and CD 137, while others belong to the B7-CD28 superfamily—CD28 and ICOS. OX40 (CD134), is involved in the expansion of effector and memory T cells. Anti-OX40 monoclonal antibodies have been shown to be effective in treating advanced cancer. MEDI0562 is a humanized OX40 agonist. GITR, Glucocorticoid-Induced T FR family Related gene, is involved in T cell expansion Several antibodies to GITR have been shown to promote an anti-tumor responses. ICOS, Inducible T-cell costimulator, is important in T cell effector function. CD27 supports antigen-specific expansion of naive T cells and is involved in the generation of T and B cell memory. Several agonistic anti-CD27 antibodies are in development. CD122 is the Interleukin-2 receptor beta sub-unit. KTR-214 is a CD122-biased immune-stimulatory cytokine.

Inhibitory checkpoint molecules include but are not limited to PD-1, TEVI-3, VISTA, A2AR, B7-H3, B7-H4, BTLA, CTLA-4, IDO, KIR and LAG3, PDL1, PDL2, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. Ligands of checkpoint proteins include but are not limited to CTLA-4, PDL1, PDL2, PD1, B7-H3, B7-H4, BTLA, HVEM, TIM3, GAL9, LAG3, VISTA, KIR, 2B4, CD 160, CGEN-15049, CHK 1, CHK2, A2aR, and B-7 family ligands. In some embodiments, the anti-PD-1 antibody is BMS-936558 (nivolumab). In other embodiments, the anti-CTLA-4 antibody is ipilimumab (trade name Yervoy, formerly known as MDX-010 and MDX-101).

The LPMP/mRNA therapeutic composition and anti-cancer therapeutic agent can be combined to enhance immune therapeutic responses even further. The LPMP/mRNA therapeutic composition and other therapeutic agent may be administered simultaneously or sequentially. When the other therapeutic agents are administered simultaneously, they can be administered in the same or separate formulations, but are administered at the same time. The other therapeutic agents are administered sequentially with one another and with the LPMP/mRNA therapeutic composition, when the administration of the other therapeutic agents and the LPMP/mRNA therapeutic composition is temporally separated. The separation in time between the administration of these compounds may be a matter of minutes or it may be longer, e.g. hours, days, weeks, months. For example, in some embodiments, the separation in time between the administration of these compounds is 1 hour, 2 hours, 3 hours 4 hours, 5 hours, 6 hours, 8 hours, 12 hours, 24 hours or more. In some embodiments, the separation in time between the administration of these compounds is 2 days, 3 days, 4 days, 5 days, 6 days, or 7 days or more. In some embodiments, the LPMP/mRNA therapeutic composition is administered before the anti-cancer therapeutic. In some embodiments, the LPMP/mRNA therapeutic composition is administered after the anti-cancer therapeutic.

Other therapeutic agents include but are not limited to anti-cancer therapeutic, adjuvants, cytokines, antibodies, antigens, etc.

The therapeutic agent may also include those that can treat and/or prevent chronic pain and/or symptoms of chronic pain, including but are not limited to:
(i) Opioid analgesics such as, morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, Buprenorphine, butorphanol, nalbuphine or pentazocine,
(ii) Non-steroidal anti-inflammatory drugs (NSAIDs) such as aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, Nabumetone, naproxen, oxaprozin, phenylbutazone, piroxicam, sulindac, tolmetin or zomepyrac, mobic (Meloxicam SR), or a pharmaceutically acceptable salt thereof
(iii) Barbiturate analgesics, such as amobarbital, aprobarbital, butabarbital, butalbital, methobarbital, metalbital, methohexital, pentobarbital, phenobarbital, secobarbital, talbutal, thiamaryl or thiopental or a pharmaceutically acceptable salt thereof,
(iv) benzodiazepines having analgesic activity, such as chlordiazepoxide, chlorazepic acid, diazepam, flurazepam, lorazepam, oxazepam, temazepam or triazolam or a pharmaceutically acceptable salt thereof;
(v) H1 antagonists having analgesic activity, for example, diphenhydramine, pyrilamine, promethazine, chlorpheniramine or chlorcyclidine or a pharmaceutically acceptable salt thereof; (vi) Analgesics, for example, glutethimide, meprobamate, methacalone or dichloralphenazone or a pharmaceutically acceptable salt thereof;
(vii) Skeletal muscle relaxant, for example, baclofen, carisoprodol, chlorzoxazone, cyclobenzaprine, methocarbamol or orfrenazine or a pharmaceutically acceptable salt thereof;
(viii) NMDA receptor antagonist such as dextromethorphan ((+)-3-hydroxy-N-methylmorphinan) or its metabolite dextrorphan ((+)-3-hydroxy-N-methylmorphinan), Ketamine, memantine, pyrroloquinoline quinone or cis-4-(phosphonomethyl)-2-piperidine carboxylic acid, or a pharmaceutically acceptable salt thereof
(ix) α-adrenergic agents such as doxazosin, tamsulosin, clonidine or 4-amino-6,7-dimethoxy-2-(5-methanesulfonamide-1,2,3,4-tetrahydroisoquinol-2-)Yl)-5-(2-pyridyl) quinazoline,
(x) tricyclic antidepressants such as desipramine, imipramine, amitriptyline or nortriptyline,
(xi) anticonvulsants such as carbamazepine or valproate,
(xii) tachykinin (NK) antagonists, in particular NK-3, NK-2 or NK-1 antagonists, for example (αR,9R)-7-[3,5-bis (trifluoromethyl) benzyl]-8,9,10,11-tetrahydro-9-methyl-5-(4-methylphenyl)-7H-[1,4] diazosino [2,1-g][1,7]naphtholidine-6-13-dione (TAK-637), 5-[[(2R,3S)-2-[(1R)-1-[3,5-bis (trifluoromethyl) phenyl]ethoxy-3-(4-fluorophenyl)-4-) Morpholynyl]methyl]-1,2-dihydro-3H-1,2,4-triazol-3-one (MK-869), ranepitant, dapitant or 3-[[2-methoxy-5-(trifluoromethoxy) phenyl] Methyl Amy]-2-phenyl-piperidine (2S,3S),
(xiii) muscarinic antagonists, for example oxybutin, tolterodine, propiverine, trospium chloride or darifenacin,
(xiv) COX-2 inhibitors, eg celecoxib, rofecoxib or valdecoxib,
(xv) non-selective COX inhibitors, preferably those with GI protection, such as nitroflurbiprofen (HCT-1026),
(xvi) coal tar analgesics, especially paracetamol,
(xvii) neuroleptics, such as droperidol,
(xviii) vanilloid receptor agonists (for example resiniferatoxin) or antagonists (for example capsazepine),
(xix) beta-adrenergic agents, such as propranolol,
(xx) a local anesthetic such as mexiletine,
(xxi) corticosteroids, eg dexamethasone,
(xxii) serotonin receptor agonists or antagonists,
(xxiii) cholinergic (nicotinic) analgesics,
(xxiv) Tramadol™,
(xxv) PDEV inhibitors such as sildenafil, vardenafil or tadalafil,

In some embodiments, provided methods include administering a LPMP/mRNA therapeutic composition in combination with an immune checkpoint modulator. In some embodiments, an immune checkpoint modulator, e.g., checkpoint inhibitor such as an anti-PD-1 antibody, is administered at a dosage level sufficient to deliver 100-300 mg to the subject. In some embodiments, an immune checkpoint modulator, e.g., checkpoint inhibitor such as an anti-PD-1 antibody, is administered at a dosage level sufficient to deliver 200 mg to the subject. In some embodiments, an immune checkpoint modulator, e.g., checkpoint inhibitor such as an anti-PD-1 antibody, is administered by intravenous infusion. In some embodiments, the immune checkpoint modulator is administered to the subject twice, three times, four times or more. In some embodiments, the immune checkpoint modulator is administered to the subject on the same day as the LPMP/mRNA therapeutic composition administration.

The LPMP/mRNA therapeutic composition may be formulated or administered in combination with one or more pharmaceutically-acceptable excipients. In some embodiments, the LPMP/mRNA therapeutic composition comprises at least one additional active substances, such as, for example, a therapeutically-active substance, a prophylactically-active substance, or a combination of both. The LPMP/mRNA therapeutic composition may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

In some embodiments, the LPMP/mRNA therapeutic compositions are administered to humans, human patients, or subjects. The phrase “active ingredient” generally refers to the LPMP/mRNA therapeutic composition or the polynucleotides contained therein, for example, RNA polynucleotides (e.g., mRNA polynucleotides) encoding antigenic or therapeutic polypeptides.

Formulations of the LPMP/mRNA therapeutic composition described herein may be prepared by any method known or hereafter developed in the art of pharmacology. In general, such preparatory methods include the step of bringing the active ingredient (e.g., mRNA polynucleotide) into association with an excipient and/or one or more other accessory ingredients, and then, if necessary and/or desirable, dividing, shaping and/or packaging the product into a desired single- or multi-dose unit.

The LPMP/mRNA therapeutic composition can be formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with cancer RNA vaccines (e.g., for transplantation into a subject), hyaluronidase, nanoparticle mimics and combinations thereof.

### Kits

The present invention also provides a kit including a container having a mRNA therapeutic composition described herein. The kit may further include instructional material for applying or delivering the mRNA therapeutic composition to a subject in accordance with a method of the present invention. The skilled artisan will appreciate that the instructions for applying the mRNA therapeutic composition in the methods of the present invention can be any form of instruction. Such instructions include, but are not limited to, written instruction material (such as, a label, a booklet, a pamphlet), oral instructional material (such as on an audio cassette or CD) or video instructions (such as on a video tape or DVD).

Embodiment 1. An mRNA therapeutic composition, comprising:
one or more polynucleotides encoding one or more antigenic (e.g., tumor antigenic) or signaling polypeptides,
formulated within a lipid reconstructed plant messenger packs (LPMPs) comprising natural lipids and an ionizable lipid, wherein the ionizable lipid has two or more of the characteristics listed below:
(i) at least 2 ionizable amines;
(ii) at least 3 lipid tails; wherein each of the lipid tails is at least 6 carbon atoms in length;
(iii) a pKa of about 4.5 to about 7.5;
(iv) an ionizable amine and a heteroorganic group separated by a chain of at least two atoms; and
(v) an N:P ratio of at least 10.
   Embodiment 2. The mRNA therapeutic composition of embodiment 1, wherein the natural lipids are extracted from lemon or algae.
   Embodiment 3. The mRNA therapeutic composition of embodiment 1, wherein the LPMPs further comprise a sterol and a polyethylene glycol (PEG)-lipid conjugate.
   Embodiment 4. The mRNA therapeutic composition of embodiment 3, wherein the LPMPs comprise ionizable lipid:natural lipids:sterol:PEG-lipid at a molar ratio of about 35:50:12.5:2.5.
   Embodiment 5. The mRNA therapeutic composition of embodiment 3, wherein the LPMPs comprise ionizable lipid:natural lipids:sterol:PEG-lipid at a molar ratio of about 35:20:42.5:2.5.
   Embodiment 6. The mRNA therapeutic composition of embodiment 1, wherein the ionizable lipid is selected from the group consisting of 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), MD1 (cKK-E12), OF2, EPC, ZA3-Ep10, TT3, LP01, 5A2-SC8, Lipid 5, SM-102 (Lipid H), and ALC-315.
   Embodiment 7. The mRNA therapeutic composition of embodiment 1, wherein the ionizable lipid is

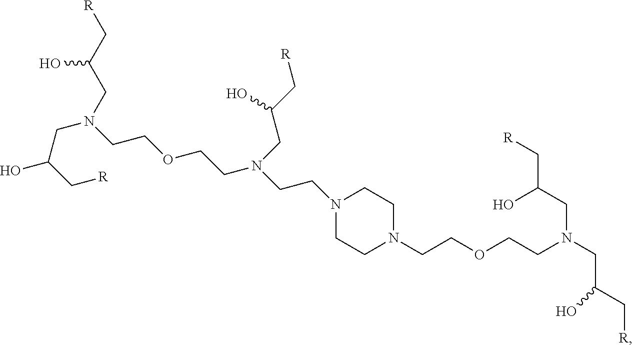

wherein R is C8-C14 alkyl group.
   Embodiment 8. The mRNA therapeutic composition of embodiment 1, wherein the polypeptide comprises a tumor specific antigen, a tumor associated antigen, a tumor neoantigen, or a combination thereof.
   Embodiment 9. The mRNA therapeutic composition of embodiment 1, wherein the polypeptide comprises p53, ART-4, BAGE, ss-catenin/m, Bcr-abL CAMEL, CAP-1, CASP-8, CDC27/m, CDK4/m, CEA, CLAUDIN-12, c-MYC, CT, Cyp-B, DAM, ELF2M, ETV6-AML1, G250, GAGE, GnT-V, Gap 100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A10, MAGE-A11, or MAGE-A12, MAGE-B, MAGE-C, MART-1/Melan-A, MC1R, Myosin/m, MUC1, MUM-1, -2, -3, NA88-A, NF1, NY-ESO-1, NY-BR-1, pI90 minor BCR-abL, Plac-1, Pml/RARa, PRAME, proteinase 3, PSA, PSM, RAGE, RU1 or RU2, SAGE, SART-1 or S ART-3, SCGB3A2, SCP1, SCP2, SCP3, SSX, SURVIVIN, TEL/AML1, TPI/m, TRP-1, TRP-2, TRP-2/INT2, TPTE, WT, WT-1, or a combination thereof.
   Embodiment 10. The mRNA therapeutic composition of embodiment 1, wherein the polypeptide comprises CD2, CD3, CD4, CD8, CD11b, CD14, CD16, CD19, CD20, CD22, CD25, CD27, CD33, CD37, CD38, CD40, CD44, CD45, CD47, CD52, CD56, CD70, CD79, CD137, 4-IBB, 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbBI, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gplOO, gpA33, GPNMB, HLA, HLA-DR, ICOS, IGF1R, Integrin av, Integrin αvβ, LAG-3, Lewis Y, Mesothelin, c-MET, MN Carbonic anhydrase IX, MUC1, MUC16, Nectin-4, KGD2, NOTCH, OX40, OX40L, PD-1, PDL1, PSCA, PSMA, RANKL, ROR1, ROR2, SLC44A4, Syndecan-1, TACI, TAG-72, Tenascin, TIM3, TRAILR1, TRAILR2, VEGFR-1, VEGFR-2, VEGFR-3, and variants thereof.
   Embodiment 11. The mRNA therapeutic composition of embodiment 1, wherein the polypeptide is IL-2 peptide, IL-2-Ra, tdTomato, Cre recombinase, GFP, eGFP, Anti-CD19, CD20, CAR-T, Anti-HER2, Etanercept (Enbrel), Humira, erythropoietin, Epogen, Filgrastim, Keytruda, Rituximab, Romiplostim, Sargramostim, or a fragment or subunit thereof.
   Embodiment 12. The mRNA therapeutic composition of embodiment 1, wherein the polynucleotide is an mRNA which encodes an IL-2 molecule comprising an amino acid sequence having at least 85%, 90%, 95%, 96%, 97%, 98%, 99% or 100% identity to the amino acid sequence of an IL-2 molecule provided in any one of Tables I-Ill.
   Embodiment 13. The mRNA therapeutic composition of embodiment 1, wherein the tumor antigenic polypeptide comprises a tumor antigen selected from the group consisting of a carcinoma, a sarcoma, a melanoma, a lymphoma, a leukemia, and a combination thereof.
   Embodiment 14. The mRNA therapeutic composition of embodiment 13, wherein the tumor antigenic polypeptide comprises a lung cancer antigen.
   Embodiment 15. The mRNA therapeutic composition of embodiment 1, wherein the polynucleotide is an mRNA, and the mRNA is derived from
(a) a DNA molecule; or
(b) an RNA molecule, wherein T is substituted with U.
   Embodiment 16. The mRNA therapeutic composition of embodiment 15, wherein the DNA molecule further comprises a promoter.
   Embodiment 17. The mRNA therapeutic composition of embodiment 16, wherein the promoter is located at the 5′ UTR.
   Embodiment 18. The mRNA therapeutic composition of embodiment 15, wherein the promoter is a T7 promoter, a T3 promoter, or an SP6 promoter.
   Embodiment 19. The mRNA therapeutic composition of embodiment 15, wherein the RNA molecule is a self-replicating RNA molecule.
   Embodiment 20. The mRNA therapeutic composition of embodiment 15 or embodiment 19, wherein the RNA molecule further comprises a 5′ cap.
   Embodiment 21. The mRNA therapeutic composition of embodiment 20, wherein the 5′ cap has a Cap 1 structure, a Cap 1 (m6A) structure, a Cap 2 structure, a Cap 3 structure, a Cap 0 structure, or any combination thereof.
   Embodiment 22. The mRNA therapeutic composition of embodiment 12, wherein the IL-2 molecule comprises a naturally occurring IL-2 molecule, a fragment of a naturally occurring IL-2 molecule, or a variant thereof.
   Embodiment 23. The mRNA therapeutic composition of embodiment 22, wherein the IL-2 molecule comprises a variant of a naturally occurring IL-2 molecule, or a fragment thereof.
   Embodiment 24. The mRNA therapeutic composition of embodiment 15, wherein the mRNA comprises a 5′ untranslated region (UTR) and/or a 3′ UTR.
   Embodiment 25. The mRNA therapeutic composition of embodiment 24, wherein the 5′ UTR comprises a Kozak sequence.
   Embodiment 26. The mRNA therapeutic composition of embodiment 24, wherein the 3′ UTR comprises a sequence derived from an amino-terminal enhancer of split (AES).
   Embodiment 27. The mRNA therapeutic composition of embodiment 24, wherein the 3′ UTR comprises a sequence derived from mitochondrially encoded 12S rRNA (mtRNRI).
   Embodiment 28. The mRNA therapeutic composition of embodiment 15, wherein the mRNA comprises a poly(A) sequence.
   Embodiment 29. The mRNA therapeutic composition of embodiment 28, wherein the poly(A) sequence is a 110-nucleotide sequence consisting of a sequence of 30 adenosine residues, a 10-nucleotide linker sequence, and a sequence of 70 adenosine residues.
   Embodiment 30. The mRNA therapeutic composition of embodiment 1, wherein the LPMP is a lipophilic moiety selected from the group consisting of a lipoplex, a liposome, a lipid nanoparticle, a polymer-based carrier, an exosome, a lamellar body, a micelle, and an emulsion.
   Embodiment 31. The mRNA therapeutic composition of embodiment 1, wherein the LPMP is a liposome selected from the group consisting of a cationic liposome, a nanoliposome, a proteoliposome, a unilamellar liposome, a multilamellar liposome, a ceramide-containing nanoliposome, and a multivesicular liposome.
   Embodiment 32. The mRNA therapeutic composition of embodiment 1, wherein the LPMP is a lipid nanoparticle.
   Embodiment 33. The mRNA therapeutic composition of embodiment 1, wherein the LPMP has a size of less than about 200 nm.
   Embodiment 34. The mRNA therapeutic composition of embodiment 33, wherein the LPMP has a size of less than about 150 nm.
   Embodiment 35. The mRNA therapeutic composition of embodiment 33, wherein the LPMP has a size of less than about 100 nm.
   Embodiment 36. The mRNA therapeutic composition of embodiment 32, wherein the lipid nanoparticle has a size of about 55 nm to about 80 nm.
   Embodiment 37. The mRNA therapeutic composition of embodiment 3, wherein the PEG-lipid conjugate is PEG-DMG or PEG-PE.
   Embodiment 38. The mRNA therapeutic composition of embodiment 37, wherein the PEG-DMG is PEG2000-DMG or PEG2000-PE.
   Embodiment 39. The mRNA therapeutic composition of any one of the preceding embodiments, wherein the LPMP comprises:
about 20 mol % to about 50 mol % of the ionizable lipid,
about 20 mol % to about 60 mol % of the natural lipids,
about 7 mol % to about 20 mol % of the sterol, and
about 0.5 mol % to about 3 mol % of the polyethylene glycol (PEG)-lipid conjugate.
   Embodiment 40. The mRNA therapeutic composition of embodiment 39, wherein the LPMP comprises:
about 35 mol % of the ionizable lipid,
about 50 mol % of the natural lipids,
about 12.5 mol % of the sterol, and
about 2.5 mol % the polyethylene glycol (PEG)-lipid conjugate.
   Embodiment 41. The mRNA therapeutic composition of any one of the preceding embodiments, wherein the mRNA therapeutic composition has a total lipid:polynucleotide weight ratio of about 50:1 to about 10:1.
   Embodiment 42. The mRNA therapeutic composition of embodiment 41, wherein the mRNA therapeutic composition has a total lipid:polynucleotide weight ratio of about 44:1 to about 24:1.
   Embodiment 43. The mRNA therapeutic composition of embodiment 41, wherein the mRNA therapeutic composition has a total lipid:polynucleotide weight ratio of about 40:1 to about 28:1.
   Embodiment 44. The mRNA therapeutic composition of embodiment 41, wherein the mRNA therapeutic composition has a total lipid:polynucleotide weight ratio of about 38:1 to about 30:1.
   Embodiment 45. The mRNA therapeutic composition of embodiment 41, wherein the mRNA therapeutic composition has a total lipid:polynucleotide weight ratio of about 37:1 to about 33:1.
   Embodiment 46. The mRNA therapeutic composition of embodiment 1, further comprising a HEPES or TRIS buffer at a pH of about 7.0 to about 8.5.
   Embodiment 47. The mRNA therapeutic composition of embodiment 46, wherein the HEPES or TRIS buffer is at a concentration of about 7 mg/mL to about 15 mg/mL.
   Embodiment 48. The mRNA therapeutic composition of embodiment 46 or 47, further comprising about 2.0 mg/mL to about 4.0 mg/mL of NaCl.
   Embodiment 49. The mRNA therapeutic composition of embodiment 1, further comprising one or more cryoprotectants.
   Embodiment 50. The mRNA therapeutic composition of embodiment 49, wherein the one or more cryoprotectants are selected from the group consisting of sucrose, glycerol, and a combination thereof.
   Embodiment 51. The mRNA therapeutic composition of embodiment 50, wherein the mRNA therapeutic composition comprises a combination of sucrose at a concentration of about 70 mg/mL to about 110 mg/mL and glycerol at a concentration of about 50 mg/mL to about 70 mg/mL.
   Embodiment 52. The mRNA therapeutic composition of embodiment 1, wherein the mRNA therapeutic is a lyophilized composition.
   Embodiment 53. The mRNA therapeutic composition of embodiment 52, wherein the lyophilized mRNA therapeutic composition comprises one or more lyoprotectants.
   Embodiment 54. The mRNA therapeutic composition of embodiment 53, wherein the lyophilized mRNA therapeutic composition comprises a poloxamer, potassium sorbate, sucrose, or any combination thereof.
   Embodiment 55. The mRNA therapeutic composition of embodiment 54, wherein the poloxamer is poloxamer 188.
   Embodiment 56. The mRNA therapeutic composition of any one of embodiments 52 to 55, wherein the lyophilized mRNA therapeutic composition comprises about 0.01 to about 1.0% w/w of the polynucleotides.
   Embodiment 57. The mRNA therapeutic composition of any one of embodiments 52 to 55, wherein the lyophilized mRNA therapeutic composition comprises about 1.0 to about 5.0% w/w lipids.
   Embodiment 58. The mRNA therapeutic composition of any one of embodiments 52 to 55, wherein the lyophilized mRNA therapeutic composition comprises about 0.5 to about 2.5% w/w of TRIS buffer.
   Embodiment 59. The mRNA therapeutic composition of any one of embodiments 52 to 55, wherein the lyophilized mRNA therapeutic composition comprises about 0.75 to about 2.75% w/w of NaCl.
   Embodiment 60. The mRNA therapeutic composition of any one of embodiments 52 to 55, wherein the lyophilized mRNA therapeutic composition comprises about 85 to about 95% w/w of a sugar.
   Embodiment 61. The mRNA therapeutic composition of embodiment 60, wherein the sugar is sucrose.
   Embodiment 62. The mRNA therapeutic composition of any one of embodiments 52 to 55, wherein the lyophilized mRNA therapeutic composition comprises about 0.01 to about 1.0% w/w of a poloxamer.
   Embodiment 63. The mRNA therapeutic composition of embodiment 62, wherein the poloxamer is poloxamer 188.
   Embodiment 64. The mRNA therapeutic composition of any one of embodiments 52 to 55, wherein the lyophilized mRNA therapeutic composition comprises about 1.0 to about 5.0% w/w of potassium sorbate.
   Embodiment 65. A method for making a mRNA therapeutic composition, comprising:
reconstituting a film comprising purified PMP lipids in the presence of an ionizable lipid to produce a lipid reconstructed plant messenger packs (LPMP) comprising the ionizable lipid, wherein the ionizable lipid has two or more of the characteristics listed below:
(i) at least 2 ionizable amines;
(ii) at least 3 lipid tails; wherein each of the lipid tails is at least 6 carbon atoms in length;
(iii) a pKa of about 4.5 to about 7.5;
(iv) an ionizable amine and a heteroorganic group separated by a chain of at least two atoms; and
(v) an N:P ratio of at least 10, and
loading into the LPMPs with one or more polynucleotides encoding one or more antigenic (e.g., tumor antigenic) or signaling polypeptides.
   Embodiment 66. A method of delivering an mRNA therapeutic in a subject, comprising:
administering to the subject the mRNA therapeutic composition of any one of embodiments 1 to 64.
   Embodiment 67. A method of inducing an immune response in a subject, comprising:
administering to the subject the mRNA therapeutic composition of any one of embodiments 1 to 64.
   Embodiment 68. A method of treating or preventing a cancer in a subject, comprising:
administering to the subject the mRNA therapeutic composition of any one of embodiments 1 to 64.
   Embodiment 69. The method of any one of embodiments 66 to 68, wherein the mRNA therapeutic composition is administered by oral, intravenous, intradermal, intramuscular, intranasal, intraocular, rectal, intrajejunal, intratumoral, and/or subcutaneous administration.
   Embodiment 70. The method of embodiment 69, wherein the mRNA therapeutic composition is administered by oral, intravenous, intramuscular, and/or subcutaneous administration.
   Embodiment 71. The method of any one of embodiments 66 to 68, wherein the mRNA therapeutic composition is administered at a dosage level sufficient to deliver about 0.006 mg/kg to about 0.5 mg/kg of the polynucleotide (e.g., mRNA) to the subject.
   Embodiment 72. The method of embodiment 71, wherein the mRNA therapeutic composition is administered at a dosage level sufficient to deliver about 0.01 mg/kg, about 0.05 mg/kg, or about 0.1 mg/kg of the polynucleotide (e.g., mRNA) to the subject.
   Embodiment 72. The method of embodiment 67 or 68, wherein the mRNA therapeutic composition is administered to the subject one time, two times, three times, four times, or more.
   Embodiment 73. The method of embodiment 72, wherein the mRNA therapeutic composition is administered to the subject once or twice.
   Embodiment 74. The method of any one of embodiments 66 to 68, further comprising: administering an additional therapeutic agent to the subject.
   Embodiment 75. The method of embodiment 74, wherein the additional therapeutic agent is an anti-cancer therapeutic agent.
   Embodiment 76. The method of embodiment 75, wherein the additional therapeutic agent is a therapeutic agent that treats and/or prevents chronic pain.
   Embodiment 77. The method of embodiment 76, wherein the additional therapeutic agent is buprenorphine, Meloxicam SR, or combinations thereof.
   Embodiment 78. The method of embodiment 74, wherein the additional therapeutic agent is administered prior to, concurrent with, or after the administration of the mRNA therapeutic composition.
   Embodiment 79. The method of embodiment 72, wherein the mRNA therapeutic composition is administered to the subject three or four times.
   Embodiment 80. The method of embodiment 71, wherein the mRNA therapeutic composition is administered at a dosage level sufficient to deliver about 0.006 mg/kg, about 0.02 mg/kg, about 0.2 mg/kg, about 0.4 mg/kg, or about 0.5 mg/kg of the polynucleotide (e.g., mRNA) to the subject.
   Embodiment 81. The method of embodiment 71, wherein the mRNA therapeutic composition is administered at a dosage level sufficient to deliver about 0.006 mg/kg, about 0.01 mg/kg, about 0.02 mg/kg, about 0.05 mg/kg, about 0.1 mg/kg, or about 0.4 mg/kg of the polynucleotide (e.g., mRNA) to the subject.
   Embodiment 82. The method of embodiment 66 to 68, wherein the mRNA therapeutic composition is administered at a dosage level sufficient to deliver about 0.0003 mg/kg to about 0.002 mg/kg of the polynucleotide (e.g., mRNA) to the subject.
   Embodiment 83. The method of embodiment 82, wherein the mRNA therapeutic composition is administered to the subject one time, two times, three times, four times, or more at a dosage level sufficient to deliver about 0.0003 mg/kg to about 0.5 mg/kg of the polynucleotides to the subject.
   Embodiment 84. The mRNA therapeutic composition of embodiment 66 to 68, wherein the polynucleotide is an mRNA which encodes an IL-15 and/or IL-15Ra molecule comprising an amino acid sequence having at least 85%, 90%, 95%, 96%, 97%, 98%, 99% or 100% identity to the amino acid sequence of an IL-15 molecule provided in any one of Table 9 or Examples 1-9.
   Embodiment 85. The mRNA therapeutic composition of embodiment 84, wherein the IL-15 molecule comprises a naturally occurring IL-15 molecule, a fragment of a naturally occurring IL-15 molecule, or a variant thereof.
   Embodiment 86. The mRNA therapeutic composition of embodiment 84, wherein the IL-15Ra molecule comprises a naturally occurring IL-15Ra molecule, a fragment of a naturally occurring IL-15Ra molecule, or a variant thereof.
   Embodiment 87. The method of embodiment 66 to 68, wherein the mRNA therapeutic composition induces proliferation or activation of T cells and/or NK cells.
   Embodiment 88. The method of embodiment 66 to 68, wherein the mRNA therapeutic composition modulates proliferation or activation of T cells and/or NK cells.
   Embodiment 89. The method of embodiment 88, wherein the mRNA therapeutic composition modulates proliferation or activation of CD4 Tcells and/or CD8 T cells.
   Embodiment 90. The method of embodiment 88, wherein the mRNA therapeutic composition modulates proliferation or activation of T cells and/or NK cells in the blood, in the lymph nodes, in the spleen or in any combination thereof.
   Embodiment 91. The method of embodiment 88, wherein the mRNA therapeutic composition modulates proliferation or activation of T cells and/or NK cells in a specific organ, for instance, in the lung, the gut, or the skin.
   Embodiment 92. The method of embodiment 88, wherein the mRNA therapeutic composition modulates proliferation or activation of T cells and/or NK cells for 6 hours, 24 hours, 2 days, 3 days, 4 days, 5 days, 6 days, 7 days, 8 days, 9 days, 10 days, 13 days, or longer post-dose.
   Embodiment 93. The method of embodiment 66 to 68, wherein the mRNA therapeutic composition modulates activity or expression of Granzyme B/perforin, CD25 (IL-2Ra), TNF-alpha, IL-15, IL-6, IL-8, IL-12, GM-CSF, G-CSF, IL-1, IL-2, IL-4, IL-5, IL-8, IL-9, IL-10, IL-13, IL-17, IL-33, PD-L1, CCL2, CCL3, CCL4, CXCL1, CXCL2, CXCL10 (IP-10), CCL20, CD40, TNF-β, LAF, TCGF, BCGF, TRF, BAF, BDG, MP, LIF, OSM, TMF, PDGF, IFN-a, IFN-β, IFN-γ or any combination thereof.
   Embodiment 94. The method of embodiment 93, wherein the mRNA therapeutic compositions modulates the immune response for 6 hours, 24 hours, 2 days, 3 days, 4 days, 5 days, 6 days, 7 days, 8 days, 9 days, 10 days, 13 days, 15 days, 20 days or longer post-dose.
   Embodiment 95. The method of embodiment 94, wherein the mRNA therapeutic compositions modulates the activity or expression of the immune response post-dose in the blood or on T cells and/or NK cells.

### EXAMPLES

The invention now being generally described, it will be more readily understood by reference to the following examples, which are included merely for purposes of illustration of certain aspects and embodiments of the present invention, and are not intended to limit the invention.

### Example 1: Preparation and Modification of LPMP, and Formulation LPMP with mRNAs

The preparation of plant messenger packs (PMP), modification of PMP to prepare lipid reconstructed plant messenger packs (LPMP), and formulation of PMP and LPMP with mRNAs may be accomplished utilizing the methods disclosed in International Patent Application Publication No. WO 2021/041301, which is incorporated herein by reference in its entirety.

In particular, all the experimental protocols disclosed in Examples 1-17 of International Patent Application Publication No. WO 2021/041301, are incorporated herein by reference in their entirety, including: Example 1. Isolation of Plant Messenger Packs from plants; Example 2. Production of purified Plant Messenger Packs (PMPs); Example 3. Plant Messenger Pack characterization; Example 4. Characterization of Plant Messenger Pack stability; Example 5. Loading PMPs with cargo; Example 6. Increasing PMP cellular uptake by formulation of PMPs with ionic liquids; Example 7. Modification of PMPs using ionizable lipids; Example 8. Formulation of LPMPs with microfluidics; Example 9. mRNA loading and delivery into lipid-reconstructed PMPs using ionizable lipids; Example 10. Cellular uptake of natural and reconstructed PMPs, with and without ionizable lipid modifications; Example 11. Increasing PMP cellular uptake by formulation of PMPs with cationic lipids; Example 12. Modification of PMPs using cationic lipids; Example 13. mRNA loading and delivery into lipid-reconstructed PMPs using cationic lipids; Example 14. Cellular uptake of natural and reconstructed PMPs, with and without cationic lipid modifications; Example 15. Improved loading using the cationic lipids GL67 and Ethyl PC; Example 16. Optimization of lipid ratios for mRNA loading; and Example 17. Optimization of lipid ratios for plasmid loading.

### Example 2: Preparation of an mRNA Therapeutic Composition

### Manufacture and Characterization of Polynucleotides

The manufacture of polynucleotides and/or parts or regions thereof may be accomplished utilizing the methods taught in International Patent Application Publication No. WO 2014/152027, which is incorporated herein by reference in its entirety. Purification methods may include those taught in International Patent Application Publication Nos. WO2014/152030 and WO2014/152031, which are incorporated herein by reference in their entirety. Detection and characterization methods of the polynucleotides may be performed as taught in International Patent Application Publication No. WO2014/144039, which is incorporated herein by reference in its entirety. Characterization of the polynucleotides may be accomplished using polynucleotide mapping, reverse transcriptase sequencing, charge distribution analysis, detection of RNA impurities, or any combination of two or more of the foregoing. “Characterizing” comprises determining the RNA transcript sequence, determining the purity of the RNA transcript, or determining the charge heterogeneity of the RNA transcript, for example. Such methods are taught in, for example, International Patent Application Publication Nos. WO2014/144711 and WO2014/144767, which are incorporated herein by reference in their entirety.

Additional details of the mRNA design and modifications may be found in WO 2021/154763 and WO 2021/188969, which are incorporated herein by reference in their entirety.

Formulation of LPMP with an mRNA

### General Protocols and Experimental Designs

The protocols and detailed experimental procedures of the preparation of plant messenger packs (PMP), and modification of PMP to prepare lipid reconstructed plant messenger packs (LPMP), and formulations of PMP and LPMP with mRNAs have been discussed in Example 1, which lists all the experimental protocols disclosed in Examples 1-17 of International Patent Application Publication No. WO 2021/041301, all of which are incorporated herein by reference in their entirety. These protocols and detailed experimental procedures were followed when preparing PMP, LPMP, and PMP or LPMP formulated with mRNAs in this example.

Briefly, the isolation and purification of crude plant messenger packs (PMPs) from lemon and algae, and characterization of these PMPs followed the experimental designs and protocols for plants sources described in Example 1: Isolation of Plant Messenger Packs from plants; Example 2: Production of purified Plant Messenger Packs (PMPs); Example 3: Plant Messenger Pack characterization; and Example 4: Characterization of Plant Messenger Pack stability, all of International Patent Application Publication No. WO 2021/041301, which is incorporated herein by reference in its entirety.

The modifications of natural PMP or reconstructed lemon or algae LPMPs with cholesterol and PEG-lipid followed the experimental designs and protocols for plants sources described in Example 6: Increasing PMP cellular uptake by formulation of PMPs with ionic liquids; Example 7: Modification of PMPs using ionizable lipids; Example 10: Cellular uptake of natural and reconstructed PMPs, with and without ionizable lipid modifications; Example 11: Increasing PMP cellular uptake by formulation of PMPs with cationic lipids; Example 12: Modification of PMPs using cationic lipids; and Example 14: Cellular uptake of natural and reconstructed PMPs, with and without cationic lipid modifications, all of International Patent Application Publication No. WO 2021/041301, which is incorporated herein by reference in its entirety.

Formulations of the PMPs and lemon-lipid- or algae-lipid-reconstructed LPMPs with mRNAs followed the experimental designs and protocols for nucleic acids loading described in Example 5: Loading PMPs with cargo; Example 8: Formulation of LPMPs with microfluidics; Example 9: mRNA loading and delivery into lipid-reconstructed PMPs using ionizable lipids; Example 13: mRNA loading and delivery into lipid-reconstructed PMPs using cationic lipids; Example 15: Improved loading using the cationic lipids GL67 and Ethyl PC; Example 16: Optimization of lipid ratios for mRNA loading; and Example 17: Optimization of lipid ratios for plasmid loading, all of International Patent Application Publication No. WO 2021/041301, which is incorporated herein by reference in its entirety.

### An Exemplary LPMP Composition

An exemplary composition for lipid reconstructed plant messenger packs (LPMP) is as follows:
93-97% of lipids extracted from (lemon, algae, or Wolffia)
0.5-2.0% of PEG-PE
2.5-5% cholesterol or sitosterol
3:1 ratio of lipids to insulin

An exemplary composition for lipid reconstructed plant messenger packs (LPMP) derived from lemon (recLemon LPMPs) is provided in Table 1, as compared to conventional LNP.

TABLE 1

Molar ratios

| Lipids | LNP | recLemon |
|---|---|---|
| C12-200 | 35 | 35 |
| DOPE | 16 | — |
| Lemon Lipids | — | 50 |
| Cholesterol | 46.5 | 12.5 |
| DMPE-PEG2k | 2.5 | 2.5 |

### Formulations for LPMP/mRNA

This example describes the formulation of lemon lipid and algae lipid reconstructed LPMP formulated with ionizable lipids, sterols, and PEG lipids, to encapsulate an mRNA for LPMP/mRNA formulation. The LPMP/mRNA formulation with lemon lipid reconstructed LPMP is typically referred to as A and the LPMP/mRNA formulation with algae lipid reconstructed LPMP is typically referred to as B, e.g., as shown in Table 2 below.

In this example, lemon and algae PMP lipids were used as the PMP natural lipids; C12-200 [1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol)] was used as the ionizable lipids; cholesterol (14:0) was used as the sterols; DMPE-PEG2k was used as model PEGylated lipids.

LNP formulations. A LNP (lipid nanoparticle) formulation, as control, was prepared to result in ionizable lipid:structural lipid:sterol:PEG-lipid (C12-200:DOPE:cholesterol (14:0):DMPE-PEG2k) at a molar ratio of 35:16:46.5:2.5, respectively. To prepare this formulation, the above lipids were solubilized in ethanol, mixed at the above molar ratios, and diluted in ethanol (organic phase) to obtain total lipid concentration of 5.5 mM. An mRNA solution (aqueous phase) was prepared with RNAse-free water and 100 mM citrate buffer pH 3 for a final concentration of 50 mM citrate buffer. The formulations were maintained at an ionizable lipid to mRNA at an ionizable lipid nitrogen:mRNA phosphate (N:P) ratio of 15:1.

LPMP formulations. Reconstructed lemon (recLemon) LPMP formulation was prepared to result in ionizable lipid:natural lipids:sterol:PEG-lipid (C12-200:lemon lipid:cholesterol (14:0):DMPE-PEG2k) at a molar ratio of 35:50:12.5:2.5, respectively. To prepare this formulation, the above lipids were solubilized in ethanol, except for lemon lipid, which was solubilized in 4:1 DMF:methanol. The lipids were then mixed at the above molar ratios, and diluted to obtain total lipid concentration of 5.5 mM. An mRNA solution (aqueous phase) was prepared with RNAse-free water and 100 mM citrate buffer pH 3 for a final concentration of 50 mM citrate buffer.

Reconstructed algae (recAlgae) LPMP formulation was prepared to result in ionizable lipid:natural lipids:sterol:PEG-lipid (C12-200:algae lipid:cholesterol (14:0):DMPE-PEG2k) at a molar ratio of 35:20:42.5:2.5, respectively. To prepare this formulation, the above lipids were solubilized in ethanol, except for alage lipid, which was solubilized in 4:1 DMF:methanol. The lipids were then mixed at the above molar ratios, and diluted to obtain total lipid concentration of 5.5 mM. An mRNA solution (aqueous phase) was prepared with RNAse-free water and 100 mM citrate buffer pH 3 for a final concentration of 50 mM citrate buffer.

The lipid mixture and mRNA solution were mixed at a 1:3 ratio by volume, respectively, on the NanoAssemblr Ignite (Precision Nanosystems) at a total flow rate of 9 mL/min. The resulting formulations were then loaded into Slide-A-Lyzer G2 dialysis cassettes (10k MWCO) and dialyzed in 200 times sample volume of 1×PBS for 4 hours at room temperature with gentle stirring. The PBS solution was refreshed, and the formulations were further dialyzed for at least 14 hours at 4° C. with gentle stirring. The dialyzed formulations were then collected and concentrated by centrifugation at 3000×g (Amicon Ultra centrifugation filters, 100k MWCO).

The concentrated particles were characterized for size, polydispersity, and particle concentration using Zetasizer Ultra (Malvern Panalytical). The mRNA encapsulation efficiency was characterized by Quant-iT RiboGreen RNA Assay Kit (ThermoFisher Scientific). The particles were diluted to the desired mRNA concentration to get a final 10% sucrose solution in PBS. The formulations were then flash frozen in liquid nitrogen.

The resulting formulations with mRNA are shown in Table 2.

TABLE 2
The characterizations of a typical LPMP formulation with mRNA

| | Ionizable: | | | | | | | | | |
|---|---|---|---|---|---|---|---|---|---|---|
| | Structural: | | | | | | | | | |
| | Sterol: | | | | | | | | | Encapsulation |
| | PEG-lipid | | | | PEG- | | | Size | | efficiency |
| Formulation | Molar ratio | Ionizable | Structural | Sterol | lipid | Cargo | N:P | (nm) | PDI | (%) |
| LNP | 35:16:46.5:2.5 | C12-200 | DOPE | Chol | DMPE- | an mRNA | 15:1 | 78.47 | 0.126 | 94.4 |
| | | | | | PEG2k | | | | | |
| LPMP/mRNA | 35:50:12.5:2.5 | C12-200 | Naturally | Chol | DMPE- | an mRNA | | 96.49 | 0.300 | 94.8 |
| A (recPMP1) | | | derived | | PEG2k | | | | | |
| | | | lemon lipid | | | | | | | |
| LPMP/mRNA | 35:20:42.5:2.5 | C12-200 | Naturally | Chol | DMPE- | an mRNA | | 93.96 | 0.206 | 96.3 |
| B (recPMP2) | | | derived | | PEG2k | | | | | |
| | | | algae lipid | | | | | | | |

### Example 3. Administration of LPMP/mRNA Formulation in Mice

If not specified, the LPMP/mRNA formulation were prepared according to those described in Example 2. The coding portion of the EPO mRNA used in this example is as follows:

AUGGGCGUGCACGAGUGCCCCGCCUGGCUGUGGCUGCUGCUGAGCCUGC
UGAGCCUGCCCCUGGGCCUGCCCGUGCUGGGCGCCCCCCCCCGGCUGAU
CUGCGACAGCCGGGUGCUGGAGCGGUACCUGCUGGAGGCCAAGGAGGCC
GAGAACAUCACCACCGGCUGCGCCGAGCACUGCAGCCUGAACGAGAACA
UCACCGUGCCCGACACCAAGGUGAACUUCUACGCCUGGAAGCGGAUGGA
GGUGGGCCAGCAGGCCGUGGAGGUGUGGCAGGGCCUGGCCCUGCUGAGC
GAGGCCGUGCUGCGGGGCCAGGCCCUGCUGGUGAACAGCAGCCAGCCCU
GGGAGCCCCUGCAGCUGCACGUGGACAAGGCCGUGAGCGGCCUGCGGAG
CCUGACCACCCUGCUGCGGGCCCUGGGCGCCCAGAAGGAGGCCAUCAGC
CCCCCCGACGCCGCCAGCGCCGCCCCCCUGCGGACCAUCACCGCCGACA
CCUUCCGGAAGCUGUUCCGGGUGUACAGCAACUUCCUGCGGGGCAAGCU
GAAGCUGUACACCGGCGAGGCCUGCCGGACCGGCGACCGGUGA

FIG. 1 shows the expression level of erythropoietin (EPO) in pg/mL in serum from Ai9 (tomato red loxP) mice treated orally with reconstructed LPMPs (recPMPs) derived from lemon and comprising Cre recombinase mRNA. Ai9 mice comprise a loxP-flanked STOP cassette, which prevents transcription of the red fluorescent protein tdTomato. These mice are used as a Cre reporter strain: tdTomato fluorescence is visible following Cre-mediated recombination. Fluorescence thus indicates that the Cre mRNA has been delivered.

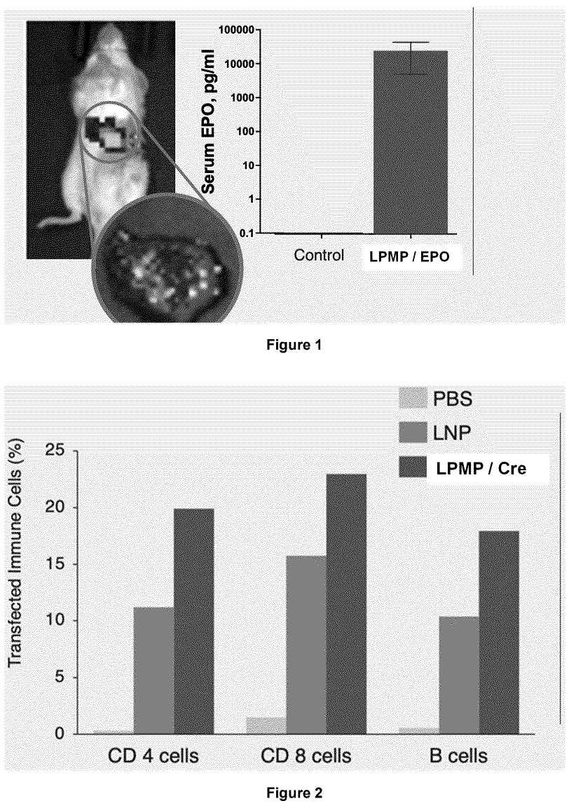

FIG. 2 shows the percentage of in vivo immune cells (CD-4 cells, CD-8 cells, or B cells) transfected from the spleens of Ai9 (tomato red loxP) mice treated with recLPMPs derived from lemon and comprising Cre recombinase mRNA. The control: a lipid nanoparticle (LNP) as control; or a phosphate-buffered saline (PBS).

FIG. 9 shows the expression of EPO in serum following oral (intra-jejunal) delivery of reconstructed lemon LPMPs/EPO mRNA to mice. Oral administration of reconstructed lemon LPMP/EPO mRNA formulation potently expressed protein at levels which exceeded required therapeutic doses for a wide range of proteins.

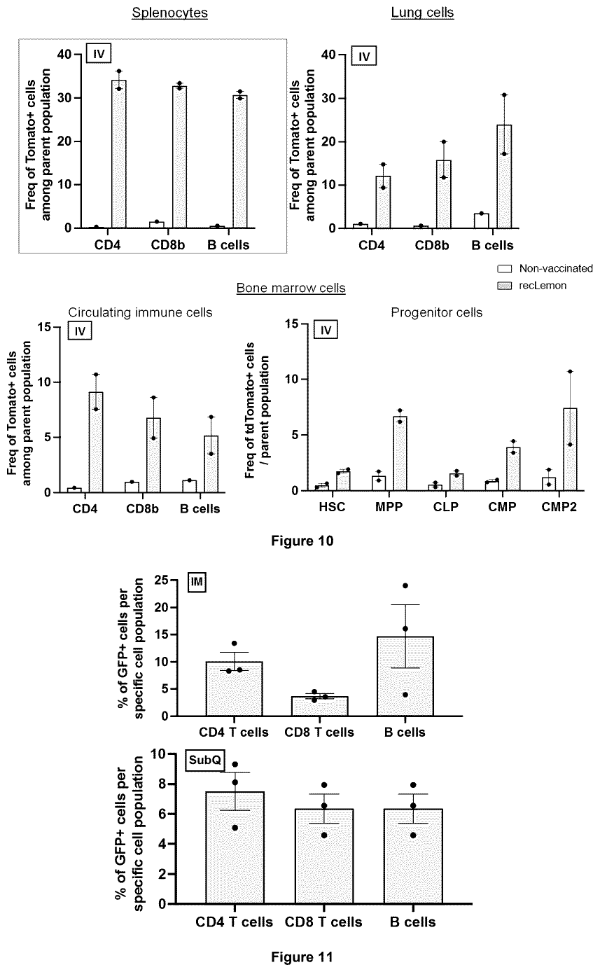

FIG. 10 shows the frequency of tomato red-positive cells (immune cells transfected in vivo) in parent populations of splenocytes, lung cells, and bone marrow cells (circulating immune cells and progenitor cells) in tomato red loxP mice cells treated with intravenously administered (IV) reconstructed lemon LPMPs comprising Cre recombinase mRNA. Intravenous administration of Cre recombinase mRNA transfected in vivo unprecedented proportions of immune cells and resident cells in the spleen, lungs and bone marrow in mice. A high level of transfection was also observed in dendritic cells, macrophages, immune cells in the airspace (alveolar macrophages), and non-immune cells (endothelial, stromal, epithelial cells).

### Example 4. Intramuscular and Subcutaneous Administrations of LPMP/EPO mRNA Formulation in Nonhuman Primates

### Dosing Regimens

Dosing regimens for a non-terminal non-human primate (NHP) study testing the efficacy of intramuscular and subcutaneous administration of reconstructed lemon LPMPs comprising EPO mRNA are provided in Tables 3-5. The study was conducted in cynomolgus macaques. Pre-dose medication comprises one dose of Buprenorphine (0.03 mg/kg/IM) and one dose of Meloxicam SR (0.6 mg/kg, SC).

TABLE 3
Dose 1

| | | | Dose | | | | | | Pre- |
|---|---|---|---|---|---|---|---|---|---|
| | No. of | Test | Level | Specified | Conc. | Dose | Dose | Sample | Dose |
| Group | Animals | formula | mg | Dose | μg/mL | Vol. | Route | Collections | Medication |
| 1 | 3 | LNP | 0.9 | 0.2 mg/kg | 1800 | 500 μL | Subcutaneous | Blood: Pre | — |
| | | | | GFP | | Per | Injection | Dose, 6, | |
| | | | | mRNA | | Injection | on Day 1 | 12, and 24 | |
| | | | | 0.1 mg/kg | | | | hours post | |
| | | | | EPO | | | | each dose | |
| | | | | mRNA | | | | Biopsy: | |
| 2 | 3 | recLemon | 0.9 | 0.2 mg/kg | 1800 | | | Injection | — |
| | | | | GFP | | | | site at~24 | |
| | | | | mRNA | | | | hours post | |
| | | | | 0.1 mg/kg | | | | Day 15 | |
| | | | | EPO | | | | dose | |
| | | | | mRNA | | | | | |
| 3 | 3 | LNP | 0.9 | 0.2 mg/kg | 1800 | | Intramuscular | | — |
| | | | | GFP | | | Injection | | |
| | | | | mRNA | | | on Day 1 | | |
| | | | | 0.1 mg/kg | | | | | |
| | | | | EPO | | | | | |
| | | | | mRNA | | | | | |
| 4 | 3 | recLemon | 0.9 | 0.2 mg/kg | 1800 | | | | — |
| | | | | GFP | | | | | |
| | | | | mRNA | | | | | |
| | | | | 0.1 mg/kg | | | | | |
| | | | | EPO | | | | | |
| | | | | mRNA | | | | | |

TABLE 4
Dose 2

| | | | Dose | | | | | | |
|---|---|---|---|---|---|---|---|---|---|
| | No. of | Test | Level | Specified | Conc. | Dose | Dose | Sample | Pre-Dose |
| Group | Animals | formula | mg | dose | μg/mL | Vol. | Route | Collections | Medication |
| 1 | 3 | recLemon | 0.45 | 0.1 mg/kg | 1800 | 250 μL | Subcutaneous | Blood: | Pre- |
| | | | | GFP | | Per | Injection | Pre | medicated |
| | | | | mRNA | | Injection | on Day 8 | Dose, 6, | |
| | | | | 0.05 mg/kg | | | | 12, and | |
| | | | | EPO | | | | 24 | |
| | | | | mRNA | | | | hours | |
| 2 | 3 | recLemon | 0.45 | 0.1 mg/kg | 1800 | | | post | — |
| | | | | GFP | | | | each | |
| | | | | mRNA | | | | dose | |
| | | | | 0.05 mg/kg | | | | Biopsy: | |
| | | | | EPO | | | | Injection | |
| | | | | mRNA | | | | site at | |
| 3 | 3 | recLemon | 0.45 | 0.1 mg/kg | 1800 | | Intramuscular | ~24 | Pre- |
| | | | | GFP | | | Injection | hours | medicated |
| | | | | mRNA | | | on Day 8 | post | |
| | | | | 0.05 mg/kg | | | | Day 15 | |
| | | | | EPO | | | | dose | |
| | | | | mRNA | | | | | |
| 4 | 3 | recLemon | 0.45 | 0.1 mg/kg | 1800 | | | | — |
| | | | | GFP | | | | | |
| | | | | mRNA | | | | | |
| | | | | 0.05 mg/kg | | | | | |
| | | | | EPO | | | | | |
| | | | | mRNA | | | | | |

TABLE 5
Dose 3

| | | | Dose | | | | | | |
|---|---|---|---|---|---|---|---|---|---|
| | No. of | Test | Level | Specified | Conc. | Dose | Dose | Sample | Pre-Dose |
| Group | Animals | formula | mg | Dose | μg/mL | Vol. | Route | Collections | Medication |
| 1 | 3 | recLemon | 0.45 | 0.05 mg/kg | 1800 | 250 μL | Subcutaneous | Blood: Pre | Pre- |
| | | | | EPO | | Per | Injection | Dose, 6, | medicated |
| | | | | mRNA | | Injection | on Day 15 | 12, and 24 | |
| | | | | only | | | | hours post | |
| 2 | 3 | recLemon | 0.09 | 0.02 mg/kg | 360 | | | each dose | — |
| | | | | GFP | | | | Biopsy: | |
| | | | | mRNA | | | | Injection | |
| | | | | 0.01 mg/kg | | | | site at~24 | |
| | | | | EPO | | | | hours post | |
| | | | | mRNA | | | | Day 15 | |
| 3 | 3 | recLemon | 0.45 | 0.05 mg/kg | 1800 | | Intramuscular | dose | Pre- |
| | | | | EPO | | | Injection | | medicated |
| | | | | mRNA | | | on Day 15 | | |
| | | | | only | | | | | |
| 4 | 3 | recLemon | 0.09 | 0.02 mg/kg | 360 | | | | — |
| | | | | GFP | | | | | |
| | | | | mRNA | | | | | |
| | | | | 0.01 mg/kg | | | | | |
| | | | | EPO | | | | | |
| | | | | mRNA | | | | | |

### Formulation and Characterization

This example describes the formulation of lemon lipid reconstructed LPMP formulated with ionizable lipids, sterols, and PEG lipids, to encapsulate mRNA (e.g., 2:1 eGFP:EPO) for LPMP/mRNA formulation.

A lipid nanoparticle (LNP) formulation was prepared according to Example 2, composed of ionizable lipid:structural lipid:sterol:PEG-lipid (C12-200:DOPE:cholesterol:14:0 PEG2000 PE) at a molar ratio of 35:16:46.5:2.5, respectively. Lipids were solubilized in ethanol. These lipids were mixed at the indicated molar ratios and diluted in ethanol (organic phase) to 12.5 mM total lipid concentration and the mRNA solution (aqueous phase) was prepared with RNAse-free water and 100 mM citrate buffer pH 3 for a final concentration of 50 mM citrate buffer. Formulations were maintained at ionizable lipid to mRNA N:P ratio of 15:1.

The reconstructed lemon LPMPs (recPMP1) was prepared according to Example 2, composed of ionizable lipid:natural lipids:sterol:PEG-lipid (C12-200:lemon lipid:cholesterol:14:0 PEG2000 PE) at a molar ratio of 35:50:12.5:2.5, respectively. Lipids were solubilized in ethanol except for lemon lipid, which was solubilized in 4:1 DMF:methanol. Formulations were then handled as above.

The lipid mix and mRNA solution were mixed at a 1:3 ratio by volume, respectively, on the NANOASSEMBLR® IGNITE™ (Precision Nanosystems) at a total flow rate of 9 mL/minute. The resulting formulations were then loaded into Slide-A-Lyzer G2 dialysis cassettes (10k MWCO) and dialyzed against 1×PBS for 2 hours at room temperature. The PBS was refreshed, and the formulations were further dialyzed for at least 14 hours at 4° C. with gentle stirring. The dialyzed formulations were then collected and concentrated by tangential flow filtration (TFF) using Sartorius VIVAFLOW® 50R cassettes (100k MWCO) at fixed feed flowrate of 150 ml/minute. The TFF-concentrated formulations were then further concentrated by centrifugation at 3000×g using AMICON® Ultra centrifugation filters (100k MWCO). The concentrated formulations were characterized for size, polydispersity, and particle concentration using a Zetasizer Ultra (Malvern Panalytical) and for mRNA encapsulation efficiency using QUANT-IT™ RIBOGREEN® RNA Assay Kit (ThermoFisher Scientific).

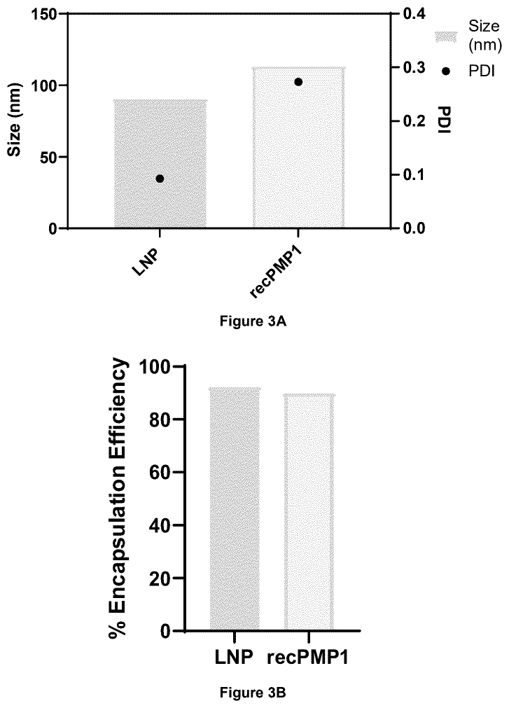

Particle characterization data are shown in FIGS. 3A and 3B and in Table 6.

TABLE 6

| | Ionizable:Structural:Sterol:PEG- | | Size | | % Encapsulation |
|---|---|---|---|---|---|
| | lipid | mRNA cargo | (nm) | PDI | Efficiency |
| LNP | 35:16:46.5:2.5 | 2:1 eGFP:EPO | 90.63 | 0.093 | 92.4 |
| recPMP1 | 35:50:12.5:2.5 | 2:1 eGFP:EPO | 113.20 | 0.273 | 90.0 |

### Dose Administration

Doses were administered according to Tables 3-5. Doses were based upon the most recently collected body weight and were rounded to the nearest 0.1 mL for dose volumes 1 mL, and to the nearest 0.01 mL for dose volumes <1 mL. The time of dose administration is used to determine target times for sample collection time points.

### Processing and Analysis for Erythropoietin (EPO) and Cytokines/Chemokines

Plasma was placed into labeled Cryo tubes and stored at nominally −80° C. until analysis. Samples were analyzed for erythropoietin (EPO) and cytokines/chemokines via MSD.
V-plex multiplex Kit for NHP proinflammatory panel 6 cytokines: IFN-γ, IL-113, IL-2, IL-6, IL-8, IL-10 (duplicate).
V-plex multiplex Kit for NHP chemokine panel 10 chemokine: Eotaxin-3, IL-8, IL-8 (HA), IP-10, MCP-1, MCP-4, MDC, MIP-1a, MIP-113, TARC (duplicate).

### Processing and Analysis for Flow Cytometry

2 mL of blood was collected and processed for flow cytometry. Whole blood samples underwent a flow cytometry staining procedure. Samples were stained with the antibodies shown in Table 7.

TABLE 7

| | | | | Blue (B) | | | | | | |
|---|---|---|---|---|---|---|---|---|---|---|
| Ultraviolet (N) | | Violet (V) | | | PE | PerCP- | Yellow (Y) | | Red (R) | |
| BUV395 | BUV661 | BV421 | BV510 | FITC | CF594 | Cy5.5 | PE | PE-Cy7 | APC | APC-Cy7 |
| CD45 | HLA-DR | CD20 | CD8 | GFP | CD11b | CD4 | CD16 | CD14 | CD3 | Live/Dead |

After staining, half of the samples were immediately run unfixed on a CytoFLEX LX flow cytometer. The data were analyzed for the following parameters: CD4 T cells (CD3+ CD4+ CD16−), CD8 T cells (CD3+ CD8+ CD16−), NKT cells (CD3+ CD16+), NK cells (CD3− CD16+ CD14+), B cells (CD20+ CD8+ CD3−), DCs (HLA-DR+ CD11b+ CD14+)/(HLA-DR+ CD11b+ CD14− CD16+), Monocytes (HLA-DR-CD16− CD14−), and Neutrophils (CD11b+ HLA-DR− CD16− CD14−).

The other half of the stained samples were fixed using BD fixation buffer or 2% PFA on ice away from light for 20 minutes, washed with PBS and resuspended in PBS and stored at 5±3° C.

### Results

Intramuscular and subcutaneous administration of reconstructed lemon LPMPs comprising EPO mRNA achieved levels of expression in non-human primates exceeding therapeutic doses for a wide range of proteins. Importantly, these results were achieved through a non-systemic route of administration.

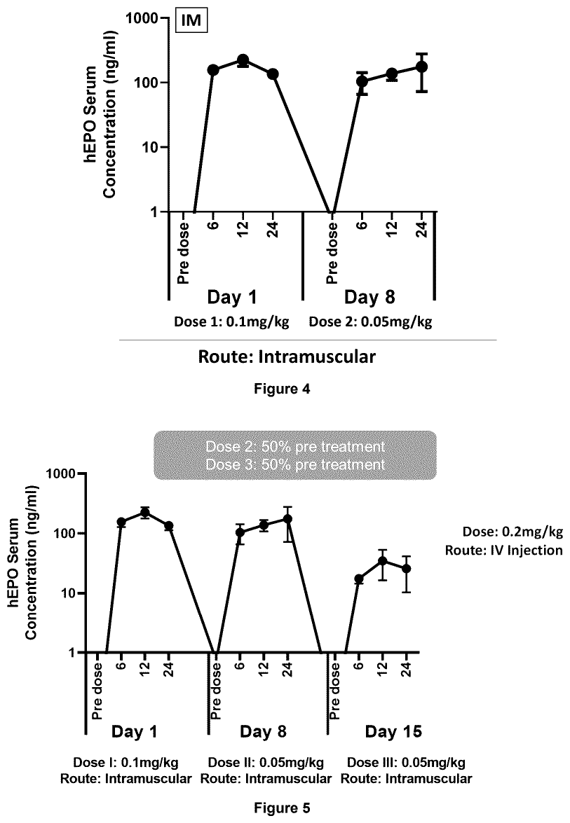

FIG. 4 shows the expression of erythropoietin (EPO) in serum following repeat doses of reconstructed lemon LPMPs comprising EPO mRNA in NHPs, wherein the reconstructed lemon LPMPs were administered intramuscularly (IM) at doses of 0.1 mg/kg on Day 1 and 0.05 mg/kg on Day 8. The concentration of human erythropoietin (hEPO) was shown pre-dose and at 6, 12, and 24 hours post-dose.

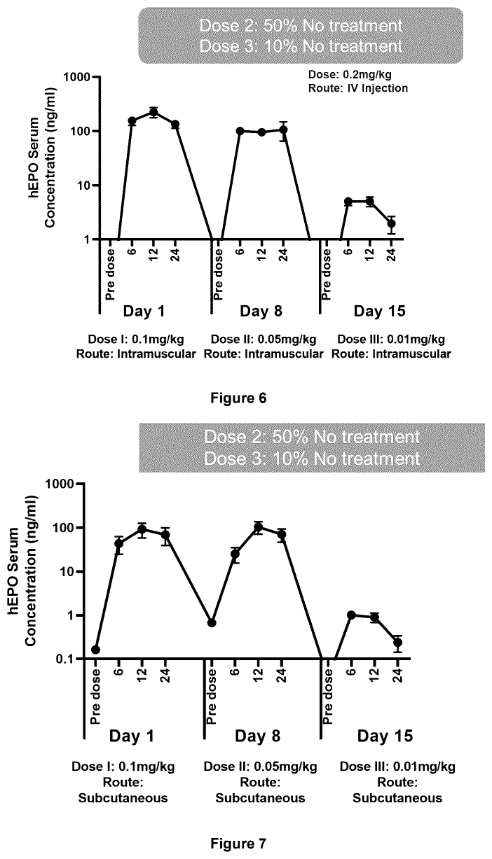

FIGS. 5 and 6 show the expression of EPO in serum following repeat doses of reconstructed lemon LPMPs comprising EPO mRNA in NHPs, wherein the reconstructed lemon LPMPs were administered intramuscularly (IM) at doses of 0.1 mg/kg on Day 1; 0.05 mg/kg on Day 8; and 0.05 mg/kg on Day 15 (FIG. 5) or 0.1 mg/kg on Day 1; 0.05 mg/kg on Day 8; and 0.01 mg/kg on Day 15 (FIG. 6).

FIG. 7 shows the expression of EPO following repeat doses of reconstructed lemon LPMPs comprising EPO mRNA, wherein the reconstructed lemon LPMPs were administered subcutaneously (SubQ) at doses of 0.1 mg/kg on Day 1, 0.05 mg/kg on Day 8, and 0.01 mg/kg on Day 15. The concentration of hEPO in serum was shown pre-dose and at 6, 12, and 24 hours post-dose.

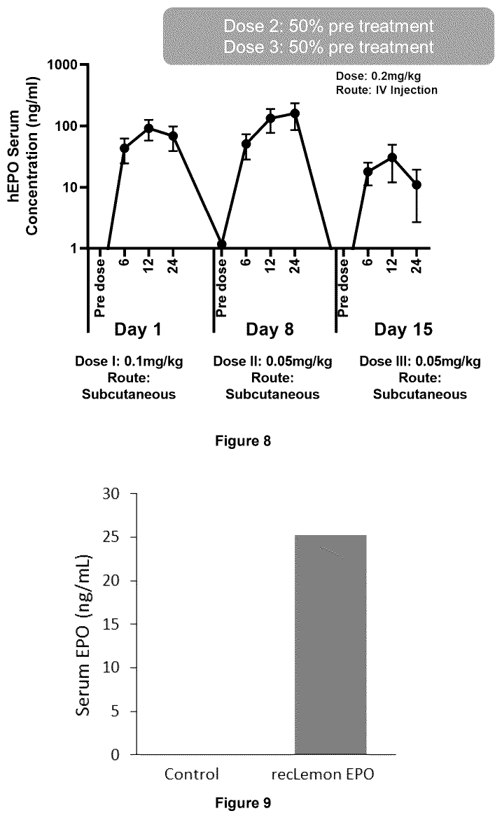

FIG. 8 shows the results of a NHP study in which the expression of EPO mRNA was measured following repeat doses of reconstructed lemon LPMPs comprising EPO, wherein the reconstructed lemon LPMPs were administered SubQ at doses of 0.1 mg/kg on Day 1, 0.05 mg/kg on Day 8, and 0.05 mg/kg on Day 15. The concentration of hEPO in serum was shown pre-dose and at 6, 12, and 24 hours post-dose.

### Example 5. Intramuscular and Subcutaneous Delivery of recLemon LPMP/GFP mRNA Formulation Transfected In Vivo Immune Cells

FIG. 11 shows the percentage of GFP-positive immune cells (CD4 T cells, CD8 T cells, and B cells) in cynomolgus macaques treated with intramuscularly administered (IM; top panel) or subcutaneously administered (SubQ; bottom panel) reconstructed lemon LPMPs comprising GFP mRNA.

### Example 6. IL-2 Induction of Anti-Tumor Responses Against MC-38 Tumors with recLemon LPMP/mRNA Formulation

This example describes a recLemon LPMP/mRNA formulation comprising a lemon lipid reconstructed with ionizable lipids (C12-200), sterols (cholesterol), and PEG lipids (DMPE-PEG2k), to encapsulate mRNA (e.g., mRNA for IL-2). The recLemon LPMP/mRNA formulation tested in this example was prepared according to Example 2. The coding portion of the IL-2 mRNA used in this example is as follows:

MYRMQLLSCI ALSLALVTNS APTSSSTKKT QLQLEHLLLD
LQMILNGINN YKNPKLTRML TFKFYMPKKA TELKHLQCLE
EELKPLEEVL NLAQSKNFHL RPRDLISNIN VIVLELKGSE
TTFMCEYADE TATIVEFLNR WITFCQSIIS TLT

### Treatment Schedule

The anti-tumor response of the recLemon LPMP/mRNA formulation was evaluated in colorectal adenocarcinoma murine model (MC38). The design of subcutaneous delivery of the recLemon LPMP/mRNA formulation and evaluation is shown in Scheme 1.

As shown in Scheme 1, MC38 tumor cells were expanded in flask then harvested for implantation in the dorsal area of the mouse (C57Bl6J mice wild type mice) subcutaneously at 1.5×10⁶ cells per 50 pI. Tumors were allowed to grow until it reached around 150 mm³ which took about 20 days. The mice (25 g mouse) were dosed peritumorally (subcutaneously, next to the tumor) at 5 μg for the first and second dose, at 2 μg for the third dose, and at 5 μg for the fourth dose.

### Tumor Measurements and Survival Rate

Tumor volume measurements were made 7 days post implantation of MC38 tumor cells and then subsequently were measured every other day.

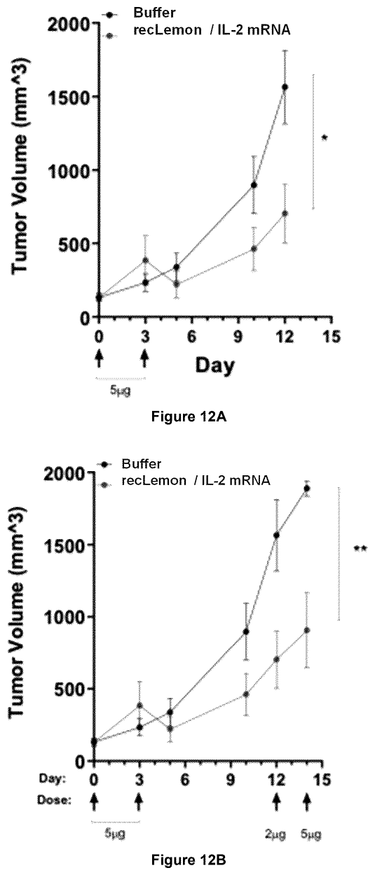

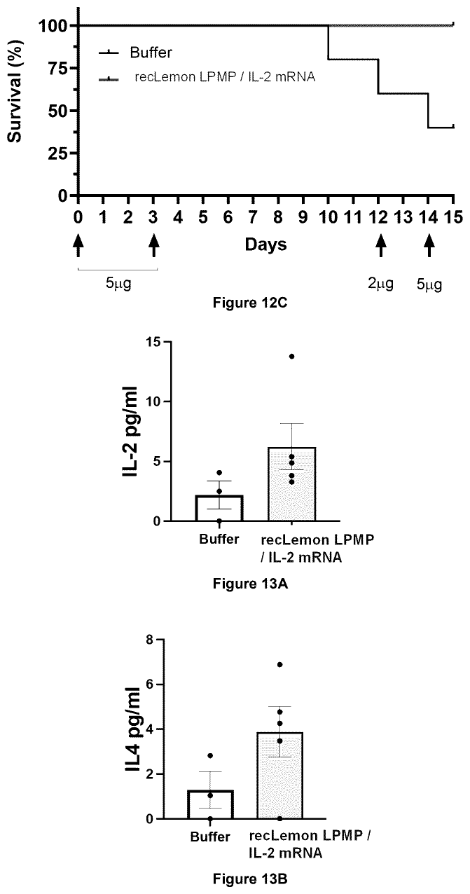

FIG. 12A shows the tumor volume measured at days post implantation of MC38 tumor cells in the mouse after the first and second peritumoral doses of the recLemon LPMP/IL-2 mRNA formulation at 5 μg. As shown in FIG. 12A, the first two doses of the peritumoral delivery of the recLemon LPMP/IL-2 mRNA formulation already induced tumor regression and had significantly decreased the tumor growth. FIG. 12B shows the tumor volume measured at days post implantation of MC38 tumor cells in the mouse after all four peritumoral doses of the recLemon LPMP/IL-2 mRNA formulation. FIG. 12C shows the survival rate of the mice at days post implantation of MC38 tumor cells after all four peritumoral doses of the recLemon LPMP/IL-2 mRNA formulation. Overall, the figures show that the peritumoral delivery of the recLemon LPMP/IL-2 mRNA formulation to the mice decreased colorectal adenocarcinoma tumors in mice by decreasing tumor growth and increasing survival.

### Serum Cytokines/Chemokines Analysis 4 Hours Post Dosing

Serum profile was skewed towards chemokines, pro-inflammatory and Th1 cytokines responses 4 hours post the 4^{th} dose by corresponding ELISA kits.

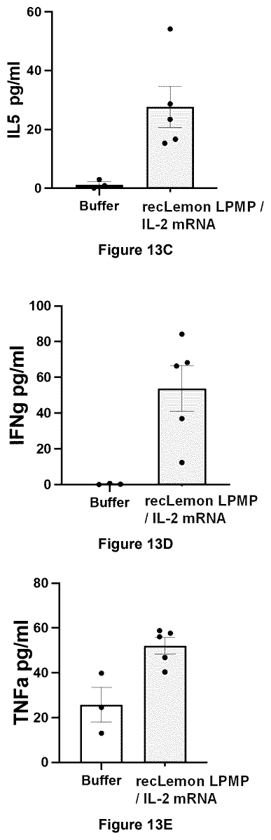

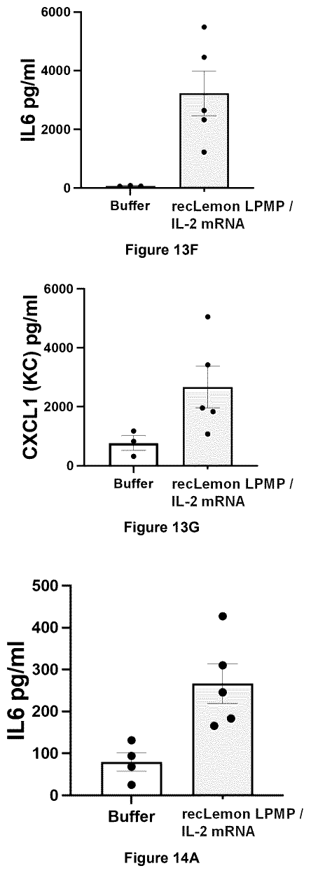

FIG. 13A shows the level of IL-2 in the serum of the mice 4 hours post the 4^{th} peritumoral dose of the recLemon LPMP/IL-2 mRNA formulation dose at 5 μg. FIGS. 13B-13G show the level of IL4 (FIG. 13B), IL5 (FIG. 13C), IFNγ (FIG. 13D), TNFα (FIG. 13E), IL6 (FIG. 13F), and CXCL1 (KC) (FIG. 13G) in the serum of the mice 4 hours post the 4^{th} peritumoral dose of the recLemon LPMP/IL-2 mRNA formulation dose at 5 μg. The results indicate that the recLemon LPMP/IL-2 mRNA formulation increased IL-2 level systemically and induced an anti-tumor cytokine profile 4 hours post injection. There were less Th2 cytokine (IL4, IL5). No differential expression of IL17 was detected.

### Serum Cytokines/Chemokines Analysis 48 Hours Post Dosing

Serum profile was skewed towards cytokines responses 48 hours post the 2^{nd} dose by corresponding ELISA kits.

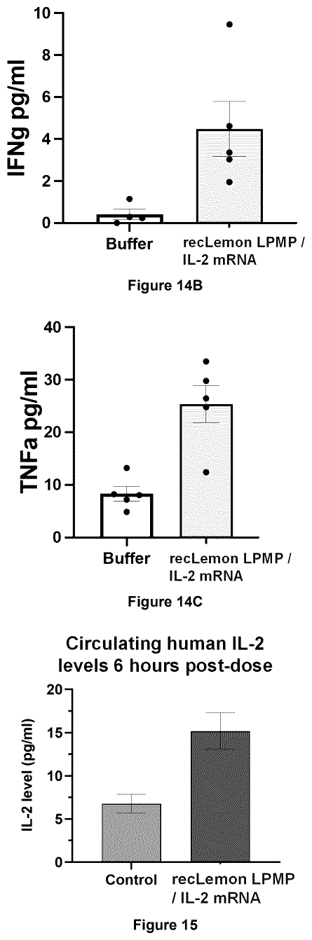

FIGS. 14A-14C show the level of IL6 (FIG. 14A), IFNγ (FIG. 14B), and TNFα (FIG. 14C) in the serum of the mice 48 hours post the 2^{nd} peritumoral dose of the recLemon LPMP/IL-2 mRNA formulation dose at 5 μg. The results indicate that the recLemon LPMP/IL-2 mRNA formulation induced anti-tumor cytokine profile that lasted 2 days post injection.

This example illustrates that the ability of the LPMP/mRNA formulation being used to deliver mRNA coding for a cytokine locally and to reach a functional systemic level. The recLemon LPMP/IL-2 mRNA formulation induced production of IL-2 in vivo (including systemic level) and mounted an anti-tumor immune response, decreasing tumor growth. In particular, a low dose of the recLemon LPMP/IL-2 mRNA formulation decreased tumor growth. The recLemon LPMP/IL-2 mRNA formulation induced a cytokine profile in tumor-bearing host which promoted immune cell recruitment and favorable for anti-tumor responses.

### Example 7. IL-2 Durability of Expression in Naïve Mice with LPMP/mRNA Formulation

In Example 6, it was shown that 3 doses of a recLemon LPMP/mRNA formulation (0.2 mg/kg) in tumor-bearing mouse model was able to express IL-2 in vivo and reach systemic level 4 hours post dosing, induce high levels of cytokines directly downstream of IL-2 signaling including IFNγ, TNFα, and IL-6 at significantly higher levels compared with the benchmark, and decrease tumor sizes and increase survival rate in treated mice.

In this example, the recLemon LPMP/mRNA formulation was prepared according to Example 6.

This example demonstrated that 1 dose of a recLemon LPMP/mRNA formulation (0.4 mg/kg) in non-tumor-bearing mouse model was able to express IL-2 in vivo and reach high systemic levels compared to an IL-2 protein up to 48 hours post dosing; was able to mount high levels of cytokines directly downstream of IL-2 signaling (IFNγ, TNFα) and, to a lesser extent, T cell anti-inflammatory cytokines; and had a durable effects on T cells lasting at least 6 days with an increase in surface IL-2Rα (~10 M more T cells and CD8 T cells ready to secrete IFNγ).

These conclusions were illustrated in FIG. 15, FIGS. 16A-16B, and FIG. 17.

### Example 8. Additional mRNA Design for Loading into LPMP/mRNA Formulation

Additional mRNA designs for loading into LPMP/mRNA formulation are shown in Scheme 2 below, which includes a sequence encoding a payload.

TABLE 8

| | Disease | | | | |
|---|---|---|---|---|---|
| Payload | Relevance | Class | Gene Length | | Route |
| Etanercept (Enbrel) | TNF Inhibitor | Fusion | 1404 | bp | SubQ |
| Humira | TNF Inhibitor | Antibody | 1356 + 645 | bp | SubQ |
| Epogen | Hematopoietic | Cytokine | 501 | bp | IV, SubQ |
| Filgrastim | Hematopoietic | Glycoprotein | 525 | bp | IV, SubQ |
| Keytruda | Anti-PD1 | Antibody | 1344 + 657 | bp | IV |
| Rituximab | Anti-CD20 | Antibody | 1356 + 642 | bp | IV |
| Romiplostim | ITP | Fusion | 807 | bp | SubQ |
| Sargramostim | G-CSF | Cytokine | 304 | bp | IV, SubQ |

| | |
|---|---|
| Anti-CD19 (Murine) | Antibody |

Exemplary sequences of payload are shown in Table 9.

TABLE 9

| Payload | Sequence |
|---|---|
| tdTomato | ATGGTGAGCAAGGGCGAGGAGGTCATCAAAGAGTTCATGCGCTTCAAGGTGCG |
| Cytosolic | CATGGAGGGCTCCATGAACGGCCACGAGTTCGAGATCGAGGGCGAGGGCGAG |
| | GGCCGCCCCTACGAGGGCACCCAGACCGCCAAGCTGAAGGTGACCAAGGGCG |
| | GCCCCCTGCCCTTCGCCTGGGACATCCTGTCCCCCCAGTTCATGTACGGCTCCA |
| | AGGCGTACGTGAAGCACCCCGCCGACATCCCCGATTACAAGAAGCTGTCCTTCC |
| | CCGAGGGCTTCAAGTGGGAGCGCGTGATGAACTTCGAGGACGGCGGTCTGGTG |
| | ACCGTGACCCAGGACTCCTCCCTGCAGGACGGCACGCTGATCTACAAGGTGAA |
| | GATGCGCGGCACCAACTTCCCCCCCGACGGCCCCGTAATGCAGAAGAAGACCA |
| | TGGGCTGGGAGGCCTCCACCGAGCGCCTGTACCCCCGCGACGGCGTGCTGAA |
| | GGGCGAGATCCACCAGGCCCTGAAGCTGAAGGACGGCGGCCACTACCTGGTG |
| | GAGTTCAAGACCATCTACATGGCCAAGAAGCCCGTGCAACTGCCCGGCTACTAC |
| | TACGTGGACACCAAGCTGGACATCACCTCCCACAACGAGGACTACACCATCGTG |
| | GAACAGTACGAGCGCTCCGAGGGCCGCCACCACCTGTTCCTGGGGCATGGCAC |
| | CGGCAGCACCGGCAGCGGCAGCTCCGGCACCGCCTCCTCCGAGGACAACAAC |
| | ATGGCCGTCATCAAAGAGTTCATGCGCTTCAAGGTGCGCATGGAGGGCTCCATG |
| | AACGGCCACGAGTTCGAGATCGAGGGCGAGGGCGAGGGCCGCCCCTACGAGG |
| | GCACCCAGACCGCCAAGCTGAAGGTGACCAAGGGCGGCCCCCTGCCCTTCGCC |
| | TGGGACATCCTGTCCCCCCAGTTCATGTACGGCTCCAAGGCGTACGTGAAGCAC |
| | CCCGCCGACATCCCCGATTACAAGAAGCTGTCCTTCCCCGAGGGCTTCAAGTGG |
| | GAGCGCGTGATGAACTTCGAGGACGGCGGTCTGGTGACCGTGACCCAGGACTC |
| | CTCCCTGCAGGACGGCACGCTGATCTACAAGGTGAAGATGCGCGGCACCAACT |
| | TCCCCCCCGACGGCCCCGTAATGCAGAAGAAGACCATGGGCTGGGAGGCCTCC |
| | ACCGAGCGCCTGTACCCCCGCGACGGCGTGCTGAAGGGCGAGATCCACCAGG |
| | CCCTGAAGCTGAAGGACGGCGGCCGCTACCTGGTGGAGTTCAAGACCATCTAC |
| | ATGGCCAAGAAGCCCGTGCAACTGCCCGGCTACTACTACGTGGACACCAAGCT |
| | GGACATCACCTCCCACAACGAGGACTACACCATCGTGGAACAGTACGAGCGCTC |
| | CGAGGGCCGCCACCACCTGTTCCTGTACGGCATGGACGAGCTGTACAAGTAA |
| tdTomato | ATGTGGTGGCGCCTGTGGTGGCTGCTGCTGCTGCTGCTGCTGCTGTGGCCCAT |
| Secreted | GGTGTGGGCCGTGAGCAAGGGCGAGGAGGTCATCAAAGAGTTCATGCGCTTCA |
| | AGGTGCGCATGGAGGGCTCCATGAACGGCCACGAGTTCGAGATCGAGGGCGAG |
| | GGCGAGGGCCGCCCCTACGAGGGCACCCAGACCGCCAAGCTGAAGGTGACCA |
| | AGGGCGGCCCCCTGCCCTTCGCCTGGGACATCCTGTCCCCCCAGTTCATGTAC |
| | GGCTCCAAGGCGTACGTGAAGCACCCCGCCGACATCCCCGATTACAAGAAGCT |
| | GTCCTTCCCCGAGGGCTTCAAGTGGGAGCGCGTGATGAACTTCGAGGACGGCG |
| | GTCTGGTGACCGTGACCCAGGACTCCTCCCTGCAGGACGGCACGCTGATCTAC |
| | AAGGTGAAGATGCGCGGCACCAACTTCCCCCCCGACGGCCCCGTAATGCAGAA |
| | GAAGACCATGGGCTGGGAGGCCTCCACCGAGCGCCTGTACCCCCGCGACGGC |
| | GTGCTGAAGGGCGAGATCCACCAGGCCCTGAAGCTGAAGGACGGCGGCCACTA |
| | CCTGGTGGAGTTCAAGACCATCTACATGGCCAAGAAGCCCGTGCAACTGCCCG |
| | GCTACTACTACGTGGACACCAAGCTGGACATCACCTCCCACAACGAGGACTACA |
| | CCATCGTGGAACAGTACGAGCGCTCCGAGGGCCGCCACCACCTGTTCCTGGGG |
| | CATGGCACCGGCAGCACCGGCAGCGGCAGCTCCGGCACCGCCTCCTCCGAGG |
| | ACAACAACATGGCCGTCATCAAAGAGTTCATGCGCTTCAAGGTGCGCATGGAGG |
| | GCTCCATGAACGGCCACGAGTTCGAGATCGAGGGCGAGGGCGAGGGCCGCCC |
| | CTACGAGGGCACCCAGACCGCCAAGCTGAAGGTGACCAAGGGCGGCCCCCTG |
| | CCCTTCGCCTGGGACATCCTGTCCCCCCAGTTCATGTACGGCTCCAAGGCGTAC |
| | GTGAAGCACCCCGCCGACATCCCCGATTACAAGAAGCTGTCCTTCCCCGAGGG |
| | CTTCAAGTGGGAGCGCGTGATGAACTTCGAGGACGGCGGTCTGGTGACCGTGA |
| | CCCAGGACTCCTCCCTGCAGGACGGCACGCTGATCTACAAGGTGAAGATGCGC |
| | GGCACCAACTTCCCCCCCGACGGCCCCGTAATGCAGAAGAAGACCATGGGCTG |
| | GGAGGCCTCCACCGAGCGCCTGTACCCCCGCGACGGCGTGCTGAAGGGCGAG |
| | ATCCACCAGGCCCTGAAGCTGAAGGACGGCGGCCGCTACCTGGTGGAGTTCAA |
| | GACCATCTACATGGCCAAGAAGCCCGTGCAACTGCCCGGCTACTACTACGTGGA |
| | CACCAAGCTGGACATCACCTCCCACAACGAGGACTACACCATCGTGGAACAGTA |
| | CGAGCGCTCCGAGGGCCGCCACCACCTGTTCCTGTACGGCATGGACGAGCTGT |
| | ACAAGTAA |
| tdTomato | ATGGGCTGCGTGTGCAGCAGCAACCCCGAGAACAACAACAACATGGTGAGCAA |
| Membrane | GGGCGAGGAGGTCATCAAAGAGTTCATGCGCTTCAAGGTGCGCATGGAGGGCT |
| | CCATGAACGGCCACGAGTTCGAGATCGAGGGCGAGGGCGAGGGCCGCCCCTA |
| | CGAGGGCACCCAGACCGCCAAGCTGAAGGTGACCAAGGGCGGCCCCCTGCCC |
| | TTCGCCTGGGACATCCTGTCCCCCCAGTTCATGTACGGCTCCAAGGCGTACGTG |
| | AAGCACCCCGCCGACATCCCCGATTACAAGAAGCTGTCCTTCCCCGAGGGCTTC |
| | AAGTGGGAGCGCGTGATGAACTTCGAGGACGGCGGTCTGGTGACCGTGACCCA |
| | GGACTCCTCCCTGCAGGACGGCACGCTGATCTACAAGGTGAAGATGCGCGGCA |
| | CCAACTTCCCCCCCGACGGCCCCGTAATGCAGAAGAAGACCATGGGCTGGGAG |
| | GCCTCCACCGAGCGCCTGTACCCCCGCGACGGCGTGCTGAAGGGCGAGATCCA |
| | CCAGGCCCTGAAGCTGAAGGACGGCGGCCACTACCTGGTGGAGTTCAAGACCA |
| | TCTACATGGCCAAGAAGCCCGTGCAACTGCCCGGCTACTACTACGTGGACACCA |
| | AGCTGGACATCACCTCCCACAACGAGGACTACACCATCGTGGAACAGTACGAGC |
| | GCTCCGAGGGCCGCCACCACCTGTTCCTGGGGCATGGCACCGGCAGCACCGG |
| | CAGCGGCAGCTCCGGCACCGCCTCCTCCGAGGACAACAACATGGCCGTCATCA |
| | AAGAGTTCATGCGCTTCAAGGTGCGCATGGAGGGCTCCATGAACGGCCACGAG |
| | TTCGAGATCGAGGGCGAGGGCGAGGGCCGCCCCTACGAGGGCACCCAGACCG |
| | CCAAGCTGAAGGTGACCAAGGGCGGCCCCCTGCCCTTCGCCTGGGACATCCTG |
| | TCCCCCCAGTTCATGTACGGCTCCAAGGCGTACGTGAAGCACCCCGCCGACATC |
| | CCCGATTACAAGAAGCTGTCCTTCCCCGAGGGCTTCAAGTGGGAGCGCGTGAT |
| | GAACTTCGAGGACGGCGGTCTGGTGACCGTGACCCAGGACTCCTCCCTGCAGG |
| | ACGGCACGCTGATCTACAAGGTGAAGATGCGCGGCACCAACTTCCCCCCCGAC |
| | GGCCCCGTAATGCAGAAGAAGACCATGGGCTGGGAGGCCTCCACCGAGCGCCT |
| | GTACCCCCGCGACGGCGTGCTGAAGGGCGAGATCCACCAGGCCCTGAAGCTGA |
| | AGGACGGCGGCCGCTACCTGGTGGAGTTCAAGACCATCTACATGGCCAAGAAG |
| | CCCGTGCAACTGCCCGGCTACTACTACGTGGACACCAAGCTGGACATCACCTCC |
| | CACAACGAGGACTACACCATCGTGGAACAGTACGAGCGCTCCGAGGGCCGCCA |
| | CCACCTGTTCCTGTACGGCATGGACGAGCTGTACAAGTAA |
| eGFP | ATGGTGAGCAAGGGCGAGGAGCTGTTCACCGGGGTGGTGCCCATCCTGGTCGA |
| | GCTGGACGGCGACGTAAACGGCCACAAGTTCAGCGTGTCCGGCGAGGGCGAG |
| | GGCGATGCCACCTACGGCAAGCTGACCCTGAAGTTCATCTGCACCACCGGCAA |
| | GCTGCCCGTGCCCTGGCCCACCCTCGTGACCACCCTGACCTACGGCGTGCAGT |
| | GCTTCAGCCGCTACCCCGACCACATGAAGCAGCACGACTTCTTCAAGTCCGCCA |
| | TGCCCGAAGGCTACGTCCAGGAGCGCACCATCTTCTTCAAGGACGACGGCAACT |
| | ACAAGACCCGCGCCGAGGTGAAGTTCGAGGGCGACACCCTGGTGAACCGCATC |
| | GAGCTGAAGGGCATCGACTTCAAGGAGGACGGCAACATCCTGGGGCACAAGCT |
| | GGAGTACAACTACAACAGCCACAACGTCTATATCATGGCCGACAAGCAGAAGAA |
| | CGGCATCAAGGTGAACTTCAAGATCCGCCACAACATCGAGGACGGCAGCGTGC |
| | AGCTCGCCGACCACTACCAGCAGAACACCCCCATCGGCGACGGCCCCGTGCTG |
| | CTGCCCGACAACCACTACCTGAGCACCCAGTCCGCCCTGAGCAAAGACCCCAA |
| | CGAGAAGCGCGATCACATGGTCCTGCTGGAGTTCGTGACCGCCGCCGGGATCA |
| | CTCTCGGCATGGACGAGCTGTACAAGTAA |
| IL-2 | ATGTACAGGATGCAACTCCTGTCTTGCATTGCACTAAGTCTTGCACTTGTCACAA |
| | ACAGTGCACCCACCAGCAGCAGCACCAAGAAGACCCAGCTGCAGCTGGAGCAC |
| | CTGCTGCTGGACCTGCAGATGATCCTGAACGGCATCAACAACTACAAGAACCCC |
| | AAGCTGACCAGAATGCTGACCTTCAAGTTCTACATGCCCAAGAAGGCCACCGAG |
| | CTGAAGCACCTGCAGTGCCTGGAGGAGGAGCTGAAGCCCCTGGAGGAGGTGCT |
| | GAACCTGGCCCAGAGCAAGAACTTCCACCTGAGACCCAGAGACCTGATCAGCAA |
| | CATCAACGTGATCGTGCTGGAGCTGAAGGGCAGCGAGACCACCTTCATGTGCG |
| | AGTACGCCGACGAGACCGCCACCATCGTGGAGTTCCTGAACAGATGGATCACCT |
| | TCAGCCAGAGCATCATCAGCACCCTGACCTGA |
| sumIL-2 | ATGTACAGGATGCAACTCCTGTCTTGCATTGCACTAAGTCTTGCACTTGTCACAA |
| | ACAGTGCACCCACCAGCAGCAGCACCAAGAAGACCCAGCTGCAGCTGGAGCAC |
| | CTGCTGCTGGACCTGCAGATGATCCTGAACGGCATCAACAACTACAAGAACCCC |
| | AAGCTGACCAGAATGCTGACCGCCAAGTTCTACATGCCCAAGAAGGCCACCGAG |
| | CTGAAGCACCTGCAGTGCCTGGAGGAGGAGCTGAAGCCCCTGGAGGAGGTGCT |
| | GAACCTGGCCCAGAGCAAGAACTTCCACTTCGACCCCAGAGACGTGGTGAGCA |
| | ACATCAACGTGTTCGTGCTGGAGCTGAAGGGCAGCGAGACCACCTTCATGTGCG |
| | AGTACGCCGACGAGACCGCCACCATCGTGGAGTTCCTGAACAGATGGATCACCT |
| | TCAGCCAGAGCATCATCAGCACCCTGACCTGA |
| IL-2 + | ATGTACAGGATGCAACTCCTGTCTTGCATTGCACTAAGTCTTGCACTTGTCACAA |
| IL-2-Ra | ACAGTAGCAAGAACTTCCACCTGAGACCCAGAGACCTGATCAGCAACATCAACG |
| | TGATCGTGCTGGAGCTGAAGGGCAGCGAGACCACCTTCATGTGCGAGTACGCC |
| | GACGAGACCGCCACCATCGTGGAGTTCCTGAACAGATGGATCACCTTCAGCCAG |
| | AGCATCATCAGCACCCTGACCGGCGGCAGCAGCAGCACCAAGAAGACCCAGCT |
| | GCAGCTGGAGCACCTGCTGCTGGACCTGCAGATGATCCTGAACGGCATCAACAA |
| | CTACAAGAACCCCAAGCTGACCAGAATGCTGACCTTCAAGTTCTACATGCCCAA |
| | GAAGGCCACCGAGCTGAAGCACCTGCAGTGCCTGGAGGAGGAGCTGAAGCCCC |
| | TGGAGGAGGTGCTGAACCTGGCCCAGGGCAGCGGCGGCGGCAGCGAGCTGTG |
| | CGACGACGACCCCCCCGAGATCCCCCACGCCACCTTCAAGGCCATGGCCTACA |
| | AGGAGGGCACCATGCTGAACTGCGAGTGCAAGAGAGGCTTCAGAAGAATCAAG |
| | AGCGGCAGCCTGTACATGCTGTGCACCGGCAACAGCAGCCACAGCAGCTGGGA |
| | CAACCAGTGCCAGTGCACCAGCAGCGCCACCAGAAACACCACCAAGCAGGTGA |
| | CCCCCCAGCCCGAGGAGCAGAAGGAGAGAAAGACCACCGAGATGCAGAGCCC |
| | CATGCAGCCCGTGGACCAGGCCAGCCTGCCCGGCCACTGCAGAGAGCCCCCC |
| | CCCTGGGAGAACGAGGCCACCGAGAGAATCTACCACTTCGTGGTGGGCCAGAT |
| | GGTGTACTACCAGTGCGTGCAGGGCTACAGAGCCCTGCACAGAGGCCCCGCCG |
| | AGAGCGTGTGCAAGATGACCCACGGCAAGACCAGATGGACCCAGCCCCAGCTG |
| | ATCTGCACCGGCTGA |
| Anti-CD19 & | ATGCTGCTGCTGGTGACCAGCCTGCTGCTGTGCGAGCTGCCCCACCCCGCCTT |
| CD20 CAR-T | CCTTTTGATTCCAGAGGTGCAATTACAGCAAAGCGGTGCAGAACTCGTGAAGCC |
| | AGGCGCTTCAGTAAAAATGTCCTGCAAAGCTTCCGGCTACACATTCACTAGCTAC |
| | AACATGCATTGGGTTAAACAAACACCTGGCCAGGGATTGGAGTGGATCGGGGCT |
| | ATATATCCGGGTAACGGTGACACTAGTTACAATCAGAAATTCAAGGGAAAGGCCA |
| | CGCTTACAGCAGACAAGAGTAGCAGTACCGCCTATATGCAACTCAGCAGCTTGA |
| | CCAGCGAAGACAGCGCAGATTATTACTGTGCCAGATCCAACTATTACGGCTCAA |
| | GCTACTGGTTCTTTGACGTGTGGGGCGCCGGAACCACTGTTACTGTCAGCAGCG |
| | GAGGCGGTGGGAGTGGCGGCGGTGGTTCCGGCGGGGGGGGGTCAGATATTGT |
| | GTTGACGCAATCCCCCGCAATCCTGTCCGCGTCCCCAGGCGAAAAAGTGACAAT |
| | GACATGCCGGGCCAGCTCAAGCGTGAATTACATGGATTGGTATCAGAAGAAACC |
| | CGGGAGCAGCCCCAAGCCGTGGATCTATGCGACGAGTAACCTGGCTTCCGGCG |
| | TCCCCGCCCGATTCAGCGGGTCCGGCAGCGGCACTAGTTACAGTTTAACCATCA |
| | GCAGGGTGGAGGCTGAAGACGCGGCAACCTATTATTGCCAGCAGTGGTCATTTA |
| | ATCCTCCCACGTTTGGCGGCGGCACCAAGCTGGAGATAAAGGGGGGGGGGGG |
| | CAGTGGAGGCGGAGGTAGCGGCGGAGGAGGATCAGGAGGGGGGGGTAGCGG |
| | AGGAGGGGGTTCAGACATACAGATGACACAGACAACCTCGAGCCTGAGCGCAT |
| | CCCTGGGGGACCGAGTCACCATCTCATGTAGAGCATCGCAGGACATCAGCAAAT |
| | ATCTCAACTGGTACCAGCAAAAACCCGATGGTACAGTGAAACTCTTAATTTACCA |
| | CACATCCCGGTTACATAGCGGAGTCCCAAGCCGCTTTAGCGGATCCGGAAGCG |
| | GGACCGACTACTCCCTCACGATCAGCAACCTCGAACAGGAGGATATAGCCACTT |
| | ATTTCTGCCAGCAGGGTAACACCCTGCCGTATACATTTGGCGGGGGCACAAAAC |
| | TAGAAATCACAGGCAGTACAAGTGGTAGTGGGAAGCCCGGTAGCGGAGAAGGT |
| | AGCACGAAAGGCGAGGTTAAACTTCAGGAATCAGGCCCAGGCCTAGTTGCCCCT |
| | TCACAAAGTCTAAGCGTCACGTGTACCGTGTCCGGCGTAAGCCTGCCAGATTAT |
| | GGAGTCAGCTGGATTAGGCAGCCTCCACGGAAGGGACTGGAGTGGCTGGGCGT |
| | GATTTGGGGGTCGGAGACCACCTATTATAACAGCGCGCTGAAATCGCGGCTGAC |
| | TATTATTAAGGATAATAGCAAGTCACAGGTGTTTCTTAAGATGAATAGCCTGCAG |
| | ACTGATGACACTGCGATATACTACTGTGCCAAGCACTACTACTACGGAGGGTCAT |
| | ATGCTATGGATTACTGGGGGCAGGGAACCTCCGTGACTGTAAGTTCCGCCGCC |
| | GCTACTACAACCCCTGCCCCACGTCCCCCGACCCCAGCACCTACTATCGCATCC |
| | CAGCCCCTTTCCCTTCGCCCTGAGGCCTGCAGGCCTGCAGCTGGAGGAGCCGT |
| | TCACACCAGGGGCCTCGACTTTGCATGCGATATTTACATCTGGGCCCCACTGGC |
| | TGGCACCTGCGGCGTCTTGCTGCTGAGCCTCGTGATCACCCTCTATTGTAAGAG |
| | GGGGCGAAAAAAGCTCCTGTACATTTTCAAACAGCCTTTCATGAGACCTGTGCA |
| | GACAACACAGGAAGAGGACGGTTGCTCCTGTAGGTTTCCGGAGGAAGAAGAGG |
| | GAGGGTGTGAATTGCGTGTAAAGTTCTCCAGATCAGCTGATGCCCCAGCCTATC |
| | AACAAGGACAGAATCAGCTGTACAACGAGCTTAATTTGGGGCGGAGAGAGGAAT |
| | ACGATGTCTTGGACAAGAGACGGGGGCGCGACCCCGAGATGGGGGGGAAGCC |
| | TAGAAGGAAGAACCCTCAGGAGGGGCTGTATAATGAGCTCCAAAAGGATAAGAT |
| | GGCCGAAGCTTACAGCGAGATCGGGATGAAAGGAGAGCGCCGCCGCGGAAAA |
| | GGCCATGATGGGTTATACCAGGGCCTTAGCACCGCCACTAAGGACACTTACGAC |
| | GCTCTACACATGCAGGCTCTCCCACCCCGATGA |
| Anti-HER2 | ATGAGGGCTTGGATCTTCTTTCTGCTCTGCCTGGCCGGGCGCGCCTTGGCCGAA |
| Heavy Chain | GTTCAACTGGTAGAGAGTGGAGGTGGGCTTGTGCAACCAGGCGGATCCTTGCG |
| | ACTGTCCTGCGCCGCTTCAGGCTTCAACATCAAGGACACCTACATCCATTGGGT |
| | CCGCCAGGCACCAGGAAAAGGTCTTGAATGGGTGGCCAGAATCTACCCTACTAA |
| | CGGTTACACCAGATATGCAGACTCCGTTAAGGGGCGATTTACCATTTCAGCAGA |
| | CACCTCTAAGAACACCGCTTACCTGCAGATGAACTCACTTCGAGCTGAGGACAC |
| | CGCCGTTTACTATTGCAGCAGATGGGGCGGTGACGGCTTCTACGCTATGGATTA |
| | CTGGGGACAGGGGACTTTGGTAACTGTGAGTAGTGCATCTACAAAGGGGCCAA |
| | GCGTGTTCCCACTTGCCCCATCTTCTAAAAGCACCTCAGGAGGGACTGCAGCCT |
| | TGGGTTGCTTGGTTAAAGATTATTTTCCAGAGCCTGTAACTGTATCCTGGAATAG |
| | TGGGGCCCTCACAAGCGGAGTACATACTTTCCCTGCAGTATTGCAGTCTAGTGG |
| | ACTCTACTCTCTCAGCAGTGTAGTGACCGTACCTTCCAGTTCACTTGGAACACAG |
| | ACCTATATTTGCAATGTGAATCATAAGCCATCTAATACTAAAGTGGATAAGAAAGT |
| | GGAGCCTAAATCTTGTGACAAGACTCATACATGCCCTCCCTGCCCTGCCCCTGA |
| | ACTGTTGGGAGGGCCCTCTGTATTTCTCTTCCCCCCTAAACCAAAGGACACCCT |
| | GATGATCAGTCGAACTCCTGAGGTGACTTGTGTGGTTGTTGACGTGTCACATGA |
| | GGATCCCGAAGTGAAATTCAACTGGTACGTCGATGGAGTAGAGGTACACAATGC |
| | AAAGACAAAACCTAGGGAGGAACAGTATAATTCTACCTATAGAGTGGTGTCTGTT |
| | CTCACAGTTCTCCATCAAGACTGGTTGAACGGTAAAGAATATAAATGCAAAGTCT |
| | CCAATAAGGCTTTGCCCGCTCCCATTGAAAAAACAATCAGTAAAGCCAAAGGCCA |
| | GCCACGCGAACCACAGGTCTACACCCTTCCACCATCTCGCGAAGAAATGACTAA |
| | GAATCAAGTGTCACTCACATGCCTCGTCAAGGGCTTTTACCCTTCTGATATTGCA |
| | GTTGAATGGGAGTCTAACGGGCAGCCCGAGAATAATTACAAGACTACCCCCCCC |
| | GTCCTTGACTCTGATGGCAGCTTTTTCCTGTACTCAAAATTGACTGTGGACAAGT |
| | CTCGATGGCAACAGGGTAATGTTTTCTCCTGTAGCGTAATGCACGAGGCCCTTC |
| | ATAACCATTATACCCAAAAATCTCTTTCATTGTCCCCTGGAAAATGA |
| Anti-HER2 | ATGAGGGCTTGGATCTTCTTTCTGCTCTGCCTGGCCGGGCGCGCCTTGGCCGAT |
| Light Chain | ATTCAGATGACTCAGAGCCCCAGCAGCCTGTCTGCTAGCGTTGGGGATAGAGTC |
| | ACTATAACATGTCGGGCTTCCCAGGATGTGAATACTGCTGTCGCTTGGTATCAAC |
| | AGAAGCCCGGCAAGGCACCAAAACTGCTGATATATAGTGCCTCTTTCCTTTACTC |
| | CGGGGTTCCCAGTCGATTCTCTGGAAGCCGCAGCGGCACTGATTTCACACTTAC |
| | TATAAGTAGTCTGCAACCTGAGGACTTTGCTACATACTACTGCCAGCAGCACTAT |
| | ACAACACCCCCAACTTTCGGGCAAGGCACTAAGGTAGAAATTAAAAGGACCGTT |
| | GCTGCTCCATCCGTCTTTATTTTTCCACCATCTGATGAACAGTTGAAGAGTGGAA |
| | CAGCAAGCGTCGTTTGTCTCCTGAACAATTTTTACCCACGAGAGGCAAAAGTTCA |
| | ATGGAAGGTAGACAATGCCCTTCAGAGCGGCAATTCCCAGGAGAGCGTAACCGA |
| | GCAGGATAGCAAAGACTCTACATACTCTTTGAGTTCAACCCTTACCCTGAGCAAG |
| | GCTGATTACGAAAAACACAAGGTGTACGCTTGCGAGGTAACCCATCAGGGATTG |
| | TCATCCCCAGTCACAAAATCATTCAATAGGGGCGAGTGCTGA |
| Keytruda | AUGGAAUUUGGACUGUCUUGGGUUUUUCUGGUAGCUCUGUUUCGUGGAGUG |
| | CAAUGCCAGGUUCAGUUGGUCCAGUCGGGUGUGGAGGUUAAAAAGCCCGGCG |
| | CCUCUGUGAAGGUUAGCUGCAAGGCUAGCGGAUAUACUUUCACCAAUUACUA |
| | CAUGUAUUGGGUGAGGCAGGCUCCAGGGCAGGGACUCGAGUGGAUGGGAGG |
| | CAUCAACCCGUCCAACGGCGGCACCAACUUCAAUGAGAAGUUCAAAAAUAGAG |
| | UGACUCUCACCACUGAUAGUUCCACGACAACCGCAUACAUGGAACUGAAAAGU |
| | CUGCAAUUCGAUGACACCGCCGUGUAUUACUGCGCCCGCAGAGACUACCGGU |
| | UUGACAUGGGAUUUGAUUACUGGGGUCAGGGCACCACAGUGACAGUCUCAUC |
| | CGCCUCUACCAAAGGACCCUCGGUUUUUCCACUCGCACCUUGCUCCCGAAGC |
| | ACCUCAGAGUCCACCGCGGCCCUUGGGUGUCUGGUGAAAGAUUAUUUCCCCG |
| | AACCAGUGACCGUGUCAUGGAAUUCAGGGGCACUGACAAGUGGGGUCCACAC |
| | GUUCCCAGCGGUGCUGCAGUCCUCCGGGCUGUAUAGCCUUAGCUCUGUAGUC |
| | ACUGUCCCGUCCAGUUCCCUGGGGACUAAGACUUACACAUGUAACGUAGAUC |
| | ACAAACCUAGUAACACCAAGGUGGACAAGCGCGUGGAGUCAAAGUACGGACCU |
| | CCAUGUCCCCCAUGUCCCGCCCCCGAAUUUUUGGGAGGACCUUCAGUGUUCC |
| | UGUUCCCUCCAAAACCUAAAGACACACUGAUGAUAAGCAGGACACCCGAGGUC |
| | ACCUGCGUCGUGGUCGACGUAAGCCAGGAAGAUCCAGAGGUACAGUUCAACU |
| | GGUACGUCGACGGUGUAGAGGUCCAUAAUGCCAAGACAAAGCCCAGGGAGGA |
| | GCAGUUUAACUCAACAUAUCGUGUUGUGUCUGUGUUGACGGUUCUACAUCAA |
| | GACUGGCUAAACGGCAAGGAGUAUAAAUGCAAGGUUAGUAAUAAGGGCCUCC |
| | CCAGCUCUAUUGAGAAAACUAUCAGCAAAGCUAAGGGCCAGCCUCGGGAACCA |
| | CAAGUUUACACUUUACCGCCCAGCCAGGAGGAAAUGACGAAGAAUCAGGUGAG |
| | CCUGACAUGUCUUGUCAAGGGGUUUUACCCCUCUGAUAUCGCUGUAGAAUGG |
| | GAAUCCAAUGGUCAACCUGAAAACAACUACAAGACCACCCCGCCUGUGCUCGA |
| | CUCUGACGGCAGCUUUUUCCUGUAUUCGAGACUUACAGUGGAUAAAAGCCGG |
| | UGGCAGGAGGGCAAUGUGUUUUCCUGCAGUGUGAUGCACGAAGCAUUACACA |
| | ACCAUUAUACUCAAAAAUCACUCAGUUUGUCUCUGGGGAAG |
| Epogen | AUGGGGGUCCACGAAUGCCCGGCAUGGCUGUGGUUACUGCUAUCGCUGCUCA |
| | GCCUGCCACUGGGCCUGCCCGUACUUGGCGCCCCUCCACGCCUCAUCUGUGA |
| | CUCCCGGGUCCUGGAGCGGUACCUUUUGGAGGCCAAAGAAGCAGAGAAUAUC |
| | ACUACAGGCUGCGCUGAGCACUGUAGUUUGAACGAAAAUAUAACAGUACCUGA |
| | CACAAAGGUGAAUUUUUACGCCUGGAAAAGAAUGGAGGUGGGCCAACAGGCA |
| | GUGGAAGUGUGGCAGGGUCUCGCUUUACUUAGCGAGGCCGUGCUGCGAGGU |
| | CAGGCACUCCUAGUUAACUCCAGCCAGCCCUGGGAGCCACUCCAGUUGCAUG |
| | UGGAUAAGGCCGUCUCUGGACUAAGAUCCCUCACCACCCUGCUGAGGGCAUU |
| | GGGAGCGCAAAAGGAGGCUAUUUCACCUCCCGAUGCCGCUAGUGCUGCGCCC |
| | CUUCGCACUAUCACCGCCGACACGUUCCGUAAGCUGUUCAGGGUUUAUUCAA |
| | ACUUUCUCCGGGGGAAGCUGAAACUGUAUACUGGGGAAGCCUGCAGAACCGG |
| | AGAUAGG |
| Enbrel | AUGAAAUGGGUUACGUUCAUCUCCUUGCUGUUCCUGUUCUCUUCCGCUUACU |
| | CCCUGCCAGCCCAGGUCGCUUUCACGCCAUACGCUCCUGAACCAGGUAGCAC |
| | UUGUCGUCUACGGGAAUACUAUGACCAGACUGCACAGAUGUGCUGUUCCAAG |
| | UGCUCUCCUGGCCAACAUGCCAAAGUGUUUUGCACUAAGACUAGCGAUACAG |
| | UGUGCGAUAGCUGCGAGGAUUCUACAUAUACCCAGCUCUGGAACUGGGUUCC |
| | CGAGUGUCUCAGCUGUGGCAGCCGGUGUAGUAGCGACCAGGUGGAGACCCA |
| | GGCCUGCACACGAGAGCAGAACCGGAUCUGCACAUGUAGACCCGGGUGGUAC |
| | UGUGCGUUGAGUAAACAGGAGGGAUGUAGACUCUGCGCCCCUCUUAGGAAGU |
| | GCCGCCCAGGGUUUGGAGUCGCACGCCCAGGCACAGAAACCAGUGACGUCGU |
| | CUGCAAGCCGUGUGCCCCGGGCACAUUCUCCAACACUACCUCAAGUACAGAUA |
| | UUUGUAGACCUCAUCAGAUCUGCAAUGUCGUGGCAAUUCCCGGUAACGCCUC |
| | GAUGGAUGCCGUGUGCACCUCUACGUCGCCCACUAGGUCCAUGGCCCCAGGA |
| | GCAGUACACUUACCUCAGCCUGUUAGCACACGCUCCCAGCACACGCAACCCAC |
| | CCCUGAGCCUUCCACUGCACCGUCCACCAGCUUCCUGCUGCCCAUGGGCCCA |
| | AGUCCUCCAGCGGAGGGCAGCACAGGGGACGAGCCAAAGUCCUGCGACAAAA |
| | CCCAUACUUGUCCCCCUUGCCCCGCACCCGAACUCCUUGGGGGACCAUCAGU |
| | AUUCUUGUUUCCCCCCAAGCCAAAGGACACAUUAAUGAUUUCUAGGACUCCGG |
| | AGGUGACUUGCGUCGUUGUUGACGUGAGCCACGAAGACCCAGAGGUGAAAUU |
| | CAACUGGUAUGUGGAUGGAGUCGAAGUUCACAAUGCGAAAACCAAACCUCGAG |
| | AGGAACAGUACAACUCAACCUAUAGGGUAGUGAGUGUUCUGACCGUGCUGCA |
| | CCAGGACUGGCUCAAUGGGAAGGAAUACAAGUGUAAGGUAUCCAAUAAAGCCC |
| | UGCCUGCCCCUAUCGAAAAGACAAUAAGCAAGGCUAAGGGGCAACCUCGCGAA |
| | CCCCAGGUGUACACGCUGCCACCAAGCAGAGAGGAGAUGACAAAAAACCAAGU |
| | GUCACUGACCUGCCUGGUGAAAGGCUUUUACCCGAGCGAUAUCGCUGUGGAG |
| | UGGGAGUCAAACGGACAACCCGAGAAUAACUAUAAAACCACGCCCCCCGUGCU |
| | GGAUUCAGACGGUUCUUUCUUCCUUUAUUCCAAGUUGACCGUGGACAAGUCU |
| | CGGUGGCAGCAGGGCAAUGUCUUUUCCUGUAGUGUCAUGCACGAAGCUCUAC |
| | ACAAUCAUUACACCCAAAAAAGCCUGUCACUCUCUCCCGGUAAA |
| Filgrastim | AUGGCAGGCCCAGCUACGCAGAGCCCAAUGAAACUGAUGGCACUGCAGCUCC |
| | UGUUAUGGCACAGCGCCCUGUGGACCGUCCAGGAAGCAACACCCCUCGGGCC |
| | UGCGUCCAGUCUCCCCCAGAGCUUUCUACUUAAAUGUCUAGAGCAGGUGCGG |
| | AAGAUCCAAGGAGACGGGGCCGCACUGCAGGAGAAGCUCUGCGCUACAUACA |
| | AGCUUUGUCACCCAGAGGAACUGGUGCUGUUAGGCCAUUCACUGGGAAUCCC |
| | UUGGGCCCCUUUGUCUAGUUGCCCGUCCCAGGCUUUACAGUUGGCCGGCUG |
| | CCUGAGCCAGCUGCACUCCGGACUAUUUCUGUAUCAGGGACUUUUGCAGGCC |
| | CUGGAGGGUAUUUCCCCUGAGCUCGGGCCGACUCUGGAUACACUGCAGCUUG |
| | AUGUCGCCGACUUUGCUACUACCAUAUGGCAACAAAUGGAGGAACUCGGUAU |
| | GGCCCCAGCGCUGCAACCCACCCAGGGGGCCAUGCCCGCUUUCGCGUCUGCC |
| | UUCCAGCGCAGAGCAGGCGGCGUGUUGGUGGCCUCACAUCUCCAAUCGUUCC |
| | UGGAAGUUUCUUACCGAGUACUCAGGCACUUGGCACAGCCC |
| Humira | AUGGAAUUUGGACUGUCUUGGGUUUUUCUGGUAGCUCUGUUUCGUGGAGUG |
| | CAAUGCGAGGUUCAGCUUGUCGAGAGCGGCGGCGGUCUCGUGCAACCCGGA |
| | CGGAGUCUGAGACUCAGCUGUGCUGCUUCAGGAUUUACCUUCGACGAUUAUG |
| | CGAUGCAUUGGGUGCGGCAGGCUCCUGGCAAAGGAUUAGAAUGGGUGAGCG |
| | CAAUUACAUGGAACUCUGGCCACAUCGACUACGCCGAUUCAGUGGAGGGCAG |
| | GUUCACCAUUAGCAGAGAUAAUGCUAAGAAUAGCUUAUACUUGCAGAUGAACA |
| | GCCUGCGCGCCGAGGAUACUGCGGUGUACUAUUGCGCCAAGGUCAGUUAUCU |
| | GUCCACCGCCAGCUCCCUAGAUUAUUGGGGGCAGGGAACCUUGGUGACAGUC |
| | AGUUCCGCAAGCACGAAAGGGCCCAGCGUUUUCCCUCUCGCACCCUCUUCAA |
| | AAUCCACUUCAGGGGGGACAGCUGCACUGGGAUGUCUUGUCAAGGAUUACUU |
| | CCCCGAACCUGUGACCGUCUCAUGGAAUUCCGGUGCCUUGACCUCAGGGGUG |
| | CAUACCUUCCCCGCAGUCCUUCAGAGUUCUGGACUCUACUCCCUGAGUUCUG |
| | UGGUAACAGUCCCUUCUAGCUCCCUCGGGACACAGACCUACAUUUGCAAUGU |
| | GAACCAUAAACCGAGUAACACUAAAGUGGACAAAAAAGUAGAACCCAAAAGCUG |
| | CGAUAAGACCCACACGUGCCCCCCGUGUCCGGCCCCCGAGCUUCUGGGCGGC |
| | CCUUCGGUGUUCCUGUUUCCACCUAAGCCCAAAGACACACUGAUGAUCUCUC |
| | GCACUCCUGAAGUAACAUGCGUGGUGGUCGACGUGAGCCACGAAGAUCCAGA |
| | AGUUAAGUUCAAUUGGUAUGUUGACGGGGUGGAGGUGCACAACGCCAAGACU |
| | AAGCCAAGGGAGGAGCAAUACAAUUCCACAUACCGAGUUGUCAGCGUACUUAC |
| | GGUUCUGCACCAAGACUGGCUAAAUGGUAAAGAGUACAAGUGUAAGGUGUCU |
| | AACAAAGCCCUGCCAGCGCCGAUAGAAAAGACUAUCUCAAAAGCAAAGGGCCA |
| | GCCCAGAGAACCUCAGGUGUACACCUUGCCACCAUCAAGGGACGAGCUCACC |
| | AAGAAUCAGGUUAGUCUCACCUGUCUGGUGAAGGGGUUCUAUCCAUCCGACA |
| | UCGCCGUGGAGUGGGAGAGUAACGGCCAGCCCGAAAACAAUUACAAGACGAC |
| | ACCACCAGUCUUGGAUAGCGACGGUUCUUUCUUUCUAUAUUCGAAGCUCACU |
| | GUCGACAAGUCGCGGUGGCAGCAGGGCAACGUAUUUAGCUGCUCCGUCAUGC |
| | AUGAGGCCCUGCACAACCACUAUACUCAAAAAUCCCUGUCCUUAUCUCCUGGC |
| | AAG |
| Rituximab | AUGAAGUAUCUACUGCCUACCGCUGCAGCUGGGCUGUUGCUUCUGGCUGCUC |
| | AACCAGCCAUGGCUCAGAUCGUGCUCUCCCAGUCCCCCGCGAUUUUGAGCGC |
| | AUCCCCAGGCGAGAAGGUAACAAUGACGUGCCGCGCGAGCAGUUCAGUGUCU |
| | UACAUCCACUGGUUCCAGCAAAAGCCAGGCUCUAGCCCCAAGCCCUGGAUUUA |
| | CGCAACCAGCAAUCUUGCCAGUGGCGUCCCGGUGAGGUUCUCGGGGUCUGG |
| | AAGCGGUACAUCUUAUAGUCUCACCAUAAGCAGGGUAGAGGCCGAGGACGCC |
| | GCUACUUACUAUUGCCAGCAGUGGACCUCUAACCCACCCACCUUCGGCGGUG |
| | GAACCAAGCUGGAAAUCAAACGAACAGUCGCCGCCCCGUCCGUGUUUAUCUU |
| | CCCUCCUUCUGACGAGCAGCUGAAAUCCGGGACCGCAUCCGUCGUUUGUCUA |
| | CUGAACAACUUUUACCCCCGGGAAGCAAAAGUCCAGUGGAAGGUUGAUAACGC |
| | CCUCCAAAGUGGAAAUAGCCAGGAAUCAGUGACUGAGCAAGAUUCCAAAGAUA |
| | GUACUUACAGCUUAUCAAGCACGCUCACUCUGUCCAAAGCCGACUAUGAAAAA |
| | CACAAGGUGUACGCCUGUGAGGUGACACAUCAGGGGCUGUCAUCACCUGUUA |
| | CAAAGUCGUUUAAUAGAGGCGAAUGC |
| Romiplostim | AUGAAAUGGGUUACGUUCAUCUCCUUGCUGUUCCUGUUCUCUUCCGCUUACU |
| | CCGAUAAAACACAUACCUGUCCCCCAUGUCCCGCCCCCGAGCUCCUCGGCGG |
| | UCCCAGUGUGUUUCUGUUUCCACCAAAACCCAAGGACACUCUCAUGAUAAGCC |
| | GCACGCCCGAGGUUACAUGCGUCGUCGUCGACGUCUCCCACGAGGAUCCGGA |
| | GGUGAAAUUUAACUGGUAUGUAGAUGGCGUGGAAGUGCACAACGCAAAAACCA |
| | AGCCCCGUGAAGAACAAUACAACUCCACAUACAGGGUCGUGAGCGUGCUGAC |
| | UGUACUCCAUCAGGAUUGGUUAAAUGGCAAGGAGUAUAAGUGUAAGGUGAGU |
| | AAUAAGGCCCUACCUGCACCUAUUGAAAAGACAAUUAGCAAAGCCAAGGGCCA |
| | GCCGAGGGAGCCACAGGUGUAUACACUGCCACCCAGCCGGGAUGAGUUAACA |
| | AAGAAUCAGGUUUCACUCACCUGCCUUGUAAAAGGUUUUUACCCAUCAGACAU |
| | CGCUGUGGAAUGGGAGUCUAACGGACAGCCUGAAAACAACUAUAAAACCACUC |
| | CUCCUGUGCUGGACUCUGACGGAUCGUUCUUCUUGUACUCAAAACUAACCGU |
| | GGACAAGAGUAGAUGGCAGCAGGGAAAUGUUUUCUCAUGCAGCGUCAUGCAC |
| | GAGGCCCUGCAUAAUCACUACACCCAGAAGAGUUUGUCUCUGAGCCCGGGUA |
| | AGGGAGGCGGCGGGGGCAUCGAAGGGCCUACUCUGAGACAAUGGCUGGCAG |
| | CCCGGGCUGGCGGGGGUGGAGGGGGGGGAGGCAUCGAGGGGCCAACCCUUC |
| | GCCAAUGGCUUGCGGCGCGAGCC |
| Sargramostim | AUGUGGCUGCAAUCCCUGCUGCUGCUUGGCACAGUGGCAUGCUCCAUCAGCG |
| | CUCCUGCCCGAUCUCCCUCACCCAGCACACAACCCUGGGAGCAUGUCAAUGC |
| | GAUACAGGAGGCCCUCCGGCUCUUAAAUCUCUCGCGCGAUACUGCAGCUGAG |
| | AUGAACGAAACCGUGGAAGUGAUCAGUGAAAUGUUCGAUCUGCAGGAACCCAC |
| | CUGUCUGCAGACCAGGCUAGAGUUGUACAAACAGGGCUUGAGAGGAUCACUA |
| | ACUAAACUUAAGGGGCCUCUGACGAUGAUGGCCUCCCACUAUAAGCAGCACU |
| | GUCCACCAACUCCAGAAACAAGUUGCGCCACACAGAUCAUUACCUUUGAGAGC |
| | UUUAAGGAGAACUUAAAAGACUUCCUCCUGGUUAUUCCUUUCGACUGCUGGG |
| | AGCCGGUACAGGAG |
| Anti-CD19 | AUGGGUGUCCCUACCCAGCUCCUGGGACUGCUCCUGCUGUGGAUCACCGACG |
| | CCAUCUGCGACAUCCAGAUGACCCAGAGCCCUGCCAGCCUGUCUACCAGCCU |
| | GGGCGAGACAGUGACCAUCCAGUGUCAGGCCAGCGAGGACAUCUACUCUGGC |
| | CUGGCUUGGUAUCAGCAGAAGCCCGGCAAGAGCCCUCAGCUGCUGAUCUACG |
| | GCGCCAGCGACCUGCAGGACGGCGUGCCUAGCAGAUUCAGCGGCAGCGGCU |
| | CCGGAACCCAGUACAGCCUGAAGAUCACCAGCAUGCAGACCGAGGACGAGGG |
| | CGUGUACUUCUGCCAGCAAGGCCUGACCUACCCUAGAACCUUCGGAGGAGGC |
| | ACCAAGCUGGAACUGAAGGGCGGAGGCGGAAGUGGAGGCGGAGGAUCUGGC |
| | GGCGGAGGCUCUGAAGUGCAGCUGCAGCAGUCUGGCGCUGAACUGGUCCGG |
| | CCUGGCACUAGCGUGAAGCUGUCCUGCAAGGUGUCCGGCGACACCAUCACCU |
| | UCUACUACAUGCACUUCGUGAAGCAGAGGCCAGGACAGGGCCUGGAAUGGAU |
| | CGGCAGAAUCGACCCUGAGGACGAGAGCACCAAGUACAGCGAGAAGUUCAAG |
| | AACAAGGCCACCCUGACCGCCGACACCAGCAGCAACACCGCCUACCUGAAGCU |
| | GUCUAGCCUGACCUCCGAGGACACCGCCACCUACUUUUGCAUCUACGGCGGC |
| | UACUACUUCGACUACUGGGGCCAGGGCGUGAUGGUCACCGUGUCCAGCAUCG |
| | AGUUCAUGUACCCCCCUCCCUACCUGGACAACGAGAGAAGCAACGGCACCAUC |
| | AUCCACAUCAAAGAAAAGCACCUGUGCCACACCCAGAGCAGCCCCAAGCUGUU |
| | CUGGGCCCUGGUGGUGGUGGCCGGCGUGCUGUUCUGUUACGGCCUGCUGGU |
| | CACAGUGGCCCUGUGCGUGAUCUGGACCAACAGCAGAAGAAACAGAGGCGGC |
| | CAGAGCGACUACAUGAACAUGACCCCCAGAAGGCCAGGCCUGACCAGAAAGCC |
| | CUACCAGCCCUACGCCCCUGCCAGAGACUUCGCCGCCUACAGACCCAGAGCC |
| | AAGUUCAGCAGAUCCGCCGAGACAGCCGCCAACCUGCAGGAUCCCAACCAGC |
| | UGUUCAACGAGCUGAACCUGGGCAGACGGGAGGAAUUCGACGUGCUGGAAAA |
| | GAAGAGAGCCAGGGACCCCGAGAUGGGGGCAAGCAGCAGAGAAGAAGAAAC |
| | CCUCAGGAAGGCGUCUACAACGCCCUGCAGAAAGACAAGAUGGCCGAGGCCU |
| | ACAGCGAGAUCGGCACCAAGGGCGAGAGAAGAAGGGGCAAGGGCCACGAUGG |
| | CCUGUUCCAGGGCCUGUCCACCGCCACCAAGGACACCUUCGACGCCCUGCAC |
| | AUGCAGACCCUGGCCCCCAGA |
| IL-15 | MRISKPHLRS ISIQCYLCLL LNSHFLTEAG IHVFILGCFS AGLPKTEANW |
| | VNVISDLKKI EDLIQSMHID ATLYTESDVH PSCKVTAMKC FLLELQVISL |
| | ESGDASIHDT VENLIILANN SLSSNGNVTE SGCKECEELE EKNIKEFLQS |
| | FVHIVQMFIN TS |
| IL-15 | ATGAGAATTTCGAAACCACATTTGAGAAGTATTTCCATCCAGTGCTACTTGTGTTT |
| | ACTTCTAAACAGTCATTTTCTAACTGAAGCTGGCATTCATGTCTTCATTTTGGGCT |
| | GTTTCAGTGCAGGGCTTCCTAAAACAGAAGCCAACTGGGTGAATGTAATAAGTG |
| | ATTTGAAAAAAATTGAAGATCTTATTCAATCTATGCATATTGATGCTACTTTATATA |
| | CGGAAAGTGATGTTCACCCCAGTTGCAAAGTAACAGCAATGAAGTGCTTTCTCTT |
| | GGAGTTACAAGTTATTTCACTTGAGTCCGGAGATGCAAGTATTCATGATACAGTA |
| | GAAAATCTGATCATCCTAGCAAACAACAGTTTGTCTTCTAATGGGAATGTAACAG |
| | AATCTGGATGCAAAGAATGTGAGGAACTGGAGGAAAAAAATATTAAAGAATTTTT |
| | GCAGAGTTTTGTACATATTGTCCAAATGTTCATCAACACTTCTTGA |
| IL-15RA | MAPRRARGCRTLGLPALLLLLLLRPPATRGITCPPPMSVEHADIWVKSYSLYSRERYI |
| | CNSGFKRKAGTSSLTECVLNKATNVAHWTTPSLKCIRDPALVHQRPAPPSTVTTAGV |
| | TPQPESLSPSGKEPAASSPSSNNTAATTAAIVPGSQLMPSKSPSTGTTEISSHESSH |
| | GTPSQTTAKNWELTASASHQPPGVYPQGHSDTTVAISTSTVLLCGLSAVSLLACYLK |
| | SRQTPPLASVEMEAMEALPVTWGTSSRDEDLENCSHHL |
| IL-15RA | ATGGCCCCGCGGCGGGCGCGCGGCTGCCGGACCCTCGGTCTCCCGGCGCTGC |
| | TACTGCTGCTGCTGCTCCGGCCGCCGGCGACGCGGGGCATCACGTGCCCTCCC |
| | CCCATGTCCGTGGAACACGCAGACATCTGGGTCAAGAGCTACAGCTTGTACTCC |
| | AGGGAGCGGTACATTTGTAACTCTGGTTTCAAGCGTAAAGCCGGCACGTCCAGC |
| | CTGACGGAGTGCGTGTTGAACAAGGCCACGAATGTCGCCCACTGGACAACCCC |
| | CAGTCTCAAATGCATTAGAGACCCTGCCCTGGTTCACCAAAGGCCAGCGCCACC |
| | CTCCACAGTAACGACGGCAGGGGTGACCCCACAGCCAGAGAGCCTCTCCCCTT |
| | CTGGAAAAGAGCCCGCAGCTTCATCTCCCAGCTCAAACAACACAGCGGCCACAA |
| | CAGCAGCTATTGTCCCGGGCTCCCAGCTGATGCCTTCAAAATCACCTTCCACAG |
| | GAACCACAGAGATAAGCAGTCATGAGTCCTCCCACGGCACCCCCTCTCAGACAA |
| | CAGCCAAGAACTGGGAACTCACAGCATCCGCCTCCCACCAGCCGCCAGGTGTG |
| | TATCCACAGGGCCACAGCGACACCACTGTGGCTATCTCCACGTCCACTGTCCTG |
| | CTGTGTGGGCTGAGCGCTGTGTCTCTCCTGGCATGCTACCTCAAGTCAAGGCAA |
| | ACTCCCCCGCTGGCCAGCGTTGAAATGGAAGCCATGGAGGCTCTGCCGGTGAC |
| | TTGGGGGACCAGCAGCAGAGATGAAGACTTGGAAAACTGCTCTCACCACCTATG |
| | A |

### Example 9. IL-15 Expression in Naïve Mice with LPMP/mRNA Formulation

This example describes a recLemon LPMP/mRNA formulation comprising a lemon lipid reconstructed with ionizable lipids (C12-200), sterols (cholesterol), and PEG lipids (DMPE-PEG2k), to encapsulate mRNA (e.g., mRNA for IL-15). The recLemon LPMP/mRNA formulation tested in this example was prepared according to Example 2. The coding portion of the IL-15 mRNA used in this example is as follows:

MRISKPHLRS ISIQCYLCLL LNSHFLTEAG IHVFILGCFS AGLPKTEANW VNVISDLKKI
EDLIQSMHID ATLYTESDVH PSCKVTAMKC FLLELQVISL ESGDASIHDT VENLIILANN
SLSSNGNVTE SGCKECEELE EKNIKEFLQS FVHIVQMFIN TS
or
ATGAGAATTTCGAAACCACATTTGAGAAGTATTTCCATCCAGTGCTACTTGTGTTTACTTC
TAAACAGTCATTTTCTAACTGAAGCTGGCATTCATGTCTTCATTTTGGGCTGTTTCAGTGC
AGGGCTTCCTAAAACAGAAGCCAACTGGGTGAATGTAATAAGTGATTTGAAAAAAATTGA
AGATCTTATTCAATCTATGCATATTGATGCTACTTTATATACGGAAAGTGATGTTCACCCC
AGTTGCAAAGTAACAGCAATGAAGTGCTTTCTCTTGGAGTTACAAGTTATTTCACTTGAGT
CCGGAGATGCAAGTATTCATGATACAGTAGAAAATCTGATCATCCTAGCAAACAACAGTT
TGTCTTCTAATGGGAATGTAACAGAATCTGGATGCAAAGAATGTGAGGAACTGGAGGAA
AAAAATATTAAAGAATTTTTGCAGAGTTTTGTACATATTGTCCAAATGTTCATCAACACTTC
TTGA

In this example, in vivo level of IL-15 and its effect on systemic cytokines upon intramuscular dosing of recLemon LPMP/mRNA formulation (10 μg of IL-15 mRNA) were measured; the effects of intramuscular delivery of recLemon LPMP/mRNA formulation (10 μg IL-15 mRNA) on T cell and NK cell in vivo proliferation and intracellular cytokine production (#CD8 T cells, CD4 T cells, and NK cells at 6 and 10 days post intramuscular dose) were assessed. The studies were done in BotaMouse_212. The treatment schedule is shown in the chart below and illustrated in Scheme 3.

| Group | Treatment | Number of Animals | Dose Conc. |
|---|---|---|---|
| A | IL-15 mRNA | 6 | 10 ug/20 ul; |
| B | IL-15 protein | 6 | IM (0.4 mg/kg) |
| C | Buffer | 6 | |

This example demonstrated that a single dose of a recLemon LPMP/mRNA formulation (IL-15) in mice was able to express IL-15 in vivo and reach high systemic levels up to 72 hours post dosing; was able to induce the release of T cell cytokine IFNγ, highlighting its role in potentiating the immune response; was able to induce the production of pro-inflammatory cytokines and chemokines in serum; and increased the absolute numbers of both CD4+ and CD8+ T cells as well as NK cells in the spleen. A single intramuscular dose of recLemon LPMP/mRNA formulation (IL-15) in mice was able to increase IL-2Ra expression and IFNγ production from NK cells in the spleen 10 days post-dose.

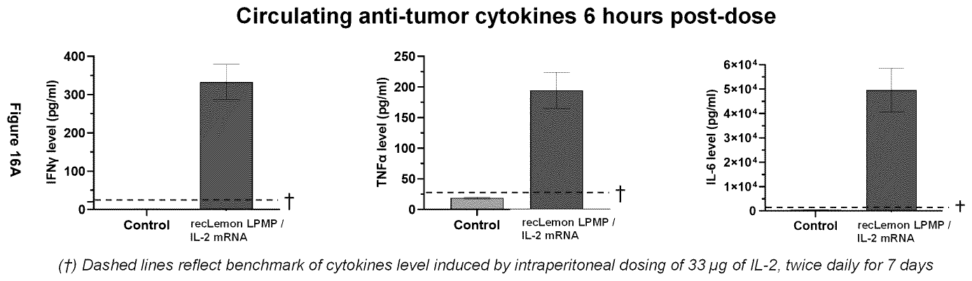

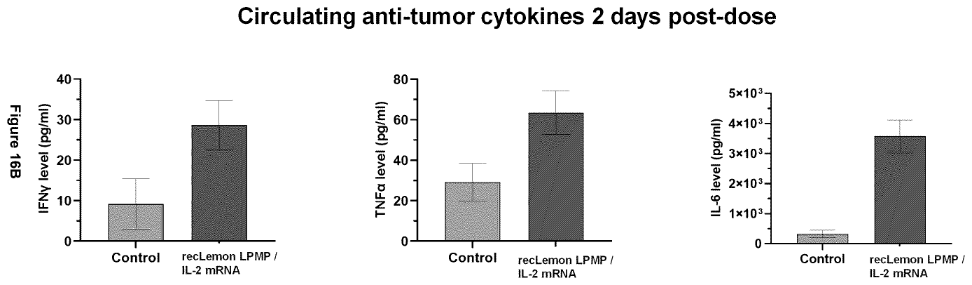

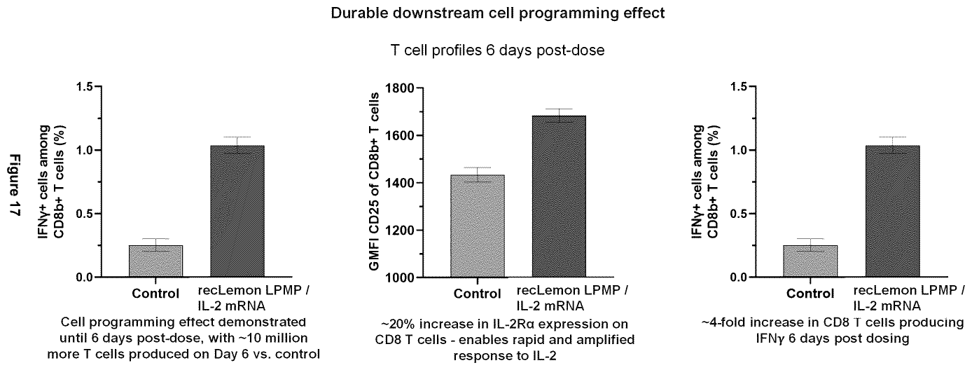

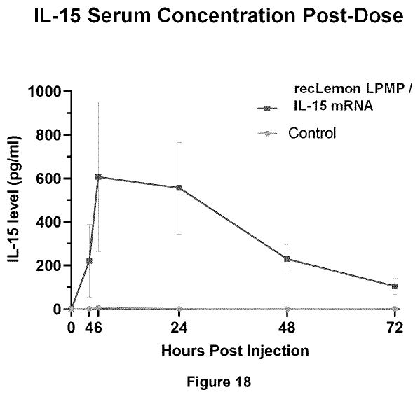

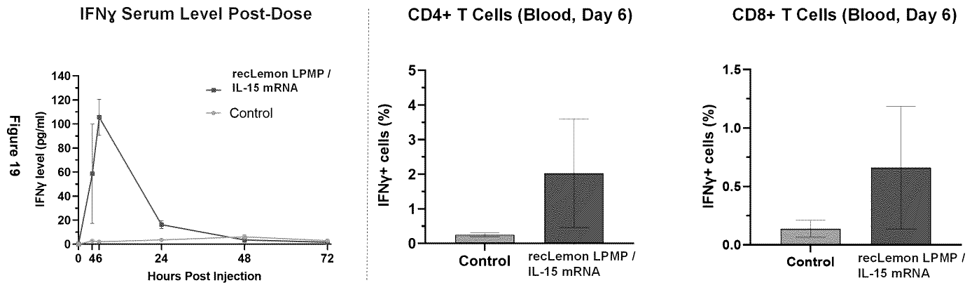

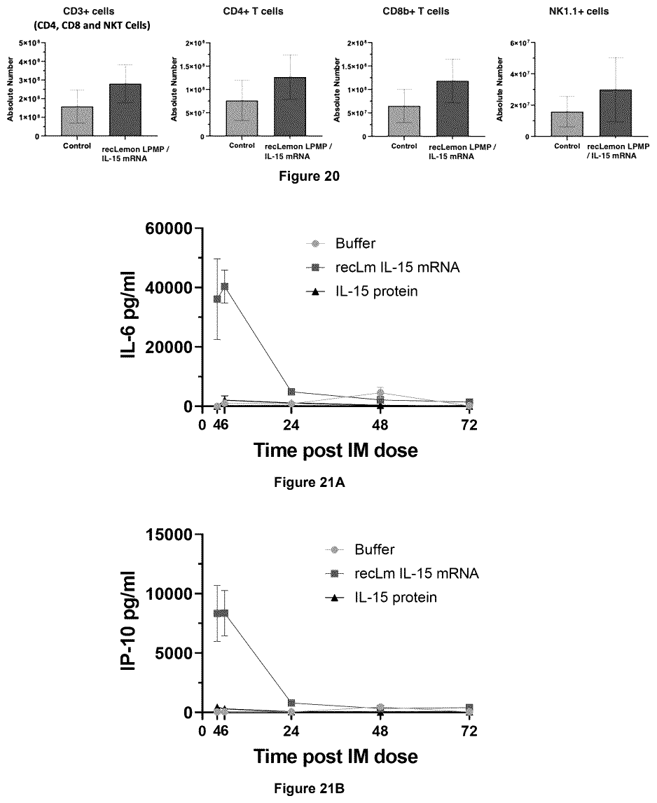

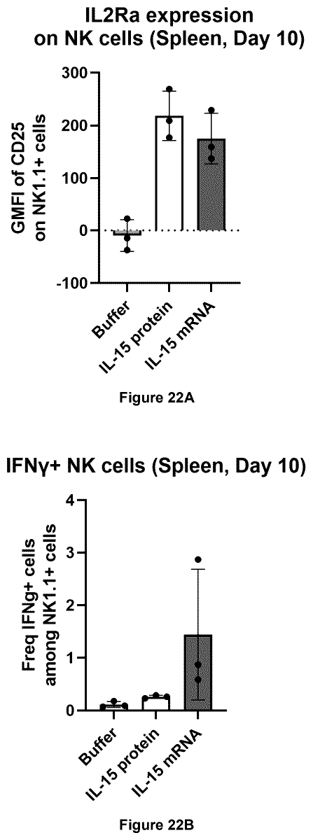

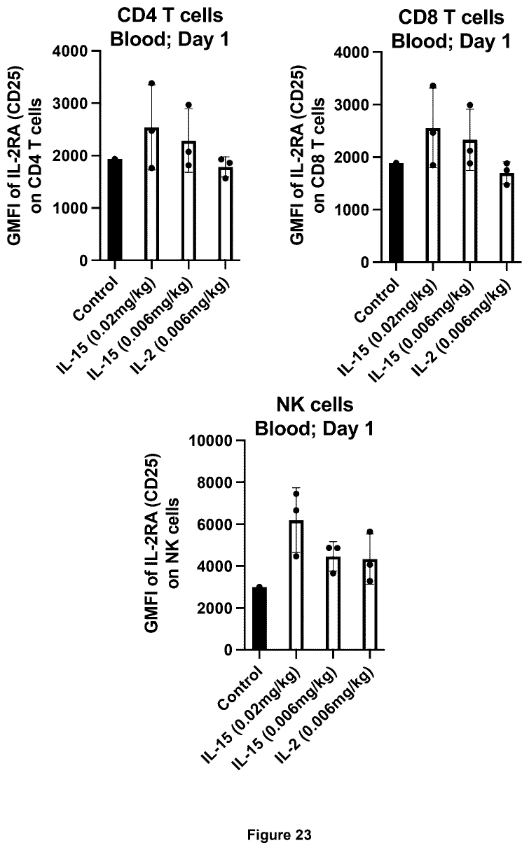

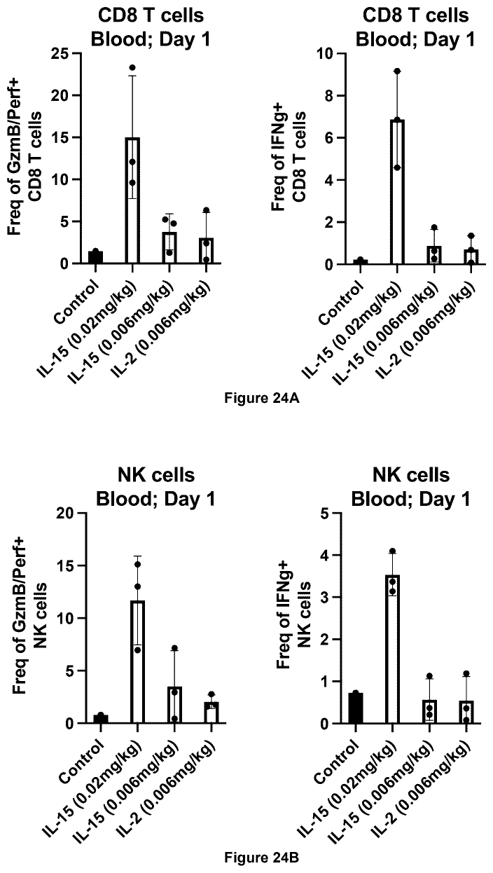

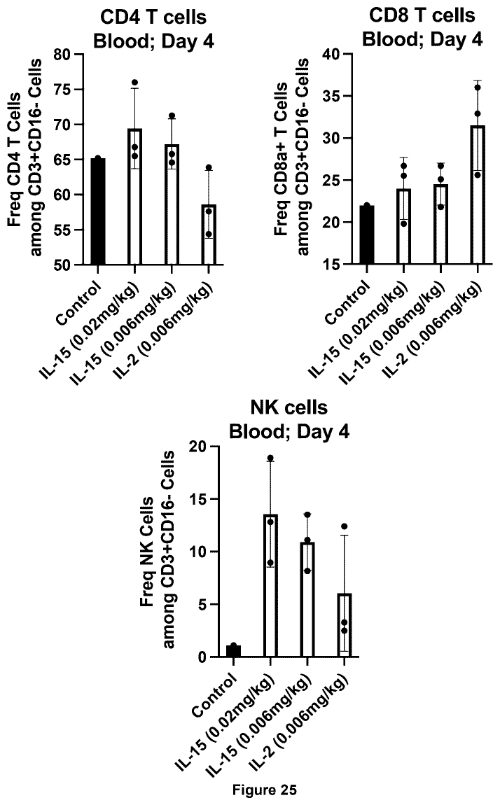

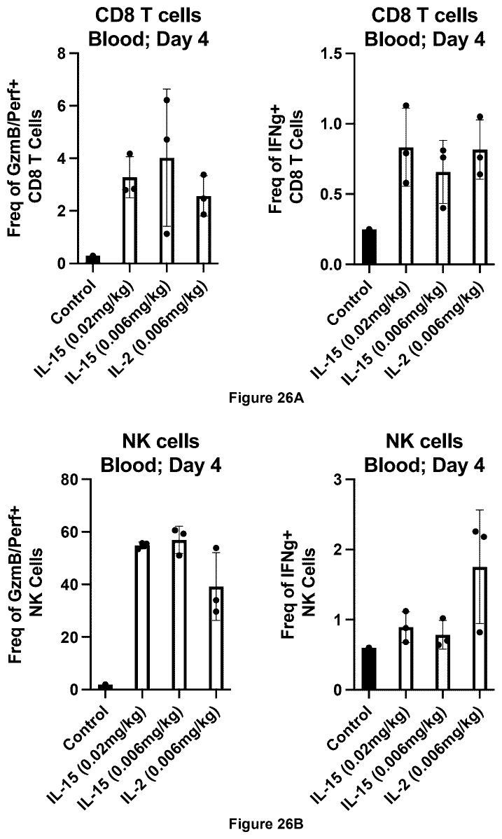

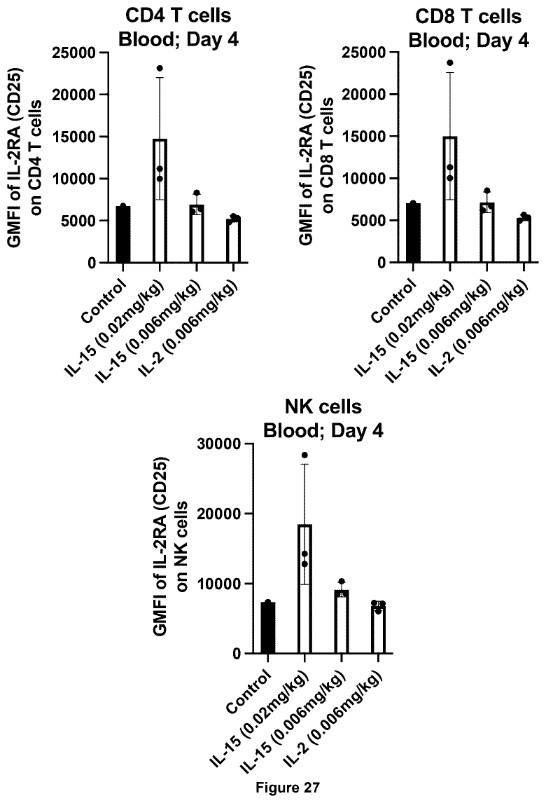

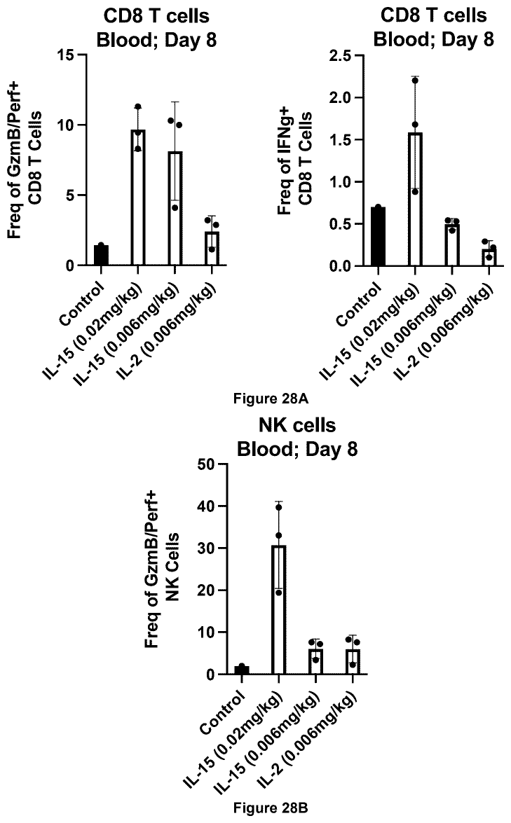

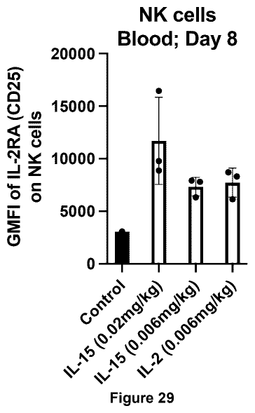

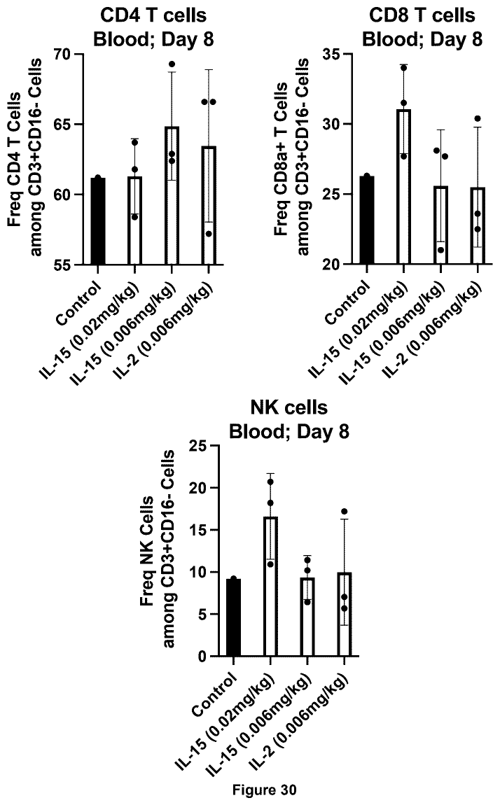

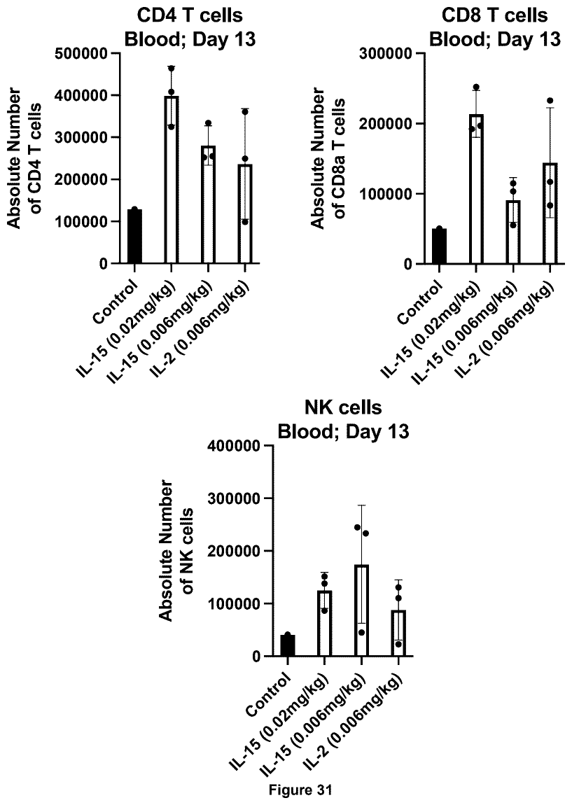

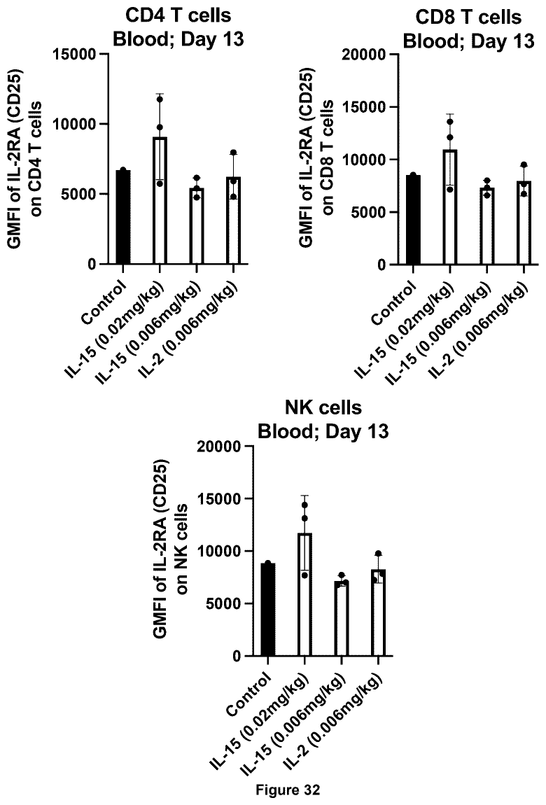

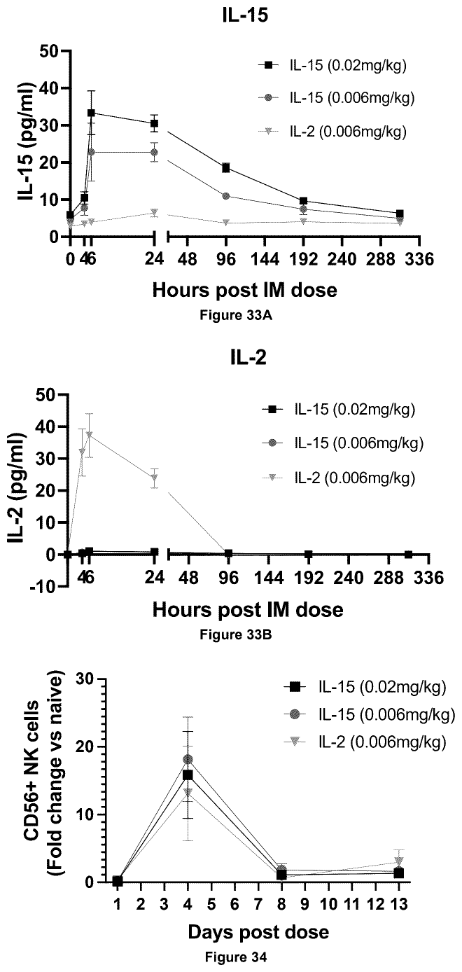

These conclusions were illustrated in FIGS. 18-22.

### Example 10. IL-15 and IL-2 Expression in Naïve NHPs with LPMP/mRNA Formulation

This example describes a recLemon LPMP/mRNA formulation comprising a lemon lipid reconstructed with ionizable lipids (C12-200), sterols (cholesterol), and PEG lipids (DMPE-PEG2k), to encapsulate mRNA (e.g., mRNA for IL-15 or IL-2). The recLemon LPMP/mRNA formulation tested in this example was prepared according to Example 2. The coding portion of the IL-2 mRNA used in this example is as follows:

MYRMQLLSCI ALSLALVTNS APTSSSTKKT QLQLEHLLLD
LQMILNGINN YKNPKLTRML TFKFYMPKKA TELKHLQCLE
EELKPLEEVL NLAQSKNFHL RPRDLISNIN VIVLELKGSE
TTFMCEYADE TATIVEFLNR WITFCQSIIS TLT

The coding portion of the IL-15 mRNA used in this example is as follows:

MRISKPHLRS ISIQCYLCLL LNSHFLTEAG IHVFILGCFS
AGLPKTEANW VNVISDLKKI EDLIQSMHID ATLYTESDVH
PSCKVTAMKC FLLELQVISL ESGDASIHDT VENLIILANN
SLSSNGNVTE SGCKECEELE EKNIKEFLQS FVHIVQMFIN TS

In this example, the effects of intramuscular dosing of recLemon LPMP/mRNA formulation (IL-15 mRNA or IL-2 mRNA) were measured. The studies were done in non-human primates (NHPs). The treatment schedule is shown in the chart below and illustrated in Scheme 4.

| Group | Treatment | Particle | Dose and RoA |
|---|---|---|---|
| 1 (N = 3) | IL-15 mRNA | Lm; C12-200 | IM; 100 ug (0.02 mg/kg) |
| 2 (N = 3) | IL-15 mRNA | Lm; C12-200 | IM; 30 ug (0.006 mg/kg) |
| 3 (N = 3) | IL-2 mRNA | Lm; C12-200 | IM; 30 ug (0.006 mg/kg) |

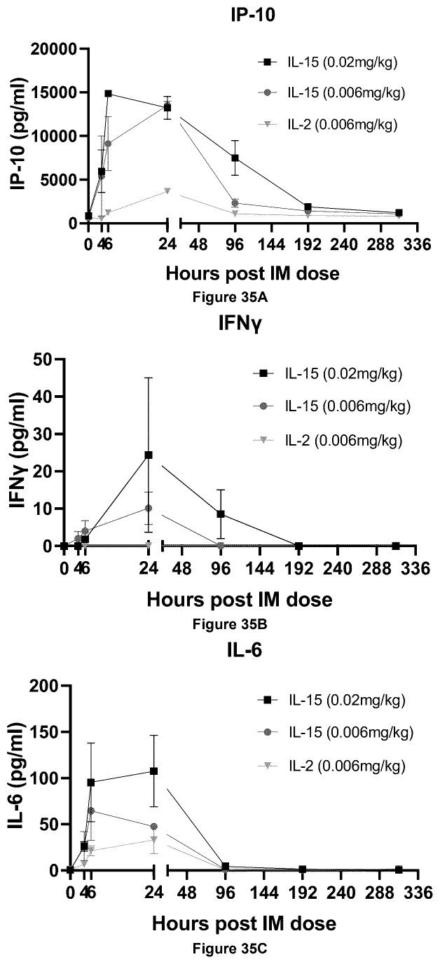

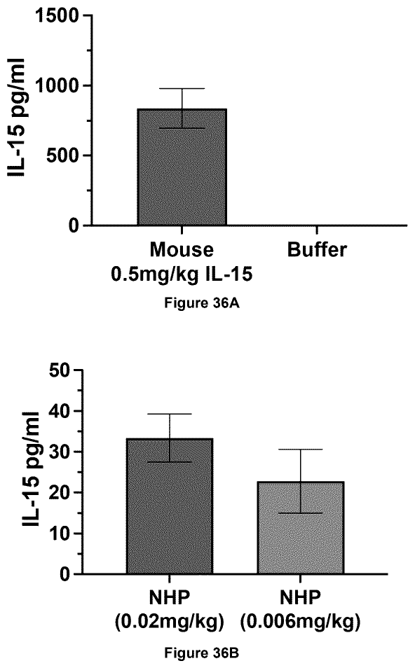

This example demonstrated that a single intramuscular dose of a recLemon LPMP/mRNA formulation (IL-15) in NHPs results in elevated levels of IL-2Ra (CD25) on T cells and NK cells and a higher frequency of CD8 T cells and NK cells producing Granzyme B/Perforin and IFNγ in blood 24 hours post-dose (Day 1). After 4 days post-dose, a single intramuscular dose of a recLemon LPMP/mRNA formulation (IL-15 and IL-2) increased proliferation of T cells and NK cells, as well as elevated levels of Granzyme B and IFNγ in these cells, in the blood. After 4 days post-dose, a single intramuscular dose of a recLemon LPMP/mRNA formulation (IL-15) increased IL-2Ra (CD25) on T cells and NK cells in the blood. After 8 days post-dose, a single intramuscular dose of a recLemon LPMP/mRNA formulation (IL-15 and IL-2) results in an increased frequency of circulating CD8 T cells and NK cells producing Granzyme B/perforin and increased expression of IL-2Ra (CD25) on NK cells in the blood. An increased frequency of IFNγ in CD8 T cells and a higher frequency of CD8 T cells and NK cells in the blood 8 days post-dose was found in response to recLemon LPMP/IL-15. After 13 days post-dose, an increased number of circulating T cells and NK cells was detected in response to a single intramuscular dose of a recLemon LPMP/mRNA formulation (IL-15 and IL-2), indicating long-lasting proliferation of immune cells. In response to a single intramuscular dose of a recLemon LPMP/mRNA formulation (IL-15), increased expression of IL-2Ra (CD25) on T cells and NK cells was found in the blood 13 days post-dose, indicating long-lasting activation of immune cells. Cytokine levels of IL-15 and IL-2 measured at 4 hours, 6 hours, 24 hours, 4 days (96 hours), 8 days (192 hours), and 13 days (312 hours) indicate that a single intramuscular dose of a recLemon LPMP/mRNA formulation (IL-15 and IL-2) increase systemic expression of their respective immune cytokines, with IL-15 mRNA producing long-lasting increased expression of IL-15 for multiple days (FIG. 33A). The expansion of CD56+ NK cells over 13 days after a single intramuscular dose of recLemon LPMP/mRNA formulation (IL-15 and IL-2) indicates a peak at 4 days post-dose in the blood of NHPs. Systemic levels of pro-inflammatory cytokines (IP-10, IFNγ, IL-6) measured at 4 hours, 6 hours, 24 hours, 4 days (96 hours), 8 days (192 hours), and 13 days (312 hours) reveal that a single intramuscular dose of a recLemon LPMP/mRNA formulation (IL-15 and IL-2) increased levels indicative of an immune response but not to expression levels indicative of toxicity (FIG. 35). Systemic levels of IL-15 production measured at 6 hours post-dose show that the expected levels based on dosage in a murine model (FIG. 36A) correspond to similar levels based on dose in a non-human primate model, indicating consistency across multiple dosages and animal models.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference. Other embodiments are within the claims.

1. An mRNA therapeutic composition, comprising:
one or more polynucleotides encoding one or more tumor antigenic or signaling polypeptides,
formulated within a lipid reconstructed plant messenger packs (LPMPs) comprising natural lipids and an ionizable lipid, wherein the ionizable lipid has two or more of the characteristics listed below:
(i) at least 2 ionizable amines;
(ii) at least 3 lipid tails; wherein each of the lipid tails is at least 6 carbon atoms in length;
(iii) a pKa of about 4.5 to about 7.5;
(iv) an ionizable amine and a heteroorganic group separated by a chain of at least two atoms; and
(v) an N:P ratio of at least 10.

2. The mRNA therapeutic composition of claim 1, wherein the natural lipids are extracted from lemon or algae.

3. The mRNA therapeutic composition of claim 1, wherein the LPMPs further comprise a sterol and a polyethylene glycol (PEG)-lipid conjugate.

4. The mRNA therapeutic composition of claim 3, wherein the LPMPs comprise ionizable lipid:natural lipids:sterol:PEG-lipid at a molar ratio of about 35:50:12.5:2.5 or about 35:20:42.5:2.5.

5. (canceled)

6. The mRNA therapeutic composition of claim 1, wherein the ionizable lipid is selected from the group consisting of 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), MD1 (cKK-E12), OF2, EPC, ZA3-Ep10, TT3, LP01, 5A2-SC8, Lipid 5, SM-102 (Lipid H), and ALC-315.

7-10. (canceled)

11. The mRNA therapeutic composition of claim 1, wherein the polypeptide is IL-2 peptide, IL-2-R^{a}, IL-15 peptide, IL-15 R^{a}, Anti-CD19, CD20, CAR-T, Anti-HER2, Etanercept (Enbrel), Humira, erythropoietin, Epogen, Filgrastim, Keytruda, Rituximab, Romiplostim, Sargramostim, or a fragment or subunit thereof.

12. The mRNA therapeutic composition of claim 1, wherein the polynucleotide is an mRNA which encodes an IL-2 molecule comprising an amino acid sequence having at least 85%, 90%, 95%, 96%, 97%, 98%, 99% or 100% identity to the amino acid sequence of an IL-2 molecule provided in any one of Tables I-III.

13-32. (canceled)

33. The mRNA therapeutic composition of claim 1, wherein the LPMP has a size of less than about 200 nm.

34-38. (canceled)

39. The mRNA therapeutic composition of claim 1, wherein the LPMP comprises:
about 20 mol % to about 50 mol % of the ionizable lipid,
about 20 mol % to about 60 mol % of the natural lipids,
about 7 mol % to about 20 mol % of the sterol, and
about 0.5 mol % to about 3 mol % of the polyethylene glycol (PEG)-lipid conjugate.

40. (canceled)

41. The mRNA therapeutic composition of claim 1, wherein the mRNA therapeutic composition has a total lipid:polynucleotide weight ratio of about 50:1 to about 10:1.

42-45. (canceled)

46. The mRNA therapeutic composition of claim 1, further comprising a HEPES or TRIS buffer at a pH of about 7.0 to about 8.5.

47-48. (canceled)

49. The mRNA therapeutic composition of claim 1, further comprising one or more cryoprotectants.

50-51. (canceled)

52. The mRNA therapeutic composition of claim 1, wherein the mRNA therapeutic is a lyophilized composition comprising one or more lyoprotectants.

53-65. (canceled)

66. A method of delivering an mRNA therapeutic in a subject, comprising:
administering to the subject the mRNA therapeutic composition of claim 1.

67. A method of inducing an immune response in a subject, comprising:
administering to the subject the mRNA therapeutic composition of claim 1.

68. A method of treating or preventing a cancer in a subject, comprising:
administering to the subject the mRNA therapeutic composition of claim 1.

69. The method of claim 66, wherein the mRNA therapeutic composition is administered by oral, intravenous, intradermal, intramuscular, intranasal, intraocular, rectal, intrajejunal, intratumoral, and/or subcutaneous administration.

70. (canceled)

71. The method of claim 66, wherein the mRNA therapeutic composition is administered at a dosage level sufficient to deliver about 0.006 mg/kg to about 0.5 mg/kg of the polynucleotides to the subject.

72. The method of claim 67, wherein the mRNA therapeutic composition is administered to the subject one time, two times, three times, four times, or more.

73. (canceled)

74. The method of claim 66, further comprising:
administering an additional therapeutic agent to the subject, wherein the additional therapeutic agent is an anti-cancer therapeutic agent or a therapeutic agent that treats and/or prevents a chronic pain.

75-81. (canceled)

82. The method of claim 66, wherein the mRNA therapeutic composition is administered at a dosage level sufficient to deliver about 0.0003 mg/kg to about 0.002 mg/kg of the polynucleotides to the subject.

83. (canceled)

84. The mRNA therapeutic composition of claim 1, wherein the polynucleotide is an mRNA which encodes an IL-15 and/or IL-15R^{a} molecule comprising an amino acid sequence having at least 85%, 90%, 95%, 96%, 97%, 98%, 99% or 100% identity to the amino acid sequence of an IL-15 molecule provided in any one of Table 9 or Examples 1-9.

85-87. (canceled)

88. The method of claim 66, wherein the mRNA therapeutic composition modulates proliferation or activation of T cells and/or NK cells.

89. he method of claim 88, wherein the mRNA therapeutic composition modulates proliferation or activation of CD4 Tcells and/or CD8 T cells.

90. The method of claim 88, wherein the mRNA therapeutic composition provides modulations in the blood, in the lymph nodes, in the spleen or in any combination thereof.

91-92. (canceled)

93. The method of claim 66, wherein the mRNA therapeutic composition modulates activity or expression of Granzyme B/perforin, CD25 (IL-2R^{a}), TNF-alpha, IL-15, IL-6, IL-8, IL-12, GM-CSF, G-CSF, IL-1, IL-2, IL-4, IL-5, IL-8, IL-9, IL-10, IL-13, IL-17, IL-33, PD-L1, CCL2, CCL3, CCL4, CXCL1, CXCL2, CXCL10 (IP-10), CCL20, CD40, TNF-β, LAF, TCGF, BCGF, TRF, BAF, BDG, MP, LIF, OSM, TMF, PDGF, IFN-a, IFN-β, IFN-γ or any combination thereof.

94-95. (canceled)
